(19) United States
(12) Patent Application Publication
(10) Pub. No.: US 2022/0144747 A1
(43) Pub. Date: May 12, 2022

(54) SHIKIMATE ANALOGUES AND METHODS OF USE

(71) Applicant: The Board of Regents of the University of Oklahoma, Norman, OK (US)

(72) Inventors: Lin Du, Norman, OK (US); Robert Cichewicz, Norman, OK (US); Kenneth M. Nicholas, Norman, OK (US); Jianlan You, New York City, NY (US)

(21) Appl. No.: 17/534,888

(22) Filed: Nov. 24, 2021

Related U.S. Application Data
(63) Continuation-in-part of application No. 16/592,340, filed on Oct. 3, 2019, now abandoned.
Application No. 16/592,340 is a continuation-in-part of application No. 16/077,389, filed on Aug. 10, 2018.
(60) Provisional application No. 62/295,343, filed on Feb. 15, 2016.

(51) Int. Cl. C07C 67/327; C07D 303/04

(52) U.S. Cl. CPC C07C 67/327; C07D 303/04

(57) ABSTRACT

The present disclosure, in at least certain embodiments, is directed to shikimate (shikimic acid) analogues and compositions thereof, devices and kits which contain the shikimate analogues or compositions thereof, and methods of use of the compounds and compositions for treating and neutralizing irritant or odoriferous compounds on animate or inanimate surfaces or in atmospheres.

### CROSS-REFERENCE TO RELATED APPLICATIONS

This application is a continuation-in-part of U.S. Ser. No. 16/592,340, filed Oct. 3, 2019, which is a continuation-in-part of U.S. Ser. No. 16/077,389, filed Aug. 10, 2018, which is a U.S. national stage application of International application PCT/US2017/017790, filed Feb. 14, 2017, which claims priority to U.S. Provisional Application No. 62/295,343, filed on Feb. 15, 2016, each of which is expressly incorporated herein by reference in its entirety.

### STATEMENT REGARDING FEDERALLY SPONSORED RESEARCH OR DEVELOPMENT

This invention was made with government support under Grant Number AI085161 awarded by the National Institutes of Health (NIH). The government has certain rights in the invention.

### BACKGROUND

Pets and people in rural and natural landscapes frequently encounter offensive, malodorous compounds such as skunk mercaptans, and dermal irritants such as urushiol produced by poison ivy, poison, sumac, poison oak and similar species. Urushiol is actually a mixture of phenolic compounds that are known as catechols, which are potent benzene ring compounds having a long side-chain of 15 or 17 carbon atoms. The side chain may be saturated or unsaturated with one, two, or three double bonds. The immune reaction and specificity of the catechol molecule may be determined by the long side-chains. Poison oak urushiol contains mostly catechols with 17 carbon side-chains (heptadecylcatechols), while poison ivy and poison sumac contain mostly 15 carbon side-chains (pentadecylcatechols). Existing approaches to handling these offensive agents include their removal (washing) or neutralization (change in chemical structure) based on a variety of acids, bases, oxidizing agents, soaps and other cleaners, sequestering compounds, and detergents. These approaches suffer from many shortcomings that include limited efficacy, the need for repeated applications, and incompatibilities with certain surfaces. These incompatibilities stem from the frequent need to remove the offensive compounds from clothing, skin, and pets (both hair and skin). It would be desirable to have safe and effective compositions that fills the important need for the neutralization of the above-mentioned malodorous and topically-irritating natural products on human skin or hair, or pet skin or hair, clothing, or furniture, or other surfaces. It is to this need that the compositions of the present disclosure are directed.

### BRIEF DESCRIPTION OF THE DRAWINGS

Several embodiments of the present disclosure are hereby illustrated in the appended drawings. It is to be noted however, that the appended drawings only illustrate several typical embodiments and are therefore not intended to be considered limiting of the scope of the present disclosure.

FIG. 1. Shows structures for maximiscin (1) and its rearrangement artifact, isomaximiscin (2). ¹³C isotope incorporation (accomplished by feeding [U-¹³C₆]-D-glucose to the fungus and analyzing the resulting ¹J_{C-C} coupling values) was used to monitor the origin of the C-2′ position in 1 and the corresponding C-2 position for the key chemical degradation product, pericosine C (3).

FIG. 2 shows importance of experimental and calculated electron capture dissociation (ECD) spectra (time-dependent density functional theory—TD-DFT) for resolving the absolute configurations of 1 and 2. The illustrated ¹³C labeling and ECD methods developed during the structure revision process for 1 were useful for understanding the structures and mechanisms of formation for the compounds described herein. Details of the rationale applied to the structure revision of 1, along with the experimental methods created for this purpose are provided below and in the appendix of U.S. Provisional Patent Application 62/295,343.

FIG. 3 shows proposed MSⁿ splitting patterns for shikimate adducts (A and B). (C) and (D) show ultra performance liquid chromatography electrospray ionization mass spectrometry (UPLC-ESIMSⁿ) profiling of selected secondary metabolites produced by Tolypocladium sp. T1. Selected ion traces for m/z 450.2 are shown in C. The corresponding MSⁿ spectra are shown in (D). *MS signals of unknown substances.

FIG. 4 shows UPLC-ESIMSⁿ profiling of selected secondary metabolites produced by coculture of Tolypocladium sp. T1 with Penicillium sp. P1 (E and F). Selected ion traces for m/z 464.2 and 507.2 are shown in (E). The corresponding MSⁿ spectra are shown in F. *MS signals of unknown substances.

FIG. 5 shows UPLC-ESIMSⁿ profiling of selected secondary metabolites produced by coculture of Tolypocladium sp. T1 with Penicillium sp. P2 (G and H). Selected ion traces for m/z 507.2 are shown in (G). The corresponding MSⁿ spectra are shown in (H), which shows the MSⁿ spectrum of 6, which is identical to that of 7. *MS signals of unknown substances.

FIG. 6 shows structures for substrate compounds 8, 9, 13, 15, 17, and 19.

FIG. 7 shows structures of substrate transformation products 4, 5, and 6 obtained from Tolypocladium sp. T1. The ¹³C-labeling patterns for the substrate-shikimate substitution products were generated by feeding T1 with [U-¹³C₆]-D-glucose.

FIG. 8 shows structures of substrate transformation product 7, an enantiomeric mixture obtained from Tolypocladium sp. T1. The ¹³C-labeling patterns for the substrate-shikimate substitution products were generated by feeding T1 with [U-¹³C₆]-D-glucose.

FIG. 9 shows structures of substrate transformation products 14, 16, and 18 obtained from Tolypocladium sp. T1. The ¹³C-labeling patterns for the substrate-shikimate substitution products were generated by feeding T1 with [U-¹³C₆]-D-glucose.

FIG. 10 shows structures of substrate transformation products 20, 21 (an enantiomeric mixture), and 22 obtained from Tolypocladium sp. T1. The ¹³C-labeling patterns for the substrate-shikimate substitution products were generated by feeding T1 with [U-¹³C₆]-D-glucose.

FIG. 11 shows a scheme for chemoassay-guided identification of shikimate analogues 10 and 11. The culture broth of T1 was subjected separately to dialysis and partitioning and treated with the P1-derived metabolite 8. Laser ablation electrospray ionization mass spectrometry (LAESIMS) was used to track the presence of 10 and 11 by monitoring the formation of 4 (m/z 464 Da).

FIG. 12 shows electrophilic natural products from fungus T1. (A)¹³C-labeling patterns for 10 and 11 generated by feeding T1 [U-¹³C₆]-D-glucose with NaCl present in the culture medium. (B) Selectivity and yields for the S_{N}2′ coupling of 12 with 10 and 11. The reaction rates of selected model substrates (13 and 17) were tested and the results provided in Figure S127 of the appendix of U.S. Provisional Patent Application 62/295,343.

FIG. 13 shows calculated transition states and relative free energies of activation for the reactions of 10 and 11 with 1-hydroxy-3-methyl-2-oxo-1,2-dihydropyridin-4-olate (23). The ^{a}B3LYP/6-31G(d) method was used to determine transition state (TS) structures; free energies were obtained from single point calculations by M06-2X and MP2 (in parenthesis) methods and 6-3 11++G(d,p) basis set.

FIG. 14 shows a comparison of the antifungal efficacy (minimum inhibitory concentration—MIC) of the shikimate-substrate substitution products 1, 4, 6, 7, 14, and 18, and their corresponding parent compounds 8, 9, 12, 13, and 17, respectively against a panel of test fungi (T1-T4, P1-P4, and A1).

FIG. 15 shows in graph A that co-treatment of several fungi by compounds 11 plus 13 (fungi T1, T2, P1, P4, and A1) reduced the antifungal efficacy of 13. Graph B shows that supplemental NaNO₃ in test medium enhanced the antifungal efficacy of 8 and 13 against T1. Media types: PDB (potato dextrose broth); PDB-N(PDB plus 2 g/L NaNO₃); PDB 8:2 (mixture of centrifuged broth of T1 grown in PDB for 5 days with fresh PDB at the ratio of 8:2); PDB-N 8:2 (mixture of centrifuged broth of T1 grown in PDB-N for 5 days with fresh PDB at the ratio of 8:2).

FIG. 16 shows an example of a dual (two-container) applicator system for delivery of a shikimate analogue of the present disclosure (disposed in a first container of the applicator) and a secondary compound (disposed in a second container of the applicator) to be combined with the shikimate analogue.

FIG. 17 shows time course toxicity results in EpiDerm™ reconstructed human epidermis tissues. All substances are safe for greater than 24 hours, at even a very high dose of 500 μM. 1% Triton X-100 is included as a control and results in epidermal death after 24 hours. Viability of EpiDerm™ tissues was determined by MTT assay.

FIG. 18 shows IL-18 cytokine response in the media of EpiDerm™ tissues after exposure to test compounds. IL-18 secretion was not elevated in response to the test compounds, indicating a low potential for allergic reactions. Cytokine levels were determined by enzyme-linked immunosorbent assay (ELISA).

FIG. 19 shows IL-1 alpha cytokine response in the media of EpiDerm™ tissues after exposure to test compounds. IL-1 alpha secretion was not elevated in response to the test compounds, indicating a low potential for allergic reactions. The only significant response is in the 1% Triton X-100 control. Cytokine levels were determined by ELISA.

FIG. 20 shows time course toxicity results in EpiDerm™ reconstructed human epidermis tissues using test compounds plus activating compound and fragrance. All substances are safe for greater than 24 hours, at even a very high dose of 500 μM. 1% Triton X-100 is included as a control and results in epidermal death after 24 hours. Viability of EpiDerm™ tissues was determined by MTT assay.

FIG. 21 shows that Pericosine A can completely neutralize a thiol on a surface (pig skin).

FIG. 22 shows the synthesis of several neutral pericosine analogues.

FIG. 23 shows the synthesis of several ammonium and phosphonium pericosine analogues.

FIG. 24 shows the synthesis of several ammonium and phosphonium pericosine analogues.

### DETAILED DESCRIPTION

The present disclosure is directed to compounds and compositions, kits, devices, and methods of using the compounds for deodorization and treating irritants. For example, the disclosure relates to deodorizing animate (living) and inanimate (non-living) surfaces and objects, and ambient air and atmospheres, and particularly to deodorizing compounds that are generally recognized as having negative connotations associated with odor, smell, scent, aroma, fragrance, stink, reeking, malodor, stench, or other disagreeable olfactory sensation, or neutralizing compounds that have properties that make them dermal or olfactory irritants. Examples of such irritant and odoriferous compounds include, respectively, urushiol from poison ivy, mercaptans of skunk spray, and amines used in hair treatments.

The present disclosure, in at least certain embodiments, is directed to shikimate (shikimic acid) analogues and compositions thereof, their production (for example, from natural or recombinant fungal sources), derivatization, and activation, and delivery methods for the use in neutralization of olfactory and dermal irritants on surfaces. In certain embodiments, the compositions can be used, for example, for deodorizing and neutralizing low molecular weight thiols generated by skunks and other mammals, as well as toxic agents such as the skin irritant urushiol (responsible for urushiol-induced contact dermatitis) and its related irritant compounds from members of the Anacardiaceae, and for deodorizing offensive chemicals and neutralizing dermal irritants from skin, pets, clothing, fabrics, furniture, and other surfaces described hereinbelow. Further, the present disclosure describes methods for the production and delivery (e.g., via systems, devices, and/or kits or other means) of such shikimate compounds. The present disclosure, in at least certain embodiments, is directed to shikimate analogue compounds and compositions containing shikimate analogues for use in treating living or non-living surfaces before and/or following exposure to irritants, allergens, and toxic agents (for example, urushiols or mercaptans). In certain embodiments, the compositions contain more than one shikimate analogue such as pericosine compounds (e.g., pericosines A-D) such as can be obtained by treating a fungal extract of Tolypocladium sp., e.g., Tolypocladium inflatum Gams, ATCC No. 42437, or other fungal source of pericosine compounds such as, but not limited to, Periconia byssoides Persoon:Schweinitz ATCC No. 22274.

Before further describing various embodiments of the compounds, compositions and methods of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning; and the embodiments and examples are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications and equivalents apparent to those having ordinary skill in the art are included within the scope of the present disclosure. All of the compounds, compositions, and methods of production and application and use thereof disclosed herein can be made and executed without undue experimentation in light of the present disclosure. Thus, while the compounds, compositions, and methods of the present disclosure have been described in terms of particular embodiments, it will be apparent to those of skill in the art that variations may be applied to the compounds, compositions and/or methods and in the steps or in the sequence of steps of the methods described herein without departing from the concept, spirit, and scope of the inventive concepts.

All patents, published patent applications, and non-patent publications mentioned in the specification or referenced in any portion of this application, including but not limited to U.S. Provisional Application No. 62/295,343, U.S. Ser. No. 16/077,389, and PCT/US2017/017790, are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. By way of further example, the range 1 wt % to 99 wt % is intended to include any sub-range therein, although that sub-range may not be explicitly designated herein. For example, since the range 1 wt % to 99 wt % includes all integers from 1 to 99, the sub-ranges therein include any range having a minimum value of 1 wt % to 98 wt % and any maximum value of 2 wt % to 99 wt %, such as but not limited to, 5 wt % to 75 wt %, 10 wt % to 50 wt %, or 15 wt % to 40 wt %.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” and “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, a temporal duration, and the like, is meant to encompass, for example, variations of ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment. The term shikimate analogue may be used interchangeably with shikimate analogue compound. The term “shikimate analogue” may refer to a single type of the shikimate analogue or to more than one type of the shikimate analogue such as may occur in a naturally produced or synthetically-formed mixture.

As used herein, the term tosyl refers to a toluenesulfonyl group, the term brosyl refers to a para-bromophenylsulfonyl group, the term mesyl refers to a methylsylfonyl group, the triphenylphosphine is also known as triphenylphosphane, the term sulfonamide refers to an amide of sulfonic acid, the term sulfonate refers to a salt or ester of sulfonic acid, the term phosphonate refers to a salt or ester of phosphonic acid, the term ammonium refers both to primary and secondary substituted amines and to primary, secondary, and tertiary substituted ammoniums. The primary, secondary, and/or tertiary substituents may be, for example, methyl, ethyl, propyl, iso-propyl, or butyl (n-, iso-, sec-, tert-isomers). For example, in non-limiting embodiments, a tertiary ammonium may be methyldiethyl, methyldipropyl, methyldibutyl, dimethylethyl, dimethylpropyl, dimethylbutyl, trimethyl, triethyl, tripropyl, or tributyl ammonium. For example, in non-limiting embodiments, a secondary ammonium may be dimethyl, diethyl, dipropyl (either isomer), dibutyl (any isomer), methylethyl, methylpropyl (either isomer), methylbutyl (any isomer), ethylpropyl (either isomer), ethylbutyl (any isomer), or propylbutyl (any isomer) ammonium.

An example of an ammonium R group is choline ion. The term choline refers to an R group comprising N,N,N-trimethyl-2-(11-oxidaneyl)ethan-1-aminium. An R group N,N,N-methyldiethyl-2-(11-oxidaneyl)ethan-1-aminium, N,N,N-dimethylethyl-2-(11-oxidaneyl)ethan-1-aminium, N,N-diethyl-2-(11-oxidaneyl)ethan-1-aminium, or N,N,N-triethyl-2-(11-oxidaneyl)ethan-1-aminium may be used in place of the choline R group in any of the structural formulas herein which comprise an ammonium.

The term “pharmaceutically acceptable” refers to compounds and compositions which are suitable for administration to humans and/or animals without undue adverse side effects such as toxicity, irritation and/or allergic response commensurate with a reasonable benefit/risk ratio. The compounds of the present disclosure may be combined with one or more pharmaceutically-acceptable excipients, including carriers, vehicles, and diluents which may improve solubility, deliverability, dispersion, stability, and/or conformational integrity of the compounds or conjugates thereof.

As used herein, “pure,” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 50% (w/w) pure, or at least 55% (w/w) pure, or at least 60% (w/w) pure, or at least 65% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure. Where used herein “% (w,w)” is used interchangeably with “wt %”. Where used herein % purity generally refers to the total % of the one or more shikimate analogues in a mixture or extract.

The terms “subject” and “patient” are used interchangeably herein and will be understood to refer to a warm blooded animal, particularly a mammal. Non-limiting examples of animals within the scope and meaning of this term include dogs, cats, rabbits, rats, mice, guinea pigs, chinchillas, horses, goats, cattle, sheep, zoo animals, Old and New World monkeys, non-human primates, and humans, and any other animal susceptible to a contact dermatitis as described herein.

The terms “deodorizing” or “controlling odoriferous substances” as used herein refers to lessening the intensity, mitigating, reducing, inhibiting, abolishing, modulating, controlling, or removing an odor such that the odor is at least partially or completely eliminated, or the olfactory profile of the odor is altered.

The terms “epithelium” or “epithelial surface” as used herein refer, for example, to the skin, hair, fur, nails, body surfaces, surfaces of body cavities, and surfaces of hollow organs that are connected to underlying tissues in living or deceased humans and other animals and constitute the corpus or partial corpus of said organisms.

The term “non-living surface” as used herein refers to synthetic materials and objects, structures and articles of manufacture, including but not limited to, textiles, fabrics, couches, chairs, beds, floors, rugs, seats, solid and porous materials composed of soils, minerals, stones, rocks, concrete, walls, woods, resins, metals, plastics, and composite materials.

The term “living surface” as used herein refers to surfaces of living organisms including, but not limited to, epithelial surfaces, fur, hair, skin, shrubs, grass, plants, gardens, and lawns.

The terms “air” or “atmosphere” as used herein refer to the gaseous component of a room, building, vehicle, outdoor environment, sealed container, box, bag, or other open or sealed or semipermeable package, container, or vessel. Air or atmosphere may be comprised of gaseous compounds at or near their natural abundance or of a modified or fabricated atmosphere created through the actions of humans, animals, or microorganisms.

The term “condition” refers to any condition caused by exposure of an epithelial surface to an agent which is toxic or otherwise undesirable (e.g., malodorous), the toxicity or undesirability of which is desired to be neutralized, inhibited, diminished, or otherwise treated. For example, in certain non-limiting embodiments, the term “condition” may refer to a contact dermatitis due to exposure to an urushiol compound, or a malodorous condition due to exposure to a mercaptan.

Where used herein, the term “irritant/odoriferous compound” refers to any compound having one or more properties which are irritating, (e.g., to an epithelial, dermal, ocular, or olfactory or other sensory tissue,) or odoriferous, or are both irritating and odoriferous.

“Treatment” refers to treatment of a condition. “Prevention” refers to prophylactic or preventative treatment measures or reducing the onset of the condition. The term “treating” refers to administering the composition to a subject for treatment of the condition. The treatment may be therapeutic, for example in the case wherein the toxicity of the agent can be harmful.

The terms “therapeutic composition” and “pharmaceutical composition” refer to a shikimate analogue-containing composition that may be administered to a subject by any method known in the art or otherwise contemplated herein, wherein administration of the composition brings about treatment of a condition such as is described elsewhere herein. In addition, the compositions of the present disclosure, which may contain one or more secondary compounds, may be designed to provide delayed, controlled, extended, and/or sustained release using formulation techniques which are well known in the art.

The term “effective amount” refers to an amount of a shikimate analogue which is sufficient to exhibit a detectable anti-toxic, anti-malodorous, or therapeutic effect against a condition (e.g., contact dermatitis) in a subject without excessive adverse side effects (such as substantial toxicity, irritation and allergic response) commensurate with a reasonable benefit/risk ratio when used in the manner of the present disclosure. The effective amount for a subject will depend upon the subject's type, size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

More particularly, an effective amount of a shikimate analogue of the present disclosure refers to an amount which is effective in controlling, reducing, or inhibiting a condition as described herein, such as contact dermatitis. The term “controlling” is intended to refer to all processes wherein there may be a slowing, interrupting, arresting, or stopping of the progression of the condition and does not necessarily indicate a total elimination of the symptoms of the condition. In at least one embodiment the shikimate analogue is effective in controlling, reducing, or inhibiting the effects of a condition, such as a contact dermatitis due to exposure to an urushiol or a malodorus condition due to exposure to a mercaptan.

The term “effective amount” is further meant to define an amount resulting in the improvement of any parameters or clinical symptoms characteristic of a condition. The actual dose will vary with the patient's overall condition, the seriousness of the condition or symptoms, and counter indications. As used herein, the term “effective amount” also means the total amount of each active component of the pharmaceutical composition or method that is sufficient to show a meaningful patient benefit, i.e., reduction of a condition. When applied to an individual active ingredient, administered alone, the term refers to that ingredient alone. When applied to a combination, the term refers to combined amounts of the active ingredients that result in the therapeutic effect, whether administered in combination, serially or simultaneously.

The term “ameliorate” means a detectable or measurable improvement in a subject's condition or symptom thereof. A detectable or measurable improvement includes a subjective or objective decrease, reduction, inhibition, suppression, limit or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one, most or all adverse symptoms, complications, consequences or underlying causes associated with the condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition (e.g., stabilizing), over a short or long duration of time (e.g., seconds, minutes, hours).

The term “alkyl” means a straight or branched hydrocarbon group having 1-10 carbon atoms and includes, for example, methyl, ethyl, n-propyl, isopropyl, n-butyl, sec-butyl, isobutyl, tert.-butyl, n-pentyl, n-hexyl, n-heptyl, n-octyl, n-nonyl, n-decyl, fluoromethyl, fluorochloromethyl, and trifluoromethyl, and the like. Alkyl groups may be optionally substituted with one or more substituents, such as halogens. The term “branched” should be understood to represent a linear straight chain hydrocarbon group having one or more lower alkyl groups such as methyl, ethyl or propyl, attached to it. The term “alkenyl” refers to an alkyl group containing at least one carbon-carbon double bond. Alkenyl groups may be optionally substituted with one or more substituents. The term “alkynyl” refers to an alkyl group containing at least one carbon-carbon triple bond. Alkynyl groups may be optionally substituted with one or more substituents. The term “halogen” (or “halo”) should be understood to include fluoro (fluorine), chloro (chlorine), bromo (bromine), and iodo (iodine). The term “hydroxypropyl” refers to three-carbon groups comprising one hydroxyl group and includes, but is not limited to, 2-hydroxypropyl and 1-hydroxypropan-2-yl. The term “dihydroxypropyl” refers to three-carbon groups comprising two hydroxyl groups and includes, but is not limited to, 1,3-dihydroxypropan-2-yl and 2,3-dihydroxypropyl.

Certain of the disclosed compounds may exist in various stereoisomeric forms. Stereoisomers are compounds that differ only in their spatial arrangement. Enantiomers are pairs of stereoisomers that are non-superimposable mirror images of one another, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. The symbol “*” in a structural formula represents the presence of a chiral carbon center. “R” and “S” represent the configuration of substituents around one or more chiral carbon atoms. Thus, “R*” and “S*” denote the relative configurations of substituents around one or more chiral carbon atoms.

The present disclosure in at least certain embodiments utilizes one or more stable shikimate electrophiles and/or shikimate electrophilic precursors (shikimate analogues) that are reactive toward a variety of offensive nucleophilic compounds including thiols (e.g., malodorous low molecular compounds produced by skunks), catechols (e.g., urushiols produced as dermal irritants by many plants), histamines, and other offensive chemical agents that contain nucleophilic groups. The shikimate analogues provide stable electrophiles or electrophilic precursors that can be reacted with offensive nucleophile-containing irritants and malodorants. The shikimate analogues include naturally produced compounds made by fungi, as well as a variety of semisynthetic analogues and derivatives and compounds with structures that are the same or analogues of natural products, but were produced through synthetic processes. The reactive shikimate analogues can be held under refrigeration, freezing, or at room temperature prior to application. The shikimate analogues can be converted ahead of time or at the time of use into more reactive electrophiles that will covalently bond to thiols, catechols, histamines, and other nucleophiles. The chemical bonding process (wherein a molecule of the shikimate analogue binds to a molecule of the toxin or irritant) produces chemically altered compounds that are no longer active (e.g., no longer an epithelial toxin or irritant) or no longer capable of eliciting an olfactory response (produce no offensive odor). The chemically altered product might also have reduced activity or reduced olfactory response. It is possible that a chemically altered product could be an irritant or olfactory response elicitor, but only at increased concentrations, i.e., after treatment the noxious agent becomes less potent. The electrophiles can be delivered as a dry powder, cream, salve, aqueous solution, or aqueous suspension with or without adjuvants. The reaction products will often have increased polarity thereby facilitating their removal with water or soap and water due to an increased polarity or amphiphilic tendencies.

Once combined, the shikimate analogue(s) and secondary compound(s) of the present disclosure can be used in treatments to partially or completely neutralize and/or deodorize irritant/odoriferous thiol-ester-containing compounds and thiol-ester containing precursors of irritant/odoriferous compounds, irritant/odoriferous amine-containing compounds and amine-containing precursors of irritant/odoriferous compounds; irritant/odoriferous carboxylic-acid-containing compounds and carboxylic-acid-containing precursors of irritant/odoriferous compounds; irritant/odoriferous organosulfur-containing compounds, such as sulfinic acids, sulfenic acids, sulfonic acids, sulfonate esters, sulfoxides, sulfones, and precursors of such irritant/organosulfur-containing compounds; irritant/odoriferous compounds such as selenols, diselenides, disulfides, hydrogen sulfide and organic sulfides, geosmin, skatole, Dipple's oil, Neatsfoot dimethyl trisulfide, and their chemical precursors; and odoriferous compounds contained in naturally occurring and human-prepared multicomponent chemical mixtures, such as skunk spray or essence, urine, feces, decomposing organic matter, non-lethal malodorants, the products and intermediates of microbial fermentation, farm practices, food manufacturing processes, and wastewater, and chemical precursors thereof.

Particular examples of irritant/odoriferous compounds that may be treated with the shikimate analogue compounds and compositions of the present disclosure include, but are not limited to (a) organosulfur and thiol-containing compounds including, but not limited to methanethiol, ethanethiol, propane-1-thiol, propane-2-thiol, thioglycolic acid, 2-naphthalenethiol, 3-methylbutane-1-thiol, 2-methylbutane-1-thiol, but-3-ene-1-thiol, (E)-but-2-ene-1-thiol, (z)-but-2-ene-1-thiol, butanethiol, benzenethiol, phenylmethanethiol, quinoline-2-thiol, quinoline-3-thiol, quinoline-4-thiol, quinolin-2-ylmethanethiol, quinolin-3-ylmethanethiol, quinolin-4-ylmethanethiol, furfuryl mercaptan, and 3-methylbut-2-ene-1-thiol, and combinations and mixtures thereof; (b) carboxylic acids and carboxylic-acid-containing compounds including, but not limited to, propionic acid, butyric acid, valeric acid, and caproic acid, and combinations and mixtures thereof; (c) amines and amine-containing compounds including, but not limited to dimethyl amine, diethyl amine, dipropyl amine, ethanolamine, putrescine, cadaverine, and skatole, and combinations and mixtures thereof; and (d) inorganic sulfur-containing and nitrogen-containing compounds including, but not limited to, hydrogen sulfide, ammonium sulfide, and ammonia, and combinations and mixtures thereof.

Various materials and substances can also be treated to neutralize or decrease the odors emanating therefrom, including, but not limited to, urine, human and animal feces, smoke, mildew, vomit, body odor, diapers, feminine hygiene products, human corpses, animal corpses, solid and liquid animal wastes, decaying animals, sewage, dog anal gland discharge, outhouses, portable toilets, port a potties, fishes, floors, fish organs, fish skin, fish oils, fish-derived refuse, cat litter, garbage, garbage cans, and garbage bags.

The present disclosure, in certain embodiments, describes a chemically driven (non-enzymatic) toxin inactivation system employed by a soil ascomycete such as Tolypocladium sp. This process was serendipitously discovered shortly after we initiated further chemical studies of the shikimate-PKS-NRPS metabolite, maximiscin (1), which our group identified from Tolypocladium sp. Salcha MEA-2 (T1) (L. Du, A. J. Robles, J. B. King, D. R. Powell, A. N. Miller, S. L. Mooberry, R. H. Cichewicz, Angew. Chem. Int. Edit. 2014, 53, 804-809). These results led us to determine that the shikimate portion of 1 is incorporated via a substitution reaction involving a chemoreactive precursor metabolite. This natural product is reactive toward a broad range of exogenous antibiotic/toxic chemicals. Herein we describe the unique balance of electrophilic promiscuity and chemical stability exhibited by the chemoreactive Tolypocladium metabolite and its analogues, as well detail how they likely serve to protect the fungus from antibiosis. All references cited herein (including U.S. Provisional Patent Application 62/295,343) are explicitly incorporated by reference herein in their entireties.

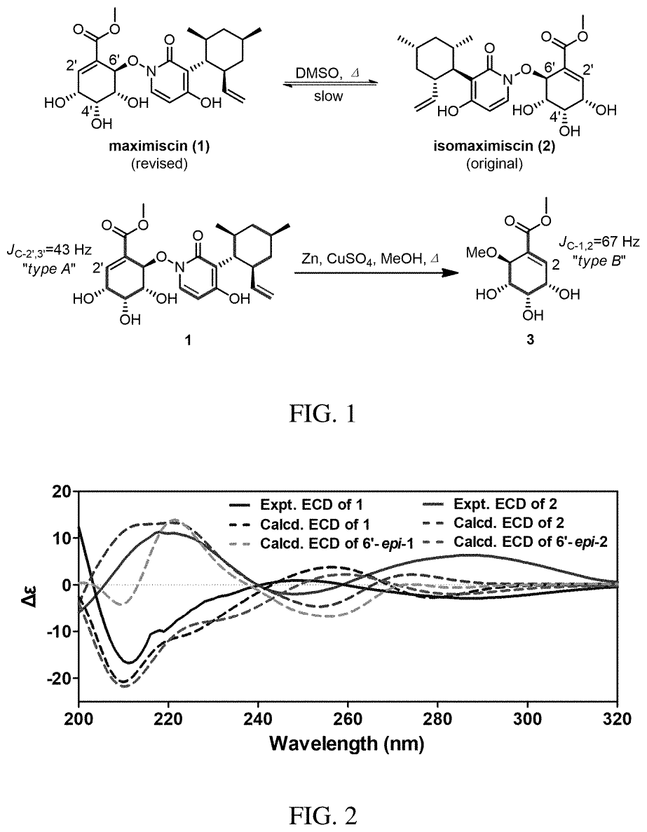

Before further discussing these new discoveries, attention is drawn to the fact that during the initial stages of our follow-up studies about the production of 1, we were confronted with data that were at odds with our original hypothesis of the metabolite's absolute configuration (details of these studies are provided in the appendix of U.S. Provisional Patent Application 62/295,343). The new results led us to realize that during the original VCD experiments, in which 1 was held for hours in DMSO with warming, the compound had rearranged into a new isomeric species, isomaximiscin (2) (FIG. 1). This result proved to be auspicious because it (i) provided an early clue regarding the remarkable process leading to the formation of 1, (ii) enabled us to couple the spectroscopically-derived absolute configuration results for 1 to data reported for synthetically prepared 3 (FIG. 1), (Y. Usami, M. Ohsugi, K. Mizuki, H. Ichikawa, M. Arimoto, Org. Lett. 2009, 11, 2699-2701; D. R. Boyd, N. D. Sharma, C. A. Acaru, J. F. Malone, C. R. O'Dowd, C. C. Allen, P. J. Stevenson, Org. Lett. 2010, 12, 2206-2209), and (iii) led to our development of new investigational tools (i.e., ¹³C isotopic labeling analysis and ECD measurements) that proved useful for probing the reactivity spectrum of the precursors of 1 with a range of antibiotics and toxins.

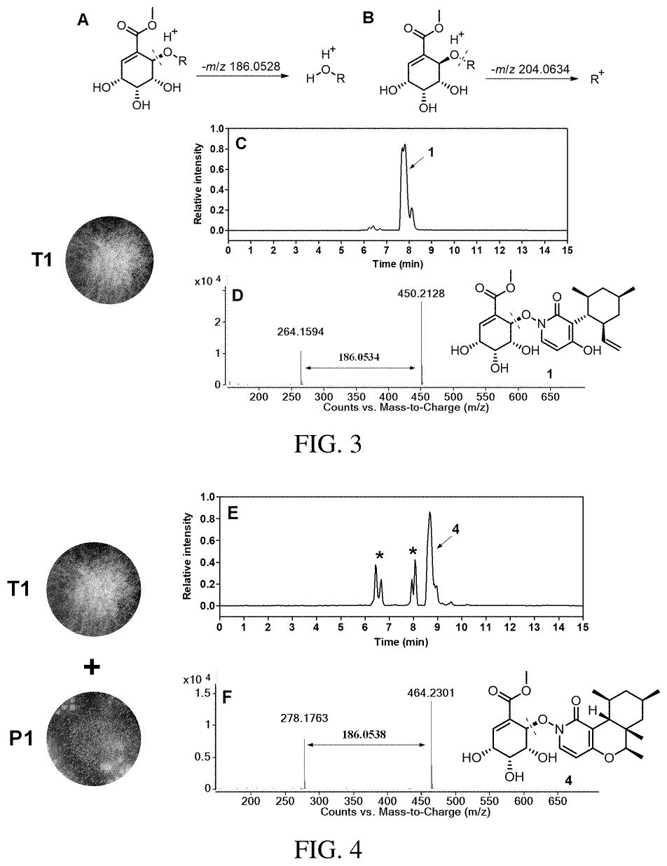

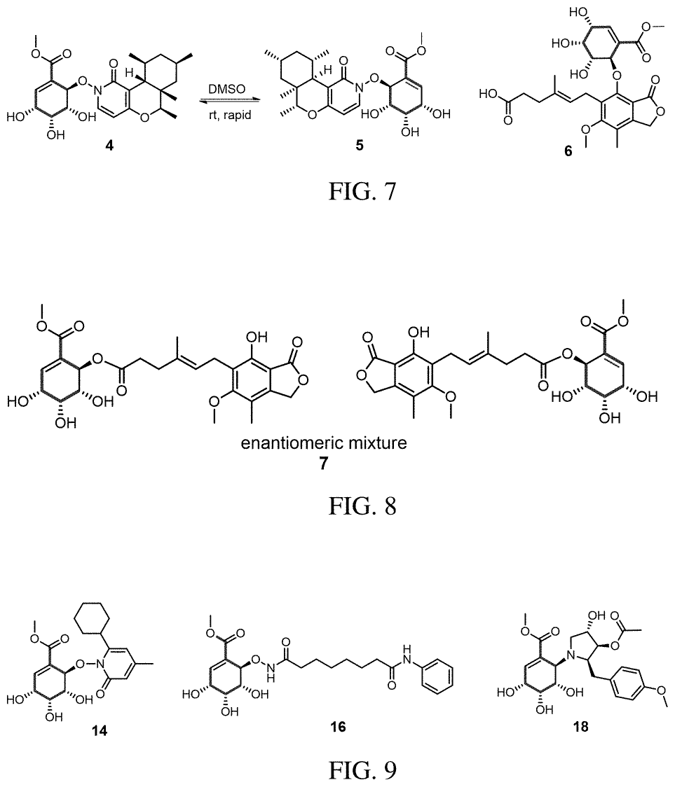

Continuing with our exploration of fungus T1, a notable attribute of its behavior was the consistent production of secondary metabolites in response to the presence of coculture microbial species (L. Du, A. J. Robles, J. B. King, D. R. Powell, A. N. Miller, S. L. Mooberry, R. H. Cichewicz, Angew. Chem. Int. Edit. 2014, 53, 804-809). Further studies examining additional fungal coculture scenarios confirmed the robustness of this response (Table S17, appendix of U.S. Provisional Patent Application 62/295,343). For example, UPLC-ESIMSⁿ analysis of a co-culture consisting of T1 and Penicillium sp. P1 provided evidence for a new compound that yielded ions at m/z 464.2301 ([M+H]⁺) and m/z 278.1763 ([M+H]±) (FIG. 4). Curiously, the difference between these two major ions (Δ m/z 186.0538) was identical to the neutral loss due to cleavage of the shikimate analogue moiety, which we had previously detected during the MSⁿ analysis of 1 (FIG. 3). This alerted us to the possibility that the new co-culture metabolite might also contain a shikimate analogue moiety. Scale-up preparation, purification, and structure determination revealed the new compound was structurally related to 1 and was named pseudomaximiscin A (4) (FIG. 4). Similar to 1, incubation of 4 in DMSO-d₆ led to its isomerization resulting in a ˜1:1 equilibrium mixture containing its diastereomeric product, pseudomaximiscin B (5) (FIG. 7).

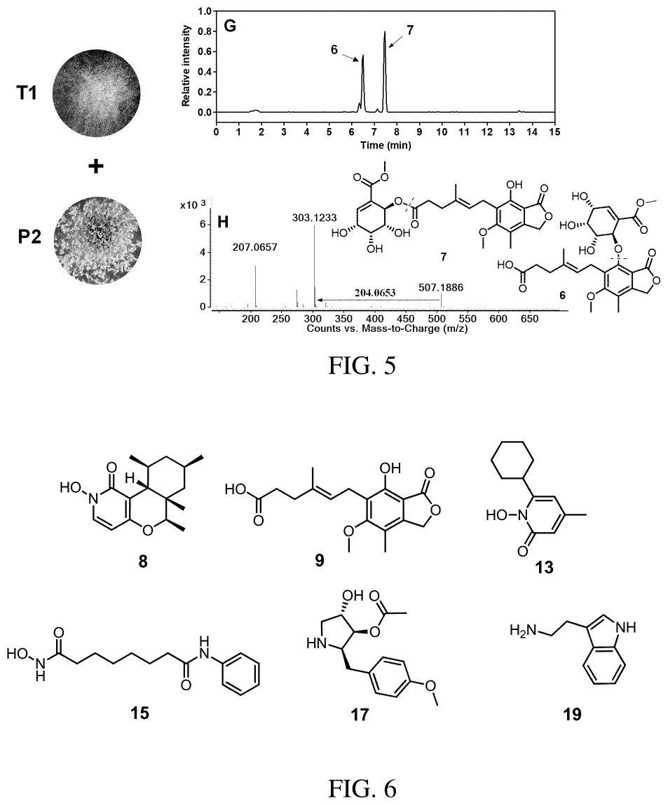

Intrigued by the discovery of 4, we reexamined the MSⁿ data from the fungal co-culture experiments and noted that in addition to the recurring A m/z 186 for several new metabolites, a second neutral loss of A m/z 204 (FIG. 3) was apparent. Using these two parameters to filter the MSⁿ data, a neutral loss event of A m/z 204 was identified for two metabolites that were generated when fungus T1 was co-cultured with Penicillium P2 (FIG. 5). Scale-up production yielded the new compounds, mycophenolic acid 3-O-pericosine (6) and mycophenolic acid 16-O-pericosine (7). Whereas 6 was optically active ([α]²⁰_{D}-126), 7 was not, indicating that it was a racemic mixture. Deliberate probing of all samples by UPLC-ESIMSⁿ and ion-selective MS revealed that the likely non-shikimate precursor metabolites of 4, 6, and 7 were coming from the co-culture partners (FIGS. 7-8). For example, fungus P1, was determined to be the source of metabolite PF1140 (8) (E. D. de Silva, A. S. Geiermann, M. I. Mitova, P. Kuegler, J. W. Blunt, A. L. Cole, M. H. Munro, J. Nat. Prod. 2009, 72, 477-479), whereas fungus P2 made mycophenolic acid (9) (X. Lu, Z. Zheng, H. Zhang, C. Huo, Y. Dong, Y. Ma, X. Ren, A. Ke, J. He, Y. Gu, Q. Shi, J. Antibiot. 2009, 62, 527-529). Therefore, compounds 4, 6, and 7 were proposed to be chimeric metabolites made from the union of 8 or 9 with a yet undetermined chemoreactive compound from fungus T1.

To determine how the T1 culture facilitated this process, an experiment was conceived using P1-derived metabolite 8 as ‘bait’ in a chemoassay-guided process meant to uncover the origins of the shikimate analogue addition (FIG. 11). Initially, purified 8 was mixed with dialysate prepared from one-week-old T1 culture broth. LAESIMS monitoring of the reaction revealed that compound 4, which yielded a [M+H]⁺ quasi molecular ion peak at m/z 464.2301 was detectable with dialysate prepared both with large (1000 kDa) and small (0.5-1 kDa) average-molecular-weight cutoff membranes. This suggested that the reaction could occur in a cell-free environment by means of a non-enzymatic process. Next, T1 cultures were successively extracted with EtOAc and n-butanol. Whereas the remaining aqueous layer was inactive, samples from both organic layers were able to generate 4 upon the addition of 8. Subsequent chemoassay-directed HPLC fractionation led to the purification of two shikimate analogues, including the new epoxide metabolite, pericoxide (10), from the EtOAc extract, as well as the known chlorinated compound, (+)-pericosine A (11) (Y. Usami, M. Ohsugi, K. Mizuki, H. Ichikawa, M. Arimoto, Org. Lett. 2009, 11, 2699-2701; D. R. Boyd, N. D. Sharma, C. A. Acaru, J. F. Malone, C. R. O'Dowd, C. C. Allen, P. J. Stevenson, Org. Lett. 2010, 12, 2206-2209), from the n-butanol extract. These results led us to determine that 10 and 11 are the probable precursors to the non-enzymatic formation of 1 in T1 cultures, not the 6-OH analogue as we had previously implicated (L. Du, A. J. Robles, J. B. King, D. R. Powell, A. N. Miller, S. L. Mooberry, R. H. Cichewicz, Angew. Chem. Int. Edit. 2014, 53, 804-809).

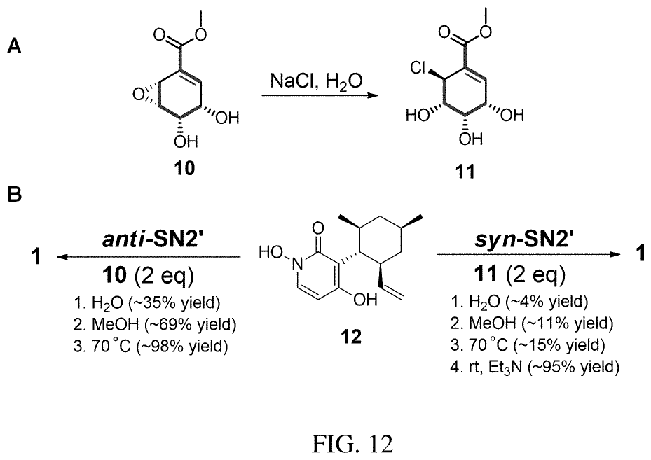

With 10 and 11 identified as candidates for the formation of the co-cultured-derived hybrid metabolites, the chain of antecedence linking the two compounds was called into question. UPLC-ESIMSⁿ analysis of the MeOH extract of the cell lysate of T1 revealed that neither 10, 11, nor any other hybrid metabolites (e.g., 1) were detectable intracellularly, implying that both 10 and 11 were either formed extracellularly or sequestered and secreted from the cells upon their formation. Further examining the ¹³C-labeled 10 and 11 (prepared by feeding fungus T1 [U-¹³C₆]-D-glucose), it was determined that both compounds were present in the spent culture broth as single enantiomers exhibiting a “type B” ¹³C-labeling patterns (FIG. 1 and supporting information in the appendix of U.S. Provisional Patent Application 62/295,343). Treatment of ¹³C-labeled 10 with NaCl in ddH₂O yielded 11 seemingly via an S_{N}2 mechanism (FIG. 12A). ECD analysis showed that 11 prepared both from 10, as well as directly from the fungal culture broth bore the same absolute configuration (FIG. S126 of the appendix of U.S. Provisional Patent Application 62/295,343). These results implied that 11 was non-enzymatically produced from 10 within the T1 culture. This hypothesis was tested by preparing fungal culture broth for T1 using Millipore water and observing that the yield of 11 was strongly correlated with the quantity of NaCl or other Cl⁻ sources that were added to the culture medium (FIG. S124 of the appendix of U.S. Provisional Patent Application 62/295,343).

The roles that 10 and 11 might play in the production of 1 were tested by assessing their reactivities toward pyridoxatin (12) (L. Du, A. J. Robles, J. B. King, D. R. Powell, A. N. Miller, S. L. Mooberry, R. H. Cichewicz, Angew. Chem. Int. Edit. 2014, 53, 804-809). The production of 1 was found to occur in Millipore water upon addition of 12 to both 10 and 11. Manipulation of selected reaction conditions (i.e., solvent, temperature, and catalyst) confirmed that epoxide 10 was generally more reactive toward 12 than its halohydrin counterpart 11. In all cases, enantiomerically pure 1 was obtained as the product indicating that a selective S_{N}2′ mechanism was involved in the formation of 1 under both synthetic, as well as in situ culture conditions (FIG. 12B).

To test the promiscuous reactivity of 10 and 11 toward other compounds, T1 cultures were treated with a panel of substrates that included chemically diverse functional groups: hydroxamic acids, phenols, carboxylic acids, alcohols, alkenes, amides, and amines (Table S14 of the appendix of U.S. Provisional Patent Application 62/295,343). Candidate products from each reaction were purified and their structures confirmed by HRESIMS and multidimensional NMR. In addition, T1 cultures were supplied with [U-¹³C₆]-D-glucose so that the labeling patterns of the resulting products (FIGS. 7-10) would afford insights regarding the probable substitution mechanisms involved in their formation (i.e., S_{N}1, S_{N}2, or S_{N}2′) (FIG. S125 of the appendix of U.S. Provisional Patent Application 62/295,343). Additionally, the absolute configuration of the C-6′ position in each product was determined by comparing its experimental and theoretical ECD data (FIG. S126 of the appendix of U.S. Provisional Patent Application 62/295,343). Based on these analyses, the stereoselectivities of the coupling reactions (particularly anti-vs syn-S_{N}2′ mechanisms) were systematically assessed (FIG. S125 of the appendix of U.S. Provisional Patent Application 62/295,343).

The results provided evidence that 10 and 11 were decidedly reactive toward diverse chemical targets. Stereoselective S_{N}2′ (anti-S_{N}2′ for 10 and syn-S_{N}2′ for 11) reaction processes were observed involving all the hydroxamic acids, including PF1140 (8), ciclopirox (13), and SAHA (15), to give optically active products 4, 14, and 16, respectively. A similar S_{N}2′ reactivity pattern was observed involving the reaction of 10 and 11 with phenol-containing [3-OH of mycophenolic acid (9)] and secondary-amine-containing [anisomycin (17)] substrates. In contrast, reaction with the carboxylic acid moiety of 9 yielded racemic 7 (FIG. S26 of the appendix of U.S. Provisional Patent Application 62/295,343). The 5′R*6′R* relative configuration of the product was assigned based on an examination of its ¹³C NMR chemical shifts in comparison with DFT calculated data (FIGS. S12-S14 of the appendix of U.S. Provisional Patent Application 62/295,343). Whether product mixture 7 arose from competing reaction processes or a rearrangement remains unknown.

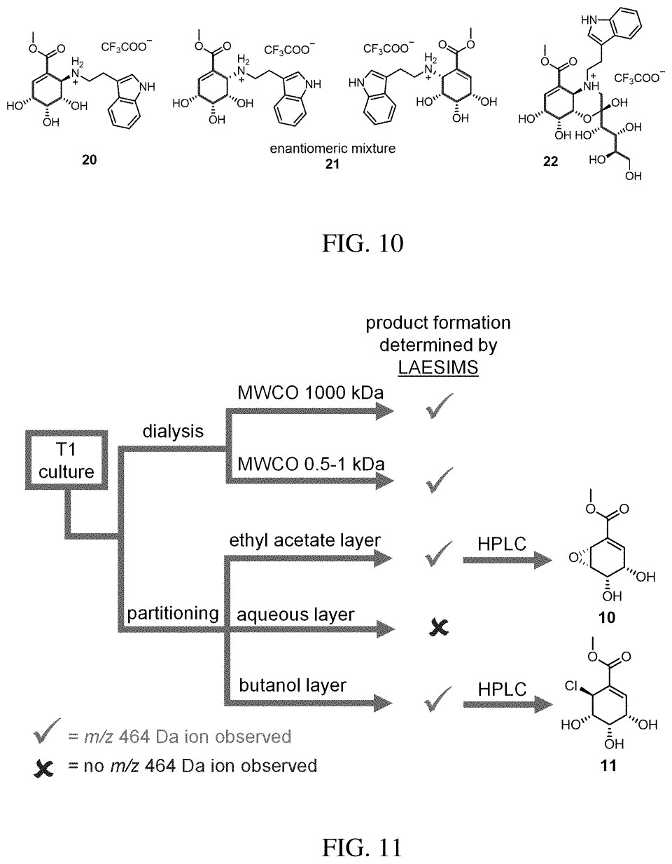

The primary amine tryptamine (19) was also administered to T1 resulting in the formation of products 20 and 21 and an unexpected novel product named mallimiscin (22) (FIG. 10). Distinct J_{H-5′,H-6′} values aided in determining the 5′,6′-relative configuration (trans >9 Hz, cis ˜5 Hz) of the products. Whereas 20 was obtained as an enantiomerically pure product, the diastereomers (21) were a racemate (FIG. S27 of the appendix of U.S. Provisional Patent Application 62/295,343). Compound 22 was seemingly formed via a Maillard reaction of 20 with D-glucose, based on its ¹³C-labeling pattern, ROESY correlations, J_{H,H} coupling constants, and ECD calculation (FIG. 10, and FIGS. S126, and S127 of the appendix of U.S. Provisional Patent Application 62/295,343).

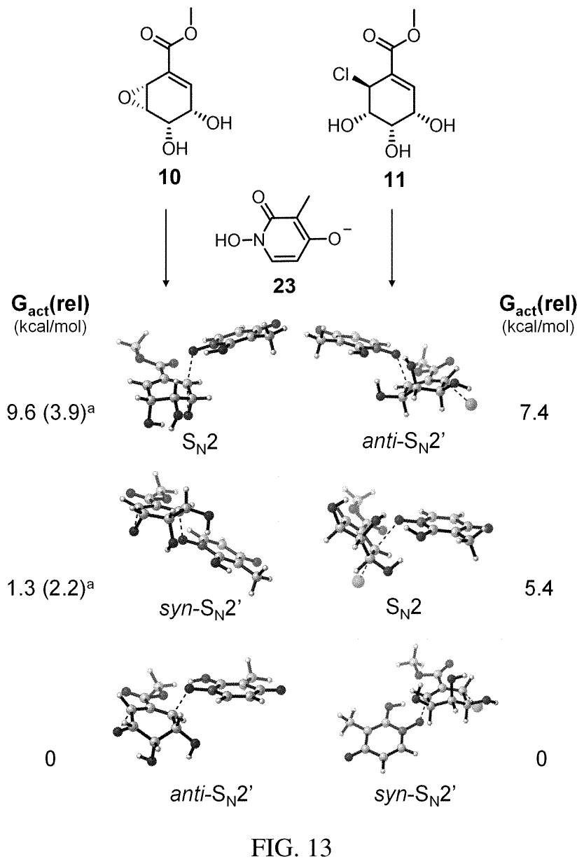

To better understand the selectivity exhibited by 10 and 11 for several of the substrates, DFT calculations were employed to determine the energies of the transition states for syn-S_{N}2′, anti-S_{N}2′ and S_{N}2 reactions of 10 and 11 with the model hydroxamic acid 23. The β-hydroxycarbonyl hydroxy group in 23 was estimated to have a pKa of about 7-9 indicating that its anionic form may be present as the reactive nucleophile in aqueous media. The computational results (FIG. 13) were in quantitative agreement with the experimentally observed regio- and stereoselectivities of the reactions of hydroxamic acids with both 10 and 11. For the reaction of 23 with 11, the syn-S_{N}2′ transition state (and its corresponding activation energy) was energetically the lowest by a substantial margin. In contrast, the anti-S_{N}2′ pathway was favored in the reaction involving epoxide 10 by a smaller margin, dependent on the computational method. Notably, each of the transition states identified in these calculations suggest that tautomerization occurs between the C-2 carbonyl and N—OH groups.

The observed (and calculated) exclusively syn-S_{N}2′ (for allyl-X) or anti-S_{N}2′ (for vinyl oxirane) selectivity detected among some of these reactions has been found in other vinylogous nucleophilic substitution reaction systems, but there are also many exceptions, depending on the substrate, nucleophile and solvent (D. Sinou, Organic Reactions in Water: Principles, Strategies and Applications (Ed.: U. M. Lindström), Wiley-Blackwell, 2007, 236-255; A. Chanda, V. V. Fokin, Chem. Rev. 2009, 109, 725-748; P. E. Savage, Chem. Rev. 1999, 99, 603-621). Numerous factors have been invoked to explain the observed selectivity in such reactions, including frontier orbital and coulombic interactions, conformational and steric effects, specific Lewis acid-base and H-bonding interactions, and solvation. The origin of the remarkable and divergent stereoselectivities in the reactions of these two allylic substrates is presently unclear and its elucidation will require additional experimental and computational investigation with a range of relevant nucleophiles.

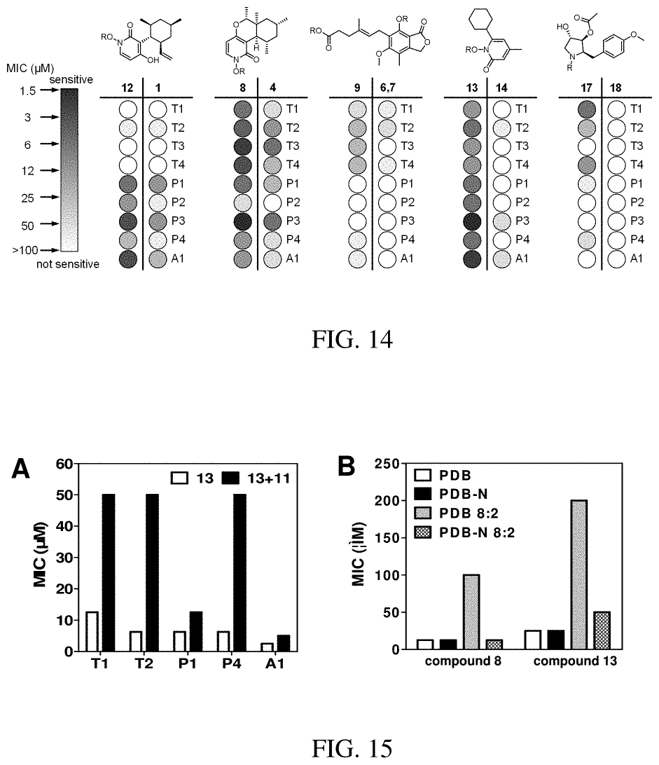

Reflecting on the potential biological roles of electrophilic 10 and 11, we noted that the natural product precursors made by T1's partner fungus had possessed antifungal activities. This prompted us to ask the question what effect the addition of the shikimate analogue moiety had on the bioactivities of compounds 8, 9, 12, and the other nucleophilic substances that we tested. A panel of fungi including four Tolypocladium spp. (T1-T4), four Penicillium spp. (P1-P4) and Aspergillus niger (A1) were selected for assessment. The test revealed a distinct trend in which the antifungal activities of the substrates were greatly diminished or abolished following the addition of the shikimate analogue moiety (FIG. 14). For example, both the P1-derived antifungal 8 and the synthetic antifungal 13 showed growth inhibition against all of the fungal strains with MIC values in the range of 1-50 μM, whereas their adducts, 4 and 14, exhibited an average >5-fold decrease in potencies. To test the capacity of 11 to block the toxicity of 13 in real time, fungal cultures were preincubated with 11 and then treated with varying doses of 13. This regimen of preadministering 11 afforded up to an 8-fold decrease in the MIC of 13 (FIG. 15A). Our prior time-course studies examining the production of 10 and 11 showed that these metabolites rapidly accumulated in T1 cultures after 96 hours. We had also noted that the addition of NaNO₃ to the culture medium thoroughly abolished the formation of 10 and 11. Using this information, we determined that fungus T1 was equally sensitive to the antifungal activities of 8 or 13 in both freshly prepared normal and NaNO₃-supplemented media during a 3-day test window. However, when 5-day-old T1 culture broth was used to prepare the test medium, the non-NaNO₃-supplemented T1 culture became increasingly resistant to 8 and 13 (FIG. 15B). In summary, these studies provide evidence for a new chemically facilitated mode of toxin resistance exhibited by a soil ascomycete. Whereas previously reported resistance mechanisms involving antibiotic modification depended on the enzymatic modification of target substrates, fungal metabolites 10 and 11 functioned as electrophilic warheads that were reactive to a wide variety of natural and synthetic organic substances. The actions of these metabolites serve to limit the deleterious effects of antibiotics/toxins against their microbial targets.

Generic Structures of the Shikimate Analogues

Certain embodiments of the present disclosure are directed to shikimate analogues as represented by Structural Formula I and Structural Formula II below. The present disclosure also includes compositions and kits containing such analogues, and methods of use of such compounds and compositions.

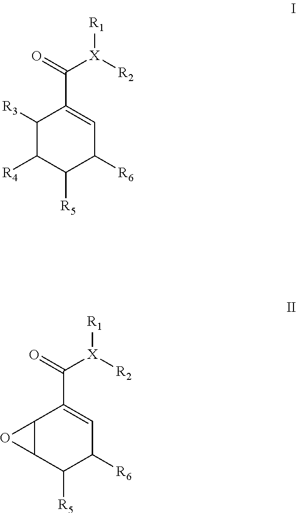

In non-limiting embodiments of Structural Formula I:

X is optionally O, N, S, or is absent;

R₁ is optionally selected from the group consisting of H, (C1-C8)alkyl, (C1-C8)alkenyl, (C1-C8)alkynyl, cyano, halo, nitro, thio, substituted phenyl, phenylmethyl, substituted naphthalenyl;

R₂ is optionally selected from the group consisting of H, (C1-C8)alkyl, (C1-C8)alkenyl, (C1-C8)alkynyl, cyano, halo, nitro, thio, substituted phenyl, phenylmethyl, substituted naphthalenyl, or is absent;

R₃ is optionally selected from the group consisting of fluoro, chloro, bromo, iodo, hydroxyl, substituted phosphate, —O-tosyl, —O-mesyl, (C1-C8)alkoxy, (C2-C8)acyloxy, substituted phenoxy, substituted naphthalenyloxy, substituted naphthalenylmethoxy, (C1-C12)primary amino, (C1-C12)secondary amino, (C1-C12)tertiary amino, and (C1-C12)cyclic amino, (C1-C8)ammonio, (C1-C8)carboxamino, (C1-C8)imino, azido, (C1-C8)azo, cyanato, isocyanato, nitrooxy, cyano, isocyano, nitrosooxy, nitro, nitroso, (C1-C8)substituted carbamoyl, hydroxyamino, morpholino, anilino, indol, pyrrol, imidazole, benzimidazol, pyrazol, guanidino, piperazino, polyamino, and N-methylated polyamino;

R₄ optionally is selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl) ethenyl]oxy, (1-carboxyethenyl)oxy;

R₅ is optionally selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl) ethenyl]oxy, (1-carboxyethenyl)oxy; and

R₆ is optionally selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl) ethenyl]oxy, (1-carboxyethenyl)oxy.

In non-limiting embodiments of Structural Formula II:

X is optionally O, N, S, or is absent;

R₁ is optionally selected from the group consisting of H, (C1-C8)alkyl, (C1-C8)alkenyl, (C1-C8)alkynyl, cyano, halo, nitro, thio, substituted phenyl, phenylmethyl, substituted naphthalenyl;

R₂ is optionally selected from the group consisting of H, (C1-C8)alkyl, (C1-C8)alkenyl, (C1-C8)alkynyl, cyano, halo, nitro, thio, substituted phenyl, phenylmethyl, substituted naphthalenyl, or is absent;

R₅ is optionally selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl) ethenyl]oxy, (1-carboxyethenyl)oxy; and

R₆ is optionally selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl) ethenyl]oxy, (1-carboxyethenyl)oxy.

Polyamino R₃ groups of I include but are not limited to, structures based on low-molecular-weight linear polyamines such as spermine, spermidine, putrescine, cadaverine, thermospermine, ethylenediamine, diethylenetriamine, and triethlenetetramine, and N-methylated forms thereof.

Certain embodiments of the present disclosure are directed to shikimate analogues as represented by Structural Formula III and Structural Formula IV below. The present disclosure also includes compositions and kits containing such analogues, and methods of use of such compounds and compositions.

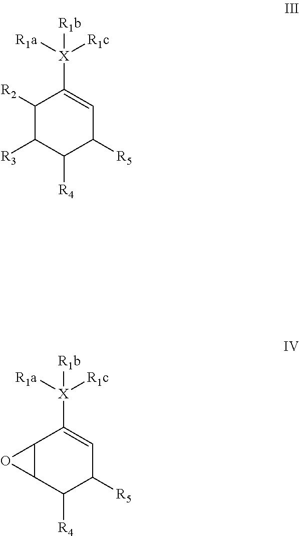

In non-limiting embodiments of Structural Formula III or of Structural Formula IV:

X is optionally be selected from the group including CN (nitrile), NO₂ (nitro), an amine salt, trifluoromethyl, difluoromethyl, trichloromethyl, dichloromethyl, a carbon, a carbon with one or more halogens attached (fluorine, chlorine, bromine, iodine) in any combination thereof (e.g., flurochloromethyl), or a carbonyl-containing (CO-containing) group including ketones, carboxylic acids (and salts thereof), esters, primary amides, secondary amides, tertiary amides, and thioesters;

R₁a is optionally be selected from the group consisting of H, hydroxy, (C1-C8)alkyl, (C1-C8)alkenyl, (C1-C8)alkynyl, cyano, halo, nitro, thio, substituted phenyl, phenylmethyl, substituted naphthalenyl, hydroxypropyl, and dihydroxypropyl, or is absent;

R₁b is optionally be selected from the group consisting of H, hydroxy, (C1-C8)alkyl, (C1-C8)alkenyl, (C1-C8)alkynyl, cyano, halo, nitro, thio, substituted phenyl, phenylmethyl, substituted naphthalenyl, hydroxypropyl, and dihydroxypropyl, or is absent;

R₁c is optionally be selected from the group consisting of H, hydroxy, (C1-C8)alkyl, (C1-C8)alkenyl, (C1-C8)alkynyl, cyano, halo, nitro, thio, substituted phenyl, phenylmethyl, substituted naphthalenyl, hydroxypropyl, and dihydroxypropyl, or is absent;

R₂ is optionally selected from the group consisting of fluoro, chloro, bromo, iodo, hydroxyl, substituted phosphate, —O-tosyl, —O-mesyl, (C1-C8)alkoxy, (C2-C8)acyloxy, substituted phenoxy, substituted naphthalenyloxy, substituted naphthalenylmethoxy, (C1-C12)primary amino, (C1-C12)secondary amino, (C1-C12)tertiary amino, and (C1-C12)cyclic amino, (C1-C8)ammonio, (C1-C8)carboxamino, (C1-C8)imino, azido, (C1-C8)azo, cyanato, isocyanato, nitrooxy, cyano, isocyano, nitrosooxy, nitro, nitroso, (C1-C8)substituted carbamoyl, hydroxyamino, morpholino, anilino, indol, pyrrol, imidazole, benzimidazol, pyrazol, guanidino, piperazino, polyamino, and N-methylated polyamino;

R₃ is optionally selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl) ethenyl]oxy, (1-carboxyethenyl)oxy;

R₄ is optionally selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl) ethenyl]oxy, (1-carboxyethenyl)oxy; and

R₅ is optionally selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl) ethenyl]oxy, (1-carboxyethenyl)oxy; and wherein
(1) when X is a carbon atom and Ria is a halo (fluorine, chlorine, bromine, or iodine), Rib and R₁c may optionally be selected from the group consisting of H, hydroxy, (C1-C8)alkyl, (C1-C8)alkenyl, (C1-C8)alkynyl, cyano, halo, nitro, thio, substituted phenyl, hydroxypropyl, and dihydroxypropyl, in any combination thereof;

(2) when X is a carbon atom, R₁a is a halo (fluorine, chlorine, bromine, or iodine), and R₁b is a halo, R₁c may optionally be selected from the group consisting of H, hydroxy, (C1-C8)alkyl, (C1-C8)alkenyl, (C1-C8)alkynyl, cyano, halo, nitro, thio, substituted phenyl, hydroxypropyl and dihydroxypropyl;

(3) when X is a carbonyl-containing ketone group, R₁a may optionally be selected from the group consisting of H, hydroxy, (C1-C8)alkyl, (C1-C8)alkenyl, (C1-C8)alkynyl, cyano, halo, nitro, thio, substituted phenyl, hydroxypropyl, and dihydroxypropyl, and R₁b and R₁c are absent;

(4) when X is a carbonyl-containing ester, secondary amide, or thioester group, R1a may optionally be selected from the group consisting of hydroxy, (C1-C8)alkyl, (C1-C8)alkenyl, (C1-C8)alkynyl, cyano, (C1-C8)alkyl cyano, (C1-C8)alkyl halide, (C1-C8)alkyl nitro, (C1-C8)alkyl thio, substituted phenyl, hydroxypropyl, and dihydroxypropyl, and R₁b and R₁c are absent; and

(5) when X is a carbonyl-containing tertiary amide group, R₁b and R₁c may optionally be selected from the group consisting of hydroxy, (C1-C8)alkyl, (C1-C8)alkenyl, (C1-C8)alkynyl, cyano, (C1-C8)alkyl cyano, (C1-C8)alkyl halide, (C1-C8)alkyl nitro, (C1-C8)alkyl thio, substituted phenyl, hydroxypropyl, and dihydroxypropyl, in any combination thereof, and R1a is absent.

Polyamino R₂ groups of III include but are not limited to, structures based on low-molecular-weight linear polyamines such as spermine, spermidine, putrescine, cadaverine, thermospermine, ethylenediamine, diethylenetriamine, and triethlenetetramine, and N-methylated forms thereof.

Certain embodiments of the present disclosure are directed to shikimate analogues as represented by Structural Formula V below. The present disclosure also includes compositions and kits containing such analogues, and methods of use of such compounds and compositions as disclosed elsewhere herein.

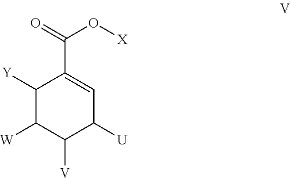

In non-limiting embodiments of Structural Formula V:

X is selected from the group consisting of hydrogen, methyl, ethyl, n-propyl, iso-propyl, N,N,N-trimethyl-2-(11-oxidaneyl)ethan-1-aminium, N,N,N-methyldiethyl-2-(11-oxidaneyl)ethan-1-aminium, N,N,N-dimethylethyl-2-(11-oxidaneyl)ethan-1-aminium, N,N-diethyl-2-(11-oxidaneyl)ethan-1-aminium, or N,N,N-triethyl-2-(11-oxidaneyl)ethan-1-aminium;
Y is selected from the group consisting of fluorine, chlorine, bromine, iodine, tosyl, brosyl, mesyl, triphenylphosphine, sulfonates, sulfonamides, ammoniums, and phosphonates;
W is selected from the group consisting of hydrogen, hydroxy, methoxy, ethoxy, methyl, and ethyl;
V is selected from the group consisting of hydrogen, hydroxy, methoxy, ethoxy, methyl, and ethyl; and
U is selected from the group consisting of hydrogen, hydroxy, methoxy, ethoxy, methyl, and ethyl.

Certain embodiments of the present disclosure are directed to shikimate analogues as represented by Structural Formula VI below. The present disclosure also includes compositions and kits containing such analogues, and methods of use of such compounds and compositions as disclosed elsewhere herein.

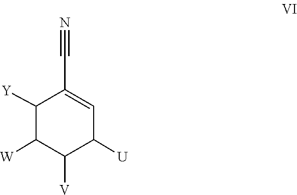

In non-limiting embodiments of Structural Formula VI:

Y is selected from the group consisting of fluorine, chlorine, bromine, iodine, tosyl, brosyl, mesyl, triphenylphosphine, sulfonates, sulfonamides, ammoniums, and phosphonates;
W is selected from the group consisting of hydrogen, hydroxy, methoxy, ethoxy, methyl, and ethyl;
V is selected from the group consisting of hydrogen, hydroxy, methoxy, ethoxy, methyl, and ethyl; and
U is selected from the group consisting of hydrogen, hydroxy, methoxy, ethoxy, methyl, and ethyl.

Certain embodiments of the present disclosure are directed to shikimate analogues as represented by Structural Formula VII below. The present disclosure also includes compositions and kits containing such analogues, and methods of use of such compounds and compositions as disclosed elsewhere herein.

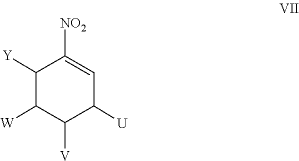

In non-limiting embodiments of Structural Formula VII:

Y is selected from the group consisting of fluorine, chlorine, bromine, iodine, tosyl, brosyl, mesyl, triphenylphosphine, sulfonates, sulfonamides, ammoniums, and phosphonates;
W is selected from the group consisting of hydrogen, hydroxy, methoxy, ethoxy, methyl, and ethyl;
V is selected from the group consisting of hydrogen, hydroxy, methoxy, ethoxy, methyl, and ethyl; and
U is selected from the group consisting of hydrogen, hydroxy, methoxy, ethoxy, methyl, and ethyl.

In certain embodiments of the compounds and compositions of the present disclosure, compounds having Structural Formula I as characterized in Table 1 (and equivalent compounds having Structural Formula III), and/or compounds having Structural Formula II as characterized in Table 2 (and equivalent compounds having Structural Formula IV) may optionally be excluded.

TABLE 1
Optional Shikimate Analogues - Structural Formula I

| | |
|---|---|
| (a) | X = O, R₁ = CH₃, R₂ is absent, R₃ = Cl, and R₄-R₆ = OH, |
| (b) | X = O, R₁ = CH₃, R₂ is absent, R₃ = OCH₃, and R₄-R₆ = OH, |
| (c) | X = O, R₁ = CH₃, R₂ is absent, R₃ = OH, and R₄-R₆ = OH, |
| (d) | X = O, R₁ = CH₃, R₂ is absent, R₃ = Cl, R₄ and R₅ = OH, and R₆ = [1-(methoxycarbonyl)ethenyl]oxy, |
| (e) | X = O, R₁ = CH₂CH₃, R₂ is absent, R₃ = Cl, and R₄-R₆ = H, |
| (f) | X = O, R₁ = CH₃, R₂ is absent, R₃ = Cl, R₄ = OH, and R₅ and R₆ = H, |
| (g) | X = O, R₁ = H, R₂ is absent, R₃ = F, and R₄-R₆ = OH, |
| (h) | X = O, R₁ = CH₃, R₂ is absent, R₃ = F, and R₄-R₆ = OH, |
| (i) | X = O, R₁ = CH₃, R₂ is absent, R₃ = F, R₄ and R₅ = OH, and R₆ = phenylmethoxy, |
| (j) | X = O, R₁ = CH₃, R₂ is absent, R₃ = Br, and R₄-R₆ = H, |
| (e) | X = O, R₁ = CH₂CH₃, R₂ is absent, R₃ = Br, and R₄-R₆ = H, |
| (f) | X = N, R₁ = methylethyl, R₂ = methylethyl, R₃ = Br, and R₄-R₆ = H, |
| (g) | X is absent, R₁ = H, R₂ is absent, R₃ = OH, and R₄-R₆ = H, |
| (h) | X is absent, R₁ = CH₃, R₂ is absent, R₃ = OH, and R₄-R₆ = H, |
| (i) | X = O, R₁ = H, R₂ is absent, R₃ = OH, and R₄-R₆ = H, |
| (j) | X is absent, R₁ = H, R₂ is absent, R₃ = OH, R₄ and R₆ = H, and R₅ = CH₃, |
| (k) | X = O, R₁ = CH₃, R₂ is absent, R₃ = OH, and R₄-R₆ = H, |
| (l) | X = O, R₁ = H, R₂ is absent, R₃ = OH, R₄ = OH, R₅ = H, and R₆ = H, |
| (m) | X = O, R₁ = H, R₂ is absent, R₃ = OH, R₄ and R₆ = H, and R₅ = CH₃, |
| (n) | X is absent, R₁ = H, R₂ is absent, R₃ = OH, R₄ and R₆ = H, and R₅ = 1-methylethenyl, |
| (o) | X is absent, R₁ = H, R₂ is absent, R₃ = OH, and R₄-R₆ = OH, |
| (p) | X = O, R₁ = CH₃, R₂ is absent, R₃ = OH, R₄ and R₆ = H, and R₅ = dimethyl |
| (q) | X = O, R₁ = CH₂CH₃, R₂ is absent, R₃ = OH, and R₄-R₆ = H, |
| (r) | X = S, R₁ = CH₂CH₃, R₂ is absent, R₃ = OH, and R₄-R₆ = H, |
| (s) | X is absent, R₁ = phenyl, R₂ is absent, R₃ = OH, and R₄-R₆ = H, |
| (t) | X = O, R₁ = CH₃, R₂ is absent, R₃ = OH, R₄ and R₆ = H, and R₅ = CH₃, |
| (u) | X = O, R₁ = CH₃, R₂ is absent, R₃ = OH, R₄ = OH, R₅ = H, and R₆ = H, |
| (v) | X is absent, R₁ = H, R₂ is absent, R₃ = OH, R₄ = OH, R₅ = 1-methylethenyl, and R₆ = H, |
| (w) | X = O, R₁ = CH₃, R₂ is absent, R₃ = OH, R₄ = H, R₅ = OH, and R₆ = H, |
| (x) | X = O, R₁ = H, R₂ is absent, R₃ = OH, R₄ = H, R₅ = OH, and R₆ = OH, |
| (y) | X = O, R₁ = dimethylethyl, R₂ is absent, R₃ = OH, and R₄-R₆ = H, |
| (z) | X = O, R₁ = CH₃, R₂ is absent, R₃ = OH, R₄ = H, R₅ = H, and R₆ = dimethyl, |
| (aa) | X = O, R₁ = CH₃, R₂ is absent, R₃ = OH, R₄ = H, R₅ = dimethyl, and R₆ = H, |
| (ab) | X = O, R₁ = CH₃, R₂ is absent, and R₃-R₆ = OH, |
| (ac) | X is absent, R₁ = 4-methylphenyl, R₂ is absent, R₃ = OH, and R₄-R₆ = H, |
| (ad) | X = O, R₁ = CH₃, R₂ is absent, R₃ = OH, R₄ = Br, R₅ = H, and R₆ = H, |
| (ae) | X = O, R₁ = CH₂CH₃, R₂ is absent, R₃ = OH, R₄ = H, R₅ = CH₃, and R₆ = H |
| (af) | X is absent, R₁ = 2-hydroxyphenyl, R₂ is absent, R₃ = OH, and R₄-R₆ = H, |
| (ag) | X = O, R₁ = CH₂CH₃, R₂ is absent, R₃ = OH, R₄ = OH, R₅ = H, and R₆ = H |
| (ah) | X = O, R₁ = CH₂CH₃, R₂ is absent, R₃ = OH, R₄ = H, R₅ = CH₃, and R₆ = H |
| (ai) | X = O, R₁ = CH₃, R₂ is absent, R₃ = OH, R₄ = OH, R₅ = OH, and R₆ = H |
| (aj) | X is absent, R₁ = 2-methylphenyl, R₂ is absent, R₃ = OH, and R₄-R₆ = H, |
| (ak) | X is absent, R₁ = 3-methylphenyl, R₂ is absent, R₃ = OH, and R₄-R₆ = H, |
| (al) | X is absent, R₁ = 2-naphthalenyl, R₂ is absent, R₃ = OH, and R₄-R₆ = H, |
| (am) | X = O, R₁ = CH₃, R₂ is absent, R₃ = OH, R₄ = H, R₅ = 1-methylethenyl, and R₆ = H, |
| (an) | X is absent, R₁ = 4-bromophenyl, R₂ is absent, R₃ = OH, and R₄-R₆ = H, |
| (ao) | X is absent, R₁ = 4-chlorophenyl, R₂ is absent, R₃ = OH, and R₄-R₆ = H, |
| (ap) | X is absent, R₁ = 4-fluorophenyl, R₂ is absent, R₃ = OH, and R₄-R₆ = H, |
| (aq) | X is absent, R₁ = 4-methoxyphenyl, R₂ is absent, R₃ = OH, and R₄-R₆ = H, |
| (ar) | X = O, R₁ = CH₃, R₂ is absent, R₃ = OH, R₄ = H, R₅ = OH, and R₆ = OH |
| (as) | X = O, R₁ = methylethyl, R₂ is absent, R₃ = OH, R₄ = OH, R₅ = H, and Re = H |
| (at) | X = N, R₁ = methylethyl, R₂ = methylethyl, R₃ = OH, and R₄-R₆ = H, |
| (au) | X = O, R₁ = CH₃, R₂ is absent, R₃ = OH, R₄ = OH, R₅ = 1-methylethenyl, and R₆ = H, |
| (av) | X = O, R₁ = propyl, R₂ is absent, R₃ = OH, R₄ = OH, R₅ = H, and Re = H |
| (aw) | X is absent, R₁ = 3-bromophenyl, R₂ is absent, R₃ = OH, and R₄-R₆ = H, |
| (ax) | X is absent, R₁ = 3-chlorophenyl, R₂ is absent, R₃ = OH, and R₄-R₆ = H, |
| (ay) | X is absent, R₁ = 2-bromophenyl, R₂ is absent, R₃ = OH, and R₄-R₆ = H, |
| (az) | X is absent, R₁ = 2-chlorophenyl, R₂ is absent, R₃ = OH, and R₄-R₆ = H, |
| (ba) | X = N, R₁ = methyl, R₂ = methoxy, R₃ = OH, and R₄-R₆ = H, |
| (bb) | X is absent, R₁ = 4-(dimethylamino)phenyl, R₂ is absent, R₃ = OH, and R₄-R₆ = H, |
| (be) | X = O, R₁ = CH₂CH₃, R₂ is absent, and R₃-R₆ = OH, |
| (bd) | X is absent, R₁ = 4-nitrophenyl, R₂ is absent, R₃ = OH, and R₄-R₆ = H, |
| (be) | X is absent, R₁ = 4-(trifluoromethyl)phenyl, R₂ is absent, R₃ = OH, and R₄-R₆ = H, |
| (bf) | X = O, R₁ = CH₃, R₂ is absent, R₃-R₅ = OH, and R₆ = NH₂, |
| (bg) | X = O, R₁ = CH₃, R₂ is absent, R₃-R₅ = OH, and R₆ = Cl, |
| (bh) | X = N, R₁ = phenylmethyl, R₂ = H, R₃ = OH, and R₄-R₆ = H, |
| (bi) | X = O, R₁ = CH₂CH₃, R₂ is absent, R₃ = (dimethoxyphosphinyl)oxy, and R₄-R₆ = H, |
| (bj) | X = O, R₁ = CH₂CH₃, R₂ is absent, R₃ = (diethoxyphosphinyl)oxy, and R₄-R₆ = H, |
| (bk) | X = O, R₁ = CH₂CH₃, R₂ is absent, R₃ = (diethoxyphosphinyl)oxy, R₄ = CH₃, R₅ = H, and R₆ = H, |
| (bl) | X = O, R₁ = CH₂CH₃, R₂ is absent, R₃ = (diethoxyphosphinyl)oxy, R₄ = H, R₅ = CH₃, and R₆ = H, |
| (bm) | X = O, R₁ = CH₂CH₃, R₂ is absent, R₃ = (diethoxyphosphinyl)oxy, R₄ = H, R₅ = H, and |
| | R₆ = dimethylethyl, |
| (bn) | X = O, R₁ = CH₂CH₃, R₂ is absent, R₃ = (diethoxyphosphinyl)oxy, R₄ = H, R₅ = H, and R₆ = phenyl, |
| (bo) | X is absent, R₁ = H, R₂ is absent, R₃ = methoxy, and R₄-R₆ = H, |
| (bp) | X = O, R₁ = H, R₂ is absent, R₃ = methoxy, and R₄-R₆ = H, |
| (bq) | X = O, R₁ = CH₃, R₂ is absent, R₃ = ethoxy, and R₄-R₆ = H, |
| (br) | X = O, R₁ = H, R₂ is absent, R₃ = ethoxy, and R₄-R₆ = H, |
| (bs) | X is absent, R₁ = CH₃, R₂ is absent, R₃ = acetyloxy, and R₄-R₆ = H, |
| (bt) | X = O, R₁ = CH₃, R₂ is absent, R₃ = acetyloxy, and R₄-R₆ = H, |
| (bu) | X = O, R₁ = CH₂CH₃, R₂ is absent, R₃ = acetyloxy, and R₄-R₆ = H, |
| (bv) | X = O, R₁ = H, R₂ is absent, R₃ = methoxy, R₄ = OH, R₅ = OH, and R₆ = H, |
| (bw) | X = O, R₁ = H, R₁ is absent, R₃ = methoxy, R₄ = H, R₅ = OH, and R₆ = OH, |
| (bx) | X = O, R₁ = dimethylethyl, R₂ is absent, R₃ = acetyloxy, and R₄-R₆ = H, |
| (by) | X = O, R₁ = CH₃, R₂ is absent, R₃ = acetyloxy, R₄ = H, R₅ = dimethyl, and R₆ = H, |
| (bz) | X = O, R₁ = CH₃, R₂ is absent, R₃ = 4-methoxyphenoxy, and R₄-R₆ = H, |
| (ca) | X = O, R₁ = CH₃, R₂ is absent, R₃ = acetyloxy, R₄ = Br, R₅ = H, and R₆ = H, |
| (cb) | X = O, R₁ = phenyl, R₂ is absent, R₃ = acetyloxy, and R₄-R₆ = H, |
| (cc) | X = N, R₁ = methylethyl, R₁ = methylethyl, R₃ = ethyl, and R₄-R₆ = H, |
| (cd) | X = O, R₁ = CH₃, R₂ is absent, R₃ = phenoxy, R₄ = H, R₅ = H, and R₆ = OH, |
| (ce) | X = O, R₁ = CH₃, R₂ is absent, R₃ = phenoxy, R₄ = OH, R₅ = H, and R₆ = H, |
| (cf) | X = O, R₁ = CH₃, R₂ is absent, R₃ = phenylmethoxy, R₄ = H, R₅ = OH, and R₆ = H, |
| (cg) | X = O, R₁ = CH₃, R₂ is absent, R₃ = acetyloxy, R₄ = H, R₅ = OH, and R₆ = OH, |
| (ch) | X = O, R₁ = CH₃, R₂ is absent, R₃ = (2,2,2-trichloroacetyl)oxy, and R₄-R₆ = H, |
| (ci) | X = O, R₁ = CH₃, R₂ is absent, R₃ = 4-methylphenoxy, R₄ = H, R₅ = H, and R₆ = OH, |
| (cj) | X = O, R₁ = CH₃, R₂ is absent, R₃ = 4-methoxyphenoxy, R₄ = H, R₅ = H, and R₆ = OH, |
| (ck) | X = O, R₁ = CH₃, R₂ is absent, R₃ = 4-methylphenoxy, R₄ = OH, R₅ = H, and R₆ = H, |
| (cl) | X is absent, R₁ = H R₂ is absent, R₃ = acetyloxy, R₄ = OH, R₅ = OH, and R₆ = acetyloxy, |
| (cm) | X = O, R₁ = CH₃, R₂ is absent, R₃ = H, R₄ = H, R₅ = OH, and R₆ = 4-methylphenoxy, |
| (cn) | X = O, R₁ = CH₃, R₂ is absent, R₃ = acetyloxy, R₄ = OH, R₅ = OH, and R₆ = OH, |
| (co) | X is absent, R₁ = H R₂ is absent, R₃ = isobutyroxy, R₄ = OH, R₅ = OH, and R₆ = OH, |
| (cp) | X = O, R₁ = CH₃, R₂ is absent, R₃ = 4-methoxyphenoxy, R₄ = OH, R₅ = H, and R₆ = H, |
| (cq) | X is absent, R₁ = H R₂ is absent, R₃ = butyroxy, R₄ = OH, R₅ = OH, and R₆ = OH, |
| (cr) | X = O, R₁ = CH₃, R₂ is absent, R₃ = methoxy, R₄ = acetyloxy, R₅ = acetyloxy, and R₆ = H, |
| (cs) | X = O, R₁ = CH₃, R₂ is absent, R₃ = 3,5-dimethylphenoxy, R₄ = H, R₅ = H, and R₆ = OH, |
| (ct) | X is absent, R₁ = H, R₂ is absent, R₃ = acetyloxy, R₄ = acetyloxy, R₅ = methylethenyl, and R₆ = H, |
| (cu) | X = O, R₁ = CH₃, R₂ is absent, R₃ = 3,5-dimethylphenoxy, R₄ = OH, R₅ = H, and R₆ = H, |
| (cv) | X = O, R₁ = CH₃, R₂ is absent, R₃ = 2,4-dimethylphenoxy, R₄ = H, R₅ = H, and R₆ = OH, |
| (cw) | X = O, R₁ = CH₃, R₂ is absent, R₃ = 2-naphthalenyloxy, R₄ = H, R₅ = H, and R₆ = OH, |
| (cx) | X = O, R₁ = CH₃, R₂ is absent, R₃ = 1-naphthalenyloxy, R₄ = H, R₅ = H, and R₆ = OH, |
| (cy) | X = O, R₁ = CH₃, R₂ is absent, R₃ = methoxy, R₄ = H, R₅ = acetyloxy, and R₆ = acetyloxy, |
| (cz) | X = O, R₁ = CH₃, R₂ is absent, R₃ = 2-naphthalenyloxy, R₄ = OH, R₅ = H, and R₆ = H, |
| (da) | X = O, R₁ = CH₃, R₂ is absent, R₃ = 1-naphthalenyloxy, R₄ = OH, R₅ = H, and R₆ = H, |
| (db) | X = O, R₁ = CH₃, R₂ is absent, R₃ = 1-naphthalenylmethoxy, R₄ = H, R₅ = OH, and R₆ = H, |
| (dc) | X is absent, R₁ = H, R₂ is absent, R₃ = pentanoyl, R₄ = OH, R₅ = OH, and R₆ = OH, |
| (dd) | X = O, R₁ = CH₃, R₂ is absent, R₃ = 4-(1,1-dimethylethyl)phenoxy, R₄ = H, R₅ = H, and R₆ = OH, |
| (de) | X = O, R₁ = CH₃, R₂ is absent, R₃ = acetyloxy, R₄ = H, R₅ = acetyloxy, and R₆ = acetyloxy, |
| (df) | X = O, R₁ = CH₃, R₂ is absent, R₃ = 4-(1,1-dimethylethyl)phenoxy, R₄ = OH, R₅ = H, and R₆ = H, |
| (dg) | X = O, R₁ = CH₃, R₂ is absent, R₃ = acetyloxy, R₄ = acetyloxy, R₅ = acetyloxy, and R₆ = acetyloxy, |
| (dh) | X = O, R₁ = CH₃, R₂ is absent, R₃ = 4-nitrophenoxy, R₄ = H, R₅ = H, and R₆ = OH, |
| (di) | X = O, R₁ = CH₃, R₂ is absent, R₃ = (4-methoxyphenyl)methoxy, R₄ = H, R₅ = NH₂, and R₆ = OH, |
| (dj) | X = O, R₁ = CH₃, R₂ is absent, R₃ = 4-nitrophenoxy, R₄ = OH, R₅ = H, and R₆ = H, |
| (dk) | X = O, R₁ = CH₃, R₂ is absent, R₃ = acetyloxy, R₄ = acetyloxy, R₅ = Br, and R₆ = Br, |
| (dl) | X = O, R₁ = H, R₂ is absent, R₃ = ethoxy, R₄ = OH, R₅ = acetylamino, and R₆ = OH, |
| (dm) | X is absent, R₁ = H, R₂ is absent, R₃ = azido, and R₄-R₆ = H, |
| (dn) | X = O, R₁ = H, R₂ is absent, R₃ = methylamino, and R₄-R₆ = H, |
| (do) | X = O, R₁ = CH₂CH₃, R₂ is absent, R₃ = NH₂, and R₄-R₆ = H, |
| (dp) | X = O, R₁ = H, R₂ is absent, R₃ = azido, and R₄-R₆ = H, |
| (dq) | X = O, R₁ = CH₃, R₂ is absent, R₃ = (1-methylethyl)amino, and R₄-R₆ = H, |
| (dr) | X = O, R₁ = CH₂CH₃, R₂ is absent, R₃ = methylamino, and R₄-R₆ = H, |
| (ds) | X = O, R₁ = CH₂CH₃, R₂ is absent, R₃ = dimethylamino, and R₄-R₆ = H, |
| (dt) | X = O, R₁ = CH₃, R₂ is absent, R₃ = 2-propen-1-ylamino, and R₄-R₆ = H, |
| (du) | X = O, R₁ = CH₂CH₃, R₂ is absent, R₃ = azido, and R₄-R₆ = H, |
| (dv) | X = O, R₁ = H, R₂ is absent, R₃ = NH₂, R₄ = OH, R₅ = OH, and R₆ = OH, |
| (dw) | X = O, R₁ = CH₂CH₃, R₂ is absent, R₃ = diethylamino, and R₄-R₆ = H, |
| (dx) | X = O, R₁ = H, R₂ is absent, R₃ = NO₂, R₄ = H, R₅ = NO₂, and R₆ = H, |
| (dy) | X = O, R₁ = CH₃, R₂ is absent, R₃ = phenylamino, and R₄-R₆ = H, |
| (dz) | X = O, R₁ = CH₃, R₂ is absent, R₃ = (phenylmethyl)amino, and R₄-R₆ = H, |
| (ea) | X = O, R₁ = CH₂CH₃, R₂ is absent, R₃ = azido, R₄ = CH₃, R₅ = H, and R₆ = H, |
| (eb) | X = O, R₁ = CH₃, R₂ is absent, R₃ = 4-morpholinyl, and R₄-R₆ = H, |
| (ec) | X = O, R₁ = CH₂CH₃, R₂ is absent, R₃ = azido, R₄ = H, R₅ = H, and R₆ = CH₃, |
| (ed) | X = O, R₁ = CH₂CH₃, R₂ is absent, R₃ = diethylamino, R₄ = H, R₅ = H, and R₆ = CH₃, |
| (ee) | X = O, R₁ = CH₂CH₃, R₂ is absent, R₃ = (phenylmethyl)amino, and R₄-R₆ = H, |
| (ef) | X = O, R₁ = CH₂CH₃, R₂ is absent, R₃ = NH₂, and R₄-R₆ = OH, |
| (eg) | X = O, R₁ = CH₃, R₂ is absent, R₃ = 4-methoxyphenyl)amino, and R₄-R₆ = H, |
| (eh) | X is absent, R₁ = phenyl, R₂ is absent, R₃ = (phenylmethyl)amino, and R₄-R₆ = H, |
| (ei) | X = O, R₁ = CH₃, R₂ is absent, R₃ = (4-chlorophenyl)amino, and R₄-R₆ = H, |
| (ej) | X = O, R₁ = CH₃, R₂ is absent, R₃ = (3-methoxyphenyl)amino, and R₄-R₆ = H, |
| (ek) | X = O, R₁ = CH₃, R₂ is absent, R₃ = [(4-methoxyphenyl)methyl]amino, and R₄-R₆ = H, |
| (el) | X = O, R₁ = CH₂CH₃, R₂ is absent, R₃ = (1-phenylethyl])amino, and R₄-R₆ = H, |
| (em) | X = O, R₁ = CH₃, R₂ is absent, R₃ = (3-bromophenyl)amino, and R₄-R₆ = H, |
| (en) | X = O, R₁ = CH₃, R₂ is absent, R₃ = (2-chlorophenyl)amino, and R₄-R₆ = H, |
| (eo) | X = O, R₁ = CH₃, R₂ is absent, R₃ = (2-bromophenyl)amino, and R₄-R₆ = H, |
| (ep) | X = O, R₁ = CH₃, R₂ is absent, R₃ = [4-(1,1-dimethylethyl)phenyl]amino, and R₄-R₆ = H, |
| (eq) | X = O, R₁ = CH₃, R₂ is absent, R₃ = 2,3-dihydro-1H-indol-1-yl, and R₄-R₆ = H, |
| (er) | X = O, R₁ = CH₂CH₃, R₂ is absent, R₃ = (phenylmethyl)amino, R₄ = H, R₅ = H, and R₆ = CH₃, |
| (es) | X = O, R₁ = CH₃, R₂ is absent, R₃ = [1,1′-biphenyl]-2-ylamino, and R₄-R₆ = H, |
| (et) | X = O, R₁ = CH₂CH₃, R₂ is absent, R₃ = diethylamino, R₄ = H, R₅ = H, and R₆ = phenyl, |
| (eu) | X = O, R₁ = CH₃, R₂ is absent, R₃ = (methoxycarbonyl)amino, R₄ = OH, R₅ = H, and R₆ = H, |
| (ev) | X = O, R₁ = CH₃, R₂ is absent, R₃ = (4-chlorophenyl)amino, R₄ = cyano, R₅ = H, and R₆ = H, |
| (ew) | X = O, R₁ = H, R₂ is absent, R₃ = NH₂, R₄ = acetylamino, R₅ = H, and R₆ = methylpropylamino, |
| (ex) | X is absent, R₁ = H, R₂ is absent, R₃ = acetylamino, R₄ = NH₂, R₅ = H, and R₆ = 1-ethylpropoxy, |
| (ey) | X = N, R₁ = phenylmethyl, R₂ = H, R₃ = (phenylmethyl)amino, and R₄-R₆ = H, |
| (ez) | X = O, R₁ = CH₂CH₃, R₂ is absent, R₃ = (phenylmethyl)amino, R₄ = phenyl, R₅ = H, and R₆ = H, |
| (fa) | X = O, R₁ = CH₃, R₂ is absent, R₃ = (4-chlorophenyl)amino, R₄ = cyano, R₅ = cyano, and R₆ = H, |
| (fb) | X = O, R₁ = CH₂CH₃, R₂ is absent, R₃ = [(1,1-dimethylethoxy)carbonyl]amino, and R₄-R₆ = OH, |
| (fc) | X = O, R₁ = H, R₂ is absent, R₃ = CN, R₄ = H, R₅ = H, and R₆ = CH₃, |
| (fd) | X = N, R₁ = methylethyl, R₂ = methylethyl, R₃ = nitrooxy, and R₄-R₆ = H, |
| (fe) | X = O, R₁ = CH₂CH₃, R₂ is absent, R₃ = [(1,1-dimethylethoxy)carbonyl]amino, R₄ = OH, R₅ = OH, |
| | and R₆ = Cl, |
| (ff) | X = O, R₁ = H, R₂ is absent, R₃ = OH, R₄ = H, R₅ = OH, and R₆ = (1-carboxyethenyl)oxy, |
| (fg) | X = O, R₁ = H, R₂ is absent, R₃ = OH, R₄ = OH, R₅ = OH, and R₆ = (1-carboxyethenyl)oxy, |
| (fh) | X = O, R₁ = H, R₂ is absent, R₃ = OH, R₄ = H, R₅ = NH₂, and R₆ = (1-carboxyethenyl)oxy, |
| (fi) | X = O, R₁ = CH₃, R₂ is absent, R₃-R₅ = OH, and R₆ = [1-(methoxycarbonyl)ethenyl]oxy, |
| (fj) | X = O, R₁ = CH₃, R₂ is absent, R₃-R₅ = acetyloxy, and R₆ = [1-(methoxycarbonyl)ethenyl]oxy, |
| (fk) | X = O, R₁ = CH₃, R₂ is absent, R₃ = OH, R₄ = H, R₅ = benzoyloxy, and R₆ = [1-(methoxycarbonyl) |
| | ethenyl]oxy, |
| (fl) | X = O, R₁ = H, R₂ is absent, R₃ = OH, R₄ = H, R₅ = [(1,1-dimethylethoxy)carbonyl]amino, and |
| | R₆ = (1-carboxyethenyl)oxy, |
| (fm) | X = O, R₁ = CH₃, R₂ is absent, R₃ = (4-methoxyphenyl)methoxy, R₄ = H, R₅ = benzoyloxy, and |
| | R₆ = [1-(methoxycarbonyl)ethenyl]oxy. |

TABLE 2
Optional Shikimate Analogues - Structural Formula II

| | |
|---|---|
| (a) | X = O, R₁ = CH₃, R₂ is absent, R₅ = OH, and R₆ = [1-(methoxycarbonyl)ethenyl]oxy, |
| (b) | X = O, R₁ = CH₃, R₂ is absent, R₅ = H, and R₆ = H, |
| (c) | X = O, R₁ = CH₃, R₂ is absent, R₅ = H, and R₆ = OH, |
| (d) | X = O, R₁ = H, R₂ is absent, R₅ = OH, and R₆ = OH, |
| (e) | X = O, R₁ = H, R₂ is absent, R₅ = NH₂, and R₆ = OH, |
| (f) | X = O, R₁ = CH₃, R₂ is absent, R₅ = OH, and R₆ = OH, |
| (g) | X = O, R₁ = H, R₂ is absent, R₅ = OH, and R₆ = (1-carboxyethenyl)oxy |

Certain embodiments of the present disclosure are directed to using the shikimate analogue compounds and compositions topically as treatments for removing, substituting, neutralizing, inhibiting, and/or attenuating various agents such as, but not limited to, odiferous (malodorous) compounds such as skunk mercaptans, and dermal irritants such as histamines caused by stinging nettle (e.g., Urtica dioica or U. urens), urushiols produced by poison ivy, poison, sumac, poison oak and similar species, that may be applied to (e.g., by accident) a subject's skin or other epithelial surface. The compounds or compositions may be applied to a subject's skin or other affected epithelial surface such as, but not limited to, eye, or respiratory epithelial surface (e.g., esophageal, lung, nasal, and/or sinus). In certain embodiments, the shikimate analogue may be stored, for example, as a dry powder or in a solution/suspension in water or alcohol (e.g., methanol, ethanol, isopropanol, or propanol), or a co-solvent mixture of water and alcohol, or other suitable mixture.

Sources of the dermal irritants which contain urushiol include, for example, members of the Anacardiaceae, such as the genus Toxicodendron (formerly known as Rhus). Examples of Toxicodendron species include, but are not limited to, T. pubescens, T. diversilobum, and T. rydbergii (poison oak species), T. radicans (poison ivy), T. vernix (poison sumac), and T. vernicifluum (Chinese (or Japanese) lacquer tree). Examples of urushiols include, but are not limited to, poison oak urushiol contains mostly catechols with 17 carbon side-chains (heptadecylcatechols), while poison ivy and poison sumac urushiols contain mostly 15 carbon side-chains (pentadecylcatechols).

The one or more shikimate analogue compounds may be delivered alone, simultaneously with, or in combination with, one or more secondary compounds as a combining, preparation, delivery and/or activating agent.

In certain embodiments of the compositions of the present disclosure, the one or more secondary compound(s) may be an organic base, an inorganic base, or an organic/inorganic base, for example an amine or a salt thereof, a pyridine or pyridinium, an imidazole or alkylated imidazole, or a pyrrole or alkylated pyrrole, or a combination thereof.

The amine may be an alkyl amine such as, for example, methyl amine, ethyl amine, dimethyl amine, diethyl amine, trimethyl amine, triethyl amine, diethanolamine, triethanolamine, and/or trimethylammonia, and combinations thereof.

The amine may be an acyclic or cyclic polyamine such as, for example, spermine, spermidine, tris(2-aminoethyl)amine, cyclen, cyclam, 1,4,7-triazacyclononane, 1,1,1-tris(aminomethyl)ethane, ethylenediamine, 1,4-diazabicyclo[2.2.2]octane (DABCO), diethylenetriamine, triethylenetetramine, 1,3-diaminopropane, putrescine, cadaverine, sym-norspermidine, sym-homospermidine, norspermine, thermospermine, carboxyspermidine, norcarboxyspermidine, caldopentamine, caldohexamine, ethylenediamine, 1,2-diaminopropane, 1,3-diaminopropane, N-methylethylenediamine, 1,4-diaminobutane, 3-(methylamino)propylamine, N,N′-dimethylethylenediamine, N-methyl-1,3-diaminopropane, 1-dimethylamino-2-propylamine, 3-(dimethylamino)-1-propylamine, N,N,N′,N′-tetramethyldiaminomethane, N,N,N′-trimethylethylenediamine, N-isopropylethylenediamine, N-propylethylenediamine, 2-(aminomethyl)-2-methyl-1,3-propanediamine, 1,2-diamino-5-bromo-3-chlorobenzene, 3,5-dichloro-1,2-diaminobenzene, 4-bromo-1,2-diaminobenzene, 4,5-dichloro-o-phenylenediamine, 4-chloro-1,3-diaminobenzene, 2-nitro-1,4-phenylenediamine, 3-nitro-1,2-phenylenediamine, 4-nitro-o-phenylenediamine, m-phenylenediamine, o-phenylenediamine, p-phenylenediamine, trans-4-cyclohexene-1,2-diamine, cis-4-cyclohexene-1,2-diamine, hexamethylenetetramine, 4-aminobenzylamine, N,N′-bis(2-aminoethyl)-1,3-propanediamine, methyl 3,4-diaminobenzoate, 1,2-diamino-3,5-dimethylbenzene, 4,5-dimethyl-1,2-phenylenediamine, 4-(2-aminoethyl)aniline, aniline, m-xylylenediamine, N-phenylethylenediamine, o-xylylenediamine, p-xylylenediamine, 1,8-diaminooctane, N,N-dimethyldipropylenetriamine, 1,2-bis(3-aminopropylamino)ethane, N-tosylethylenediamine, 2,2,4(2,4,4)-trimethyl-1,6-hexanediamine, 1,4-diaminonaphthalene, 1,5-diaminonaphthalene, 1,8-diaminonaphthalene, 4-tert-butyl-2,6-diaminoanisole, 2,2′-oxydianiline, 4,4′-oxydianiline, 3,3′-diaminobenzidine, 3,4′-diaminodiphenylmethane, 4,4′-diaminodiphenylmethane, 4,4′-ethylenedianiline, 2,4,6-triethyl-1,3,5-benzenetrimethanamine, and/or 1,8-anthracenedimethanamine.

The amine may be a monoamine, polyamine, and/or polymeric amine coated on or crosslinked with a polymer particle; or a monoamine, polyamine, and/or polymeric amine coated on or crosslinked with a polymeric film; or a monoamine, polyamine, and/or polymeric amine functionalized on or infused into a hydrogel; or a cyclic polyamine homopolymer and/or heteropolymer comprising at least one of polyvinylamine, polyethyleneimine, polyvicinalamine, polyamidoamine, polyallyamines, and polyetheramines.

The pyridine or pyridinium may be, for example, pyridine, alkylated pyridines, 2,6-lutidine, 2,4-lutidine, pyridazine, pyrimidine, pyrazine, 4-bromopyridine, 2,2′-bipyridine, 2,6-di-tert-butylpyridine, pyridine-2,6-dicarboxylic acid, pyridinium cations, and/or salts of the above.

In certain embodiments, the one or more secondary compounds may be, for example, sodium metasilicate, chlorhexidine, borates, zinc pyrithione, trimethyl barium hydroxide, 9-Azajulolidine, sodium iodide, potassium iodide, ammonia; ammonium hydroxide; tetramethylammonium hydroxide; tetrabutylammonium hydroxide; tetrabutylammonium hydroxide; sodium hydroxide; soda lime; potassium hydroxide; magnesium hydroxide; magnesium carbonate; calcium carbonate; aluminum hydroxide; sodium bicarbonate, sodium carbonate, potassium carbonate, potassium bicarbonate, or other carbonates or bicarbonates, choline; histamine; lysine; tris; and linear or branched dendrimers; calcium hydroxide, Lugol's Iodine, iodine tincture, povidone-iodine, benzalkonium chloride, cetrimonium bromide, Brilliant Green, triarylmethane dyes, Malachite green, octenidine dihydrochloride, phenoxyethanol, USP Tincture of Iodine, USP Strong Iodine Tincture, 1,3-dibromo-5,5-dimethylhydantoin (DBDMH), morpholine, and/or 3-(N-morpholino)propane sulfonic acid (MOPS), and combinations thereof.

In certain embodiments, the one or more secondary compounds may be, for example, an alcohols, alditol, glycol, polyol, saccharide, and/or polysaccharide, and combinations thereof.

The alcohols, alditols, glycols, polyols, saccharides, and polysaccharides may be, for example, cyclitol, acarviocin, aminocyclitol, bornesitol, ciceritol, conduritol, decahydroxycyclopentane, 5-deoxyinositol, dodecahydroxycyclohexane, ononitol, pinitol, pinpollitol, quebrachitol, theogallin, 3,4,5-tri-O-galloylquinic acid, inositol, inositol pentakisphosphate, cis-inositol, D-chiro-inositol, L-chiro-inositol, epi-inositol, neo-inositol, muco-inositol, neo-inositol, scyllo-inositol, sorbitol, threitol, arabitol, galactitol, iditol, volemitol, sorbitol, fucitol, xylitol, lactitol, erythritol, lactitol, maltitol, phytic acid, quinic acid, propylene glycol, 1,2-propanediol, ethylene glycol, low molecular weight polyethylene glycols (e.g., C2-C10), vegetable glycerine, dipropylene glycol, erythulose, glycerol, panthenol, arabinose, bis-HPPP, cellobiose, mannitol, mannose, glucose, allose, altrose, gulose, idose, lactose, maltose, dextrose, galactose, talose, psicose, fructose, sorbose, tagatose, β-d-ribopyranose, α-d-ribopyranose, β-d-ribofuranose, α-d-ribofuranose, sucrose, xylose, trehalose, cytosine glycol, cyclohexane-1,2-diol, aminomethanol, ethyleneglycol, 1,3-propanediol, 1,4-butanediol, 2,2-dimethyl-1-butanol, ethanol, propanol, butanol, pentanol, hexanol, ethynol, acetylenediol, fenticlor, fucitol, gluconic acid, glucic acid, 2-heptanol, 3-heptanol, 2-hexanol, 3-hexanol, ribitol, ethylhexylglycerin, octoxyglycerin, glucuronic acid, glyceraldehyde, glyceric acid, glycerol 3-phosphate, glycerol monostearate, 2-octanediol, pinacol, racemic acid, tartaric acid, uronic acid, xylosan, 1,2-butanediol, 1,3-butanediol, 1,4-butanediol, 2,3-butanediol, 2-methyl-2,4-pentanediol, neopentyl glycol, maltodextrins, raffinose, stachyose, fructo-oligosaccharides, amylose, amylopectin, starch, glycogen, cellulose, hemicellulose, methyl cellulose, methyl ethyl cellulose, pectins, hydrocolloids, sucralose, isomalt, isomaltooligosaccharide, isomaltulose, maltodextrin, and/or polydextrose, and combinations thereof.

In certain embodiments, the composition may comprise or be disposed in a carrier(s) or vehicle(s) such as, for example, sticks, soaps, bars of soap, balms, creams, pastes, gums, lotions, gels, foams, ointments, emulsions, suspensions, aqueous solutions, eye drops, aerosols, sprays, inhalants, body washes, face washes, rinses, oral tinctures, gel beads, pellets, cat litter, pet wipes, sheets, sachets, towelettes, breath strips, paper towels, napkins, sponges, sanitary napkins, tampons, baby diapers, adult diapers, undershirts and other items of clothing, electrically-activated deodorizing devices, air-drawn filtering devices. Non-limiting examples of formulations of such carriers and vehicles include, but are not limited to, those shown in “Remington, The Science and Practice of Pharmacy, 22nd ed., 2012, Edited by Loyd V. Allen, Jr”.

In certain embodiments, the compositions of the present disclosures may be applied topically on the surface of the affected skin area in adequate quantity and in the manner conventional in the relevant field. The composition for topical application may be in a solid, semi-solid, or liquid form. Suitable solid topical compositions include, for example, sticks or bars similar to deodorant sticks, or bars of soap. Suitable semi-solid mixtures topical compositions may include, for example, gels, lotions, pastes, balms, creams and ointments. Suitable liquid topical compositions include, for example, body or face washes, foams, rinses, and sprays. In at least one non-limiting embodiment, the at least one secondary compound may comprise a solid, semi-solid, or liquid soap mixture, including for example the ingredients propylene glycol, sodium stearate, glycerin, a surfactant (e.g., sodium laurate, sodium laureth sulfate, and/or sodium lauryl sulfate), and water, and optionally, sucrose, sorbitol, sodium chloride, stearic acid, lauric acid, aloe vera leaf extract, pentasodium penetrate, and/or tetrasodium etidronate.

Creams are emulsions of water in oil (w/o), or oil in water (o/w). O/w creams spread easily and do not leave the skin greasy and sticky. W/o creams tend to be more greasy and more emollient. Ointments are semi-solid preparations of hydrocarbons and the strong emollient effect makes it useful in cases of dry skin. The occlusive effect enhances penetration of the active agent and improves efficacy. Pastes are mixtures of powder and ointment. The addition of the powder improves porosity thus breathability. The addition of the powder to the ointment also increases consistency so the preparation is more difficult to rub off or contact non-affected areas of the skin. Lotions are liquid preparations in which inert or active medications are suspended or dissolved. For example, an o/w emulsion with a high water content gives the preparation a liquid consistency of a lotion. Most lotions are aqueous of hydroalcoholic systems wherein small amounts of alcohol are added to aid in solubilization of the active agent and to hasten evaporation of the solvent from the skin surface. Gels are transparent preparations containing cellulose ethers or carbomer in water, or a water-alcohol mixture. Gels liquefy on contact with the skin, dry, and leave a thin film of active medication.

A person with ordinary skill in the art will be capable of determining the effective amount of the composition needed for a particular treatment. Such amount may depend on the strength of the composition or extent of the epithelial condition. Although a person with ordinary skill in the art will know how to select a treatment regimen for a specific condition. In a non-limiting example, a dosage of the composition comprising about 0.01 mg to about 1000 mg of the active agent (shikimate analogue compound) per ml may be applied 1 to 2 to 3 to 4 to 5 to 6 times per day or more to the affected area. It is foreseeable in some embodiments that the composition is administered over a period of time. The composition may be applied for a day, multiple days, a week, multiple weeks, a month, or even multiple months in severe circumstances. Alternatively, the composition may be applied only once when the skin condition is mild.

The composition may comprise the active agent (i.e., the shikimate analogue) in a concentration of, but is not limited to, 0.0001 M to 1 M, for example, or 0.001 M to 0.1 M. The composition may comprise about 0.01 to about 1000 milligrams of the active agent (compound) per ml of at least one secondary compound with which the active agent is combined in a composition or mixture. The composition may comprise about 1 wt % to about 90 wt % (or 1 mass % to about 90 mass %) of one or more shikimate analogues and about 10 wt % to about 99 wt % (or 10 mass % to about 99 mass %) of one or more secondary compounds (where “wt %” is defined as the percentage by weight of a particular compound in a solid or liquid composition, and “mass %” is defined as the percentage by mass of a particular compound in a solid or liquid composition).

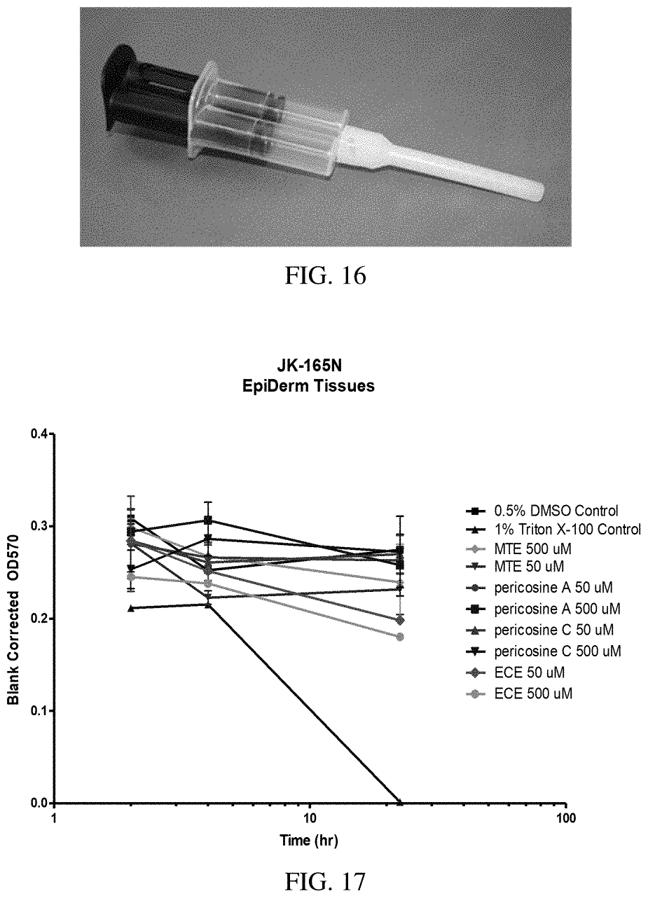

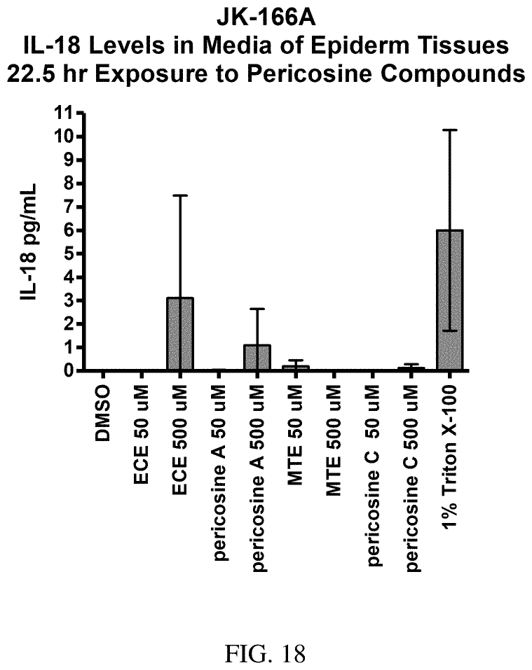

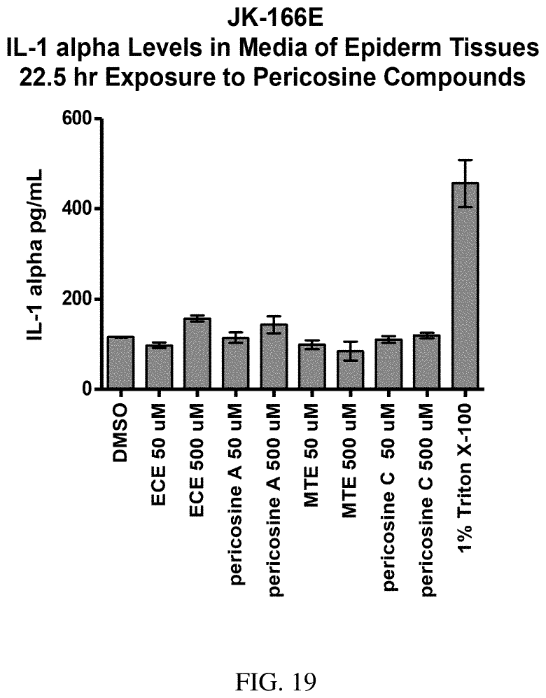

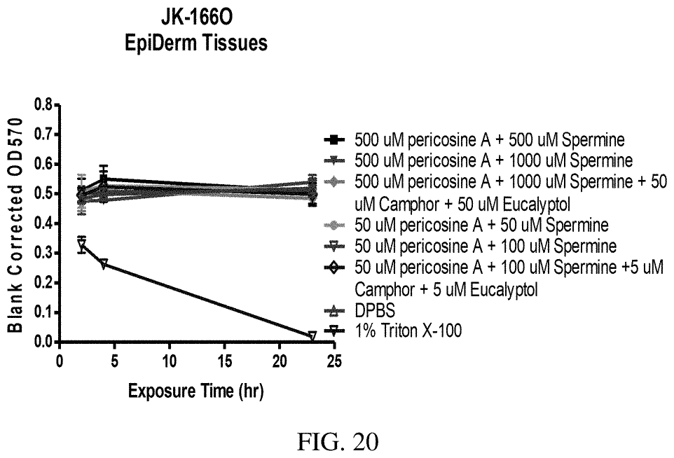

The shikimate analogue compound may be stored separately from the one or more secondary compounds (such as listed above) in a kit before being combined into a composition, and mixed as required at a point of use, for example using a dual syringe or applicator system (such as shown in FIG. 16) or another comparable delivery device as discussed in further detail below. In one embodiment of a single-use device of the present disclosure, the syringe may be constructed of a single cylinder which contains two or more compartments, including one compartment containing the shikimate analogue and one compartment containing the secondary compound which are arranged such that when a piston of the syringe is compressed in the cylinder, the contents of the at least two compartments are combined to form a mixture (composition) comprising the shikimate analogue and the secondary compound. The mixture is then applied to the epithelial surface to treat the epithelial condition.

For severe cases of allergic contact dermatitis, the shikimate analogue compound can be administered topically and/or concomitantly in a systemic oral, parenteral, intraperitoneal, or sublingual preparation. For example, it can be administered via ingestion of a food substance containing the compound in an amount sufficient to achieve therapeutic levels. Alternatively, it can be enclosed in capsules, compressed into tablets, microencapsulated, entrapped in liposomes, in solution or suspension, alone or in combination with a substrate immobilizing material such as starch or salts.

Pharmaceutically compatible binding agents and/or adjuvant materials can be used as part of a composition. Tablets or capsules can contain any of the following ingredients, or compounds of similar nature: a binder such as microcrystalline cellulose, gum tragacanth or gelatin; an excipient such as starch or lactose; an integrating agent such as alginic acid; corn starch; a lubricant such as magnesium stearate; a glidant such as colloidal silicon dioxide; and additional sweetening and flavoring agents. When a capsule form is used, a liquid carrier such as a fatty oil may be used. Capsules and tablets can be coated with sugar, shellac and other enteric agents as is known, or in a controlled-release formulation. The topical compositions may be formulated with liquid or solid emollients, solvents, thickeners, or humectants. Emollients include, but are not limited to, stearyl alcohol, mink oil, cetyl alcohol, oleyl alcohol, isopropyl laurate, polyethylene glycol, olive oil, petroleum jelly, palmitic acid, oleic acid, and myristyl myristate. Emollients may also include natural butters extracted from various plants, trees, roots, or seeds. Examples of such butters include, but are not limited to, shea butter, cocoa butter, avocado butter, aloe butter, coffee butter, mango butter, or combination thereof.

Suitable materials which may be used in the compositions as secondary compounds, carriers, vehicles, or solvents include, without limitation, propylene glycol, ethyl alcohol, isopropanol, acetone, diethylene glycol, ethylene glycol, dimethyl sulfoxide, and dimethyl formamide. Suitable humectants include, but are not limited to, acetyl arginine, algae extract, aloe barbadensis leaf extract, 2,3-butanediol, chitosan lauroyl glycinate, diglycereth-7 malate, diglycerin, diglycol guanidine succinate, erythritol, fructose, glucose, glycerin, honey, hydrolyzed wheat protein/polyethylene glycol-20 acetate copolymer, hydroxypropyltrimonium hyaluronate, inositol, lactitol, maltitol, maltose, mannitol, mannose, methoxypolyethylene glycol, myristamidobutyl guanidine acetate, polyglyceryl sorbitol, potassium pyrollidone carboxylic acid (PCA), propylene glycol (PGA), sodium pyrollidone carboxylic acid (PCA), sorbitol, and sucrose. Other humectants may be used for yet additional embodiments of the compositions of the present disclosure.

Suitable thickeners include, but are not limited to, polysaccharides, in particular xantham gum, guar-guar, agar-agar, alginates, carboxymethylcellulose, relatively high molecular weight polyethylene glycol mono- and diesters of fatty acids, polyacrylates, polyvinyl alcohol and polyvinylpyrrolidone, surfactants such as, for example, ethoxylated fatty acid glycerides, esters of fatty acids with polyols such as, for example, pentaerythritol or trimethylpropane, fatty alcohol ethoxylates or alkyl oligoglucosides, and electrolytes, such as sodium chloride and ammonium chloride.

The compositions may further comprise one or more penetrants, compounds facilitating penetration of active ingredients into the skin of a patient. Non-limiting examples of suitable penetrants include isopropanol, polyoxyethylene ethers, terpenes, cis-fatty acids (oleic acid, palmitoleic acid), acetone, laurocapram dimethyl sulfoxide, 2-pyrrolidone, oleyl alcohol, glyceryl-3-stearate, cholesterol, myristic acid isopropyl ester, and propylene glycol. Additionally, the compositions may include surfactants or emulsifiers for forming emulsions. Either a water-in-oil or oil-in-water emulsion may be formulated. Examples of suitable emulsifiers include, but are not limited to, stearic acid, cetyl alcohol, PEG-100, stearate and glyceryl stearate, cetearyl glucoside, polysorbate 20, methylcellulose, sodium carboxymethylcellulose, glycerin, bentonite, ceteareth-20, cetyl alcohol, cetearyl alcohol, lanolin alcohol, riconyl alcohol, self-emulsifying wax (e.g., Lipowax P), cetyl palmitate, stearyl alcohol, lecithin, hydrogenated lecithin, steareth-2, steareth-20, and polyglyceryl-2 stearate.

In some formulations, such as in aerosol form, the composition may also include a propellant. Preferably, hydrofluoroalkanes (HFA) such as either HFA 134a (1,1,1,2-tetrafluoroethane) or HFA 227 (1,1,1,2,3,3,3-heptafluoropropane) or combinations of the two, may be used since they are widely used in medical applications. Other suitable propellants include, but are not limited to, mixtures of volatile hydrocarbons, typically propane, n-butane and isobutane, dimethyl ether (DME), methylethyl ether, nitrous oxide, and carbon dioxide. Those skilled in the art will readily appreciate that emollients, solvents, thickeners, humectants, penetrants, surfactants or emulsifiers, and propellants, other than those listed may also be employed.

The compositions of the present disclosure may also be administered orally either in solid or a liquid form. For oral administration, the compositions may be presented in the form of tablets, lozenges, gums such as chewing gums, pills, capsules, elixirs, powders, lyophilized powders, solutions, granules, suspensions, emulsions, syrups, and tinctures. Conventionally known methods may be used to prepare the composition in different forms.

Solid forms for oral administration may contain binders acceptable in human and veterinary pharmaceutical practice, sweeteners, disintegrating agents, diluents, flavorings, coating agents, preservatives, lubricants and/or time delay agents. Suitable binders include, but are not limited to gum acacia, gelatin, corn starch, gum tragacanth, sodium alginate, carboxymethylcellulose or polyethylene glycol. Suitable sweeteners include, but are not limited to, sucrose, lactose, glucose, aspartame, or saccharin. Suitable disintegrating agents include, but are not limited to, corn starch, methylcellulose, polyvinylpyrrolidone, guar gum, xanthan gum, bentonite, alginic acid or agar. Suitable diluents include, but are not limited to, lactose, sorbitol, mannitol, dextrose, kaolin, cellulose, calcium carbonate, calcium silicate or dicalcium phosphate. Suitable flavoring agents include, but are not limited to, peppermint oil, oil of wintergreen, cherry, orange or raspberry flavoring. Suitable coating agents include, but are not limited to, polymers or copolymers of acrylic acid and/or methacrylic acid and/or their esters, waxes, fatty alcohols, zein, shellac, or gluten. Suitable preservatives include, but are not limited to, sodium benzoate, vitamin E, alpha-tocopherol, ascorbic acid, methyl paraben, propyl paraben or sodium bisulphite. Suitable lubricants include, but are not limited to, magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include, but are not limited to, water, oils such as olive oil, peanut oil, sesame oil, sunflower oil, safflower oil, coconut oil, liquid paraffin, ethylene glycol, propylene glycol, polyethylene glycol, ethanol, propanol, isopropanol, glycerol, fatty alcohols, triglycerides or mixtures thereof. In one embodiment, the composition is in the tincture form. Tinctures are herbal extracts. They may be prepared by using solvents to extract oils from herbs by either percolation or maceration techniques. Suitable solvents for forming tinctures may include, but are not limited to, water, glycerin, propylene glycol, alcohol, vegetable oil, mineral oil, or combinations thereof. Processes for preparing tinctures are well known in the art and are disclosed, for example, in U.S. Pat. Nos. 4,952,603, 6,555,074 and 6,656,437, which are expressly incorporated herein by reference in their entirety.

In certain embodiments of the present disclosure, it may be desirable to extend the shelf life of the shikimate analogue(s) by maintaining it in a stable, chemically unaltered, or substantially inert form prior to its use. In such cases, the shikimate analogue (e.g., pericosine(s)) can be packaged or stored in a device, kit, or system that sequesters or compartmentalizes the shikimate analogue apart from at least one secondary compound which initiates or enhances the activity of the shikimate analogue. The sequestered secondary compound may be any compound described elsewhere herein as a secondary compound. The shikimate analogue and the sequestered secondary compound are thus maintained separately in the system, device, or kit prior to their combination for use or delivery. The shikimate analogue may be in a shikimate composition containing a secondary compound which is different from the sequestered secondary compound. In certain embodiments, the shikimate analogue (or shikimate composition), also referred to herein, for convenience, as component A, is stored in a bag, container, receptacle, compartment, or chamber allowing for its physical separation from the sequestered secondary compound, also referred to herein, for convenience, as component B, which may also be stored in a bag, container, receptacle, compartment, or chamber.

In certain embodiments, components A and B are within a common container, but are separated by an impermeable membrane or other physical barrier that can be readily broken to allow mixing of the two components, to cause activation of the shikimate analogue. Thus, the present disclosure encompasses use of any system, device, or kit in which components A and B are physically separated by any effective barrier such as a membrane or wall between the two components. In various embodiments, the shelf life of the shikimate analogue can be extended to, for example, 15 years or longer. In various embodiments the shelf life of the shikimate analogue stored in the system, device, or kit can be 30 minutes, to 1, 2, 3, 4, 5, 6, 7, 8, 9, 10, 11, 12, 13, 14, 15, 16, 17, 18, 19, 20, 21, 22, 23, 24, 30, 36, 42, or 48 hours, to 3, 4, 5, 6, 7, 8, 9, 10, 11, 12, 13, 14, 15, 16, 17, 18, 19, 20, 21, 22, 23, 24, 30, 36, 42, 48, 56, 64, 72, 80, 90, 100, 120, 150, 180, 210, 240, 270, 300, 330, or 360 days, to 1.5, 2, 3, 4, 5, 6, 7, 8, 9, 10, 11, 12, 13, 14, or 15 or more years.

Non-limiting examples of such systems, devices, and kits that utilize barrier to separate components A and B can be similar to (a) “instant cold packs” (wherein components are separated using a bag within a bag; the barrier provided by the inner bag is broken allowing for the mixing of its contents), such as shown in U.S. Pat. Nos. 5,723,002 and 6,438,965, (b) “glow sticks” (wherein components are separated within an inner chamber, which is broken to allow the mixing of components) such as shown in U.S. Pat. No. 3,933,118, and (c) dual-chamber or multi-chamber aerosol dispensing or dispersal, and related devices and kits such as shown in U.S. Pat. Nos. 3,366,279, 6,732,887, 5,409,141, US patent publications 2012/0031925 and 2007/0233012, and European patent document EP0424068A2. In one embodiment, the device is a small bag, pouch, packet, or sachet designed for a single use, wherein the inner space of the device contains either component A or B as a first component, and a smaller, crushable bag, pouch, packet, or sachet containing the second component is disposed within the inner space of the device which holds the first component. When the device is compressed, the crushable bag, pouch, packet, or sachet containing the second component bursts open, releasing the second component into the first component, which causes the two components to mix, thereby causing activation of the shikimate analogue. The device can then be opened and the contents applied to the desired treatment surface, for example by squeezing the contents of the bag, pouch, packet, or sachet onto the surface.

In various embodiments of the devices disclosed herein, the mixing of components A and B may take place external to the system, device, or kit or mixing may occur within the system, device, or kit, e.g., as described for the bag embodiment. In cases of systems, devices, or kits which utilize external mixing of components A and B, the mixing of the components may occur within a separate bag, container, compartment, receptacle, or chamber, during the delivery or application process, or mixing may occur directly on the treatment surface. For example, in one embodiment, the system, device, or kit comprises two separate bags, pouches, packets, or sachets which are connected or unconnected. One of the bags, pouches, packets, or sachets contains component A and the other component B. When the contents of the two separate bags, pouches, packets, or sachets are squeezed onto the surface to be treated they may combine and mix as the squeezing process occurs, or they may be mixed by the user after they have been squeezed onto the treatment surface. When components A and B are mixed within the system, device, or kit, the mixture must be expelled or released therefrom in order to be applied to the treatment surface. As noted elsewhere herein, in certain embodiments, the system, device, or kit contains a propulsive gas which causes expulsion of the mixture therefrom, such as is used in an aerosol can. In certain embodiments, the system, device, or kit includes a two-compartment sprayer such as a spray bottle, can, or container, wherein component A is contained within one compartment and component B is contained within the second compartment. When the sprayer is activated (e.g., by pressing a nozzle or hand-pumping the spayer), portions of components A and B are either drawn into a common chamber where they are mixed before being sprayed, or they are mixed as they are being sprayed, for example onto the treatment surface or into an atmosphere. The spray device (such as the aerosol can or a bottle with a hand-pumped sprayer) may be a single use or multiple use device.

An alternative method of applying the shikimate analogue compounds or compositions of the present disclosure is by using a “wet wipe.” Such an applicator has the added convenience of portability since such wipes are typically provided in a tear-open foil or pouch container. The container can include a single wipe or multiple wipes for added convenience, particularly if, in the latter case, the container can be closed or resealed. “Wet wipes” are well known in the art and are used to provide various ingredients for application to the skin, for example, sun screens, moisturizers, insect repellants, lotions for dry skin, and lubricants for shaving. The wipes are typically treated cloths and comprise materials such as cellulosic fibrous sheet, non-woven fabric or porous sheet that is saturated with a compound or composition described herein. Useful materials include paper, air-laid and non-woven webs, melt blown, spun-bonded and spun-lace webs as well as foam sheets. Techniques for moistening the wipes and packaging them in moisture impervious packages are well known in the art and need not be described herein.

Alternatively, treated sheets, tissues, cloths or articles comprising the composition of the present disclosure can be delivered from a sequential dispenser, in which articles are provided as individual interleaved or detachably-connected (e.g., perforated) sheets and can pop-up from the dispenser when the preceding article is removed. Suitable containers can include a closure or lid for the sheet dispenser opening in order to reduce the loss of liquid by evaporation or otherwise. Dispensers for such articles typically have a box-like shape. The dispenser has an opening, typically at the top, through which individual articles or sheets are removed by the user. In one embodiment the shikimate analogue is embedded in a sheet or wrap which also contains the secondary compound in degradable beads. When the sheet or wrap is applied to the skin for example, and rubbed thereon, the beads burst, releasing the secondary compound which is mixed with the shikimate analogue and enhances its neutralizing effectiveness.

In alternate embodiments, the shikimate analogues can be contained in or on or delivered via odor neutralizing gel beads, sprays, pellets, cat litter, atomizers, foggers, sachets, towelettes, breath strips, and pet wipes.

In another aspect, a kit for treating skin conditions is provided. According to one embodiment, the kit comprises a container containing a composition comprising the shikimate analogue and/or the at least one secondary component. The kit (which may be an applicator) may comprise two containers or more. A person skilled in the art will be able to select a container based on the form of the composition and its intended use. For example, an aerosol spray may be supplied in a pressurized can or in a hand-pumped sprayer, wherein the shikimate analogue is contained within the can separately from the at least one secondary compound and the two compounds are combined as they are sprayed from the spray can or spray bottle. In at least one embodiment the first container of the kit contains the shikimate analogue with or without a solvent such as water and the second container contains the one or more secondary compounds described elsewhere herein in any suitable combination. A lotion may be provided in a plastic bottle. In some embodiments of the kit, an applicator, such as a gauze, a cotton swab or a brush, may also be included.

Any of the systems, devices, or kits of the present disclosure may be supplied with a set of instructions for use. The set of instructions preferably includes information necessary for proper use of the system, device, or kit, such as mixing instructions and/or dosage and timing of administration of the composition disclosed herein. The set of instruction may comprise instructions on treating any of the skin conditions or disease described herein including but not limited to rashes, blisters, contact dermatitis caused by urushiol and/or stinging nettle toxins, infections, burns, insect bites, microbial or bacterial infections, sunburn, scabies, scrapes, cuts, surgical incisions, skin irritations, chapped lips, cracked skin, and skin odors caused by spraying by skunks and other mustelids, and combinations thereof. A person of ordinary skill in the art will appreciate that the set of instructions can be in any suitable medium, including, without limitation, printed, video-taped, digital, and audio-recorded. The kit may provide a practitioner with tools necessary to treat skin having the condition to be treated. These methods comprise administering an effective amount of composition as described above to the affected epithelial surface. Skin conditions that can be treated by these methods include, but are not limited to, rashes and blisters due to contact dermatitis caused by urushiol and/or stinging nettle toxins, acne, fungal infections such as athlete's foot, ringworm, burns, insect bites, microbial or bacterial infections, sunburn, scabies, scrapes, cuts, surgical incisions, skin irritations, chapped lips, cracked skin, and skin odors caused by spraying by skunks and other mustelids, and combinations thereof.

General Experimental Methods

Optical rotations were measured on a Rudolph Research Autopol III automatic polarimeter. UV data were measured with a Hewlett Packard 8452A diode array spectrophotometer. IR spectra were measured on a Shimadzu IRAffinity FTIR spectrometer. NMR data were obtained on Varian VNMR spectrometers (400 and 500 MHz for ¹H, 100 and 125 MHz for ¹³C) with broad band and triple resonance probes at 25±0.5° C. Electrospray-ionization mass spectrometry and the UPLC-HRESIMSMS data were collected on an Agilent 6538 high-mass-resolution QTOF mass spectrometer. LAESIMS spectrometry data were collected on a Thermo LTQ XL™ Linear Ion Trap Mass Spectrometer equipped with a Protea LAESI DP-1000 system. Preparative HPLC separations were performed on a Shimadzu system using a SCL-10A VP controller and a Gemini 5 μm C₁₈ column (110 Å, 250×21.2 mm) with flow rate of 10 mL/min. Semi-preparative HPLC separations were performed on a Waters 1525 system using a 2998 PDA detector and Luna 5 μm C₁₈ columns (110 Å, 250×10.0 mm) with flow rate of 4 mL/min. The experimental VCD spectrum was measured in DMSO-d₆ with a ChiralJR-2X VCD spectrometer (Biotools, Inc.). The experimental ECD spectrum was measured with a model 202-01 AVIV circular dichroism spectrometer. All solvents were of ACS grade or better.

Strain Information

The isolation and identification of the Tolypocladium sp. MEA-2 (GenBank accession KC840044) and Tolypocladium sp. Sup5-1 (GenBank accession KJ571609) were previously described. Fungal isolates from our lab were identified based on the sequence data generated for the ribosomal internal transcribed spacer region and the 5.8S rRNA gene (ITS1-5.8S-ITS2). The sources of other fungal strains used in this study are listed in Table S17 of the appendix of U.S. Provisional Patent Application 62/295,343. All strains were stored in 20% glycerol at −80° C. The fungi were recovered on plates with potato dextrose agar (PDA) (10 g/L Great Value® mashed potatoes, 5 g/L glucose, 15 g/L agar).

Isomerization of Compound 1

A DMSO-d₆ solution (500 μL) of 1 (15 mg) was applied for VCD analysis. Isomerization of 1 took place under the VCD experimental condition. The new isomer 2 (4.5 mg) was purified using semi-preparative reversed-phase HPLC (Luna 5 μm, C18, 110 Å, 250×10.00 mm, 40% MeCN in H₂O, 4.0 mL/min).

Cleavage Reaction of ¹³C-Labeled 1

¹³C-labeled 1 (20 mg) derived from feeding with [U-¹³C6]-D-glucose was stirred in 2 mL MeOH with 50 mg CuSO₄ and 10 mg Zn powder at 45° C. overnight. The resulting mixture was passed through a small C18 column and further purified using semi-preparative reversed-phase HPLC (Luna 5 μm, C₁₈, 110 Å, 250×10.00 mm, 10% MeCN in H₂O, 4.0 mL/min) to yield ¹³C-labeled 3 (3.4 mg). (+)-Pericosine C (3): colorless solid; [α]²⁰_{D} 94 (c 0.2, MeOH); CD (MeOH) λₘₐₓ (Δε) 219 (9.9), 251 (−2.2).

Fungal Co-Culture

Fungi for co-culture studies were inoculated in 200 mL PDB medium (10 g/L Great Value® mashed potatoes, 5 g/L D-glucose) at room temperature on a rotary shaker (130 rpm) for 4 days. For co-culture experiments, Tolypocladium sp. T1 (100 mL) culture broth was mixed separately with the broth of the co-culture fungus (100 mL) in autoclaved flasks. The co-culture mixtures were further grown at room temperature on a rotary shaker (130 rpm) for 4 days. The co-culture broths were extracted with 400 ml ethyl acetate and the total organic extract were analyzed by UPLC-HRESIMSMS. The large-scale co-culture fermentation of Tolypocladium sp. T1 with Penicillium sp. P1 or Penicillium sp. P2 was performed as described at a 5 L scale.

Detection of Secondary Metabolites from Fermentation Broth with LAESIMS

Fungal fermentation broth (10 μL) was loaded onto a Protea® 96-well dimple plate and submitted for LAESIMS analysis. The electrospray was set with the high-voltage at 3500 V using 50% MeOH-0.1% formic acid as the spray solvent. The flow rate was 1 μL/min. Each well was analyzed with thirty laser pulses applied at 75% energy. The chromatograms and MS spectra were visualized with Thermo Xcalibur software. Heat maps of detected ions were generated using Protea Plot software.

Purification of Compounds 4, 6, and 7 from the Co-Culture

Five liters of Tolypocladium sp. T1/Penicillium sp. P1 co-culture broth was extracted three times with EtOAc. The combined crude extract (1.5 g) was subjected to HP20SS vacuum column chromatography (eluted with gradients of 30%, 50%, 70%, 90%, and 100% MeOH in H₂O) to generate five fractions. Fractions Fr. 3 and 4 were combined and further separated by prep-HPLC (eluted with 70%-100% MeOH in H₂O) to give 9 fractions. Fraction Fr. 3-3-4 was further purified by semi-prep HPLC (eluted with 55% MeCN in H₂O) to yield compound 4 (22.5 mg). Compound 4 underwent isomerization in DMSO-d₆ during NMR analysis. The equilibrium mixture was separated using semi-prep HPLC (eluted with 75% MeOH) to provide pure compounds 4 (16.5 mg) and 5 (3.7 mg). Both compounds were stabilized in CDCl₃ for NMR analysis.

Five liters of Tolypocladium sp. T1/Penicillium sp. P2 co-culture broth was extracted with EtOAc for three times. The combined crude extract (2.0 g) was subjected to HP20SS vacuum column chromatography (eluted with 30%, 70%, and 100% step gradients of MeOH in H₂O), which provided three fractions. Fraction Fr. 2 was purified by semi-prep HPLC (eluted with 30%-100% MeCN in 0.1% formic acid) to yield compounds 6 (4.0 mg) and 7 (17.0 mg).

Purification of compounds 10 and 11 from Tolypocladium sp. T1

Spores were inoculated into 60 Erlenmeyer flasks (1 L) containing 200 mL PDB broth. The flasks were shaken at 135 rpm for 8 days at room temperature on an Innova 5000 shaker. The combined culture broth and mycelium was successively extracted with EtOAc and n-BuOH. The EtOAc extract (2.4 g) was separated into five fractions (1-5) by HP20ss column chromatography (eluted with a gradient of MeOH—H₂O). Fractions Fr. 1 and 2 eluted with 10% and 30% MeOH in H₂O were combined and further separated by semi-prep reversed-phase HPLC (Gemini 5 μm, C18, 110 Å, 250×10.0 mm, 5%-30% MeCN in 0.1% formic acid, 4.0 mL/min) to give 10 subfractions. Subfraction 2 was further subjected to semi-preparative reversed-phase HPLC (Gemini 5 μm, C₁₈, 110 Å, 250×10.00 mm, 10% MeOH in H₂O, 4.0 mL/min) to yield 10 (51 mg). The n-BuOH extract (9.1 g) was subjected to HP20SS vacuum column chromatography (eluted with gradients of 5%, 10%, 20%, 30%, and 100% MeOH in H₂O) to generate four fractions. Fractions Fr. 2-4 were combined and purified by semi-prep HPLC (eluted with 10% MeCN) to produce 11 (6.6 mg).

Small-Scale Nucleophilic/Toxin Treatment Experiments

Aliquots (200 μL) of Tolypocladium sp. T1 culture broth from a 200 mL culture (8 day) were incubated with 1 mM of each nucleophilic precursors at room temperature overnight. The broths were evaporated in vacuo and the residues were redissolved in 90% MeOH for FPLC-ESIMS analysis.

Purification of Compound 14 from the Ciclopirox Treatment Experiment

Flasks containing Tolypocladium sp. T1 cultures (200 mL×10 flask, 8 day) were incubated with 50 mg ciclopirox olamine (13) at room temperature overnight. The broth was extracted with equal volumes of EtOAc (×3). The combined crude extract (0.8 g) was subjected to HP20SS vacuum column chromatography (eluted with a step gradient consisting of 30%, 50%, 70%, and 100% MeOH in H₂O) to generate four fractions. Fraction Fr. 3 was purified by semi-prep HPLC (eluted with 50%-100% MeOH) to provide compound 14 (50.0 mg).

Purification of Compound 16 from the SAHA Treatment Experiment

Flasks containing Tolypocladium sp. T1 cultures (200 mL×8 flask, 8 day) were incubated with 200 mg suberanilohydroxamic acid (SAHA, 15) at room temperature overnight. The broth was extracted with equal volumes of EtOAc (×3). The combined crude extract (0.9 g) was subjected to HP20SS vacuum column chromatography (eluted with a step gradient consisting of 30%, 50%, 70%, and 100% MeOH in H₂O) to generate four fractions. Fractions Fr. 2 and 3 were combined and further purified by semi-prep HPLC (eluted with 40%-100% MeOH in 0.1% formic acid) to yield compound 16 (68.0 mg).

Purification of compound 18 from the anisomycin treatment experiment

Flasks containing Tolypocladium sp. T1 cultures (200 mL×10 flask, 8 day) were incubated with 25 mg anisomycin (17) at room temperature overnight. The broth was extracted with equal volumes of EtOAc (×3). The combined crude extract (1.2 g) was subjected to HP20SS vacuum column chromatography (eluted with a step gradient consisting of 30%, 50%, 70%, and 100% MeOH in H₂O) to generate four fractions. Fraction Fr. 2 was purified by semi-prep HPLC (eluted with 20% MeCN in 0.1% formic acid) to provide compound 18 (20.4 mg).

Purification of Compound 20-22 from the Tryptamine Treatment Experiment

Flasks containing Tolypocladium sp. T1 cultures (200 mL×30 flask, 8 day) were incubated with 200 mg tryptamine (19) at room temperature overnight. The conditioned broth was extracted with equal volumes of n-BuOH (×3). The combined crude extract (3.9 g) was subjected to HP20SS vacuum column chromatography (eluted with gradients of 5%, 30%, 50%, and 100% MeOH in H₂O) to generate four fractions. Fraction Fr. 3 was purified by semi-prep HPLC (eluted with 25% MeCN in 0.1% TFA) to yield compounds 20 (15.0 mg), 21 (7.0 mg), and 22 (3.2 mg).

Physicochemical Properties

Isomaximiscin (2): pale yellow solid; [α]²⁰_{D} 165 (c 0.16, MeOH); UV (MeOH) λₘₐₓ (log ε) 216 (4.43), 288 (3.71); CD (MeOH) λₘₐₓ (Δε) 220 (12.1), 248 (−2.6), 284 (6.7); HRESIMS m/z 450.2120, [M+H]⁺ (calcd for C₂₃H₃₂NO₈, 450.2122).

Pseudomaximiscin A (4): white solid; [α]²⁰_{D}−232 (c 0.83, CHCl₃), UV (MeOH) λₘₐₓ (log ε) 216 (4.41), 290 (3.58); CD (MeOH) λₘₐₓ (Δε) 215 (−11.8), 250 (1.1), 287 (−3.0); IR (film) νₘₐₓ 3360, 1721, 1633, 1587, 1554, 1459, 1437, 1382, 1259, 1235, 1083, 1045, 975 cm⁻¹; HRESIMS m/z 486.2092, [M+Na]⁺ (calcd for C₂₄H₃₃NO₈Na, 486.2098).

Pseudomaximiscin B (5): white solid; [α]²⁰_{D} 63 (c 0.19, CHCl₃), UV (MeOH) λₘₐₓ (log ε) 218 (4.49), 288 (3.73); CD (MeOH) λₘₐₓ (Δε) 223 (6.0), 280 (1.0); IR (film) νₘₐₓ 3367, 1722, 1636, 1590, 1561, 1460, 1441, 1382, 1258, 1235, 1082, 1044, 975 cm⁻¹; HRESIMS m/z 486.2095, [M+Na]⁺ (calcd for C₂₄H₃₃NO₈Na, 486.2098).

Mycophenolic acid 3-O-pericosine (6): white solid; [α]²⁰_{D}−126 (c 0.27, MeOH); UV (MeOH) λₘₐₓ (log e) 222 (4.52), 250 (4.13), 300 (3.72); CD (MeOH) λₘₐₓ (Δε) 213 (40.9), 240 (−30.7), 296 (−3.3); IR (film) νₘₐₓ 3361, 2939, 1722, 1651, 1604, 1548, 1531, 1514, 1460, 1392, 1369, 1255, 1138, 1078, 1037, 968 cm⁻¹; HRESIMS m/z 529.1693, [M+Na]⁺ (calcd for C₂₅H₃₀O₁₁Na, 529.1680).

Mycophenolic acid 16-O-pericosine (7): white solid; [α]²⁰_{D}-4 (c 0.20, MeOH); UV (MeOH) λₘₐₓ (log ε) 217 (4.44), 251 (3.99), 302 (3.62); IR (film) νₘₐₓ 3419, 2943, 1728, 1622, 1448, 1367, 1263, 1139, 1078, 1033, 970 cm⁻¹; HRESIMS m/z 529.1691, [M+Na]⁺ (calcd for C₂₅H₃₀O₁₁Na, 529.1680).

Pericoxide (10): colorless solid; [α]²⁰_{D} 74 (c 0.13, MeOH); UV (MeOH) λₘₐₓ (log ε) 210 (4.34); CD (MeOH) λₘₐₓ (Δε) 207 (6.7), 248 (−1.3); HRESIMS m/z 209.0416, [M+Na]⁺ (calcd for C₈H₁₀O₅Na, 209.0420).

Ciclopriox 1-N—O-pericosine (14): white solid; [α]²⁰_{D}−206 (c 1.5, MeOH); UV (MeOH) λₘₐₓ (log ε) 206 (3.78), 302 (2.98); CD (MeOH) λₘₐₓ (Δε) 220 (−6.6), 259 (0.6), 300 (−1.9); IR (film) νₘₐₓ 3300, 2929, 2852, 1716, 1653, 1558, 1543, 1456, 1435, 1242, 1101, 1080, 1035, 933, 752 cm⁻¹; HRESIMS m/z 416.1690, [M+Na]⁺ (calcd for C₂₀H₂₇NO₇Na, 416.1680).

Suberanilohydroxamic acid 1-O-pericosine (16): white solid; [α]²⁰_{D}-93 (c 0.9, MeOH); UV (MeOH) λₘₐₓ (log ε) 204 (3.68), 242 (3.18); CD (MeOH) λₘₐₓ (Δε) 218 (−2.2), 246 (0.6); IR (film) νₘₐₓ, 3271, 2933, 2858, 1714, 1660, 1599, 1543, 1498, 1442, 1253, 1151, 1082, 1049, 1028, 904, 760, 694 cm⁻¹; HRESIMS m/z 473.1908, [M+Na]⁺ (calcd for C₂₂H₃₀N₂O₈Na, 473.1984).

Anisomycin 1-N-pericosine (18): white solid; [α]²⁰_{D}-178 (c 1.4, MeOH); UV (MeOH) λₘₐₓ (log ε) 208 (4.13), 278 (3.17); CD (MeOH) λₘₐₓ (Δε) 219 (−1.8), 236 (0.6), 290 (−2.9); IR (film) νₘₐₓ 3427, 3010, 2951, 2837, 1722, 1714, 1612, 1583, 1514, 1440, 1373, 1248, 1178, 1151, 1074, 1037, 979, 964, 894, 819, 790, 754 cm⁻¹; ¹H and ¹³C NMR data, see Table 1; HRESIMS m/z 452.1930, [M+H]⁺ (calcd for C₂₂H₃₀NO₉, 452.1921).

(3′R,4′R,5′S,6′R)-Tryptamine 1-N-pericosine (20): white solid; [α]²⁰_{D}−35 (c 1.0, EtOH); UV (MeOH) λₘₐₓ (log ε) 214 (4.30), 260 (3.73, sh), 291 (3.47, sh); CD (MeOH) λₘₐₓ (Δε) 231 (−1.7), 259 (−0.9); IR (film) νₘₐₓ 3307, 1678, 1436, 1338, 1276, 1201, 1138, 1041, 839, 800, 746, 721 cm⁻¹; HRESIMS m/z 347.1610, [M+H]⁺ (calcd for C₁₈H₂₃N₂O₅, 347.1601).

rac-(3′R*,4′R*,5'S*,6'S*)-Tryptamine 1-N-pericosine (21): white solid; [α]²⁰_{D}−2 (c 0.5, EtOH); UV (MeOH) λₘₐₓ (log ε) 214 (4.31), 260 (3.70, sh), 291 (3.42, sh); IR (film) νₘₐₓ 3296, 1676, 1433, 1263, 1199, 1138, 1055, 839, 800, 748, 723 cm⁻¹; HRESIMS m/z 347.1611, [M+H]+(calcd for C₁₈H₂₃N₂O₅, 347.1601).

Mallimiscin (22): white solid; [α]²⁰_{D}−18 (c 0.65, EtOH); UV (MeOH) λₘₐₓ (log ε) 210 (4.36), 260 (3.78, sh), 291 (3.51, sh); CD (MeOH) λₘₐₓ (Δε) 207 (4.4), 225 (−6.9), 250 (0.4), 306 (−0.7); IR (film) νₘₐₓ 3332, 1676, 1438, 1342, 1278, 1199, 1138, 1087, 1037, 941, 800, 748, 721, 677 cm⁻¹; HRESIMS m/z 509.2144, [M+H]⁺ (calcd for C₂₄H₃₃N₂O₁₀, 509.2130).

Chlorination of Compound 10

Compound 10 (10 mg) was stirred in 2 mL ddH₂O with 55 mg NaCl overnight. The water was removed from the resulting solution in vacuo. The residue was re-dissolved in 1 mL MeOH, passed through a small C18 column, and further purified using semi-preparative reversed-phase HPLC (Luna 5 μm, C18, 110 Å, 250×10.00 mm, 15% MeCN in H₂O, 4.0 mL/min) to yield 11 (1.1 mg), [α]²⁰_{D} 112 (c 0.04, MeOH).

Nucleophilic Substitution of 10 and 11

To obtain pyridoxatin (12), compound 1 (110 mg) was stirred in 5 mL DMF with the addition of 1 mL HCl (12 N) at 75° C. for 24 h. The HCl was removed in vacuo. The residue was purified using semi-preparative reversed-phase HPLC (Luna 5 μm, C18, 110 Å, 250×10.00 mm, 60% MeCN in H₂O, 4.0 mL/min) to yield 12 (42 mg), [α]²⁰_{D}−20 (c 0.5, MeOH). Compounds 10 (7 mg) and 12 (5 mg) were stirred in 2 mL ddH₂O overnight. The production of 1 was observed in a low yield. To maximize the yield, 0.5 mL MeOH was added to help dissolve 12. The mixture was heated at 70° C. for 1 h to complete the reaction. The solvent was removed and the resulting residue purified using semi-preparative reversed-phase HPLC (Luna 5 μm, C18, 110 Å, 250×10.00 mm, 55% MeCN in 0.1 formic acid, 4.0 mL/min) to yield 1 (5.0 mg), [α]²⁰_{D}−198 (c 0.25, MeOH). The production of 1 was also detectable after compounds 11 (16 mg) and 9 (10 mg) were stirred in 2 mL ddH₂O overnight. MeOH (0.5 mL) was added and the solution was heated at 70° C. for 1 h. However, the yield of 1 was not significantly improved. The solution was then cooled down to room temperature followed by the addition of 100 μL Et₃N. The reaction was completed after stirring for 1 h. HPLC purification was performed to yield 1 (15 mg), [α]²⁰_{D}−195 (c 0.75, MeOH).

Treatment of Cultures with [U-¹³C₆]-D-Glucose and Nucleophilic Precursors

For the isotope labeling experiments, 2 g of [U-¹³C₆]-D-glucose (Cambridge Isotope Laboratories, Inc., USA) was dissolved in water and filter sterilized. Spores were inoculated into flasks containing autoclaved PDB medium [60 Erlenmeyer flasks (1 L) with each flask containing 200 mL of medium]. The total quantity of [U-¹³C6]-D-glucose was divided into three equal parts and administered sequentially to each flask at three time points (48 h, 96 h, and 120 h). The flasks were shaken for a total of 8 days on a rotary shaker. Twenty-five culture flask (5 L) were successively extracted with EtOAc and n-BuOH. The ¹³C-labelled 10 (10.2 mg) and 11 (2.2 mg) were purified from the EtOAc and the n-BuOH extracts, respectively. The remaining 35 flasks of culture (7 L) were treated with nucleophilic precursors overnight: 20 mg of 8 in 5 flasks, 100 mg of 9 in 12 flasks, 40 mg of 13 in 5 flasks, 40 mg of 15 in 5 flasks, 25 mg of 17 in 5 flasks, and 60 mg of 19 in 12 flasks. Similar extraction, purification, and manipulation procedures were used as previously described to obtain ¹³C-labelled compounds 4 (6 mg), 5 (5 mg), 6 (0.8 mg), 7 (2.0 mg), 14 (30.0 mg), 16 (18.0 mg), 18 (14.0 mg), 20 (4.1 mg), 21 (2.5 mg), and 22 (1.2 mg).

Computational Details

Conformational analyses were carried out using Spartan′10 and ComputeVOA™ v1.1. Geometry, frequency, ¹³C NMR, ECD, IR and VCD intensity, and specific rotation were applied at the DFT and TD-DFT levels [B3LYP functional/6-31G(d) or 6-31+G(d,p) or 6-311+G(2d,p) or DGDZVP basis set] with Gaussian′09 carried out in gas phase or in MeOH. For each substance, subsets of the lowest energy conformers in the gas phase were obtained by selecting only those conformers with energies predicted to be within 2.0 kcal/mol of their respective lowest-energy conformers. The ECD, IR and VCD spectra, ¹³C NMR data, and specific rotation values of these conformers were summed after a Boltzmann statistical weighting. Single UV and CD spectra of the calculated conformers were determined using SpecDis 1.60 using a sigma value of 0.2-0.3 eV. After applying a UV-shift correction, the computed CD spectra were compared with the experimentally determined CD curves. The calculated frequencies were scaled by 0.975 and the IR and VCD intensities were converted to Lorentzian bands with 6 cm⁻¹ half-width for comparison to experimental data. ComputeVOA™ v1.1 was used to sum IR or VCD spectra.

The B3LYP/6-31G(d) method resident in Gaussian 09 was used to determine the structures and locate transition states (TS). The transition states were found by a two-step process utilizing first the Mod Redundant function to determine structure/energies at various fixed Nu-C distances and then executing a Berny TS optimization calculation. Each TS exhibited a negative vibrational frequency of greater than −100 cm⁻¹ on the reaction coordinate connecting reactants to products (values given below). Final energies were determined from a single point calculation with the M06-2X functional and/or the MP2 method and the 6-311++G(d,p) basis set. Electronic energies were corrected for zero point vibrational energy, temperature (298 K), and entropy by frequency calculations and for solvation (in H₂O) by the CPCM solvation model to afford the free energy values. The graphics for the calculated transition states were produced from the Gaussian output files with the CYLview software application.

Antifungal Test

The effects of compounds on fungal growth of fungi were tested using the method described in the NCCLS M38-A guidelines with following modifications. Fungi were cultured on PDA plates (potato dextrose agar, Becton Dickinson and Company) at 25° C. for 6-10 days. The spores/mycelia were disrupted by mechanical agitation and diluted in RPMI 1640 medium (Sigma Chemical Corporation) buffered to pH 7.0 with MOPS (0.165 M, Sigma). Test compounds were prepared in DMSO or EtOH at stock concentrations of 10 mM before being serially diluted in 50 μL RPMI 1640 plus MOPS medium for testing. Aliquots of spore suspension were added to the medium containing the diluted compounds or vehicle (<1% by vol.). After 72 h of incubation at 25° C., the optical densities of fungi were measured using a microplate reader (Infinite M200, Tecan Group Ltd.). The minimum inhibitory concentration (MIC) for growth was defined as the lowest concentration causing prominent growth reduction (>80%). The RPMI 1640 medium was replaced by PDB medium or PDB-N(PDB with 2 g/L NaNO₃) for the indicated antifungal tests.

Structure Revision of Compound 1

Shortly after the disclosure of 1, we determined that its isomeric product, isomaximiscin (2) was formed when 1 was held in DMSO-d6 under the experimental conditions employed for VCD spectroscopy. The unexpected generation of isomer 2 led to a misinterpretation of the experimental VCD data and an incorrect assignment for the absolute configuration of 1.

To determine the correct absolute configuration of 1, a combination of ¹³C-isotope labeling and TD-DFT ECD calculation was used. ¹³C-labeled 1 (generated by feeding [U-¹³C6]-D-glucose to fungus T1) was chemically cleaved to obtain the labeled product (+)-pericosine C (3). The absolute configuration of (+)-pericosine C (3) was confirmed by ECD calculation. Further insight concerning the isomeric relationship of 1 and 3 was assessed via analysis of their ¹³C-labeling patterns (FIG. 1A of the appendix of U.S. Provisional Patent Application 62/295,343). The coupling constant for C-2 (pattern A, J_{C-2,C-3}=43 Hz; pattern B, J_{C-1,C-2}=67 Hz) was used to track the fate of each carbon during the generation of 3 and 1. The results indicated that the methoxy group in 3 replaced what had been the C-2′ allylic hydrogen of 1 with the concomitant migration of the olefinic bond and loss of the PKS-NRPS unit from C-6′. Thus, the absolute configuration of C-3′-C-5′ in 1 was determined to be 3′R,4′R,5′R.

To determine the absolute configuration of C-6′, the theoretical ECD spectra of four possible isomers (3′R,4′R,5′R,6′R, 3′R,4′R,5′R,6'S, 3'S,4'S,5'S,6'S, and 3'S,4'S,5'S,6′R) were generated and compared with the experimental data obtained for 1 and 2 (FIG. 1B of the appendix of U.S. Provisional Patent Application 62/295,343). The most striking revelation was the large influence that C-6′ epimerization contributed to the virtual inversion of all major Cotton effects throughout the calculated spectra. These results enabled us to ascertain that the absolute configuration of the shikimate-analogue moiety in 1 was 3′R,4′R,5′R,6′R.

Structure Elucidation of New Compounds 2, 4-7, 10, 14, 16, 18, and 20-22

Compound 2 was assigned with the molecular formula C₂₃H₃₁NO₈ by analysis of its HRESIMS data. The ¹H and ¹³C NMR data (Table 51 of the appendix of U.S. Provisional Patent Application 62/295,343) of 2 were almost identical to those of 1 indicating 2 had the same planar structure as that of 1. The planar structure of 2 was also supported by the analysis of its 2D NMR (HSQC and HMBC) data. The almost identical NMR data of the shikimate analogue moieties (C-1′˜C-8′) in 1 and 2, including the ¹H and ¹³C NMR chemical shifts and J_{H,H} coupling constants (Table S1 of the appendix of U.S. Provisional Patent Application 62/295,343), were rationalized to have arisen from the migration of the O-N bridge from C-6′ to C-2′ (S_{N}2′ process) resulting in the inverted configuration for C-3′, C-4′, C-5′, and C-6′ of 2. According to the revised absolute configuration of 1, the absolute configuration of shikimate analogue moiety in 2 was deduced as 3'S,4'S,5'S,6'S. To confirm the absolute configuration, DFT calculation of specific rotation values and ECD spectra were performed for 1, 2, and their 6′-epimers. The positive specific rotation value ([α]²⁰_{D} 165) of 2 matched well with the calculated value ([α]²⁰_{D} 152) while the specific rotation values of 1, 6′-epi-1, and 6′-epi-2 were all determined to be negative (−272 for 1, −20 for 6′-epi-1, and −93 for 6′-epi-2). Furthermore, the observed positive Cottons effects at 220 and 284 nm showed good agreement with the TD-DFT calculated ECD spectrum of 2 (FIG. 1B of the appendix of U.S. Provisional Patent Application 62/295,343). Thus, the structure and absolute configuration of 2 were secured.

Compound 4 was obtained as a white powder. The molecular formula was determined to be C₂₄H₃₃NO₈ based on HRESIMS data. The UV and IR spectra indicated its structure was related to maximiscin (1). Comparisons of the ¹H and ¹³C NMR chemical shifts of 4 with 1 indicated the presence of an identical shikimate analogue moiety (C-1′-C-8′, Table S2 of the appendix of U.S. Provisional Patent Application 62/295,343). The rest of the structure, a PKS-NRPS hybrid, and its relative configuration were established by analysis of the 1D (¹H and ¹³C) and 2D (¹H-¹H COSY, qHSQC, qHMBC and ROESY) NMR data (Table S2 of the appendix of U.S. Provisional Patent Application 62/295,343). A search for the PKS-NRPS unit in Scifinder returned a hit, PF1140 (8),^{[ref 1]} which was a metabolite of the Penicillium sp. P1. The absolute configuration of 8 was determined by DFT calculation of its VCD spectrum (FIG. S25 of the appendix of U.S. Provisional Patent Application 62/295,343) as 7R,8S,10R,12S,13R. Thus, the absolute configuration of the PKS-NRPS unit in 4 was established as 7R,8S,10R,12S,13R. The absolute configuration of the shikimate analogue was established based on the ¹³C-labeling experiment and the DFT-ECD calculation. When compared to 10 and 11, the ¹³C-labeling pattern of 4 (FIG. 3 of the appendix of U.S. Provisional Patent Application 62/295,343) indicated a S_{N}2′ mechanism was involved in its formation. Thus, the absolute configuration of C-3′-C-5′ was deduced as 3′R,4′R,5′R. Furthermore, the ECD spectrum of 4 (FIG. 5126 of the appendix of U.S. Provisional Patent Application 62/295,343) exhibited a strong negative cotton effect at 215 nm which led to the assignment of the 6′R configuration.

Rapid isomerization of 4 was observed when it was dissolved in DMSO-d₆. The resulting mixture was purified on HPLC yielding 4 and its diastereomer 5. While both compounds were stable for several days in CH₂Cl₂, CHCl₃, MeOH, and EtOH at room temperature, their prolonged storage in alcohol over several weeks ultimately led to modest levels of compound rearrangement. The ¹H and ¹³C NMR data of 4 and 5 were almost identical (Tables S2 and S3 of the appendix of U.S. Provisional Patent Application 62/295,343). The ¹³C-isotope-labeling pattern for 5 (FIG. 3 of the appendix of U.S. Provisional Patent Application 62/295,343) suggested that it was also formed through a S_{N}2′ reaction process. Thus, the absolute configuration of C-3′-C-5′ was deduced as 3'S,4'S,5'S, opposite to that of 4. Furthermore, the ECD spectrum of 5 (FIG. S126 of the appendix of U.S. Provisional Patent Application 62/295,343) exhibited a strong positive Cotton effect at 218 nm which led to the assessment of its 6'S configuration.

Compound 6 was purified as a white solid bearing the molecular formula C₂₅H₃₀O₁₁. Analysis of the 1D (¹H and ¹³C) and 2D (¹H-¹H COSY, HSQC, HMBC, and ROESY) NMR data (Table S4 of the appendix of U.S. Provisional Patent Application 62/295,343) indicated the shikimate analogue moiety was attached to the mycophenolic acid through a C-6′-O—C-3 bridge. This assignment was supported by the HMBC correlation from H-6′ to C-3. The E configuration of the C-12/C-13 olefin was assigned by ROESY correlations between H-17 and H-11, as well as between H-12 and H-14. The ¹³C-labeling pattern (FIG. 3 of the appendix of U.S. Provisional Patent Application 62/295,343) of the shikimate analogue unit indicated the absolute configuration of C-3′-C-6′ would be the same as that for 4. The 6′R configuration was confirmed by comparison of its experimental and calculated ECD spectra (FIG. 5126 of the appendix of U.S. Provisional Patent Application 62/295,343).

Compound 7 bore the same molecular formula as 6. Comparison of their 1D and 2D NMR data (Table S4 and S5 of the appendix of U.S. Provisional Patent Application 62/295,343) indicated the shikimate analogue moiety was esterified with the carboxylic acid group, which was supported by an HMBC correlation from H-6′ to C-16. The mixed isotope pattern of the ¹³C-labeled 7 (FIG. 3 of the appendix of U.S. Provisional Patent Application 62/295,343) and its negligible specific rotation ([α]²⁰_{D}−4) indicated 7 was an enantiomeric mixture, which was separated using chiral HPLC (FIG. S26 of the appendix of U.S. Provisional Patent Application 62/295,343). In order to determine the relative configuration of 7, the ¹³C NMR data of the shikimate analogue portion for two model (acetate bearing) compounds, 7a (3′R*,4′R*,5′R*,6′R*, FIG. S12 of the appendix of U.S. Provisional Patent Application 62/295,343) and 6′-epi-7a (3′R*,4′R*,5′R*,6′S*, FIG. S13 of the appendix of U.S. Provisional Patent Application 62/295,343), were calculated. The chemical shift of C-3 was used as a reference and the Δ_{C} values (Δ_{C4-C3}, Δ_{C5-C3}, and Δ_{C6-C3}) were calculated for 7 (exptl.), 7a (calcd.), and 6′-epi-7a (calcd.) (FIG. S14 of the appendix of U.S. Provisional Patent Application 62/295,343). The observed Δ_{C} trend of 7 was consistent with the calculated Δ_{C} trend of 7a but distinct from that of 6′-epi-7a. Thus, 7 and 7a should share the same relative configuration as 3′R*,4′R*,5′R*,6′R*.

The molecular formula C₈H₁₀O₅ was assigned to 10 based on its HRESIMS data. Analysis of its 1D (¹H and ¹³C NMR, Tables S6) and 2D (¹H,¹H-COSY, HSQC, and HMBC) NMR data established the planar structure of 10 as containing an epoxy group located on C-5 (δ_{C} 58.6)/C-6 (δ_{C} 50.1). Since the epoxy ring-opening reaction of 10 with Cl⁻ favored a S_{N}2 mechanism to form pericosine A (11) (FIG. 4 of the appendix of U.S. Provisional Patent Application 62/295,343), the absolute configuration of 10 was deduced as 3S,4S,5S,6R. The assessment was also supported by comparing the DFT-calculated and experimental ECD spectra of 10 (FIG. S126 of the appendix of U.S. Provisional Patent Application 62/295,343).

Both compounds 14 and 16 were isolated as pale yellow solids. Their molecular formulae were assigned as C₂₀H₂₇NO₇ and C₂₂H₃₀N₂O₈, respectively, by analyses of their HRESIMS data. Examination of their 1D and 2D NMR data (Tables S7 and S8 of the appendix of U.S. Provisional Patent Application 62/295,343) revealed that N—O—C bridges in both compounds were formed from C-6′ of their shikimate analogue moieties as observed for 4. The absolute configurations of the shikimate analogue moieties were found to be consistent with that of 4 based on the presence of same ¹³C-labeling patterns (FIG. 3 of the appendix of U.S. Provisional Patent Application 62/295,343) and negative Cotton effects at 220 nm (14) and 218 nm (16) (FIG. S126 of the appendix of U.S. Provisional Patent Application 62/295,343).

Compound 18 was obtained as a pale brown solid. Its molecular formula was determined to be C₂₂H₂₉NO₉ based on its HRESIMS data. Analysis of its 1D (¹H and ¹³C) and 2D (¹H-¹H COSY, HSQC, HMBC, ROESY) NMR data (Table S9 of the appendix of U.S. Provisional Patent Application 62/295,343) indicated the shikimate analogue moiety was linked to the nitrogen atom of anisomycin. The absolute configurations of C-3′, C-4′, and C-5′ were determined to be 3′R,4′R,5'S based on the ¹³C labeling pattern (FIG. 3 of the appendix of U.S. Provisional Patent Application 62/295,343). The key ROESY correlations from H-4′ to H-2 supported a 5′,6′-trans configuration because these ROESY correlations were impossible to be observed for 5′,6′-cis configuration based on the DFT-calculation for the lowest-energy conformers (FIGS. S22 and S23 of the appendix of U.S. Provisional Patent Application 62/295,343). The 6′R configuration was also confirmed by comparison of the experimental and calculated ECD spectra (FIG. S126 of the appendix of U.S. Provisional Patent Application 62/295,343).

Compounds 20 and 21 were obtained as pale brown solids. Both compounds 20 and 21 were isolated as TFA salts (0.1% TFA was used in the HPLC solvent) which were confirmed by ¹⁹F NMR. The quaternary NH protons were observed by ¹H NMR in DMSO-d₆. The same molecular formula was established for both compounds in their free-base form as C₁₈H₂₂N₂O₅ according to their HRESIMS data. Comparisons of their 1D and 2D NMR data (Tables S10 and S11 of the appendix of U.S. Provisional Patent Application 62/295,343) indicated that both compounds were tryptamine-shikmate adducts with the primary amine bonded to C-6′. This was supported by the HMBC correlation from H-6′ to C-1. The only difference in their structures was the relative configuration between C-5′ and C-6′. The large J_{5′,6′} value (9.2 Hz) supported a 5'S*,6′R* relative configuration for 20, while a 5'S*,6'S* relative configuration was assigned to 21 due to its small J_{5′,6′} value (5.3 Hz). Based on the isotope labeling pattern (FIG. 3 of the appendix of U.S. Provisional Patent Application 62/295,343) of 20, the absolute configuration of the shikimate analogue unit was determined to be 3′R,4′R,5'S,6′R. In contrast, 21 was determined to be an enantiomeric mixture due to its negligible specific rotation ([α]²⁰_{D}−2) and the mixed ¹³C labeling pattern from the isotope-labeling experiment (FIG. 3 of the appendix of U.S. Provisional Patent Application 62/295,343). The two enantiomers were successfully separated using chiral HPLC.

Compound 22 was also isolated as a TFA salt with molecular formula C₂₄H₃₂N₂O₁₀ for its base form based on HRESIMS data. Comparison of the ¹H and ¹³C NMR data (Table S12 of the appendix of U.S. Provisional Patent Application 62/295,343) with those of 20 (Table S10 of the appendix of U.S. Provisional Patent Application 62/295,343) and 21(Table S11 of the appendix of U.S. Provisional Patent Application 62/295,343) indicated the structure of 22 contained the same tryptamine-shikimate conjugated moiety as that in the structure of 20. The key HMBC correlations from H-5′ to the hemiketal C-2″ (δ_{C} 98.7), from H-1 to C-6′ and C-1″, and from H-1″ to C-6′ and C-2″ indicated the formation of a morpholine ring. A butane-1,2,3,4-tetraol chain was attached to C-2″ supported by the key ¹H-¹H COSY correlations between H-4″ and H-5″ and between H-6″ and H-5″, as well as the key HMBC correlations from H-3″ to C-1″, C-2″, and C-5″. The relative configuration of 22 was partially established based on interpretation of ROESY correlations and J_{H,H} coupling constant values (FIG. 6 of the appendix of U.S. Provisional Patent Application 62/295,343). The key ROESY correlations between H-3′ and H-5′, the small J_{H-4′,H-5′} value (1.9 Hz), and the large J_{H-6′,H-5′} value (9.7 Hz) revealed the 3′,4′-cis/4′,5′-cis/5′,6′-trans configuration. The key ROESY correlations between H-5′ and H-1a, between H-1″a and H-6′, between H-1″a and H-3″, and between H-1″b and H-2 indicated H-5′, OH-2″, and the indole side chain were on the same side of the morpholine ring. The tiny J_{H-4″,H-3″} value (0.85 Hz) and the large J_{H-4″,H-5″} value (8.5 Hz) supported a 3″,4″-cis/4″,5″-trans (3″S*, 4″R*, 5″R*) configuration by comparison against a J-coupling constant library of synthetic polyols.^{[ref m]} However, the relative configuration between C-2″ and C-3″ could not be determined by analysis of NMR data. The ¹³C-isotope labeling experiment for 22 by feeding with [U-¹³C6]-D-glucose (FIG. 3 of the appendix of U.S. Provisional Patent Application 62/295,343) established a type A labeling pattern (J_{C-2,C-3}=43 Hz, FIG. 1A of the appendix of U.S. Provisional Patent Application 62/295,343) for the shikimate analogue moiety. Thus, the absolute configuration of the chiral centers on the dual-ring systems were determined as 3′ R,4′ R,5'S,6′ R,2″R which was supported by comparison of the experimental ECD spectrum of 22 with the calculated spectrum of the 2″-methyl model compound 22a (FIG. S126 of the appendix of U.S. Provisional Patent Application 62/295,343). The ¹³C-isotope labeling pattern of 22 also revealed that the skeleton from C-1″ to C-6″ was derived from an intact D-glucose. Comprehensive literature research indicated that 22 was derived from a reaction involving a unit of 20 and a unit of D-glucose via the mechanism similar to the Maillard reaction (FIG. 6 of the appendix of U.S. Provisional Patent Application 62/295,343). Thus, as C-3″˜C-5″ were not involved in the Maillard reaction, they should retain the same absolute configuration of C-3˜C-5 in D-glucose. So the absolute configuration of C-3″˜C-5″ were deduced as 3″S, 4″R, 5″R, which matched the analysis of the relative configuration based on J_{H,H} coupling constants.

### Example 1

Demonstration of Thiol Neutralization by Pericosine A

This example shows that the shikimate analogue Pericosine A completely neutralizes a thiol (2-phenylethanthiol) that has been applied to pig skin. The data shows that a composition containing Pericosine A and spermine disposed in a propylene glycol vehicle was able to neutralize the thiol disposed on pig skin. After the Pericosine composition was applied to the thiol on the pig skin and allowed to react for 10 minutes, an eluate from the treated area was analyzed with LCMS. Only a pericosine-thiol conjugate was detected. No free pericosine or thiol was detected, indicating that the thiol had been completely neutralized/consumed by the pericosine. This demonstrates how the shikimate analogue can neutralize a thiol.

Method

Pericosine A, spermine, and the model thiol compound 2-phenylethanethiol were prepared into 100 mM stock solutions in dimethyl sulfoxide (DMSO). 5 uL pericosine A and 5 uL spermine solutions were pre-mixed into 50 uL 50% propylene glycol to form a test composition. A piece of pig skin (1 inch×1 inch) was placed in a 10-cm ID petri dish. 5 uL of the thiol solution was dispensed onto the pig skin sample. The pericosine test composition was applied to the thiol-treated spot on the pig skin sample. The petri dish was covered and gently agitated to cause the thiol sample to mix with the pericosine test composition. After 10 min incubation, the solution on the pig skin was transferred using a pipet into an Eppendorf tube. The treated spot on the pig skin was extracted with 100 uL MeOH and the MeOH solution was combined with the solution in the Eppendorf tube. After mixing and centrifuging, the mixed solution was subjected to LCMS analysis.

Results:

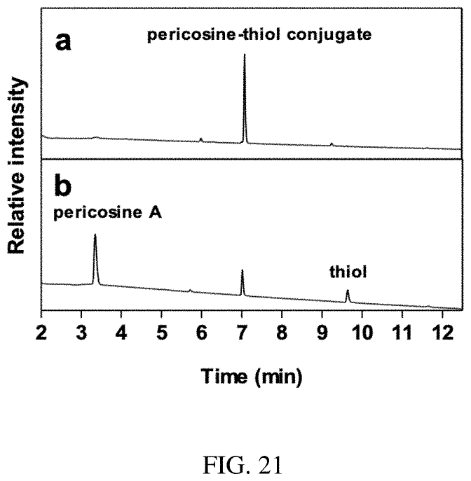

FIG. 21(b) shows standard pericosine A and thiol peaks for a 50% methanol/50% water control solution comprising pericosine A and the thiol. As shown in FIG. 21(a), a major pericosine-thiol conjugated product was detected by the LCMS analysis in the solution removed from the pig skin. Neither pericosine A nor the model thiol compound could be detected in FIG. 21(a) by LCMS, indicating the quantitative consumption of both compounds in the reaction and thus neutralization of the thiol deposited on the pig skin sample.

### Example 2

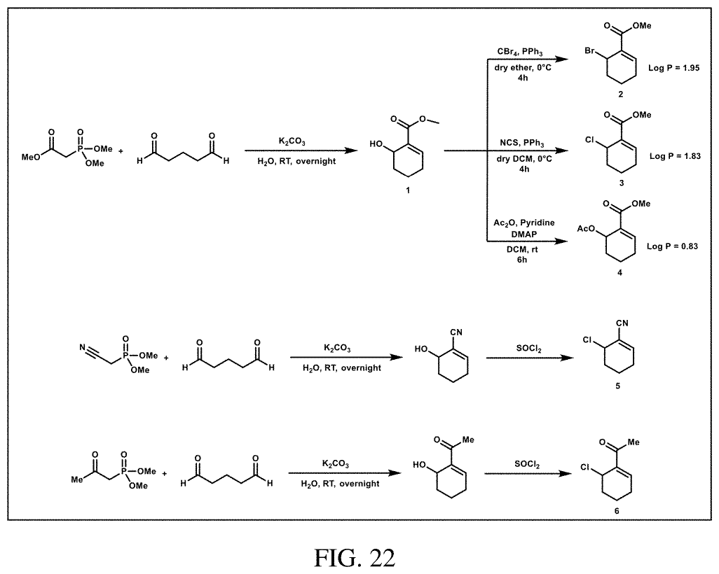

FIG. 22 shows the synthesis of several neutral pericosine analogues.

### Example 3

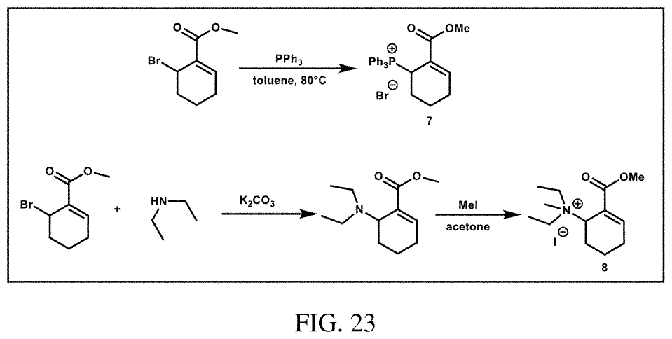

FIG. 23 shows the synthesis of several ammonium and phosphonium pericosine analogues.

### Example 4

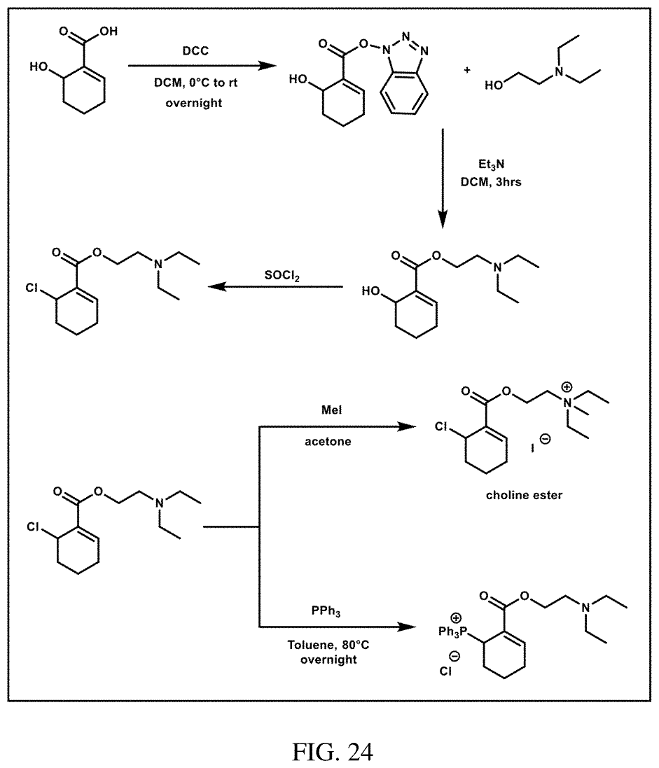

FIG. 24 shows the synthesis of several ammonium and phosphonium pericosine analogues.

Certain non-limiting examples of compounds (pericosine analogues) having Structural Formula V are shown below in Tables 3-49. In certain embodiments of the compounds and compositions of the present disclosure, particular compounds which fall within the metes and bounds of Structural Formula V, such as those characterized as non-limiting examples in Tables 3-49, may optionally be excluded from claims of the present disclosure. Tables 3-49 show non-limiting embodiments of structures which may comprise the X, Y, W, V, and U R groups of Structural Formula V. The Y, W, V, and U R group combinations shown in Tables 3-49 also represent possible R group combinations for compounds represented by Structural Formulas VI and VII (wherein the “X” R group is not included).

TABLE 3
Pericosine Analogues - Structural Formulas V, VI, and VII

| X | Y | W | V | U |
|---|---|---|---|---|
| methyl | OH | H | H | H |
| methyl | Br | H | H | H |
| methyl | Cl | H | H | H |
| methyl | F | H | H | H |
| methyl | acetyloxy | H | H | H |
| methyl | PH₃P | H | H | H |
| methyl | N,N,N-methyldiethyl-2- | H | H | H |
| | (11-oxidaneyl)ethan-1- | | | |
| | aminium | | | |
| methyl | I | H | H | H |
| methyl | tosyl | H | H | H |
| methyl | brosyl | H | H | H |
| methyl | mesyl | H | H | H |
| methyl | sulfonate | H | H | H |
| methyl | sulfonamide | H | H | H |
| methyl | choline | H | H | H |
| methyl | phosphonate | H | H | H |
| ethyl | OH | H | H | H |
| ethyl | Br | H | H | H |
| ethyl | Cl | H | H | H |
| ethyl | F | H | H | H |
| ethyl | I | H | H | H |
| ethyl | acetyloxy | H | H | H |
| ethyl | PH₃P | H | H | H |
| ethyl | choline | H | H | H |
| ethyl | N,N,N-methyldiethyl-2- | H | H | H |
| | (11-oxidaneyl)ethan-1- | | | |
| | aminium | | | |
| ethyl | tosyl | H | H | H |
| ethyl | brosyl | H | H | H |
| ethyl | mesyl | H | H | H |
| ethyl | sulfonate | H | H | H |
| ethyl | sulfonamide | H | H | H |
| ethyl | phosphonate | H | H | H |
| n-propyl | OH | H | H | H |
| n-propyl | Br | H | H | H |
| n-propyl | Cl | H | H | H |
| n-propyl | F | H | H | H |
| n-propyl | I | H | H | H |
| n-propyl | acetyloxy | H | H | H |
| n-propyl | PH₃P | H | H | H |
| n-propyl | choline | H | H | H |
| n-propyl | N,N,N-methyldiethyl-2- | H | H | H |
| | (11-oxidaneyl)ethan-1- | | | |
| | aminium | | | |
| n-propyl | tosyl | H | H | H |
| n-propyl | brosyl | H | H | H |
| n-propyl | mesyl | H | H | H |
| n-propyl | sulfonate | H | H | H |
| n-propyl | sulfonamide | H | H | H |
| n-propyl | phosphonate | H | H | H |
| choline | OH | H | H | H |
| choline | Br | H | H | H |
| choline | Cl | H | H | H |
| choline | F | H | H | H |
| choline | I | H | H | H |
| choline | acetyloxy | H | H | H |
| choline | PH₃P | H | H | H |
| choline | choline | H | H | H |
| choline | N,N,N-methyldiethyl-2- | H | H | H |
| | (11-oxidaneyl)ethan-1- | | | |
| | aminium | | | |
| choline | tosyl | H | H | H |
| choline | brosyl | H | H | H |
| choline | mesyl | H | H | H |
| choline | sulfonate | H | H | H |
| choline | sulfonamide | H | H | H |
| choline | phosphonate | H | H | H |
| H | OH | H | H | H |
| H | Br | H | H | H |
| H | Cl | H | H | H |
| H | F | H | H | H |
| H | I | H | H | H |
| H | acetyloxy | H | H | H |
| H | PH₃P | H | H | H |
| H | choline | H | H | H |
| H | N,N,N-methyldiethyl-2- | H | H | H |
| | (11-oxidaneyl)ethan-1- | | | |
| | aminium | | | |
| H | tosyl | H | H | H |
| H | brosyl | H | H | H |
| H | mesyl | H | H | H |
| H | sulfonate | H | H | H |
| H | sulfonamide | H | H | H |
| H | phosphonate | H | H | H |

TABLE 4
Pericosine Analogues - Structural Formulas V, VI, and VII

| X | Y | W | V | U |
|---|---|---|---|---|
| methyl | OH | H | OH | H |
| methyl | Br | H | OH | H |
| methyl | Cl | H | OH | H |
| methyl | F | H | OH | H |
| methyl | acetyloxy | H | OH | H |
| methyl | PH₃P | H | OH | H |
| methyl | N,N,N-methyldiethyl-2- | H | OH | H |
| | (11-oxidaneyl)ethan-1- | | | |
| | aminium | | | |
| methyl | I | H | OH | H |
| methyl | tosyl | H | OH | H |
| methyl | brosyl | H | OH | H |
| methyl | mesyl | H | OH | H |
| methyl | sulfonate | H | OH | H |
| methyl | sulfonamide | H | OH | H |
| methyl | choline | H | OH | H |
| methyl | phosphonate | H | OH | H |
| ethyl | OH | H | OH | H |
| ethyl | Br | H | OH | H |
| ethyl | Cl | H | OH | H |
| ethyl | F | H | OH | H |
| ethyl | I | H | OH | H |
| ethyl | acetyloxy | H | OH | H |
| ethyl | PH₃P | H | OH | H |
| ethyl | choline | H | OH | H |
| ethyl | N,N,N-methyldiethyl-2- | H | OH | H |
| | (11-oxidaneyl)ethan-1- | | | |
| | aminium | | | |
| ethyl | tosyl | H | OH | H |
| ethyl | brosyl | H | OH | H |
| ethyl | mesyl | H | OH | H |
| ethyl | sulfonate | H | OH | H |
| ethyl | sulfonamide | H | OH | H |
| ethyl | phosphonate | H | OH | H |
| n-propyl | OH | H | OH | H |
| n-propyl | Br | H | OH | H |
| n-propyl | Cl | H | OH | H |
| n-propyl | F | H | OH | H |
| n-propyl | I | H | OH | H |
| n-propyl | acetyloxy | H | OH | H |
| n-propyl | PH₃P | H | OH | H |
| n-propyl | choline | H | OH | H |
| n-propyl | N,N,N-methyldiethyl-2- | H | OH | H |
| | (11-oxidaneyl)ethan-1- | | | |
| | aminium | | | |
| n-propyl | tosyl | H | OH | H |
| n-propyl | brosyl | H | OH | H |
| n-propyl | mesyl | H | OH | H |
| n-propyl | sulfonate | H | OH | H |
| n-propyl | sulfonamide | H | OH | H |
| n-propyl | phosphonate | H | OH | H |
| choline | OH | H | OH | H |
| choline | Br | H | OH | H |
| choline | Cl | H | OH | H |
| choline | F | H | OH | H |
| choline | I | H | OH | H |
| choline | acetyloxy | H | OH | H |
| choline | PH₃P | H | OH | H |
| choline | choline | H | OH | H |
| choline | N,N,N-methyldiethyl-2- | H | OH | H |
| | (11-oxidaneyl)ethan-1- | | | |
| | aminium | | | |
| choline | tosyl | H | OH | H |
| choline | brosyl | H | OH | H |
| choline | mesyl | H | OH | H |
| choline | sulfonate | H | OH | H |
| choline | sulfonamide | H | OH | H |
| choline | phosphonate | H | OH | H |
| H | OH | H | OH | H |
| H | Br | H | OH | H |
| H | Cl | H | OH | H |
| H | F | H | OH | H |
| H | I | H | OH | H |
| H | acetyloxy | H | OH | H |
| H | PH₃P | H | OH | H |
| H | choline | H | OH | H |
| H | N,N,N-methyldiethyl-2- | H | OH | H |
| | (11-oxidaneyl)ethan-1- | | | |
| | aminium | | | |
| H | tosyl | H | OH | H |
| H | brosyl | H | OH | H |
| H | mesyl | H | OH | H |
| H | sulfonate | H | OH | H |
| H | sulfonamide | H | OH | H |
| H | phosphonate | H | OH | H |

TABLE 5
Pericosine Analogues - Structural Formulas V, VI, and VII

| X | Y | W | V | U |
|---|---|---|---|---|
| methyl | OH | methoxy | H | H |
| methyl | Br | methoxy | H | H |
| methyl | Cl | methoxy | H | H |
| methyl | F | methoxy | H | H |
| methyl | acetyloxy | methoxy | H | H |
| methyl | PH₃P | methoxy | H | H |
| methyl | N,N,N-methyldiethyl-2- | methoxy | H | H |
| | (11-oxidaneyl)ethan-1- | | | |
| | aminium | | | |
| methyl | I | methoxy | H | H |
| methyl | tosyl | methoxy | H | H |
| methyl | brosyl | methoxy | H | H |
| methyl | mesyl | methoxy | H | H |
| methyl | sulfonate | methoxy | H | H |
| methyl | sulfonamide | methoxy | H | H |
| methyl | choline | methoxy | H | H |
| methyl | phosphonate | methoxy | H | H |
| ethyl | OH | methoxy | H | H |
| ethyl | Br | methoxy | H | H |
| ethyl | Cl | methoxy | H | H |
| ethyl | F | methoxy | H | H |
| ethyl | I | methoxy | H | H |
| ethyl | acetyloxy | methoxy | H | H |
| ethyl | PH₃P | methoxy | H | H |
| ethyl | choline | methoxy | H | H |
| ethyl | N,N,N-methyldiethyl-2- | methoxy | H | H |
| | (11-oxidaneyl)ethan-1- | | | |
| | aminium | | | |
| ethyl | tosyl | methoxy | H | H |
| ethyl | brosyl | methoxy | H | H |
| ethyl | mesyl | methoxy | H | H |
| ethyl | sulfonate | methoxy | H | H |
| ethyl | sulfonamide | methoxy | H | H |
| ethyl | phosphonate | methoxy | H | H |
| n-propyl | OH | methoxy | H | H |
| n-propyl | Br | methoxy | H | H |
| n-propyl | Cl | methoxy | H | H |
| n-propyl | F | methoxy | H | H |
| n-propyl | I | methoxy | H | H |
| n-propyl | acetyloxy | methoxy | H | H |
| n-propyl | PH₃P | methoxy | H | H |
| n-propyl | choline | methoxy | H | H |
| n-propyl | N,N,N-methyldiethyl-2- | methoxy | H | H |
| | (11-oxidaneyl)ethan-1- | | | |
| | aminium | | | |
| n-propyl | tosyl | methoxy | H | H |
| n-propyl | brosyl | methoxy | H | H |
| n-propyl | mesyl | methoxy | H | H |
| n-propyl | sulfonate | methoxy | H | H |
| n-propyl | sulfonamide | methoxy | H | H |
| n-propyl | phosphonate | methoxy | H | H |
| choline | OH | methoxy | H | H |
| choline | Br | methoxy | H | H |
| choline | Cl | methoxy | H | H |
| choline | F | methoxy | H | H |
| choline | I | methoxy | H | H |
| choline | acetyloxy | methoxy | H | H |
| choline | PH₃P | methoxy | H | H |
| choline | choline | methoxy | H | H |
| choline | N,N,N-methyldiethyl-2- | methoxy | H | H |
| | (11-oxidaneyl)ethan-1- | | | |
| | aminium | | | |
| choline | tosyl | methoxy | H | H |
| choline | brosyl | methoxy | H | H |
| choline | mesyl | methoxy | H | H |
| choline | sulfonate | methoxy | H | H |
| choline | sulfonamide | methoxy | H | H |
| choline | phosphonate | methoxy | H | H |
| H | OH | methoxy | H | H |
| H | Br | methoxy | H | H |
| H | Cl | methoxy | H | H |
| H | F | methoxy | H | H |
| H | I | methoxy | H | H |
| H | acetyloxy | methoxy | H | H |
| H | PH₃P | methoxy | H | H |
| H | choline | methoxy | H | H |
| H | N,N,N-methyldiethyl-2- | methoxy | H | H |
| | (11-oxidaneyl)ethan-1- | | | |
| | aminium | | | |
| H | tosyl | methoxy | H | H |
| H | brosyl | methoxy | H | H |
| H | mesyl | methoxy | H | H |
| H | sulfonate | methoxy | H | H |
| H | sulfonamide | methoxy | H | H |
| H | phosphonate | methoxy | H | H |

TABLE 6
Pericosine Analogues - Structural Formulas V, VI, and VII

| X | Y | W | V | U |
|---|---|---|---|---|
| methyl | OH | H | H | ethoxy |
| methyl | Br | H | H | ethoxy |
| methyl | Cl | H | H | ethoxy |
| methyl | F | H | H | ethoxy |
| methyl | acetyloxy | H | H | ethoxy |
| methyl | PH₃P | H | H | ethoxy |
| methyl | N,N,N-methyldiethyl-2- | H | H | ethoxy |
| | (11-oxidaneyl)ethan-1- | | | |
| | aminium | | | |
| methyl | I | H | H | ethoxy |
| methyl | tosyl | H | H | ethoxy |
| methyl | brosyl | H | H | ethoxy |
| methyl | mesyl | H | H | ethoxy |
| methyl | sulfonate | H | H | ethoxy |
| methyl | sulfonamide | H | H | ethoxy |
| methyl | choline | H | H | ethoxy |
| methyl | phosphonate | H | H | ethoxy |
| ethyl | OH | H | H | ethoxy |
| ethyl | Br | H | H | ethoxy |
| ethyl | Cl | H | H | ethoxy |
| ethyl | F | H | H | ethoxy |
| ethyl | I | H | H | ethoxy |
| ethyl | acetyloxy | H | H | ethoxy |
| ethyl | PH₃P | H | H | ethoxy |
| ethyl | choline | H | H | ethoxy |
| ethyl | N,N,N-methyldiethyl-2- | H | H | ethoxy |
| | (11-oxidaneyl)ethan-1- | | | |
| | aminium | | | |
| ethyl | tosyl | H | H | ethoxy |
| ethyl | brosyl | H | H | ethoxy |
| ethyl | mesyl | H | H | ethoxy |
| ethyl | sulfonate | H | H | ethoxy |
| ethyl | sulfonamide | H | H | ethoxy |
| ethyl | phosphonate | H | H | ethoxy |
| n-propyl | OH | H | H | ethoxy |
| n-propyl | Br | H | H | ethoxy |
| n-propyl | Cl | H | H | ethoxy |
| n-propyl | F | H | H | ethoxy |
| n-propyl | I | H | H | ethoxy |
| n-propyl | acetyloxy | H | H | ethoxy |
| n-propyl | PH₃P | H | H | ethoxy |
| n-propyl | choline | H | H | ethoxy |
| n-propyl | N,N,N-methyldiethyl-2- | H | H | ethoxy |
| | (11-oxidaneyl)ethan-1- | | | |
| | aminium | | | |
| n-propyl | tosyl | H | H | ethoxy |
| n-propyl | brosyl | H | H | ethoxy |
| n-propyl | mesyl | H | H | ethoxy |
| n-propyl | sulfonate | H | H | ethoxy |
| n-propyl | sulfonamide | H | H | ethoxy |
| n-propyl | phosphonate | H | H | ethoxy |
| choline | OH | H | H | ethoxy |
| choline | Br | H | H | ethoxy |
| choline | Cl | H | H | ethoxy |
| choline | F | H | H | ethoxy |
| choline | I | H | H | ethoxy |
| choline | acetyloxy | H | H | ethoxy |
| choline | PH₃P | H | H | ethoxy |
| choline | choline | H | H | ethoxy |
| choline | N,N,N-methyldiethyl-2- | H | H | ethoxy |
| | (11-oxidaneyl)ethan-1- | | | |
| | aminium | | | |
| choline | tosyl | H | H | ethoxy |
| choline | brosyl | H | H | ethoxy |
| choline | mesyl | H | H | ethoxy |
| choline | sulfonate | H | H | ethoxy |
| choline | sulfonamide | H | H | ethoxy |
| choline | phosphonate | H | H | ethoxy |
| H | OH | H | H | ethoxy |
| H | Br | H | H | ethoxy |
| H | Cl | H | H | ethoxy |
| H | F | H | H | ethoxy |
| H | I | H | H | ethoxy |
| H | acetyloxy | H | H | ethoxy |
| H | PH₃P | H | H | ethoxy |
| H | choline | H | H | ethoxy |
| H | N,N,N-methyldiethyl-2- | H | H | ethoxy |
| | (11-oxidaneyl)ethan-1- | | | |
| | aminium | | | |
| H | tosyl | H | H | ethoxy |
| H | brosyl | H | H | ethoxy |
| H | mesyl | H | H | ethoxy |
| H | sulfonate | H | H | ethoxy |
| H | sulfonamide | H | H | ethoxy |
| H | phosphonate | H | H | ethoxy |

TABLE 7
Pericosine Analogues - Structural Formulas V, VI, and VII

| X | Y | W | V | U |
|---|---|---|---|---|
| methyl | OH | methyl | H | H |
| methyl | Br | methyl | H | H |
| methyl | Cl | methyl | H | H |
| methyl | F | methyl | H | H |
| methyl | acetyloxy | methyl | H | H |
| methyl | PH₃P | methyl | H | H |
| methyl | N,N,N-methyldiethyl-2- | methyl | H | H |
| | (11-oxidaneyl)ethan-1- | | | |
| | aminium | | | |
| methyl | I | methyl | H | H |
| methyl | tosyl | methyl | H | H |
| methyl | brosyl | methyl | H | H |
| methyl | mesyl | methyl | H | H |
| methyl | sulfonate | methyl | H | H |
| methyl | sulfonamide | methyl | H | H |
| methyl | choline | methyl | H | H |
| methyl | phosphonate | methyl | H | H |
| ethyl | OH | methyl | H | H |
| ethyl | Br | methyl | H | H |
| ethyl | Cl | methyl | H | H |
| ethyl | F | methyl | H | H |
| ethyl | I | methyl | H | H |
| ethyl | acetyloxy | methyl | H | H |
| ethyl | PH₃P | methyl | H | H |
| ethyl | choline | methyl | H | H |
| ethyl | N,N,N-methyldiethyl-2- | methyl | H | H |
| | (11-oxidaneyl)ethan-1- | | | |
| | aminium | | | |
| ethyl | tosyl | methyl | H | H |
| ethyl | brosyl | methyl | H | H |
| ethyl | mesyl | methyl | H | H |
| ethyl | sulfonate | methyl | H | H |
| ethyl | sulfonamide | methyl | H | H |
| ethyl | phosphonate | methyl | H | H |
| n-propyl | OH | methyl | H | H |
| n-propyl | Br | methyl | H | H |
| n-propyl | Cl | methyl | H | H |
| n-propyl | F | methyl | H | H |
| n-propyl | I | methyl | H | H |
| n-propyl | acetyloxy | methyl | H | H |
| n-propyl | PH₃P | methyl | H | H |
| n-propyl | choline | methyl | H | H |
| n-propyl | N,N,N-methyldiethyl-2- | methyl | H | H |
| | (11-oxidaneyl)ethan-1- | | | |
| | aminium | | | |
| n-propyl | tosyl | methyl | H | H |
| n-propyl | brosyl | methyl | H | H |
| n-propyl | mesyl | methyl | H | H |
| n-propyl | sulfonate | methyl | H | H |
| n-propyl | sulfonamide | methyl | H | H |
| n-propyl | phosphonate | methyl | H | H |
| choline | OH | methyl | H | H |
| choline | Br | methyl | H | H |
| choline | Cl | methyl | H | H |
| choline | F | methyl | H | H |
| choline | I | methyl | H | H |
| choline | acetyloxy | methyl | H | H |
| choline | PH₃P | methyl | H | H |
| choline | choline | methyl | H | H |
| choline | N,N,N-methyldiethyl-2- | methyl | H | H |
| | (11-oxidaneyl)ethan-1- | | | |
| | aminium | | | |
| choline | tosyl | methyl | H | H |
| choline | brosyl | methyl | H | H |
| choline | mesyl | methyl | H | H |
| choline | sulfonate | methyl | H | H |
| choline | sulfonamide | methyl | H | H |
| choline | phosphonate | methyl | H | H |
| H | OH | methyl | H | H |
| H | Br | methyl | H | H |
| H | Cl | methyl | H | H |
| H | F | methyl | H | H |
| H | I | methyl | H | H |
| H | acetyloxy | methyl | H | H |
| H | PH₃P | methyl | H | H |
| H | choline | methyl | H | H |
| H | N,N,N-methyldiethyl-2- | methyl | H | H |
| | (11-oxidaneyl)ethan-1- | | | |
| | aminium | | | |
| H | tosyl | methyl | H | H |
| H | brosyl | methyl | H | H |
| H | mesyl | methyl | H | H |
| H | sulfonate | methyl | H | H |
| H | sulfonamide | methyl | H | H |
| H | phosphonate | methyl | H | H |

TABLE 8
Pericosine Analogues - Structural Formulas V, VI, and VII

| X | Y | W | V | U |
|---|---|---|---|---|
| methyl | OH | H | H | ethyl |
| methyl | Br | H | H | ethyl |
| methyl | Cl | H | H | ethyl |
| methyl | F | H | H | ethyl |
| methyl | acetyloxy | H | H | ethyl |
| methyl | PH₃P | H | H | ethyl |
| methyl | N,N,N-methyldiethyl-2- | H | H | ethyl |
| | (11-oxidaneyl)ethan-1- | | | |
| | aminium | | | |
| methyl | I | H | H | ethyl |
| methyl | tosyl | H | H | ethyl |
| methyl | brosyl | H | H | ethyl |
| methyl | mesyl | H | H | ethyl |
| methyl | sulfonate | H | H | ethyl |
| methyl | sulfonamide | H | H | ethyl |
| methyl | choline | H | H | ethyl |
| methyl | phosphonate | H | H | ethyl |
| ethyl | OH | H | H | ethyl |
| ethyl | Br | H | H | ethyl |
| ethyl | Cl | H | H | ethyl |
| ethyl | F | H | H | ethyl |
| ethyl | I | H | H | ethyl |
| ethyl | acetyloxy | H | H | ethyl |
| ethyl | PH₃P | H | H | ethyl |
| ethyl | choline | H | H | ethyl |
| ethyl | N,N,N-methyldiethyl-2- | H | H | ethyl |
| | (11-oxidaneyl)ethan-1- | | | |
| | aminium | | | |
| ethyl | tosyl | H | H | ethyl |
| ethyl | brosyl | H | H | ethyl |
| ethyl | mesyl | H | H | ethyl |
| ethyl | sulfonate | H | H | ethyl |
| ethyl | sulfonamide | H | H | ethyl |
| ethyl | phosphonate | H | H | ethyl |
| n-propyl | OH | H | H | ethyl |
| n-propyl | Br | H | H | ethyl |
| n-propyl | Cl | H | H | ethyl |
| n-propyl | F | H | H | ethyl |
| n-propyl | I | H | H | ethyl |
| n-propyl | acetyloxy | H | H | ethyl |
| n-propyl | PH₃P | H | H | ethyl |
| n-propyl | choline | H | H | ethyl |
| n-propyl | N,N,N-methyldiethyl-2- | H | H | ethyl |
| | (11-oxidaneyl)ethan-1- | | | |
| | aminium | | | |
| n-propyl | tosyl | H | H | ethyl |
| n-propyl | brosyl | H | H | ethyl |
| n-propyl | mesyl | H | H | ethyl |
| n-propyl | sulfonate | H | H | ethyl |
| n-propyl | sulfonamide | H | H | ethyl |
| n-propyl | phosphonate | H | H | ethyl |
| choline | OH | H | H | ethyl |
| choline | Br | H | H | ethyl |
| choline | Cl | H | H | ethyl |
| choline | F | H | H | ethyl |
| choline | I | H | H | ethyl |
| choline | acetyloxy | H | H | ethyl |
| choline | PH₃P | H | H | ethyl |
| choline | choline | H | H | ethyl |
| choline | N,N,N-methyldiethyl-2- | H | H | ethyl |
| | (11-oxidaneyl)ethan-1- | | | |
| | aminium | | | |
| choline | tosyl | H | H | ethyl |
| choline | brosyl | H | H | ethyl |
| choline | mesyl | H | H | ethyl |
| choline | sulfonate | H | H | ethyl |
| choline | sulfonamide | H | H | ethyl |
| choline | phosphonate | H | H | ethyl |
| H | OH | H | H | ethyl |
| H | Br | H | H | ethyl |
| H | Cl | H | H | ethyl |
| H | F | H | H | ethyl |
| H | I | H | H | ethyl |
| H | acetyloxy | H | H | ethyl |
| H | PH₃P | H | H | ethyl |
| H | choline | H | H | ethyl |
| H | N,N,N-methyldiethyl-2- | H | H | ethyl |
| | (11-oxidaneyl)ethan-1- | | | |
| | aminium | | | |
| H | tosyl | H | H | ethyl |
| H | brosyl | H | H | ethyl |
| H | mesyl | H | H | ethyl |
| H | sulfonate | H | H | ethyl |
| H | sulfonamide | H | H | ethyl |
| H | phosphonate | H | H | ethyl |

TABLE 9
Pericosine Analogues - Structural Formulas V, VI, and VII

| X | Y | W | V | U |
|---|---|---|---|---|
| methyl | OH | OH | OH | OH |
| methyl | Br | OH | OH | OH |
| methyl | Cl | OH | OH | OH |
| methyl | F | OH | OH | OH |
| methyl | acetyloxy | OH | OH | OH |
| methyl | PH₃P | OH | OH | OH |
| methyl | N,N,N-methyldiethyl-2- | OH | OH | OH |
| | (11-oxidaneyl)ethan-1- | | | |
| | aminium | | | |
| methyl | I | OH | OH | OH |
| methyl | tosyl | OH | OH | OH |
| methyl | brosyl | OH | OH | OH |
| methyl | mesyl | OH | OH | OH |
| methyl | sulfonate | OH | OH | OH |
| methyl | sulfonamide | OH | OH | OH |
| methyl | choline | OH | OH | OH |
| methyl | phosphonate | OH | OH | OH |
| ethyl | OH | OH | OH | OH |
| ethyl | Br | OH | OH | OH |
| ethyl | Cl | OH | OH | OH |
| ethyl | F | OH | OH | OH |
| ethyl | I | OH | OH | OH |
| ethyl | acetyloxy | OH | OH | OH |
| ethyl | PH₃P | OH | OH | OH |
| ethyl | choline | OH | OH | OH |
| ethyl | N,N,N-methyldiethyl-2- | OH | OH | OH |
| | (11-oxidaneyl)ethan-1- | | | |
| | aminium | | | |
| ethyl | tosyl | OH | OH | OH |
| ethyl | brosyl | OH | OH | OH |
| ethyl | mesyl | OH | OH | OH |
| ethyl | sulfonate | OH | OH | OH |
| ethyl | sulfonamide | OH | OH | OH |
| ethyl | phosphonate | OH | OH | OH |
| n-propyl | OH | OH | OH | OH |
| n-propyl | Br | OH | OH | OH |
| n-propyl | Cl | OH | OH | OH |
| n-propyl | F | OH | OH | OH |
| n-propyl | I | OH | OH | OH |
| n-propyl | acetyloxy | OH | OH | OH |
| n-propyl | PH₃P | OH | OH | OH |
| n-propyl | choline | OH | OH | OH |
| n-propyl | N,N,N-methyldiethyl-2- | OH | OH | OH |
| | (11-oxidaneyl)ethan-1- | | | |
| | aminium | | | |
| n-propyl | tosyl | OH | OH | OH |
| n-propyl | brosyl | OH | OH | OH |
| n-propyl | mesyl | OH | OH | OH |
| n-propyl | sulfonate | OH | OH | OH |
| n-propyl | sulfonamide | OH | OH | OH |
| n-propyl | phosphonate | OH | OH | OH |
| choline | OH | OH | OH | OH |
| choline | Br | OH | OH | OH |
| choline | Cl | OH | OH | OH |
| choline | F | OH | OH | OH |
| choline | I | OH | OH | OH |
| choline | acetyloxy | OH | OH | OH |
| choline | PH₃P | OH | OH | OH |
| choline | choline | OH | OH | OH |
| choline | N,N,N-methyldiethyl-2- | OH | OH | OH |
| | (11-oxidaneyl)ethan-1- | | | |
| | aminium | | | |
| choline | tosyl | OH | OH | OH |
| choline | brosyl | OH | OH | OH |
| choline | mesyl | OH | OH | OH |
| choline | sulfonate | OH | OH | OH |
| choline | sulfonamide | OH | OH | OH |
| choline | phosphonate | OH | OH | OH |
| H | OH | OH | OH | OH |
| H | Br | OH | OH | OH |
| H | Cl | OH | OH | OH |
| H | F | OH | OH | OH |
| H | I | OH | OH | OH |
| H | acetyloxy | OH | OH | OH |
| H | PH₃P | OH | OH | OH |
| H | choline | OH | OH | OH |
| H | N,N,N-methyldiethyl-2- | OH | OH | OH |
| | (11-oxidaneyl)ethan-1- | | | |
| | aminium | | | |
| H | tosyl | OH | OH | OH |
| H | brosyl | OH | OH | OH |
| H | mesyl | OH | OH | OH |
| H | sulfonate | OH | OH | OH |
| H | sulfonamide | OH | OH | OH |
| H | phosphonate | OH | OH | OH |

TABLE 10
Pericosine Analogues - Structural Formulas V, VI, and VII

| X | Y | W | V | U |
|---|---|---|---|---|
| methyl | OH | methoxy | methoxy | methoxy |
| methyl | Br | methoxy | methoxy | methoxy |
| methyl | Cl | methoxy | methoxy | methoxy |
| methyl | F | methoxy | methoxy | methoxy |
| methyl | acetyloxy | methoxy | methoxy | methoxy |
| methyl | PH₃P | methoxy | methoxy | methoxy |
| methyl | N,N,N-methyldiethyl-2- | methoxy | methoxy | methoxy |
| | (11-oxidaneyl)ethan-1- | | | |
| | aminium | | | |
| methyl | I | methoxy | methoxy | methoxy |
| methyl | tosyl | methoxy | methoxy | methoxy |
| methyl | brosyl | methoxy | methoxy | methoxy |
| methyl | mesyl | methoxy | methoxy | methoxy |
| methyl | sulfonate | methoxy | methoxy | methoxy |
| methyl | sulfonamide | methoxy | methoxy | methoxy |
| methyl | choline | methoxy | methoxy | methoxy |
| methyl | phosphonate | methoxy | methoxy | methoxy |
| ethyl | OH | methoxy | methoxy | methoxy |
| ethyl | Br | methoxy | methoxy | methoxy |
| ethyl | Cl | methoxy | methoxy | methoxy |
| ethyl | F | methoxy | methoxy | methoxy |
| ethyl | I | methoxy | methoxy | methoxy |
| ethyl | acetyloxy | methoxy | methoxy | methoxy |
| ethyl | PH₃P | methoxy | methoxy | methoxy |
| ethyl | choline | methoxy | methoxy | methoxy |
| ethyl | N,N,N-methyldiethyl-2- | methoxy | methoxy | methoxy |
| | (11-oxidaneyl)ethan-1- | | | |
| | aminium | | | |
| ethyl | tosyl | methoxy | methoxy | methoxy |
| ethyl | brosyl | methoxy | methoxy | methoxy |
| ethyl | mesyl | methoxy | methoxy | methoxy |
| ethyl | sulfonate | methoxy | methoxy | methoxy |
| ethyl | sulfonamide | methoxy | methoxy | methoxy |
| ethyl | phosphonate | methoxy | methoxy | methoxy |
| n-propyl | OH | methoxy | methoxy | methoxy |
| n-propyl | Br | methoxy | methoxy | methoxy |
| n-propyl | Cl | methoxy | methoxy | methoxy |
| n-propyl | F | methoxy | methoxy | methoxy |
| n-propyl | I | methoxy | methoxy | methoxy |
| n-propyl | acetyloxy | methoxy | methoxy | methoxy |
| n-propyl | PH₃P | methoxy | methoxy | methoxy |
| n-propyl | choline | methoxy | methoxy | methoxy |
| n-propyl | N,N,N-methyldiethyl-2- | methoxy | methoxy | methoxy |
| | (11-oxidaneyl)ethan-1- | | | |
| | aminium | | | |
| n-propyl | tosyl | methoxy | methoxy | methoxy |
| n-propyl | brosyl | methoxy | methoxy | methoxy |
| n-propyl | mesyl | methoxy | methoxy | methoxy |
| n-propyl | sulfonate | methoxy | methoxy | methoxy |
| n-propyl | sulfonamide | methoxy | methoxy | methoxy |
| n-propyl | phosphonate | methoxy | methoxy | methoxy |
| choline | OH | methoxy | methoxy | methoxy |
| choline | Br | methoxy | methoxy | methoxy |
| choline | Cl | methoxy | methoxy | methoxy |
| choline | F | methoxy | methoxy | methoxy |
| choline | I | methoxy | methoxy | methoxy |
| choline | acetyloxy | methoxy | methoxy | methoxy |
| choline | PH₃P | methoxy | methoxy | methoxy |
| choline | choline | methoxy | methoxy | methoxy |
| choline | N,N,N-methyldiethyl-2- | methoxy | methoxy | methoxy |
| | (11-oxidaneyl)ethan-1- | | | |
| | aminium | | | |
| choline | tosyl | methoxy | methoxy | methoxy |
| choline | brosyl | methoxy | methoxy | methoxy |
| choline | mesyl | methoxy | methoxy | methoxy |
| choline | sulfonate | methoxy | methoxy | methoxy |
| choline | sulfonamide | methoxy | methoxy | methoxy |
| choline | phosphonate | methoxy | methoxy | methoxy |
| H | OH | methoxy | methoxy | methoxy |
| H | Br | methoxy | methoxy | methoxy |
| H | Cl | methoxy | methoxy | methoxy |
| H | F | methoxy | methoxy | methoxy |
| H | I | methoxy | methoxy | methoxy |
| H | acetyloxy | methoxy | methoxy | methoxy |
| H | PH₃P | methoxy | methoxy | methoxy |
| H | choline | methoxy | methoxy | methoxy |
| H | N,N,N-methyldiethyl-2- | methoxy | methoxy | methoxy |
| | (11-oxidaneyl)ethan-1- | | | |
| | aminium | | | |
| H | tosyl | methoxy | methoxy | methoxy |
| H | brosyl | methoxy | methoxy | methoxy |
| H | mesyl | methoxy | methoxy | methoxy |
| H | sulfonate | methoxy | methoxy | methoxy |
| H | sulfonamide | methoxy | methoxy | methoxy |
| H | phosphonate | methoxy | methoxy | methoxy |

TABLE 11
Pericosine Analogues - Structural Formulas V, VI, and VII

| X | Y | W | V | U |
|---|---|---|---|---|
| methyl | OH | ethoxy | ethoxy | ethoxy |
| methyl | Br | ethoxy | ethoxy | ethoxy |
| methyl | Cl | ethoxy | ethoxy | ethoxy |
| methyl | F | ethoxy | ethoxy | ethoxy |
| methyl | acetyloxy | ethoxy | ethoxy | ethoxy |
| methyl | PH₃P | ethoxy | ethoxy | ethoxy |
| methyl | N,N,N-methyldiethyl-2- | ethoxy | ethoxy | ethoxy |
| | (11-oxidaneyl)ethan-1- | | | |
| | aminium | | | |
| methyl | I | ethoxy | ethoxy | ethoxy |
| methyl | tosyl | ethoxy | ethoxy | ethoxy |
| methyl | brosyl | ethoxy | ethoxy | ethoxy |
| methyl | mesyl | ethoxy | ethoxy | ethoxy |
| methyl | sulfonate | ethoxy | ethoxy | ethoxy |
| methyl | sulfonamide | ethoxy | ethoxy | ethoxy |
| methyl | choline | ethoxy | ethoxy | ethoxy |
| methyl | phosphonate | ethoxy | ethoxy | ethoxy |
| ethyl | OH | ethoxy | ethoxy | ethoxy |
| ethyl | Br | ethoxy | ethoxy | ethoxy |
| ethyl | Cl | ethoxy | ethoxy | ethoxy |
| ethyl | F | ethoxy | ethoxy | ethoxy |
| ethyl | I | ethoxy | ethoxy | ethoxy |
| ethyl | acetyloxy | ethoxy | ethoxy | ethoxy |
| ethyl | PH₃P | ethoxy | ethoxy | ethoxy |
| ethyl | choline | ethoxy | ethoxy | ethoxy |
| ethyl | N,N,N-methyldiethyl-2- | ethoxy | ethoxy | ethoxy |
| | (11-oxidaneyl)ethan-1- | | | |
| | aminium | | | |
| ethyl | tosyl | ethoxy | ethoxy | ethoxy |
| ethyl | brosyl | ethoxy | ethoxy | ethoxy |
| ethyl | mesyl | ethoxy | ethoxy | ethoxy |
| ethyl | sulfonate | ethoxy | ethoxy | ethoxy |
| ethyl | sulfonamide | ethoxy | ethoxy | ethoxy |
| ethyl | phosphonate | ethoxy | ethoxy | ethoxy |
| n-propyl | OH | ethoxy | ethoxy | ethoxy |
| n-propyl | Br | ethoxy | ethoxy | ethoxy |
| n-propyl | Cl | ethoxy | ethoxy | ethoxy |
| n-propyl | F | ethoxy | ethoxy | ethoxy |
| n-propyl | I | ethoxy | ethoxy | ethoxy |
| n-propyl | acetyloxy | ethoxy | ethoxy | ethoxy |
| n-propyl | PH₃P | ethoxy | ethoxy | ethoxy |
| n-propyl | choline | ethoxy | ethoxy | ethoxy |
| n-propyl | N,N,N-methyldiethyl-2- | ethoxy | ethoxy | ethoxy |
| | (11-oxidaneyl)ethan-1- | | | |
| | aminium | | | |
| n-propyl | tosyl | ethoxy | ethoxy | ethoxy |
| n-propyl | brosyl | ethoxy | ethoxy | ethoxy |
| n-propyl | mesyl | ethoxy | ethoxy | ethoxy |
| n-propyl | sulfonate | ethoxy | ethoxy | ethoxy |
| n-propyl | sulfonamide | ethoxy | ethoxy | ethoxy |
| n-propyl | phosphonate | ethoxy | ethoxy | ethoxy |
| choline | OH | ethoxy | ethoxy | ethoxy |
| choline | Br | ethoxy | ethoxy | ethoxy |
| choline | Cl | ethoxy | ethoxy | ethoxy |
| choline | F | ethoxy | ethoxy | ethoxy |
| choline | I | ethoxy | ethoxy | ethoxy |
| choline | acetyloxy | ethoxy | ethoxy | ethoxy |
| choline | PH₃P | ethoxy | ethoxy | ethoxy |
| choline | choline | ethoxy | ethoxy | ethoxy |
| choline | N,N,N-methyldiethyl-2- | ethoxy | ethoxy | ethoxy |
| | (11-oxidaneyl)ethan-1- | | | |
| | aminium | | | |
| choline | tosyl | ethoxy | ethoxy | ethoxy |
| choline | brosyl | ethoxy | ethoxy | ethoxy |
| choline | mesyl | ethoxy | ethoxy | ethoxy |
| choline | sulfonate | ethoxy | ethoxy | ethoxy |
| choline | sulfonamide | ethoxy | ethoxy | ethoxy |
| choline | phosphonate | ethoxy | ethoxy | ethoxy |
| H | OH | ethoxy | ethoxy | ethoxy |
| H | Br | ethoxy | ethoxy | ethoxy |
| H | Cl | ethoxy | ethoxy | ethoxy |
| H | F | ethoxy | ethoxy | ethoxy |
| H | I | ethoxy | ethoxy | ethoxy |
| H | acetyloxy | ethoxy | ethoxy | ethoxy |
| H | PH₃P | ethoxy | ethoxy | ethoxy |
| H | choline | ethoxy | ethoxy | ethoxy |
| H | N,N,N-methyldiethyl-2- | ethoxy | ethoxy | ethoxy |
| | (11-oxidaneyl)ethan-1- | | | |
| | aminium | | | |
| H | tosyl | ethoxy | ethoxy | ethoxy |
| H | brosyl | ethoxy | ethoxy | ethoxy |
| H | mesyl | ethoxy | ethoxy | ethoxy |
| H | sulfonate | ethoxy | ethoxy | ethoxy |
| H | sulfonamide | ethoxy | ethoxy | ethoxy |
| H | phosphonate | ethoxy | ethoxy | ethoxy |

TABLE 12
Pericosine Analogues - Structural Formulas V, VI, and VII

| X | Y | W | V | U |
|---|---|---|---|---|
| methyl | OH | methyl | methyl | methyl |
| methyl | Br | methyl | methyl | methyl |
| methyl | Cl | methyl | methyl | methyl |
| methyl | F | methyl | methyl | methyl |
| methyl | acetyloxy | methyl | methyl | methyl |
| methyl | PH₃P | methyl | methyl | methyl |
| methyl | N,N,N-methyldiethyl-2- | methyl | methyl | methyl |
| | (11-oxidaneyl)ethan-1- | | | |
| | aminium | | | |
| methyl | I | methyl | methyl | methyl |
| methyl | tosyl | methyl | methyl | methyl |
| methyl | brosyl | methyl | methyl | methyl |
| methyl | mesyl | methyl | methyl | methyl |
| methyl | sulfonate | methyl | methyl | methyl |
| methyl | sulfonamide | methyl | methyl | methyl |
| methyl | choline | methyl | methyl | methyl |
| methyl | phosphonate | methyl | methyl | methyl |
| ethyl | OH | methyl | methyl | methyl |
| ethyl | Br | methyl | methyl | methyl |
| ethyl | Cl | methyl | methyl | methyl |
| ethyl | F | methyl | methyl | methyl |
| ethyl | I | methyl | methyl | methyl |
| ethyl | acetyloxy | methyl | methyl | methyl |
| ethyl | PH₃P | methyl | methyl | methyl |
| ethyl | choline | methyl | methyl | methyl |
| ethyl | N,N,N-methyldiethyl-2- | methyl | methyl | methyl |
| | (11-oxidaneyl)ethan-1- | | | |
| | aminium | | | |
| ethyl | tosyl | methyl | methyl | methyl |
| ethyl | brosyl | methyl | methyl | methyl |
| ethyl | mesyl | methyl | methyl | methyl |
| ethyl | sulfonate | methyl | methyl | methyl |
| ethyl | sulfonamide | methyl | methyl | methyl |
| ethyl | phosphonate | methyl | methyl | methyl |
| n-propyl | OH | methyl | methyl | methyl |
| n-propyl | Br | methyl | methyl | methyl |
| n-propyl | Cl | methyl | methyl | methyl |
| n-propyl | F | methyl | methyl | methyl |
| n-propyl | I | methyl | methyl | methyl |
| n-propyl | acetyloxy | methyl | methyl | methyl |
| n-propyl | PH₃P | methyl | methyl | methyl |
| n-propyl | choline | methyl | methyl | methyl |
| n-propyl | N,N,N-methyldiethyl-2- | methyl | methyl | methyl |
| | (11-oxidaneyl)ethan-1- | | | |
| | aminium | | | |
| n-propyl | tosyl | methyl | methyl | methyl |
| n-propyl | brosyl | methyl | methyl | methyl |
| n-propyl | mesyl | methyl | methyl | methyl |
| n-propyl | sulfonate | methyl | methyl | methyl |
| n-propyl | sulfonamide | methyl | methyl | methyl |
| n-propyl | phosphonate | methyl | methyl | methyl |
| choline | OH | methyl | methyl | methyl |
| choline | Br | methyl | methyl | methyl |
| choline | Cl | methyl | methyl | methyl |
| choline | F | methyl | methyl | methyl |
| choline | I | methyl | methyl | methyl |
| choline | acetyloxy | methyl | methyl | methyl |
| choline | PH₃P | methyl | methyl | methyl |
| choline | choline | methyl | methyl | methyl |
| choline | N,N,N-methyldiethyl-2- | methyl | methyl | methyl |
| | (11-oxidaneyl)ethan-1- | | | |
| | aminium | | | |
| choline | tosyl | methyl | methyl | methyl |
| choline | brosyl | methyl | methyl | methyl |
| choline | mesyl | methyl | methyl | methyl |
| choline | sulfonate | methyl | methyl | methyl |
| choline | sulfonamide | methyl | methyl | methyl |
| choline | phosphonate | methyl | methyl | methyl |
| H | OH | methyl | methyl | methyl |
| H | Br | methyl | methyl | methyl |
| H | Cl | methyl | methyl | methyl |
| H | F | methyl | methyl | methyl |
| H | I | methyl | methyl | methyl |
| H | acetyloxy | methyl | methyl | methyl |
| H | PH₃P | methyl | methyl | methyl |
| H | choline | methyl | methyl | methyl |
| H | N,N,N-methyldiethyl-2- | methyl | methyl | methyl |
| | (11-oxidaneyl)ethan-1- | | | |
| | aminium | | | |
| H | tosyl | methyl | methyl | methyl |
| H | brosyl | methyl | methyl | methyl |
| H | mesyl | methyl | methyl | methyl |
| H | sulfonate | methyl | methyl | methyl |
| H | sulfonamide | methyl | methyl | methyl |
| H | phosphonate | methyl | methyl | methyl |

TABLE 13
Pericosine Analogues - Structural Formulas V, VI, and VII

| X | Y | W | V | U |
|---|---|---|---|---|
| methyl | OH | ethyl | ethyl | ethyl |
| methyl | Br | ethyl | ethyl | ethyl |
| methyl | Cl | ethyl | ethyl | ethyl |
| methyl | F | ethyl | ethyl | ethyl |
| methyl | acetyloxy | ethyl | ethyl | ethyl |
| methyl | PH₃P | ethyl | ethyl | ethyl |
| methyl | N,N,N-methyldiethyl-2- | ethyl | ethyl | ethyl |
| | (11-oxidaneyl)ethan-1- | | | |
| | aminium | | | |
| methyl | I | ethyl | ethyl | ethyl |
| methyl | tosyl | ethyl | ethyl | ethyl |
| methyl | brosyl | ethyl | ethyl | ethyl |
| methyl | mesyl | ethyl | ethyl | ethyl |
| methyl | sulfonate | ethyl | ethyl | ethyl |
| methyl | sulfonamide | ethyl | ethyl | ethyl |
| methyl | choline | ethyl | ethyl | ethyl |
| methyl | phosphonate | ethyl | ethyl | ethyl |
| ethyl | OH | ethyl | ethyl | ethyl |
| ethyl | Br | ethyl | ethyl | ethyl |
| ethyl | Cl | ethyl | ethyl | ethyl |
| ethyl | F | ethyl | ethyl | ethyl |
| ethyl | I | ethyl | ethyl | ethyl |
| ethyl | acetyloxy | ethyl | ethyl | ethyl |
| ethyl | PH₃P | ethyl | ethyl | ethyl |
| ethyl | choline | ethyl | ethyl | ethyl |
| ethyl | N,N,N-methyldiethyl-2- | ethyl | ethyl | ethyl |
| | (11-oxidaneyl)ethan-1- | | | |
| | aminium | | | |
| ethyl | tosyl | ethyl | ethyl | ethyl |
| ethyl | brosyl | ethyl | ethyl | ethyl |
| ethyl | mesyl | ethyl | ethyl | ethyl |
| ethyl | sulfonate | ethyl | ethyl | ethyl |
| ethyl | sulfonamide | ethyl | ethyl | ethyl |
| ethyl | phosphonate | ethyl | ethyl | ethyl |
| n-propyl | OH | ethyl | ethyl | ethyl |
| n-propyl | Br | ethyl | ethyl | ethyl |
| n-propyl | Cl | ethyl | ethyl | ethyl |
| n-propyl | F | ethyl | ethyl | ethyl |
| n-propyl | I | ethyl | ethyl | ethyl |
| n-propyl | acetyloxy | ethyl | ethyl | ethyl |
| n-propyl | PH₃P | ethyl | ethyl | ethyl |
| n-propyl | choline | ethyl | ethyl | ethyl |
| n-propyl | N,N,N-methyldiethyl-2- | ethyl | ethyl | ethyl |
| | (11-oxidaneyl)ethan-1- | | | |
| | aminium | | | |
| n-propyl | tosyl | ethyl | ethyl | ethyl |
| n-propyl | brosyl | ethyl | ethyl | ethyl |
| n-propyl | mesyl | ethyl | ethyl | ethyl |
| n-propyl | sulfonate | ethyl | ethyl | ethyl |
| n-propyl | sulfonamide | ethyl | ethyl | ethyl |
| n-propyl | phosphonate | ethyl | ethyl | ethyl |
| choline | OH | ethyl | ethyl | ethyl |
| choline | Br | ethyl | ethyl | ethyl |
| choline | Cl | ethyl | ethyl | ethyl |
| choline | F | ethyl | ethyl | ethyl |
| choline | I | ethyl | ethyl | ethyl |
| choline | acetyloxy | ethyl | ethyl | ethyl |
| choline | PH₃P | ethyl | ethyl | ethyl |
| choline | choline | ethyl | ethyl | ethyl |
| choline | N,N,N-methyldiethyl-2- | ethyl | ethyl | ethyl |
| | (11-oxidaneyl)ethan-1- | | | |
| | aminium | | | |
| choline | tosyl | ethyl | ethyl | ethyl |
| choline | brosyl | ethyl | ethyl | ethyl |
| choline | mesyl | ethyl | ethyl | ethyl |
| choline | sulfonate | ethyl | ethyl | ethyl |
| choline | sulfonamide | ethyl | ethyl | ethyl |
| choline | phosphonate | ethyl | ethyl | ethyl |
| H | OH | ethyl | ethyl | ethyl |
| H | Br | ethyl | ethyl | ethyl |
| H | Cl | ethyl | ethyl | ethyl |
| H | F | ethyl | ethyl | ethyl |
| H | I | ethyl | ethyl | ethyl |
| H | acetyloxy | ethyl | ethyl | ethyl |
| H | PH₃P | ethyl | ethyl | ethyl |
| H | choline | ethyl | ethyl | ethyl |
| H | N,N,N-methyldiethyl-2- | ethyl | ethyl | ethyl |
| | (11-oxidaneyl)ethan-1- | | | |
| | aminium | | | |
| H | tosyl | ethyl | ethyl | ethyl |
| H | brosyl | ethyl | ethyl | ethyl |
| H | mesyl | ethyl | ethyl | ethyl |
| H | sulfonate | ethyl | ethyl | ethyl |
| H | sulfonamide | ethyl | ethyl | ethyl |
| H | phosphonate | ethyl | ethyl | ethyl |

TABLE 14
Pericosine Analogues - Structural Formulas V, VI, and VII

| X | Y | W | V | U |
|---|---|---|---|---|
| methyl | OH | OH | H | methoxy |
| methyl | Br | OH | H | methoxy |
| methyl | Cl | OH | H | methoxy |
| methyl | F | OH | H | methoxy |
| methyl | acetyloxy | OH | H | methoxy |
| methyl | PH₃P | OH | H | methoxy |
| methyl | N,N,N-methyldiethyl-2- | OH | H | methoxy |
| | (11-oxidaneyl)ethan-1- | | | |
| | aminium | | | |
| methyl | I | OH | H | methoxy |
| methyl | tosyl | OH | H | methoxy |
| methyl | brosyl | OH | H | methoxy |
| methyl | mesyl | OH | H | methoxy |
| methyl | sulfonate | OH | H | methoxy |
| methyl | sulfonamide | OH | H | methoxy |
| methyl | choline | OH | H | methoxy |
| methyl | phosphonate | OH | H | methoxy |
| ethyl | OH | OH | H | methoxy |
| ethyl | Br | OH | H | methoxy |
| ethyl | Cl | OH | H | methoxy |
| ethyl | F | OH | H | methoxy |
| ethyl | I | OH | H | methoxy |
| ethyl | acetyloxy | OH | H | methoxy |
| ethyl | PH₃P | OH | H | methoxy |
| ethyl | choline | OH | H | methoxy |
| ethyl | N,N,N-methyldiethyl-2- | OH | H | methoxy |
| | (11-oxidaneyl)ethan-1- | | | |
| | aminium | | | |
| ethyl | tosyl | OH | H | methoxy |
| ethyl | brosyl | OH | H | methoxy |
| ethyl | mesyl | OH | H | methoxy |
| ethyl | sulfonate | OH | H | methoxy |
| ethyl | sulfonamide | OH | H | methoxy |
| ethyl | phosphonate | OH | H | methoxy |
| n-propyl | OH | OH | H | methoxy |
| n-propyl | Br | OH | H | methoxy |
| n-propyl | Cl | OH | H | methoxy |
| n-propyl | F | OH | H | methoxy |
| n-propyl | I | OH | H | methoxy |
| n-propyl | acetyloxy | OH | H | methoxy |
| n-propyl | PH₃P | OH | H | methoxy |
| n-propyl | choline | OH | H | methoxy |
| n-propyl | N,N,N-methyldiethyl-2- | OH | H | methoxy |
| | (11-oxidaneyl)ethan-1- | | | |
| | aminium | | | |
| n-propyl | tosyl | OH | H | methoxy |
| n-propyl | brosyl | OH | H | methoxy |
| n-propyl | mesyl | OH | H | methoxy |
| n-propyl | sulfonate | OH | H | methoxy |
| n-propyl | sulfonamide | OH | H | methoxy |
| n-propyl | phosphonate | OH | H | methoxy |
| choline | OH | OH | H | methoxy |
| choline | Br | OH | H | methoxy |
| choline | Cl | OH | H | methoxy |
| choline | F | OH | H | methoxy |
| choline | I | OH | H | methoxy |
| choline | acetyloxy | OH | H | methoxy |
| choline | PH₃P | OH | H | methoxy |
| choline | choline | OH | H | methoxy |
| choline | N,N,N-methyldiethyl-2- | OH | H | methoxy |
| | (11-oxidaneyl)ethan-1- | | | |
| | aminium | | | |
| choline | tosyl | OH | H | methoxy |
| choline | brosyl | OH | H | methoxy |
| choline | mesyl | OH | H | methoxy |
| choline | sulfonate | OH | H | methoxy |
| choline | sulfonamide | OH | H | methoxy |
| choline | phosphonate | OH | H | methoxy |
| H | OH | OH | H | methoxy |
| H | Br | OH | H | methoxy |
| H | Cl | OH | H | methoxy |
| H | F | OH | H | methoxy |
| H | I | OH | H | methoxy |
| H | acetyloxy | OH | H | methoxy |
| H | PH₃P | OH | H | methoxy |
| H | choline | OH | H | methoxy |
| H | N,N,N-methyldiethyl-2- | OH | H | methoxy |
| | (11-oxidaneyl)ethan-1- | | | |
| | aminium | | | |
| H | tosyl | OH | H | methoxy |
| H | brosyl | OH | H | methoxy |
| H | mesyl | OH | H | methoxy |
| H | sulfonate | OH | H | methoxy |
| H | sulfonamide | OH | H | methoxy |
| H | phosphonate | OH | H | methoxy |

TABLE 15
Pericosine Analogues - Structural Formulas V, VI, and VII

| X | Y | W | V | U |
|---|---|---|---|---|
| methyl | OH | OH | H | ethoxy |
| methyl | Br | OH | H | ethoxy |
| methyl | Cl | OH | H | ethoxy |
| methyl | F | OH | H | ethoxy |
| methyl | acetyloxy | OH | H | ethoxy |
| methyl | PH₃P | OH | H | ethoxy |
| methyl | N,N,N-methyldiethyl-2- | OH | H | ethoxy |
| | (11 -oxidaneyl)ethan-1 - | | | |
| | aminium | | | |
| methyl | I | OH | H | ethoxy |
| methyl | tosyl | OH | H | ethoxy |
| methyl | brosyl | OH | H | ethoxy |
| methyl | mesyl | OH | H | ethoxy |
| methyl | sulfonate | OH | H | ethoxy |
| methyl | sulfonamide | OH | H | ethoxy |
| methyl | choline | OH | H | ethoxy |
| methyl | phosphonate | OH | H | ethoxy |
| ethyl | OH | OH | H | ethoxy |
| ethyl | Br | OH | H | ethoxy |
| ethyl | Cl | OH | H | ethoxy |
| ethyl | F | OH | H | ethoxy |
| ethyl | I | OH | H | ethoxy |
| ethyl | acetyloxy | OH | H | ethoxy |
| ethyl | PH₃P | OH | H | ethoxy |
| ethyl | choline | OH | H | ethoxy |
| ethyl | N,N,N-methyldiethyl-2- | OH | H | ethoxy |
| | (11 -oxidaneyl)ethan-1 - | | | |
| | aminium | | | |
| ethyl | tosyl | OH | H | ethoxy |
| ethyl | brosyl | OH | H | ethoxy |
| ethyl | mesyl | OH | H | ethoxy |
| ethyl | sulfonate | OH | H | ethoxy |
| ethyl | sulfonamide | OH | H | ethoxy |
| ethyl | phosphonate | OH | H | ethoxy |
| n-propyl | OH | OH | H | ethoxy |
| n-propyl | Br | OH | H | ethoxy |
| n-propyl | Cl | OH | H | ethoxy |
| n-propyl | F | OH | H | ethoxy |
| n-propyl | I | OH | H | ethoxy |
| n-propyl | acetyloxy | OH | H | ethoxy |
| n-propyl | PH₃P | OH | H | ethoxy |
| n-propyl | choline | OH | H | ethoxy |
| n-propyl | N,N,N-methyldiethyl-2- | OH | H | ethoxy |
| | (11-oxidaneyl)ethan-1- | | | |
| | aminium | | | |
| n-propyl | tosyl | OH | H | ethoxy |
| n-propyl | brosyl | OH | H | ethoxy |
| n-propyl | mesyl | OH | H | ethoxy |
| n-propyl | sulfonate | OH | H | ethoxy |
| n-propyl | sulfonamide | OH | H | ethoxy |
| n-propyl | phosphonate | OH | H | ethoxy |
| choline | OH | OH | H | ethoxy |
| choline | Br | OH | H | ethoxy |
| choline | Cl | OH | H | ethoxy |
| choline | F | OH | H | ethoxy |
| choline | I | OH | H | ethoxy |
| choline | acetyloxy | OH | H | ethoxy |
| choline | PH₃P | OH | H | ethoxy |
| choline | choline | OH | H | ethoxy |
| choline | N,N,N-methyldiethyl-2- | OH | H | ethoxy |
| | (11-oxidaneyl)ethan-1- | | | |
| | aminium | | | |
| choline | tosyl | OH | H | ethoxy |
| choline | brosyl | OH | H | ethoxy |
| choline | mesyl | OH | H | ethoxy |
| choline | sulfonate | OH | H | ethoxy |
| choline | sulfonamide | OH | H | ethoxy |
| choline | phosphonate | OH | H | ethoxy |
| H | OH | OH | H | ethoxy |
| H | Br | OH | H | ethoxy |
| H | Cl | OH | H | ethoxy |
| H | F | OH | H | ethoxy |
| H | I | OH | H | ethoxy |
| H | acetyloxy | OH | H | ethoxy |
| H | PH₃P | OH | H | ethoxy |
| H | choline | OH | H | ethoxy |
| H | N,N,N-methyldiethyl-2- | OH | H | ethoxy |
| | (11-oxidaneyl)ethan-1- | | | |
| | aminium | | | |
| H | tosyl | OH | H | ethoxy |
| H | brosyl | OH | H | ethoxy |
| H | mesyl | OH | H | ethoxy |
| H | sulfonate | OH | H | ethoxy |
| H | sulfonamide | OH | H | ethoxy |
| H | phosphonate | OH | H | ethoxy |

TABLE 16
Pericosine Analogues - Structural Formulas V, VI, and VII

| X | Y | W | V | U |
|---|---|---|---|---|
| methyl | OH | OH | H | methyl |
| methyl | Br | OH | H | methyl |
| methyl | Cl | OH | H | methyl |
| methyl | F | OH | H | methyl |
| methyl | acetyloxy | OH | H | methyl |
| methyl | PH₃P | OH | H | methyl |
| methyl | N,N,N-methyldiethyl-2- | OH | H | methyl |
| | (11-oxidaneyl)ethan-1- | | | |
| | aminium | | | |
| methyl | I | OH | H | methyl |
| methyl | tosyl | OH | H | methyl |
| methyl | brosyl | OH | H | methyl |
| methyl | mesyl | OH | H | methyl |
| methyl | sulfonate | OH | H | methyl |
| methyl | sulfonamide | OH | H | methyl |
| methyl | choline | OH | H | methyl |
| methyl | phosphonate | OH | H | methyl |
| ethyl | OH | OH | H | methyl |
| ethyl | Br | OH | H | methyl |
| ethyl | Cl | OH | H | methyl |
| ethyl | F | OH | H | methyl |
| ethyl | I | OH | H | methyl |
| ethyl | acetyloxy | OH | H | methyl |
| ethyl | PH₃P | OH | H | methyl |
| ethyl | choline | OH | H | methyl |
| ethyl | N,N,N-methyldiethyl-2- | OH | H | methyl |
| | (11-oxidaneyl)ethan-1- | | | |
| | aminium | | | |
| ethyl | tosyl | OH | H | methyl |
| ethyl | brosyl | OH | H | methyl |
| ethyl | mesyl | OH | H | methyl |
| ethyl | sulfonate | OH | H | methyl |
| ethyl | sulfonamide | OH | H | methyl |
| ethyl | phosphonate | OH | H | methyl |
| n-propyl | OH | OH | H | methyl |
| n-propyl | Br | OH | H | methyl |
| n-propyl | Cl | OH | H | methyl |
| n-propyl | F | OH | H | methyl |
| n-propyl | I | OH | H | methyl |
| n-propyl | acetyloxy | OH | H | methyl |
| n-propyl | PH₃P | OH | H | methyl |
| n-propyl | choline | OH | H | methyl |
| n-propyl | N,N,N-methyldiethyl-2- | OH | H | methyl |
| | (11-oxidaneyl)ethan-1- | | | |
| | aminium | | | |
| n-propyl | tosyl | OH | H | methyl |
| n-propyl | brosyl | OH | H | methyl |
| n-propyl | mesyl | OH | H | methyl |
| n-propyl | sulfonate | OH | H | methyl |
| n-propyl | sulfonamide | OH | H | methyl |
| n-propyl | phosphonate | OH | H | methyl |
| choline | OH | OH | H | methyl |
| choline | Br | OH | H | methyl |
| choline | Cl | OH | H | methyl |
| choline | F | OH | H | methyl |
| choline | I | OH | H | methyl |
| choline | acetyloxy | OH | H | methyl |
| choline | PH₃P | OH | H | methyl |
| choline | choline | OH | H | methyl |
| choline | N,N,N-methyldiethyl-2- | OH | H | methyl |
| | (11-oxidaneyl)ethan-1- | | | |
| | aminium | | | |
| choline | tosyl | OH | H | methyl |
| choline | brosyl | OH | H | methyl |
| choline | mesyl | OH | H | methyl |
| choline | sulfonate | OH | H | methyl |
| choline | sulfonamide | OH | H | methyl |
| choline | phosphonate | OH | H | methyl |
| H | OH | OH | H | methyl |
| H | Br | OH | H | methyl |
| H | Cl | OH | H | methyl |
| H | F | OH | H | methyl |
| H | I | OH | H | methyl |
| H | acetyloxy | OH | H | methyl |
| H | PH₃P | OH | H | methyl |
| H | choline | OH | H | methyl |
| H | N,N,N-methyldiethyl-2- | OH | H | methyl |
| | (11-oxidaneyl)ethan-1- | | | |
| | aminium | | | |
| H | tosyl | OH | H | methyl |
| H | brosyl | OH | H | methyl |
| H | mesyl | OH | H | methyl |
| H | sulfonate | OH | H | methyl |
| H | sulfonamide | OH | H | methyl |
| H | phosphonate | OH | H | methyl |

TABLE 17
Pericosine Analogues - Structural Formulas V, VI, and VII

| X | Y | W | V | U |
|---|---|---|---|---|
| methyl | OH | OH | H | ethyl |
| methyl | Br | OH | H | ethyl |
| methyl | Cl | OH | H | ethyl |
| methyl | F | OH | H | ethyl |
| methyl | acetyloxy | OH | H | ethyl |
| methyl | PH₃P | OH | H | ethyl |
| methyl | N,N,N-methyldiethyl-2- | OH | H | ethyl |
| | (11-oxidaneyl)ethan-1- | | | |
| | aminium | | | |
| methyl | I | OH | H | ethyl |
| methyl | tosyl | OH | H | ethyl |
| methyl | brosyl | OH | H | ethyl |
| methyl | mesyl | OH | H | ethyl |
| methyl | sulfonate | OH | H | ethyl |
| methyl | sulfonamide | OH | H | ethyl |
| methyl | choline | OH | H | ethyl |
| methyl | phosphonate | OH | H | ethyl |
| ethyl | OH | OH | H | ethyl |
| ethyl | Br | OH | H | ethyl |
| ethyl | Cl | OH | H | ethyl |
| ethyl | F | OH | H | ethyl |
| ethyl | I | OH | H | ethyl |
| ethyl | acetyloxy | OH | H | ethyl |
| ethyl | PH₃P | OH | H | ethyl |
| ethyl | choline | OH | H | ethyl |
| ethyl | N,N,N-methyldiethyl-2- | OH | H | ethyl |
| | (11-oxidaneyl)ethan-1- | | | |
| | aminium | | | |
| ethyl | tosyl | OH | H | ethyl |
| ethyl | brosyl | OH | H | ethyl |
| ethyl | mesyl | OH | H | ethyl |
| ethyl | sulfonate | OH | H | ethyl |
| ethyl | sulfonamide | OH | H | ethyl |
| ethyl | phosphonate | OH | H | ethyl |
| n-propyl | OH | OH | H | ethyl |
| n-propyl | Br | OH | H | ethyl |
| n-propyl | Cl | OH | H | ethyl |
| n-propyl | F | OH | H | ethyl |
| n-propyl | I | OH | H | ethyl |
| n-propyl | acetyloxy | OH | H | ethyl |
| n-propyl | PH₃P | OH | H | ethyl |
| n-propyl | choline | OH | H | ethyl |
| n-propyl | N,N,N-methyldiethyl-2- | OH | H | ethyl |
| | (11-oxidaneyl)ethan-1- | | | |
| | aminium | | | |
| n-propyl | tosyl | OH | H | ethyl |
| n-propyl | brosyl | OH | H | ethyl |
| n-propyl | mesyl | OH | H | ethyl |
| n-propyl | sulfonate | OH | H | ethyl |
| n-propyl | sulfonamide | OH | H | ethyl |
| n-propyl | phosphonate | OH | H | ethyl |
| choline | OH | OH | H | ethyl |
| choline | Br | OH | H | ethyl |
| choline | Cl | OH | H | ethyl |
| choline | F | OH | H | ethyl |
| choline | I | OH | H | ethyl |
| choline | acetyloxy | OH | H | ethyl |
| choline | PH₃P | OH | H | ethyl |
| choline | choline | OH | H | ethyl |
| choline | N,N,N-methyldiethyl-2- | OH | H | ethyl |
| | (11-oxidaneyl)ethan-1- | | | |
| | aminium | | | |
| choline | tosyl | OH | H | ethyl |
| choline | brosyl | OH | H | ethyl |
| choline | mesyl | OH | H | ethyl |
| choline | sulfonate | OH | H | ethyl |
| choline | sulfonamide | OH | H | ethyl |
| choline | phosphonate | OH | H | ethyl |
| H | OH | OH | H | ethyl |
| H | Br | OH | H | ethyl |
| H | Cl | OH | H | ethyl |
| H | F | OH | H | ethyl |
| H | I | OH | H | ethyl |
| H | acetyloxy | OH | H | ethyl |
| H | PH₃P | OH | H | ethyl |
| H | choline | OH | H | ethyl |
| H | N,N,N-methyldiethyl-2- | OH | H | ethyl |
| | (11-oxidaneyl)ethan-1- | | | |
| | aminium | | | |
| H | tosyl | OH | H | ethyl |
| H | brosyl | OH | H | ethyl |
| H | mesyl | OH | H | ethyl |
| H | sulfonate | OH | H | ethyl |
| H | sulfonamide | OH | H | ethyl |
| H | phosphonate | OH | H | ethyl |

TABLE 18
Pericosine Analogues - Structural Formulas V, VI, and VII

| X | Y | W | V | U |
|---|---|---|---|---|
| methyl | OH | methoxy | H | OH |
| methyl | Br | methoxy | H | OH |
| methyl | Cl | methoxy | H | OH |
| methyl | F | methoxy | H | OH |
| methyl | acetyloxy | methoxy | H | OH |
| methyl | PH₃P | methoxy | H | OH |
| methyl | N,N,N-methyldiethyl-2- | methoxy | H | OH |
| | (11-oxidaneyl)ethan-1- | | | |
| | aminium | | | |
| methyl | I | methoxy | H | OH |
| methyl | tosyl | methoxy | H | OH |
| methyl | brosyl | methoxy | H | OH |
| methyl | mesyl | methoxy | H | OH |
| methyl | sulfonate | methoxy | H | OH |
| methyl | sulfonamide | methoxy | H | OH |
| methyl | choline | methoxy | H | OH |
| methyl | phosphonate | methoxy | H | OH |
| ethyl | OH | methoxy | H | OH |
| ethyl | Br | methoxy | H | OH |
| ethyl | Cl | methoxy | H | OH |
| ethyl | F | methoxy | H | OH |
| ethyl | I | methoxy | H | OH |
| ethyl | acetyloxy | methoxy | H | OH |
| ethyl | PH₃P | methoxy | H | OH |
| ethyl | choline | methoxy | H | OH |
| ethyl | N,N,N-methyldiethyl-2- | methoxy | H | OH |
| | (11-oxidaneyl)ethan-1- | | | |
| | aminium | | | |
| ethyl | tosyl | methoxy | H | OH |
| ethyl | brosyl | methoxy | H | OH |
| ethyl | mesyl | methoxy | H | OH |
| ethyl | sulfonate | methoxy | H | OH |
| ethyl | sulfonamide | methoxy | H | OH |
| ethyl | phosphonate | methoxy | H | OH |
| n-propyl | OH | methoxy | H | OH |
| n-propyl | Br | methoxy | H | OH |
| n-propyl | Cl | methoxy | H | OH |
| n-propyl | F | methoxy | H | OH |
| n-propyl | I | methoxy | H | OH |
| n-propyl | acetyloxy | methoxy | H | OH |
| n-propyl | PH₃P | methoxy | H | OH |
| n-propyl | choline | methoxy | H | OH |
| n-propyl | N,N,N-methyldiethyl-2- | methoxy | H | OH |
| | (11-oxidaneyl)ethan-1- | | | |
| | aminium | | | |
| n-propyl | tosyl | methoxy | H | OH |
| n-propyl | brosyl | methoxy | H | OH |
| n-propyl | mesyl | methoxy | H | OH |
| n-propyl | sulfonate | methoxy | H | OH |
| n-propyl | sulfonamide | methoxy | H | OH |
| n-propyl | phosphonate | methoxy | H | OH |
| choline | OH | methoxy | H | OH |
| choline | Br | methoxy | H | OH |
| choline | Cl | methoxy | H | OH |
| choline | F | methoxy | H | OH |
| choline | I | methoxy | H | OH |
| choline | acetyloxy | methoxy | H | OH |
| choline | PH₃P | methoxy | H | OH |
| choline | choline | methoxy | H | OH |
| choline | N,N,N-methyldiethyl-2- | methoxy | H | OH |
| | (11-oxidaneyl)ethan-1- | | | |
| | aminium | | | |
| choline | tosyl | methoxy | H | OH |
| choline | brosyl | methoxy | H | OH |
| choline | mesyl | methoxy | H | OH |
| choline | sulfonate | methoxy | H | OH |
| choline | sulfonamide | methoxy | H | OH |
| choline | phosphonate | methoxy | H | OH |
| H | OH | methoxy | H | OH |
| H | Br | methoxy | H | OH |
| H | Cl | methoxy | H | OH |
| H | F | methoxy | H | OH |
| H | I | methoxy | H | OH |
| H | acetyloxy | methoxy | H | OH |
| H | PH₃P | methoxy | H | OH |
| H | choline | methoxy | H | OH |
| H | N,N,N-methyldiethyl-2- | methoxy | H | OH |
| | (11-oxidaneyl)ethan-1- | | | |
| | aminium | | | |
| H | tosyl | methoxy | H | OH |
| H | brosyl | methoxy | H | OH |
| H | mesyl | methoxy | H | OH |
| H | sulfonate | methoxy | H | OH |
| H | sulfonamide | methoxy | H | OH |
| H | phosphonate | methoxy | H | OH |

TABLE 19
Pericosine Analogues - Structural Formulas V, VI, and VII

| X | Y | W | V | U |
|---|---|---|---|---|
| methyl | OH | ethoxy | H | OH |
| methyl | Br | ethoxy | H | OH |
| methyl | Cl | ethoxy | H | OH |
| methyl | F | ethoxy | H | OH |
| methyl | acetyloxy | ethoxy | H | OH |
| methyl | PH₃P | ethoxy | H | OH |
| methyl | N,N,N-methyldiethyl-2- | ethoxy | H | OH |
| | (11-oxidaneyl)ethan-1- | | | |
| | aminium | | | |
| methyl | I | ethoxy | H | OH |
| methyl | tosyl | ethoxy | H | OH |
| methyl | brosyl | ethoxy | H | OH |
| methyl | mesyl | ethoxy | H | OH |
| methyl | sulfonate | ethoxy | H | OH |
| methyl | sulfonamide | ethoxy | H | OH |
| methyl | choline | ethoxy | H | OH |
| methyl | phosphonate | ethoxy | H | OH |
| ethyl | OH | ethoxy | H | OH |
| ethyl | Br | ethoxy | H | OH |
| ethyl | Cl | ethoxy | H | OH |
| ethyl | F | ethoxy | H | OH |
| ethyl | I | ethoxy | H | OH |
| ethyl | acetyloxy | ethoxy | H | OH |
| ethyl | PH₃P | ethoxy | H | OH |
| ethyl | choline | ethoxy | H | OH |
| ethyl | N,N,N-methyldiethyl-2- | ethoxy | H | OH |
| | (11-oxidaneyl)ethan-1- | | | |
| | aminium | | | |
| ethyl | tosyl | ethoxy | H | OH |
| ethyl | brosyl | ethoxy | H | OH |
| ethyl | mesyl | ethoxy | H | OH |
| ethyl | sulfonate | ethoxy | H | OH |
| ethyl | sulfonamide | ethoxy | H | OH |
| ethyl | phosphonate | ethoxy | H | OH |
| n-propyl | OH | ethoxy | H | OH |
| n-propyl | Br | ethoxy | H | OH |
| n-propyl | Cl | ethoxy | H | OH |
| n-propyl | F | ethoxy | H | OH |
| n-propyl | I | ethoxy | H | OH |
| n-propyl | acetyloxy | ethoxy | H | OH |
| n-propyl | PH₃P | ethoxy | H | OH |
| n-propyl | choline | ethoxy | H | OH |
| n-propyl | N,N,N-methyldiethyl-2- | ethoxy | H | OH |
| | (11-oxidaneyl)ethan-1- | | | |
| | aminium | | | |
| n-propyl | tosyl | ethoxy | H | OH |
| n-propyl | brosyl | ethoxy | H | OH |
| n-propyl | mesyl | ethoxy | H | OH |
| n-propyl | sulfonate | ethoxy | H | OH |
| n-propyl | sulfonamide | ethoxy | H | OH |
| n-propyl | phosphonate | ethoxy | H | OH |
| choline | OH | ethoxy | H | OH |
| choline | Br | ethoxy | H | OH |
| choline | Cl | ethoxy | H | OH |
| choline | F | ethoxy | H | OH |
| choline | I | ethoxy | H | OH |
| choline | acetyloxy | ethoxy | H | OH |
| choline | PH₃P | ethoxy | H | OH |
| choline | choline | ethoxy | H | OH |
| choline | N,N,N-methyldiethyl-2- | ethoxy | H | OH |
| | (11-oxidaneyl)ethan-1- | | | |
| | aminium | | | |
| choline | tosyl | ethoxy | H | OH |
| choline | brosyl | ethoxy | H | OH |
| choline | mesyl | ethoxy | H | OH |
| choline | sulfonate | ethoxy | H | OH |
| choline | sulfonamide | ethoxy | H | OH |
| choline | phosphonate | ethoxy | H | OH |
| H | OH | ethoxy | H | OH |
| H | Br | ethoxy | H | OH |
| H | Cl | ethoxy | H | OH |
| H | F | ethoxy | H | OH |
| H | I | ethoxy | H | OH |
| H | acetyloxy | ethoxy | H | OH |
| H | PH₃P | ethoxy | H | OH |
| H | choline | ethoxy | H | OH |
| H | N,N,N-methyldiethyl-2- | ethoxy | H | OH |
| | (11-oxidaneyl)ethan-1- | | | |
| | aminium | | | |
| H | tosyl | ethoxy | H | OH |
| H | brosyl | ethoxy | H | OH |
| H | mesyl | ethoxy | H | OH |
| H | sulfonate | ethoxy | H | OH |
| H | sulfonamide | ethoxy | H | OH |
| H | phosphonate | ethoxy | H | OH |

TABLE 20
Pericosine Analogues - Structural Formulas V, VI, and VII

| X | Y | W | V | U |
|---|---|---|---|---|
| methyl | OH | methyl | H | OH |
| methyl | Br | methyl | H | OH |
| methyl | Cl | methyl | H | OH |
| methyl | F | methyl | H | OH |
| methyl | acetyloxy | methyl | H | OH |
| methyl | PH₃P | methyl | H | OH |
| methyl | N,N,N-methyldiethyl-2- | methyl | H | OH |
| | (11-oxidaneyl)ethan-1- | | | |
| | aminium | | | |
| methyl | I | methyl | H | OH |
| methyl | tosyl | methyl | H | OH |
| methyl | brosyl | methyl | H | OH |
| methyl | mesyl | methyl | H | OH |
| methyl | sulfonate | methyl | H | OH |
| methyl | sulfonamide | methyl | H | OH |
| methyl | choline | methyl | H | OH |
| methyl | phosphonate | methyl | H | OH |
| ethyl | OH | methyl | H | OH |
| ethyl | Br | methyl | H | OH |
| ethyl | Cl | methyl | H | OH |
| ethyl | F | methyl | H | OH |
| ethyl | I | methyl | H | OH |
| ethyl | acetyloxy | methyl | H | OH |
| ethyl | PH₃P | methyl | H | OH |
| ethyl | choline | methyl | H | OH |
| ethyl | N,N,N-methyldiethyl-2- | methyl | H | OH |
| | (11-oxidaneyl)ethan-1- | | | |
| | aminium | | | |
| ethyl | tosyl | methyl | H | OH |
| ethyl | brosyl | methyl | H | OH |
| ethyl | mesyl | methyl | H | OH |
| ethyl | sulfonate | methyl | H | OH |
| ethyl | sulfonamide | methyl | H | OH |
| ethyl | phosphonate | methyl | H | OH |
| n-propyl | OH | methyl | H | OH |
| n-propyl | Br | methyl | H | OH |
| n-propyl | Cl | methyl | H | OH |
| n-propyl | F | methyl | H | OH |
| n-propyl | I | methyl | H | OH |
| n-propyl | acetyloxy | methyl | H | OH |
| n-propyl | PH₃P | methyl | H | OH |
| n-propyl | choline | methyl | H | OH |
| n-propyl | N,N,N-methyldiethyl-2- | methyl | H | OH |
| | (11-oxidaneyl)ethan-1- | | | |
| | aminium | | | |
| n-propyl | tosyl | methyl | H | OH |
| n-propyl | brosyl | methyl | H | OH |
| n-propyl | mesyl | methyl | H | OH |
| n-propyl | sulfonate | methyl | H | OH |
| n-propyl | sulfonamide | methyl | H | OH |
| n-propyl | phosphonate | methyl | H | OH |
| choline | OH | methyl | H | OH |
| choline | Br | methyl | H | OH |
| choline | Cl | methyl | H | OH |
| choline | F | methyl | H | OH |
| choline | I | methyl | H | OH |
| choline | acetyloxy | methyl | H | OH |
| choline | PH₃P | methyl | H | OH |
| choline | choline | methyl | H | OH |
| choline | N,N,N-methyldiethyl-2- | methyl | H | OH |
| | (11-oxidaneyl)ethan-1- | | | |
| | aminium | | | |
| choline | tosyl | methyl | H | OH |
| choline | brosyl | methyl | H | OH |
| choline | mesyl | methyl | H | OH |
| choline | sulfonate | methyl | H | OH |
| choline | sulfonamide | methyl | H | OH |
| choline | phosphonate | methyl | H | OH |
| H | OH | methyl | H | OH |
| H | Br | methyl | H | OH |
| H | Cl | methyl | H | OH |
| H | F | methyl | H | OH |
| H | I | methyl | H | OH |
| H | acetyloxy | methyl | H | OH |
| H | PH₃P | methyl | H | OH |
| H | choline | methyl | H | OH |
| H | N,N,N-methyldiethyl-2- | methyl | H | OH |
| | (11-oxidaneyl)ethan-1- | | | |
| | aminium | | | |
| H | tosyl | methyl | H | OH |
| H | brosyl | methyl | H | OH |
| H | mesyl | methyl | H | OH |
| H | sulfonate | methyl | H | OH |
| H | sulfonamide | methyl | H | OH |
| H | phosphonate | methyl | H | OH |

TABLE 21
Pericosine Analogues - Structural Formulas V, VI, and VII

| X | Y | W | V | U |
|---|---|---|---|---|
| methyl | OH | ethyl | H | OH |
| methyl | Br | ethyl | H | OH |
| methyl | Cl | ethyl | H | OH |
| methyl | F | ethyl | H | OH |
| methyl | acetyloxy | ethyl | H | OH |
| methyl | PH₃P | ethyl | H | OH |
| methyl | N,N,N-methyldiethyl-2- | ethyl | H | OH |
| | (11-oxidaneyl)ethan-1- | | | |
| | aminium | | | |
| methyl | I | ethyl | H | OH |
| methyl | tosyl | ethyl | H | OH |
| methyl | brosyl | ethyl | H | OH |
| methyl | mesyl | ethyl | H | OH |
| methyl | sulfonate | ethyl | H | OH |
| methyl | sulfonamide | ethyl | H | OH |
| methyl | choline | ethyl | H | OH |
| methyl | phosphonate | ethyl | H | OH |
| ethyl | OH | ethyl | H | OH |
| ethyl | Br | ethyl | H | OH |
| ethyl | Cl | ethyl | H | OH |
| ethyl | F | ethyl | H | OH |
| ethyl | I | ethyl | H | OH |
| ethyl | acetyloxy | ethyl | H | OH |
| ethyl | PH₃P | ethyl | H | OH |
| ethyl | choline | ethyl | H | OH |
| ethyl | N,N,N-methyldiethyl-2- | ethyl | H | OH |
| | (11-oxidaneyl)ethan-1- | | | |
| | aminium | | | |
| ethyl | tosyl | ethyl | H | OH |
| ethyl | brosyl | ethyl | H | OH |
| ethyl | mesyl | ethyl | H | OH |
| ethyl | sulfonate | ethyl | H | OH |
| ethyl | sulfonamide | ethyl | H | OH |
| ethyl | phosphonate | ethyl | H | OH |
| n-propyl | OH | ethyl | H | OH |
| n-propyl | Br | ethyl | H | OH |
| n-propyl | Cl | ethyl | H | OH |
| n-propyl | F | ethyl | H | OH |
| n-propyl | I | ethyl | H | OH |
| n-propyl | acetyloxy | ethyl | H | OH |
| n-propyl | PH₃P | ethyl | H | OH |
| n-propyl | choline | ethyl | H | OH |
| n-propyl | N,N,N-methyldiethyl-2- | ethyl | H | OH |
| | (11-oxidaneyl)ethan-1- | | | |
| | aminium | | | |
| n-propyl | tosyl | ethyl | H | OH |
| n-propyl | brosyl | ethyl | H | OH |
| n-propyl | mesyl | ethyl | H | OH |
| n-propyl | sulfonate | ethyl | H | OH |
| n-propyl | sulfonamide | ethyl | H | OH |
| n-propyl | phosphonate | ethyl | H | OH |
| choline | OH | ethyl | H | OH |
| choline | Br | ethyl | H | OH |
| choline | Cl | ethyl | H | OH |
| choline | F | ethyl | H | OH |
| choline | I | ethyl | H | OH |
| choline | acetyloxy | ethyl | H | OH |
| choline | PH₃P | ethyl | H | OH |
| choline | choline | ethyl | H | OH |
| choline | N,N,N-methyldiethyl-2- | ethyl | H | OH |
| | (11-oxidaneyl)ethan-1- | | | |
| | aminium | | | |
| choline | tosyl | ethyl | H | OH |
| choline | brosyl | ethyl | H | OH |
| choline | mesyl | ethyl | H | OH |
| choline | sulfonate | ethyl | H | OH |
| choline | sulfonamide | ethyl | H | OH |
| choline | phosphonate | ethyl | H | OH |
| H | OH | ethyl | H | OH |
| H | Br | ethyl | H | OH |
| H | Cl | ethyl | H | OH |
| H | F | ethyl | H | OH |
| H | I | ethyl | H | OH |
| H | acetyloxy | ethyl | H | OH |
| H | PH₃P | ethyl | H | OH |
| H | choline | ethyl | H | OH |
| H | N,N,N-methyldiethyl-2- | ethyl | H | OH |
| | (11-oxidaneyl)ethan-1- | | | |
| | aminium | | | |
| H | tosyl | ethyl | H | OH |
| H | brosyl | ethyl | H | OH |
| H | mesyl | ethyl | H | OH |
| H | sulfonate | ethyl | H | OH |
| H | sulfonamide | ethyl | H | OH |
| H | phosphonate | ethyl | H | OH |

TABLE 22
Pericosine Analogues - Structural Formulas V, VI, and VII

| X | Y | W | V | U |
|---|---|---|---|---|
| methyl | OH | OH | methoxy | OH |
| methyl | Br | OH | methoxy | OH |
| methyl | Cl | OH | methoxy | OH |
| methyl | F | OH | methoxy | OH |
| methyl | acetyloxy | OH | methoxy | OH |
| methyl | PH₃P | OH | methoxy | OH |
| methyl | N,N,N-methyldiethyl-2- | OH | methoxy | OH |
| | (11-oxidaneyl)ethan-1- | | | |
| | aminium | | | |
| methyl | I | OH | methoxy | OH |
| methyl | tosyl | OH | methoxy | OH |
| methyl | brosyl | OH | methoxy | OH |
| methyl | mesyl | OH | methoxy | OH |
| methyl | sulfonate | OH | methoxy | OH |
| methyl | sulfonamide | OH | methoxy | OH |
| methyl | choline | OH | methoxy | OH |
| methyl | phosphonate | OH | methoxy | OH |
| ethyl | OH | OH | methoxy | OH |
| ethyl | Br | OH | methoxy | OH |
| ethyl | Cl | OH | methoxy | OH |
| ethyl | F | OH | methoxy | OH |
| ethyl | I | OH | methoxy | OH |
| ethyl | acetyloxy | OH | methoxy | OH |
| ethyl | PH₃P | OH | methoxy | OH |
| ethyl | choline | OH | methoxy | OH |
| ethyl | N,N,N-methyldiethyl-2- | OH | methoxy | OH |
| | (11-oxidaneyl)ethan-1- | | | |
| | aminium | | | |
| ethyl | tosyl | OH | methoxy | OH |
| ethyl | brosyl | OH | methoxy | OH |
| ethyl | mesyl | OH | methoxy | OH |
| ethyl | sulfonate | OH | methoxy | OH |
| ethyl | sulfonamide | OH | methoxy | OH |
| ethyl | phosphonate | OH | methoxy | OH |
| n-propyl | OH | OH | methoxy | OH |
| n-propyl | Br | OH | methoxy | OH |
| n-propyl | Cl | OH | methoxy | OH |
| n-propyl | F | OH | methoxy | OH |
| n-propyl | I | OH | methoxy | OH |
| n-propyl | acetyloxy | OH | methoxy | OH |
| n-propyl | PH₃P | OH | methoxy | OH |
| n-propyl | choline | OH | methoxy | OH |
| n-propyl | N,N,N-methyldiethyl-2- | OH | methoxy | OH |
| | (11-oxidaneyl)ethan-1- | | | |
| | aminium | | | |
| n-propyl | tosyl | OH | methoxy | OH |
| n-propyl | brosyl | OH | methoxy | OH |
| n-propyl | mesyl | OH | methoxy | OH |
| n-propyl | sulfonate | OH | methoxy | OH |
| n-propyl | sulfonamide | OH | methoxy | OH |
| n-propyl | phosphonate | OH | methoxy | OH |
| choline | OH | OH | methoxy | OH |
| choline | Br | OH | methoxy | OH |
| choline | Cl | OH | methoxy | OH |
| choline | F | OH | methoxy | OH |
| choline | I | OH | methoxy | OH |
| choline | acetyloxy | OH | methoxy | OH |
| choline | PH₃P | OH | methoxy | OH |
| choline | choline | OH | methoxy | OH |
| choline | N,N,N-methyldiethyl-2- | OH | methoxy | OH |
| | (11-oxidaneyl)ethan-1- | | | |
| | aminium | | | |
| choline | tosyl | OH | methoxy | OH |
| choline | brosyl | OH | methoxy | OH |
| choline | mesyl | OH | methoxy | OH |
| choline | sulfonate | OH | methoxy | OH |
| choline | sulfonamide | OH | methoxy | OH |
| choline | phosphonate | OH | methoxy | OH |
| H | OH | OH | methoxy | OH |
| H | Br | OH | methoxy | OH |
| H | Cl | OH | methoxy | OH |
| H | F | OH | methoxy | OH |
| H | I | OH | methoxy | OH |
| H | acetyloxy | OH | methoxy | OH |
| H | PH₃P | OH | methoxy | OH |
| H | choline | OH | methoxy | OH |
| H | N,N,N-methyldiethyl-2- | OH | methoxy | OH |
| | (11-oxidaneyl)ethan-1- | | | |
| | aminium | | | |
| H | tosyl | OH | methoxy | OH |
| H | brosyl | OH | methoxy | OH |
| H | mesyl | OH | methoxy | OH |
| H | sulfonate | OH | methoxy | OH |
| H | sulfonamide | OH | methoxy | OH |
| H | phosphonate | OH | methoxy | OH |

TABLE 23
Pericosine Analogues - Structural Formulas V, VI, and VII

| X | Y | W | V | U |
|---|---|---|---|---|
| methyl | OH | OH | ethoxy | OH |
| methyl | Br | OH | ethoxy | OH |
| methyl | Cl | OH | ethoxy | OH |
| methyl | F | OH | ethoxy | OH |
| methyl | acetyloxy | OH | ethoxy | OH |
| methyl | PH₃P | OH | ethoxy | OH |
| methyl | N,N,N-methyldiethyl-2- | OH | ethoxy | OH |
| | (11-oxidaneyl)ethan-1- | | | |
| | aminium | | | |
| methyl | I | OH | ethoxy | OH |
| methyl | tosyl | OH | ethoxy | OH |
| methyl | brosyl | OH | ethoxy | OH |
| methyl | mesyl | OH | ethoxy | OH |
| methyl | sulfonate | OH | ethoxy | OH |
| methyl | sulfonamide | OH | ethoxy | OH |
| methyl | choline | OH | ethoxy | OH |
| methyl | phosphonate | OH | ethoxy | OH |
| ethyl | OH | OH | ethoxy | OH |
| ethyl | Br | OH | ethoxy | OH |
| ethyl | Cl | OH | ethoxy | OH |
| ethyl | F | OH | ethoxy | OH |
| ethyl | I | OH | ethoxy | OH |
| ethyl | acetyloxy | OH | ethoxy | OH |
| ethyl | PH₃P | OH | ethoxy | OH |
| ethyl | choline | OH | ethoxy | OH |
| ethyl | N,N,N-methyldiethyl-2- | OH | ethoxy | OH |
| | (11-oxidaneyl)ethan-1- | | | |
| | aminium | | | |
| ethyl | tosyl | OH | ethoxy | OH |
| ethyl | brosyl | OH | ethoxy | OH |
| ethyl | mesyl | OH | ethoxy | OH |
| ethyl | sulfonate | OH | ethoxy | OH |
| ethyl | sulfonamide | OH | ethoxy | OH |
| ethyl | phosphonate | OH | ethoxy | OH |
| n-propyl | OH | OH | ethoxy | OH |
| n-propyl | Br | OH | ethoxy | OH |
| n-propyl | Cl | OH | ethoxy | OH |
| n-propyl | F | OH | ethoxy | OH |
| n-propyl | I | OH | ethoxy | OH |
| n-propyl | acetyloxy | OH | ethoxy | OH |
| n-propyl | PH₃P | OH | ethoxy | OH |
| n-propyl | choline | OH | ethoxy | OH |
| n-propyl | N,N,N-methyldiethyl-2- | OH | ethoxy | OH |
| | (11-oxidaneyl)ethan-1- | | | |
| | aminium | | | |
| n-propyl | tosyl | OH | ethoxy | OH |
| n-propyl | brosyl | OH | ethoxy | OH |
| n-propyl | mesyl | OH | ethoxy | OH |
| n-propyl | sulfonate | OH | ethoxy | OH |
| n-propyl | sulfonamide | OH | ethoxy | OH |
| n-propyl | phosphonate | OH | ethoxy | OH |
| choline | OH | OH | ethoxy | OH |
| choline | Br | OH | ethoxy | OH |
| choline | Cl | OH | ethoxy | OH |
| choline | F | OH | ethoxy | OH |
| choline | I | OH | ethoxy | OH |
| choline | acetyloxy | OH | ethoxy | OH |
| choline | PH₃P | OH | ethoxy | OH |
| choline | choline | OH | ethoxy | OH |
| choline | N,N,N-methyldiethyl-2- | OH | ethoxy | OH |
| | (11-oxidaneyl)ethan-1- | | | |
| | aminium | | | |
| choline | tosyl | OH | ethoxy | OH |
| choline | brosyl | OH | ethoxy | OH |
| choline | mesyl | OH | ethoxy | OH |
| choline | sulfonate | OH | ethoxy | OH |
| choline | sulfonamide | OH | ethoxy | OH |
| choline | phosphonate | OH | ethoxy | OH |
| H | OH | OH | ethoxy | OH |
| H | Br | OH | ethoxy | OH |
| H | Cl | OH | ethoxy | OH |
| H | F | OH | ethoxy | OH |
| H | I | OH | ethoxy | OH |
| H | acetyloxy | OH | ethoxy | OH |
| H | PH₃P | OH | ethoxy | OH |
| H | choline | OH | ethoxy | OH |
| H | N,N,N-methyldiethyl-2- | OH | ethoxy | OH |
| | (11-oxidaneyl)ethan-1- | | | |
| | aminium | | | |
| H | tosyl | OH | ethoxy | OH |
| H | brosyl | OH | ethoxy | OH |
| H | mesyl | OH | ethoxy | OH |
| H | sulfonate | OH | ethoxy | OH |
| H | sulfonamide | OH | ethoxy | OH |
| H | phosphonate | OH | ethoxy | OH |

TABLE 24
Pericosine Analogues - Structural Formulas V, VI, and VII

| X | Y | W | V | U |
|---|---|---|---|---|
| methyl | OH | OH | methyl | OH |
| methyl | Br | OH | methyl | OH |
| methyl | Cl | OH | methyl | OH |
| methyl | F | OH | methyl | OH |
| methyl | acetyloxy | OH | methyl | OH |
| methyl | PH₃P | OH | methyl | OH |
| methyl | N,N,N-methyldiethyl-2- | OH | methyl | OH |
| | (11-oxidaneyl)ethan-1- | | | |
| | aminium | | | |
| methyl | I | OH | methyl | OH |
| methyl | tosyl | OH | methyl | OH |
| methyl | brosyl | OH | methyl | OH |
| methyl | mesyl | OH | methyl | OH |
| methyl | sulfonate | OH | methyl | OH |
| methyl | sulfonamide | OH | methyl | OH |
| methyl | choline | OH | methyl | OH |
| methyl | phosphonate | OH | methyl | OH |
| ethyl | OH | OH | methyl | OH |
| ethyl | Br | OH | methyl | OH |
| ethyl | Cl | OH | methyl | OH |
| ethyl | F | OH | methyl | OH |
| ethyl | I | OH | methyl | OH |
| ethyl | acetyloxy | OH | methyl | OH |
| ethyl | PH₃P | OH | methyl | OH |
| ethyl | choline | OH | methyl | OH |
| ethyl | N,N,N-methyldiethyl-2- | OH | methyl | OH |
| | (11-oxidaneyl)ethan-1- | | | |
| | aminium | | | |
| ethyl | tosyl | OH | methyl | OH |
| ethyl | brosyl | OH | methyl | OH |
| ethyl | mesyl | OH | methyl | OH |
| ethyl | sulfonate | OH | methyl | OH |
| ethyl | sulfonamide | OH | methyl | OH |
| ethyl | phosphonate | OH | methyl | OH |
| n-propyl | OH | OH | methyl | OH |
| n-propyl | Br | OH | methyl | OH |
| n-propyl | Cl | OH | methyl | OH |
| n-propyl | F | OH | methyl | OH |
| n-propyl | I | OH | methyl | OH |
| n-propyl | acetyloxy | OH | methyl | OH |
| n-propyl | PH₃P | OH | methyl | OH |
| n-propyl | choline | OH | methyl | OH |
| n-propyl | N,N,N-methyldiethyl-2- | OH | methyl | OH |
| | (11-oxidaneyl)ethan-1- | | | |
| | aminium | | | |
| n-propyl | tosyl | OH | methyl | OH |
| n-propyl | brosyl | OH | methyl | OH |
| n-propyl | mesyl | OH | methyl | OH |
| n-propyl | sulfonate | OH | methyl | OH |
| n-propyl | sulfonamide | OH | methyl | OH |
| n-propyl | phosphonate | OH | methyl | OH |
| choline | OH | OH | methyl | OH |
| choline | Br | OH | methyl | OH |
| choline | Cl | OH | methyl | OH |
| choline | F | OH | methyl | OH |
| choline | I | OH | methyl | OH |
| choline | acetyloxy | OH | methyl | OH |
| choline | PH₃P | OH | methyl | OH |
| choline | choline | OH | methyl | OH |
| choline | N,N,N-methyldiethyl-2- | OH | methyl | OH |
| | (11-oxidaneyl)ethan-1- | | | |
| | aminium | | | |
| choline | tosyl | OH | methyl | OH |
| choline | brosyl | OH | methyl | OH |
| choline | mesyl | OH | methyl | OH |
| choline | sulfonate | OH | methyl | OH |
| choline | sulfonamide | OH | methyl | OH |
| choline | phosphonate | OH | methyl | OH |
| H | OH | OH | methyl | OH |
| H | Br | OH | methyl | OH |
| H | Cl | OH | methyl | OH |
| H | F | OH | methyl | OH |
| H | I | OH | methyl | OH |
| H | acetyloxy | OH | methyl | OH |
| H | PH₃P | OH | methyl | OH |
| H | choline | OH | methyl | OH |
| H | N,N,N-methyldiethyl-2- | OH | methyl | OH |
| | (11-oxidaneyl)ethan-1- | | | |
| | aminium | | | |
| H | tosyl | OH | methyl | OH |
| H | brosyl | OH | methyl | OH |
| H | mesyl | OH | methyl | OH |
| H | sulfonate | OH | methyl | OH |
| H | sulfonamide | OH | methyl | OH |
| H | phosphonate | OH | methyl | OH |

TABLE 25
Pericosine Analogues - Structural Formulas V, VI, and VII

| X | Y | W | V | U |
|---|---|---|---|---|
| methyl | OH | OH | ethyl | OH |
| methyl | Br | OH | ethyl | OH |
| methyl | Cl | OH | ethyl | OH |
| methyl | F | OH | ethyl | OH |
| methyl | acetyloxy | OH | ethyl | OH |
| methyl | PH₃P | OH | ethyl | OH |
| methyl | N,N,N-methyldiethyl-2- | OH | ethyl | OH |
| | (11-oxidaneyl)ethan-1- | | | |
| | aminium | | | |
| methyl | I | OH | ethyl | OH |
| methyl | tosyl | OH | ethyl | OH |
| methyl | brosyl | OH | ethyl | OH |
| methyl | mesyl | OH | ethyl | OH |
| methyl | sulfonate | OH | ethyl | OH |
| methyl | sulfonamide | OH | ethyl | OH |
| methyl | choline | OH | ethyl | OH |
| methyl | phosphonate | OH | ethyl | OH |
| ethyl | OH | OH | ethyl | OH |
| ethyl | Br | OH | ethyl | OH |
| ethyl | Cl | OH | ethyl | OH |
| ethyl | F | OH | ethyl | OH |
| ethyl | I | OH | ethyl | OH |
| ethyl | acetyloxy | OH | ethyl | OH |
| ethyl | PH₃P | OH | ethyl | OH |
| ethyl | choline | OH | ethyl | OH |
| ethyl | N,N,N-methyldiethyl-2- | OH | ethyl | OH |
| | (11-oxidaneyl)ethan-1- | | | |
| | aminium | | | |
| ethyl | tosyl | OH | ethyl | OH |
| ethyl | brosyl | OH | ethyl | OH |
| ethyl | mesyl | OH | ethyl | OH |
| ethyl | sulfonate | OH | ethyl | OH |
| ethyl | sulfonamide | OH | ethyl | OH |
| ethyl | phosphonate | OH | ethyl | OH |
| n-propyl | OH | OH | ethyl | OH |
| n-propyl | Br | OH | ethyl | OH |
| n-propyl | Cl | OH | ethyl | OH |
| n-propyl | F | OH | ethyl | OH |
| n-propyl | I | OH | ethyl | OH |
| n-propyl | acetyloxy | OH | ethyl | OH |
| n-propyl | PH₃P | OH | ethyl | OH |
| n-propyl | choline | OH | ethyl | OH |
| n-propyl | N,N,N-methyldiethyl-2- | OH | ethyl | OH |
| | (11-oxidaneyl)ethan-1- | | | |
| | aminium | | | |
| n-propyl | tosyl | OH | ethyl | OH |
| n-propyl | brosyl | OH | ethyl | OH |
| n-propyl | mesyl | OH | ethyl | OH |
| n-propyl | sulfonate | OH | ethyl | OH |
| n-propyl | sulfonamide | OH | ethyl | OH |
| n-propyl | phosphonate | OH | ethyl | OH |
| choline | OH | OH | ethyl | OH |
| choline | Br | OH | ethyl | OH |
| choline | Cl | OH | ethyl | OH |
| choline | F | OH | ethyl | OH |
| choline | I | OH | ethyl | OH |
| choline | acetyloxy | OH | ethyl | OH |
| choline | PH₃P | OH | ethyl | OH |
| choline | choline | OH | ethyl | OH |
| choline | N,N,N-methyldiethyl-2- | OH | ethyl | OH |
| | (11-oxidaneyl)ethan-1- | | | |
| | aminium | | | |
| choline | tosyl | OH | ethyl | OH |
| choline | brosyl | OH | ethyl | OH |
| choline | mesyl | OH | ethyl | OH |
| choline | sulfonate | OH | ethyl | OH |
| choline | sulfonamide | OH | ethyl | OH |
| choline | phosphonate | OH | ethyl | OH |
| H | OH | OH | ethyl | OH |
| H | Br | OH | ethyl | OH |
| H | Cl | OH | ethyl | OH |
| H | F | OH | ethyl | OH |
| H | I | OH | ethyl | OH |
| H | acetyloxy | OH | ethyl | OH |
| H | PH₃P | OH | ethyl | OH |
| H | choline | OH | ethyl | OH |
| H | N,N,N-methyldiethyl-2- | OH | ethyl | OH |
| | (11-oxidaneyl)ethan-1- | | | |
| | aminium | | | |
| H | tosyl | OH | ethyl | OH |
| H | brosyl | OH | ethyl | OH |
| H | mesyl | OH | ethyl | OH |
| H | sulfonate | OH | ethyl | OH |
| H | sulfonamide | OH | ethyl | OH |
| H | phosphonate | OH | ethyl | OH |

TABLE 26
Pericosine Analogues - Structural Formulas V, VI, and VII

| X | Y | W | V | U |
|---|---|---|---|---|
| methyl | OH | methoxy | OH | methoxy |
| methyl | Br | methoxy | OH | methoxy |
| methyl | Cl | methoxy | OH | methoxy |
| methyl | F | methoxy | OH | methoxy |
| methyl | acetyloxy | methoxy | OH | methoxy |
| methyl | PH₃P | methoxy | OH | methoxy |
| methyl | N,N,N-methyldiethyl-2- | methoxy | OH | methoxy |
| | (11-oxidaneyl)ethan-1- | | | |
| | aminium | | | |
| methyl | I | methoxy | OH | methoxy |
| methyl | tosyl | methoxy | OH | methoxy |
| methyl | brosyl | methoxy | OH | methoxy |
| methyl | mesyl | methoxy | OH | methoxy |
| methyl | sulfonate | methoxy | OH | methoxy |
| methyl | sulfonamide | methoxy | OH | methoxy |
| methyl | choline | methoxy | OH | methoxy |
| methyl | phosphonate | methoxy | OH | methoxy |
| ethyl | OH | methoxy | OH | methoxy |
| ethyl | Br | methoxy | OH | methoxy |
| ethyl | Cl | methoxy | OH | methoxy |
| ethyl | F | methoxy | OH | methoxy |
| ethyl | I | methoxy | OH | methoxy |
| ethyl | acetyloxy | methoxy | OH | methoxy |
| ethyl | PH₃P | methoxy | OH | methoxy |
| ethyl | choline | methoxy | OH | methoxy |
| ethyl | N,N,N-methyldiethyl-2- | methoxy | OH | methoxy |
| | (11-oxidaneyl)ethan-1- | | | |
| | aminium | | | |
| ethyl | tosyl | methoxy | OH | methoxy |
| ethyl | brosyl | methoxy | OH | methoxy |
| ethyl | mesyl | methoxy | OH | methoxy |
| ethyl | sulfonate | methoxy | OH | methoxy |
| ethyl | sulfonamide | methoxy | OH | methoxy |
| ethyl | phosphonate | methoxy | OH | methoxy |
| n-propyl | OH | methoxy | OH | methoxy |
| n-propyl | Br | methoxy | OH | methoxy |
| n-propyl | Cl | methoxy | OH | methoxy |
| n-propyl | F | methoxy | OH | methoxy |
| n-propyl | I | methoxy | OH | methoxy |
| n-propyl | acetyloxy | methoxy | OH | methoxy |
| n-propyl | PH₃P | methoxy | OH | methoxy |
| n-propyl | choline | methoxy | OH | methoxy |
| n-propyl | N,N,N-methyldiethyl-2- | methoxy | OH | methoxy |
| | (11-oxidaneyl)ethan-1- | | | |
| | aminium | | | |
| n-propyl | tosyl | methoxy | OH | methoxy |
| n-propyl | brosyl | methoxy | OH | methoxy |
| n-propyl | mesyl | methoxy | OH | methoxy |
| n-propyl | sulfonate | methoxy | OH | methoxy |
| n-propyl | sulfonamide | methoxy | OH | methoxy |
| n-propyl | phosphonate | methoxy | OH | methoxy |
| choline | OH | methoxy | OH | methoxy |
| choline | Br | methoxy | OH | methoxy |
| choline | Cl | methoxy | OH | methoxy |
| choline | F | methoxy | OH | methoxy |
| choline | I | methoxy | OH | methoxy |
| choline | acetyloxy | methoxy | OH | methoxy |
| choline | PH₃P | methoxy | OH | methoxy |
| choline | choline | methoxy | OH | methoxy |
| choline | N,N,N-methyldiethyl-2- | methoxy | OH | methoxy |
| | (11-oxidaneyl)ethan-1- | | | |
| | aminium | | | |
| choline | tosyl | methoxy | OH | methoxy |
| choline | brosyl | methoxy | OH | methoxy |
| choline | mesyl | methoxy | OH | methoxy |
| choline | sulfonate | methoxy | OH | methoxy |
| choline | sulfonamide | methoxy | OH | methoxy |
| choline | phosphonate | methoxy | OH | methoxy |
| H | OH | methoxy | OH | methoxy |
| H | Br | methoxy | OH | methoxy |
| H | Cl | methoxy | OH | methoxy |
| H | F | methoxy | OH | methoxy |
| H | I | methoxy | OH | methoxy |
| H | acetyloxy | methoxy | OH | methoxy |
| H | PH₃P | methoxy | OH | methoxy |
| H | choline | methoxy | OH | methoxy |
| H | N,N,N-methyldiethyl-2- | methoxy | OH | methoxy |
| | (11-oxidaneyl)ethan-1- | | | |
| | aminium | | | |
| H | tosyl | methoxy | OH | methoxy |
| H | brosyl | methoxy | OH | methoxy |
| H | mesyl | methoxy | OH | methoxy |
| H | sulfonate | methoxy | OH | methoxy |
| H | sulfonamide | methoxy | OH | methoxy |
| H | phosphonate | methoxy | OH | methoxy |

TABLE 27
Pericosine Analogues - Structural Formulas V, VI, and VII

| X | Y | W | V | U |
|---|---|---|---|---|
| methyl | OH | ethoxy | OH | ethoxy |
| methyl | Br | ethoxy | OH | ethoxy |
| methyl | Cl | ethoxy | OH | ethoxy |
| methyl | F | ethoxy | OH | ethoxy |
| methyl | acetyloxy | ethoxy | OH | ethoxy |
| methyl | PH₃P | ethoxy | OH | ethoxy |
| methyl | N,N,N-methyldiethyl-2- | ethoxy | OH | ethoxy |
| | (11-oxidaneyl)ethan-1- | | | |
| | aminium | | | |
| methyl | I | ethoxy | OH | ethoxy |
| methyl | tosyl | ethoxy | OH | ethoxy |
| methyl | brosyl | ethoxy | OH | ethoxy |
| methyl | mesyl | ethoxy | OH | ethoxy |
| methyl | sulfonate | ethoxy | OH | ethoxy |
| methyl | sulfonamide | ethoxy | OH | ethoxy |
| methyl | choline | ethoxy | OH | ethoxy |
| methyl | phosphonate | ethoxy | OH | ethoxy |
| ethyl | OH | ethoxy | OH | ethoxy |
| ethyl | Br | ethoxy | OH | ethoxy |
| ethyl | Cl | ethoxy | OH | ethoxy |
| ethyl | F | ethoxy | OH | ethoxy |
| ethyl | I | ethoxy | OH | ethoxy |
| ethyl | acetyloxy | ethoxy | OH | ethoxy |
| ethyl | PH₃P | ethoxy | OH | ethoxy |
| ethyl | choline | ethoxy | OH | ethoxy |
| ethyl | N,N,N-methyldiethyl-2- | ethoxy | OH | ethoxy |
| | (11-oxidaneyl)ethan-1- | | | |
| | aminium | | | |
| ethyl | tosyl | ethoxy | OH | ethoxy |
| ethyl | brosyl | ethoxy | OH | ethoxy |
| ethyl | mesyl | ethoxy | OH | ethoxy |
| ethyl | sulfonate | ethoxy | OH | ethoxy |
| ethyl | sulfonamide | ethoxy | OH | ethoxy |
| ethyl | phosphonate | ethoxy | OH | ethoxy |
| n-propyl | OH | ethoxy | OH | ethoxy |
| n-propyl | Br | ethoxy | OH | ethoxy |
| n-propyl | Cl | ethoxy | OH | ethoxy |
| n-propyl | F | ethoxy | OH | ethoxy |
| n-propyl | I | ethoxy | OH | ethoxy |
| n-propyl | acetyloxy | ethoxy | OH | ethoxy |
| n-propyl | PH₃P | ethoxy | OH | ethoxy |
| n-propyl | choline | ethoxy | OH | ethoxy |
| n-propyl | N,N,N-methyldiethyl-2- | ethoxy | OH | ethoxy |
| | (11-oxidaneyl)ethan-1- | | | |
| | aminium | | | |
| n-propyl | tosyl | ethoxy | OH | ethoxy |
| n-propyl | brosyl | ethoxy | OH | ethoxy |
| n-propyl | mesyl | ethoxy | OH | ethoxy |
| n-propyl | sulfonate | ethoxy | OH | ethoxy |
| n-propyl | sulfonamide | ethoxy | OH | ethoxy |
| n-propyl | phosphonate | ethoxy | OH | ethoxy |
| choline | OH | ethoxy | OH | ethoxy |
| choline | Br | ethoxy | OH | ethoxy |
| choline | Cl | ethoxy | OH | ethoxy |
| choline | F | ethoxy | OH | ethoxy |
| choline | I | ethoxy | OH | ethoxy |
| choline | acetyloxy | ethoxy | OH | ethoxy |
| choline | PH₃P | ethoxy | OH | ethoxy |
| choline | choline | ethoxy | OH | ethoxy |
| choline | N,N,N-methyldiethyl-2- | ethoxy | OH | ethoxy |
| | (11-oxidaneyl)ethan-1- | | | |
| | aminium | | | |
| choline | tosyl | ethoxy | OH | ethoxy |
| choline | brosyl | ethoxy | OH | ethoxy |
| choline | mesyl | ethoxy | OH | ethoxy |
| choline | sulfonate | ethoxy | OH | ethoxy |
| choline | sulfonamide | ethoxy | OH | ethoxy |
| choline | phosphonate | ethoxy | OH | ethoxy |
| H | OH | ethoxy | OH | ethoxy |
| H | Br | ethoxy | OH | ethoxy |
| H | Cl | ethoxy | OH | ethoxy |
| H | F | ethoxy | OH | ethoxy |
| H | I | ethoxy | OH | ethoxy |
| H | acetyloxy | ethoxy | OH | ethoxy |
| H | PH₃P | ethoxy | OH | ethoxy |
| H | choline | ethoxy | OH | ethoxy |
| H | N,N,N-methyldiethyl-2- | ethoxy | OH | ethoxy |
| | (11-oxidaneyl)ethan-1- | | | |
| | aminium | | | |
| H | tosyl | ethoxy | OH | ethoxy |
| H | brosyl | ethoxy | OH | ethoxy |
| H | mesyl | ethoxy | OH | ethoxy |
| H | sulfonate | ethoxy | OH | ethoxy |
| H | sulfonamide | ethoxy | OH | ethoxy |
| H | phosphonate | ethoxy | OH | ethoxy |

TABLE 28
Pericosine Analogues - Structural Formulas V, VI, and VII

| X | Y | W | V | U |
|---|---|---|---|---|
| methyl | OH | methyl | OH | methyl |
| methyl | Br | methyl | OH | methyl |
| methyl | Cl | methyl | OH | methyl |
| methyl | F | methyl | OH | methyl |
| methyl | acetyloxy | methyl | OH | methyl |
| methyl | PH₃P | methyl | OH | methyl |
| methyl | N,N,N-methyldiethyl-2- | methyl | OH | methyl |
| | (11-oxidaneyl)ethan-1- | | | |
| | aminium | | | |
| methyl | I | methyl | OH | methyl |
| methyl | tosyl | methyl | OH | methyl |
| methyl | brosyl | methyl | OH | methyl |
| methyl | mesyl | methyl | OH | methyl |
| methyl | sulfonate | methyl | OH | methyl |
| methyl | sulfonamide | methyl | OH | methyl |
| methyl | choline | methyl | OH | methyl |
| methyl | phosphonate | methyl | OH | methyl |
| ethyl | OH | methyl | OH | methyl |
| ethyl | Br | methyl | OH | methyl |
| ethyl | Cl | methyl | OH | methyl |
| ethyl | F | methyl | OH | methyl |
| ethyl | I | methyl | OH | methyl |
| ethyl | acetyloxy | methyl | OH | methyl |
| ethyl | PH₃P | methyl | OH | methyl |
| ethyl | choline | methyl | OH | methyl |
| ethyl | N,N,N-methyldiethyl-2- | methyl | OH | methyl |
| | (11-oxidaneyl)ethan-1- | | | |
| | aminium | | | |
| ethyl | tosyl | methyl | OH | methyl |
| ethyl | brosyl | methyl | OH | methyl |
| ethyl | mesyl | methyl | OH | methyl |
| ethyl | sulfonate | methyl | OH | methyl |
| ethyl | sulfonamide | methyl | OH | methyl |
| ethyl | phosphonate | methyl | OH | methyl |
| n-propyl | OH | methyl | OH | methyl |
| n-propyl | Br | methyl | OH | methyl |
| n-propyl | Cl | methyl | OH | methyl |
| n-propyl | F | methyl | OH | methyl |
| n-propyl | I | methyl | OH | methyl |
| n-propyl | acetyloxy | methyl | OH | methyl |
| n-propyl | PH₃P | methyl | OH | methyl |
| n-propyl | choline | methyl | OH | methyl |
| n-propyl | N,N,N-methyldiethyl-2- | methyl | OH | methyl |
| | (11-oxidaneyl)ethan-1- | | | |
| | aminium | | | |
| n-propyl | tosyl | methyl | OH | methyl |
| n-propyl | brosyl | methyl | OH | methyl |
| n-propyl | mesyl | methyl | OH | methyl |
| n-propyl | sulfonate | methyl | OH | methyl |
| n-propyl | sulfonamide | methyl | OH | methyl |
| n-propyl | phosphonate | methyl | OH | methyl |
| choline | OH | methyl | OH | methyl |
| choline | Br | methyl | OH | methyl |
| choline | Cl | methyl | OH | methyl |
| choline | F | methyl | OH | methyl |
| choline | I | methyl | OH | methyl |
| choline | acetyloxy | methyl | OH | methyl |
| choline | PH₃P | methyl | OH | methyl |
| choline | choline | methyl | OH | methyl |
| choline | N,N,N-methyldiethyl-2- | methyl | OH | methyl |
| | (11-oxidaneyl)ethan-1- | | | |
| | aminium | | | |
| choline | tosyl | methyl | OH | methyl |
| choline | brosyl | methyl | OH | methyl |
| choline | mesyl | methyl | OH | methyl |
| choline | sulfonate | methyl | OH | methyl |
| choline | sulfonamide | methyl | OH | methyl |
| choline | phosphonate | methyl | OH | methyl |
| H | OH | methyl | OH | methyl |
| H | Br | methyl | OH | methyl |
| H | Cl | methyl | OH | methyl |
| H | F | methyl | OH | methyl |
| H | I | methyl | OH | methyl |
| H | acetyloxy | methyl | OH | methyl |
| H | PH₃P | methyl | OH | methyl |
| H | choline | methyl | OH | methyl |
| H | N,N,N-methyldiethyl-2- | methyl | OH | methyl |
| | (11-oxidaneyl)ethan-1- | | | |
| | aminium | | | |
| H | tosyl | methyl | OH | methyl |
| H | brosyl | methyl | OH | methyl |
| H | mesyl | methyl | OH | methyl |
| H | sulfonate | methyl | OH | methyl |
| H | sulfonamide | methyl | OH | methyl |
| H | phosphonate | methyl | OH | methyl |

TABLE 29
Pericosine Analogues - Structural Formulas V, VI, and VII

| X | Y | W | V | U |
|---|---|---|---|---|
| methyl | OH | ethyl | OH | ethyl |
| methyl | Br | ethyl | OH | ethyl |
| methyl | Cl | ethyl | OH | ethyl |
| methyl | F | ethyl | OH | ethyl |
| methyl | acetyloxy | ethyl | OH | ethyl |
| methyl | PH₃P | ethyl | OH | ethyl |
| methyl | N,N,N-methyldiethyl-2- | ethyl | OH | ethyl |
| | (11-oxidaneyl)ethan-1- | | | |
| | aminium | | | |
| methyl | I | ethyl | OH | ethyl |
| methyl | tosyl | ethyl | OH | ethyl |
| methyl | brosyl | ethyl | OH | ethyl |
| methyl | mesyl | ethyl | OH | ethyl |
| methyl | sulfonate | ethyl | OH | ethyl |
| methyl | sulfonamide | ethyl | OH | ethyl |
| methyl | choline | ethyl | OH | ethyl |
| methyl | phosphonate | ethyl | OH | ethyl |
| ethyl | OH | ethyl | OH | ethyl |
| ethyl | Br | ethyl | OH | ethyl |
| ethyl | Cl | ethyl | OH | ethyl |
| ethyl | F | ethyl | OH | ethyl |
| ethyl | I | ethyl | OH | ethyl |
| ethyl | acetyloxy | ethyl | OH | ethyl |
| ethyl | PH₃P | ethyl | OH | ethyl |
| ethyl | choline | ethyl | OH | ethyl |
| ethyl | N,N,N-methyldiethyl-2- | ethyl | OH | ethyl |
| | (11-oxidaneyl)ethan-1- | | | |
| | aminium | | | |
| ethyl | tosyl | ethyl | OH | ethyl |
| ethyl | brosyl | ethyl | OH | ethyl |
| ethyl | mesyl | ethyl | OH | ethyl |
| ethyl | sulfonate | ethyl | OH | ethyl |
| ethyl | sulfonamide | ethyl | OH | ethyl |
| ethyl | phosphonate | ethyl | OH | ethyl |
| n-propyl | OH | ethyl | OH | ethyl |
| n-propyl | Br | ethyl | OH | ethyl |
| n-propyl | Cl | ethyl | OH | ethyl |
| n-propyl | F | ethyl | OH | ethyl |
| n-propyl | I | ethyl | OH | ethyl |
| n-propyl | acetyloxy | ethyl | OH | ethyl |
| n-propyl | PH₃P | ethyl | OH | ethyl |
| n-propyl | choline | ethyl | OH | ethyl |
| n-propyl | N,N,N-methyldiethyl-2- | ethyl | OH | ethyl |
| | (11-oxidaneyl)ethan-1- | | | |
| | aminium | | | |
| n-propyl | tosyl | ethyl | OH | ethyl |
| n-propyl | brosyl | ethyl | OH | ethyl |
| n-propyl | mesyl | ethyl | OH | ethyl |
| n-propyl | sulfonate | ethyl | OH | ethyl |
| n-propyl | sulfonamide | ethyl | OH | ethyl |
| n-propyl | phosphonate | ethyl | OH | ethyl |
| choline | OH | ethyl | OH | ethyl |
| choline | Br | ethyl | OH | ethyl |
| choline | Cl | ethyl | OH | ethyl |
| choline | F | ethyl | OH | ethyl |
| choline | I | ethyl | OH | ethyl |
| choline | acetyloxy | ethyl | OH | ethyl |
| choline | PH₃P | ethyl | OH | ethyl |
| choline | choline | ethyl | OH | ethyl |
| choline | N,N,N-methyldiethyl-2- | ethyl | OH | ethyl |
| | (11-oxidaneyl)ethan-1- | | | |
| | aminium | | | |
| choline | tosyl | ethyl | OH | ethyl |
| choline | brosyl | ethyl | OH | ethyl |
| choline | mesyl | ethyl | OH | ethyl |
| choline | sulfonate | ethyl | OH | ethyl |
| choline | sulfonamide | ethyl | OH | ethyl |
| choline | phosphonate | ethyl | OH | ethyl |
| H | OH | ethyl | OH | ethyl |
| H | Br | ethyl | OH | ethyl |
| H | Cl | ethyl | OH | ethyl |
| H | F | ethyl | OH | ethyl |
| H | I | ethyl | OH | ethyl |
| H | acetyloxy | ethyl | OH | ethyl |
| H | PH₃P | ethyl | OH | ethyl |
| H | choline | ethyl | OH | ethyl |
| H | N,N,N-methyldiethyl-2- | ethyl | OH | ethyl |
| | (11-oxidaneyl)ethan-1- | | | |
| | aminium | | | |
| H | tosyl | ethyl | OH | ethyl |
| H | brosyl | ethyl | OH | ethyl |
| H | mesyl | ethyl | OH | ethyl |
| H | sulfonate | ethyl | OH | ethyl |
| H | sulfonamide | ethyl | OH | ethyl |
| H | phosphonate | ethyl | OH | ethyl |

TABLE 30
Pericosine Analogues - Structural Formulas V, VI, and VII

| X | Y | W | V | U |
|---|---|---|---|---|
| methyl | OH | OH | OH | methoxy |
| methyl | Br | OH | OH | methoxy |
| methyl | Cl | OH | OH | methoxy |
| methyl | F | OH | OH | methoxy |
| methyl | acetyloxy | OH | OH | methoxy |
| methyl | PH₃P | OH | OH | methoxy |
| methyl | N,N,N-methyldiethyl-2- | OH | OH | methoxy |
| | (11-oxidaneyl)ethan-1- | | | |
| | aminium | | | |
| methyl | I | OH | OH | methoxy |
| methyl | tosyl | OH | OH | methoxy |
| methyl | brosyl | OH | OH | methoxy |
| methyl | mesyl | OH | OH | methoxy |
| methyl | sulfonate | OH | OH | methoxy |
| methyl | sulfonamide | OH | OH | methoxy |
| methyl | choline | OH | OH | methoxy |
| methyl | phosphonate | OH | OH | methoxy |
| ethyl | OH | OH | OH | methoxy |
| ethyl | Br | OH | OH | methoxy |
| ethyl | Cl | OH | OH | methoxy |
| ethyl | F | OH | OH | methoxy |
| ethyl | I | OH | OH | methoxy |
| ethyl | acetyloxy | OH | OH | methoxy |
| ethyl | PH₃P | OH | OH | methoxy |
| ethyl | choline | OH | OH | methoxy |
| ethyl | N,N,N-methyldiethyl-2- | OH | OH | methoxy |
| | (11-oxidaneyl)ethan-1- | | | |
| | aminium | | | |
| ethyl | tosyl | OH | OH | methoxy |
| ethyl | brosyl | OH | OH | methoxy |
| ethyl | mesyl | OH | OH | methoxy |
| ethyl | sulfonate | OH | OH | methoxy |
| ethyl | sulfonamide | OH | OH | methoxy |
| ethyl | phosphonate | OH | OH | methoxy |
| n-propyl | OH | OH | OH | methoxy |
| n-propyl | Br | OH | OH | methoxy |
| n-propyl | Cl | OH | OH | methoxy |
| n-propyl | F | OH | OH | methoxy |
| n-propyl | I | OH | OH | methoxy |
| n-propyl | acetyloxy | OH | OH | methoxy |
| n-propyl | PH₃P | OH | OH | methoxy |
| n-propyl | choline | OH | OH | methoxy |
| n-propyl | N,N,N-methyldiethyl-2- | OH | OH | methoxy |
| | (11-oxidaneyl)ethan-1- | | | |
| | aminium | | | |
| n-propyl | tosyl | OH | OH | methoxy |
| n-propyl | brosyl | OH | OH | methoxy |
| n-propyl | mesyl | OH | OH | methoxy |
| n-propyl | sulfonate | OH | OH | methoxy |
| n-propyl | sulfonamide | OH | OH | methoxy |
| n-propyl | phosphonate | OH | OH | methoxy |
| choline | OH | OH | OH | methoxy |
| choline | Br | OH | OH | methoxy |
| choline | Cl | OH | OH | methoxy |
| choline | F | OH | OH | methoxy |
| choline | I | OH | OH | methoxy |
| choline | acetyloxy | OH | OH | methoxy |
| choline | PH₃P | OH | OH | methoxy |
| choline | choline | OH | OH | methoxy |
| choline | N,N,N-methyldiethyl-2- | OH | OH | methoxy |
| | (11-oxidaneyl)ethan-1- | | | |
| | aminium | | | |
| choline | tosyl | OH | OH | methoxy |
| choline | brosyl | OH | OH | methoxy |
| choline | mesyl | OH | OH | methoxy |
| choline | sulfonate | OH | OH | methoxy |
| choline | sulfonamide | OH | OH | methoxy |
| choline | phosphonate | OH | OH | methoxy |
| H | OH | OH | OH | methoxy |
| H | Br | OH | OH | methoxy |
| H | Cl | OH | OH | methoxy |
| H | F | OH | OH | methoxy |
| H | I | OH | OH | methoxy |
| H | acetyloxy | OH | OH | methoxy |
| H | PH₃P | OH | OH | methoxy |
| H | choline | OH | OH | methoxy |
| H | N,N,N-methyldiethyl-2- | OH | OH | methoxy |
| | (11-oxidaneyl)ethan-1- | | | |
| | aminium | | | |
| H | tosyl | OH | OH | methoxy |
| H | brosyl | OH | OH | methoxy |
| H | mesyl | OH | OH | methoxy |
| H | sulfonate | OH | OH | methoxy |
| H | sulfonamide | OH | OH | methoxy |
| H | phosphonate | OH | OH | methoxy |

TABLE 31
Pericosine Analogues - Structural Formulas V, VI, and VII

| X | Y | W | V | U |
|---|---|---|---|---|
| methyl | OH | OH | OH | ethoxy |
| methyl | Br | OH | OH | ethoxy |
| methyl | Cl | OH | OH | ethoxy |
| methyl | F | OH | OH | ethoxy |
| methyl | acetyloxy | OH | OH | ethoxy |
| methyl | PH₃P | OH | OH | ethoxy |
| methyl | N,N,N-methyldiethyl-2- | OH | OH | ethoxy |
| | (11-oxidaneyl)ethan-1- | | | |
| | aminium | | | |
| methyl | I | OH | OH | ethoxy |
| methyl | tosyl | OH | OH | ethoxy |
| methyl | brosyl | OH | OH | ethoxy |
| methyl | mesyl | OH | OH | ethoxy |
| methyl | sulfonate | OH | OH | ethoxy |
| methyl | sulfonamide | OH | OH | ethoxy |
| methyl | choline | OH | OH | ethoxy |
| methyl | phosphonate | OH | OH | ethoxy |
| ethyl | OH | OH | OH | ethoxy |
| ethyl | Br | OH | OH | ethoxy |
| ethyl | Cl | OH | OH | ethoxy |
| ethyl | F | OH | OH | ethoxy |
| ethyl | I | OH | OH | ethoxy |
| ethyl | acetyloxy | OH | OH | ethoxy |
| ethyl | PH₃P | OH | OH | ethoxy |
| ethyl | choline | OH | OH | ethoxy |
| ethyl | N,N,N-methyldiethyl-2- | OH | OH | ethoxy |
| | (11-oxidaneyl)ethan-1- | | | |
| | aminium | | | |
| ethyl | tosyl | OH | OH | ethoxy |
| ethyl | brosyl | OH | OH | ethoxy |
| ethyl | mesyl | OH | OH | ethoxy |
| ethyl | sulfonate | OH | OH | ethoxy |
| ethyl | sulfonamide | OH | OH | ethoxy |
| ethyl | phosphonate | OH | OH | ethoxy |
| n-propyl | OH | OH | OH | ethoxy |
| n-propyl | Br | OH | OH | ethoxy |
| n-propyl | Cl | OH | OH | ethoxy |
| n-propyl | F | OH | OH | ethoxy |
| n-propyl | I | OH | OH | ethoxy |
| n-propyl | acetyloxy | OH | OH | ethoxy |
| n-propyl | PH₃P | OH | OH | ethoxy |
| n-propyl | choline | OH | OH | ethoxy |
| n-propyl | N,N,N-methyldiethyl-2- | OH | OH | ethoxy |
| | (11-oxidaneyl)ethan-1- | | | |
| | aminium | | | |
| n-propyl | tosyl | OH | OH | ethoxy |
| n-propyl | brosyl | OH | OH | ethoxy |
| n-propyl | mesyl | OH | OH | ethoxy |
| n-propyl | sulfonate | OH | OH | ethoxy |
| n-propyl | sulfonamide | OH | OH | ethoxy |
| n-propyl | phosphonate | OH | OH | ethoxy |
| choline | OH | OH | OH | ethoxy |
| choline | Br | OH | OH | ethoxy |
| choline | Cl | OH | OH | ethoxy |
| choline | F | OH | OH | ethoxy |
| choline | I | OH | OH | ethoxy |
| choline | acetyloxy | OH | OH | ethoxy |
| choline | PH₃P | OH | OH | ethoxy |
| choline | choline | OH | OH | ethoxy |
| choline | N,N,N-methyldiethyl-2- | OH | OH | ethoxy |
| | (11-oxidaneyl)ethan-1- | | | |
| | aminium | | | |
| choline | tosyl | OH | OH | ethoxy |
| choline | brosyl | OH | OH | ethoxy |
| choline | mesyl | OH | OH | ethoxy |
| choline | sulfonate | OH | OH | ethoxy |
| choline | sulfonamide | OH | OH | ethoxy |
| choline | phosphonate | OH | OH | ethoxy |
| H | OH | OH | OH | ethoxy |
| H | Br | OH | OH | ethoxy |
| H | Cl | OH | OH | ethoxy |
| H | F | OH | OH | ethoxy |
| H | I | OH | OH | ethoxy |
| H | acetyloxy | OH | OH | ethoxy |
| H | PH₃P | OH | OH | ethoxy |
| H | choline | OH | OH | ethoxy |
| H | N,N,N-methyldiethyl-2- | OH | OH | ethoxy |
| | (11-oxidaneyl)ethan-1- | | | |
| | aminium | | | |
| H | tosyl | OH | OH | ethoxy |
| H | brosyl | OH | OH | ethoxy |
| H | mesyl | OH | OH | ethoxy |
| H | sulfonate | OH | OH | ethoxy |
| H | sulfonamide | OH | OH | ethoxy |
| H | phosphonate | OH | OH | ethoxy |

TABLE 32
Pericosine Analogues - Structural Formulas V, VI, and VII

| X | Y | W | V | U |
|---|---|---|---|---|
| methyl | OH | OH | OH | methyl |
| methyl | Br | OH | OH | methyl |
| methyl | Cl | OH | OH | methyl |
| methyl | F | OH | OH | methyl |
| methyl | acetyloxy | OH | OH | methyl |
| methyl | PH₃P | OH | OH | methyl |
| methyl | N,N,N-methyldiethyl-2- | OH | OH | methyl |
| | (11-oxidaneyl)ethan-1- | | | |
| | aminium | | | |
| methyl | I | OH | OH | methyl |
| methyl | tosyl | OH | OH | methyl |
| methyl | brosyl | OH | OH | methyl |
| methyl | mesyl | OH | OH | methyl |
| methyl | sulfonate | OH | OH | methyl |
| methyl | sulfonamide | OH | OH | methyl |
| methyl | choline | OH | OH | methyl |
| methyl | phosphonate | OH | OH | methyl |
| ethyl | OH | OH | OH | methyl |
| ethyl | Br | OH | OH | methyl |
| ethyl | Cl | OH | OH | methyl |
| ethyl | F | OH | OH | methyl |
| ethyl | I | OH | OH | methyl |
| ethyl | acetyloxy | OH | OH | methyl |
| ethyl | PH₃P | OH | OH | methyl |
| ethyl | choline | OH | OH | methyl |
| ethyl | N,N,N-methyldiethyl-2- | OH | OH | methyl |
| | (11-oxidaneyl)ethan-1- | | | |
| | aminium | | | |
| ethyl | tosyl | OH | OH | methyl |
| ethyl | brosyl | OH | OH | methyl |
| ethyl | mesyl | OH | OH | methyl |
| ethyl | sulfonate | OH | OH | methyl |
| ethyl | sulfonamide | OH | OH | methyl |
| ethyl | phosphonate | OH | OH | methyl |
| n-propyl | OH | OH | OH | methyl |
| n-propyl | Br | OH | OH | methyl |
| n-propyl | Cl | OH | OH | methyl |
| n-propyl | F | OH | OH | methyl |
| n-propyl | I | OH | OH | methyl |
| n-propyl | acetyloxy | OH | OH | methyl |
| n-propyl | PH₃P | OH | OH | methyl |
| n-propyl | choline | OH | OH | methyl |
| n-propyl | N,N,N-methyldiethyl-2- | OH | OH | methyl |
| | (11-oxidaneyl)ethan-1- | | | |
| | aminium | | | |
| n-propyl | tosyl | OH | OH | methyl |
| n-propyl | brosyl | OH | OH | methyl |
| n-propyl | mesyl | OH | OH | methyl |
| n-propyl | sulfonate | OH | OH | methyl |
| n-propyl | sulfonamide | OH | OH | methyl |
| n-propyl | phosphonate | OH | OH | methyl |
| choline | OH | OH | OH | methyl |
| choline | Br | OH | OH | methyl |
| choline | Cl | OH | OH | methyl |
| choline | F | OH | OH | methyl |
| choline | I | OH | OH | methyl |
| choline | acetyloxy | OH | OH | methyl |
| choline | PH₃P | OH | OH | methyl |
| choline | choline | OH | OH | methyl |
| choline | N,N,N-methyldiethyl-2- | OH | OH | methyl |
| | (11-oxidaneyl)ethan-1- | | | |
| | aminium | | | |
| choline | tosyl | OH | OH | methyl |
| choline | brosyl | OH | OH | methyl |
| choline | mesyl | OH | OH | methyl |
| choline | sulfonate | OH | OH | methyl |
| choline | sulfonamide | OH | OH | methyl |
| choline | phosphonate | OH | OH | methyl |
| H | OH | OH | OH | methyl |
| H | Br | OH | OH | methyl |
| H | Cl | OH | OH | methyl |
| H | F | OH | OH | methyl |
| H | I | OH | OH | methyl |
| H | acetyloxy | OH | OH | methyl |
| H | PH₃P | OH | OH | methyl |
| H | choline | OH | OH | methyl |
| H | N,N,N-methyldiethyl-2- | OH | OH | methyl |
| | (11-oxidaneyl)ethan-1- | | | |
| | aminium | | | |
| H | tosyl | OH | OH | methyl |
| H | brosyl | OH | OH | methyl |
| H | mesyl | OH | OH | methyl |
| H | sulfonate | OH | OH | methyl |
| H | sulfonamide | OH | OH | methyl |
| H | phosphonate | OH | OH | methyl |

TABLE 33
Pericosine Analogues - Structural Formulas V, VI, and VII

| X | Y | W | V | U |
|---|---|---|---|---|
| methyl | OH | OH | OH | ethyl |
| methyl | Br | OH | OH | ethyl |
| methyl | Cl | OH | OH | ethyl |
| methyl | F | OH | OH | ethyl |
| methyl | acetyloxy | OH | OH | ethyl |
| methyl | PH₃P | OH | OH | ethyl |
| methyl | N,N,N-methyldiethyl-2- | OH | OH | ethyl |
| | (11-oxidaneyl)ethan-1- | | | |
| | aminium | | | |
| methyl | I | OH | OH | ethyl |
| methyl | tosyl | OH | OH | ethyl |
| methyl | brosyl | OH | OH | ethyl |
| methyl | mesyl | OH | OH | ethyl |
| methyl | sulfonate | OH | OH | ethyl |
| methyl | sulfonamide | OH | OH | ethyl |
| methyl | choline | OH | OH | ethyl |
| methyl | phosphonate | OH | OH | ethyl |
| ethyl | OH | OH | OH | ethyl |
| ethyl | Br | OH | OH | ethyl |
| ethyl | Cl | OH | OH | ethyl |
| ethyl | F | OH | OH | ethyl |
| ethyl | I | OH | OH | ethyl |
| ethyl | acetyloxy | OH | OH | ethyl |
| ethyl | PH₃P | OH | OH | ethyl |
| ethyl | choline | OH | OH | ethyl |
| ethyl | N,N,N-methyldiethyl-2- | OH | OH | ethyl |
| | (11-oxidaneyl)ethan-1- | | | |
| | aminium | | | |
| ethyl | tosyl | OH | OH | ethyl |
| ethyl | brosyl | OH | OH | ethyl |
| ethyl | mesyl | OH | OH | ethyl |
| ethyl | sulfonate | OH | OH | ethyl |
| ethyl | sulfonamide | OH | OH | ethyl |
| ethyl | phosphonate | OH | OH | ethyl |
| n-propyl | OH | OH | OH | ethyl |
| n-propyl | Br | OH | OH | ethyl |
| n-propyl | Cl | OH | OH | ethyl |
| n-propyl | F | OH | OH | ethyl |
| n-propyl | I | OH | OH | ethyl |
| n-propyl | acetyloxy | OH | OH | ethyl |
| n-propyl | PH₃P | OH | OH | ethyl |
| n-propyl | choline | OH | OH | ethyl |
| n-propyl | N,N,N-methyldiethyl-2- | OH | OH | ethyl |
| | (11-oxidaneyl)ethan-1- | | | |
| | aminium | | | |
| n-propyl | tosyl | OH | OH | ethyl |
| n-propyl | brosyl | OH | OH | ethyl |
| n-propyl | mesyl | OH | OH | ethyl |
| n-propyl | sulfonate | OH | OH | ethyl |
| n-propyl | sulfonamide | OH | OH | ethyl |
| n-propyl | phosphonate | OH | OH | ethyl |
| choline | OH | OH | OH | ethyl |
| choline | Br | OH | OH | ethyl |
| choline | Cl | OH | OH | ethyl |
| choline | F | OH | OH | ethyl |
| choline | I | OH | OH | ethyl |
| choline | acetyloxy | OH | OH | ethyl |
| choline | PH₃P | OH | OH | ethyl |
| choline | choline | OH | OH | ethyl |
| choline | N,N,N-methyldiethyl-2- | OH | OH | ethyl |
| | (11-oxidaneyl)ethan-1- | | | |
| | aminium | | | |
| choline | tosyl | OH | OH | ethyl |
| choline | brosyl | OH | OH | ethyl |
| choline | mesyl | OH | OH | ethyl |
| choline | sulfonate | OH | OH | ethyl |
| choline | sulfonamide | OH | OH | ethyl |
| choline | phosphonate | OH | OH | ethyl |
| H | OH | OH | OH | ethyl |
| H | Br | OH | OH | ethyl |
| H | Cl | OH | OH | ethyl |
| H | F | OH | OH | ethyl |
| H | I | OH | OH | ethyl |
| H | acetyloxy | OH | OH | ethyl |
| H | PH₃P | OH | OH | ethyl |
| H | choline | OH | OH | ethyl |
| H | N,N,N-methyldiethyl-2- | OH | OH | ethyl |
| | (11-oxidaneyl)ethan-1- | | | |
| | aminium | | | |
| H | tosyl | OH | OH | ethyl |
| H | brosyl | OH | OH | ethyl |
| H | mesyl | OH | OH | ethyl |
| H | sulfonate | OH | OH | ethyl |
| H | sulfonamide | OH | OH | ethyl |
| H | phosphonate | OH | OH | ethyl |

TABLE 34
Pericosine Analogues - Structural Formulas V, VI, and VII

| X | Y | W | V | U |
|---|---|---|---|---|
| methyl | OH | H | methoxy | OH |
| methyl | Br | H | methoxy | OH |
| methyl | Cl | H | methoxy | OH |
| methyl | F | H | methoxy | OH |
| methyl | acetyloxy | H | methoxy | OH |
| methyl | PH₃P | H | methoxy | OH |
| methyl | N,N,N-methyldiethyl-2- | H | methoxy | OH |
| | (11-oxidaneyl)ethan-1- | | | |
| | aminium | | | |
| methyl | I | H | methoxy | OH |
| methyl | tosyl | H | methoxy | OH |
| methyl | brosyl | H | methoxy | OH |
| methyl | mesyl | H | methoxy | OH |
| methyl | sulfonate | H | methoxy | OH |
| methyl | sulfonamide | H | methoxy | OH |
| methyl | choline | H | methoxy | OH |
| methyl | phosphonate | H | methoxy | OH |
| ethyl | OH | H | methoxy | OH |
| ethyl | Br | H | methoxy | OH |
| ethyl | Cl | H | methoxy | OH |
| ethyl | F | H | methoxy | OH |
| ethyl | I | H | methoxy | OH |
| ethyl | acetyloxy | H | methoxy | OH |
| ethyl | PH₃P | H | methoxy | OH |
| ethyl | choline | H | methoxy | OH |
| ethyl | N,N,N-methyldiethyl-2- | H | methoxy | OH |
| | (11-oxidaneyl)ethan-1- | | | |
| | aminium | | | |
| ethyl | tosyl | H | methoxy | OH |
| ethyl | brosyl | H | methoxy | OH |
| ethyl | mesyl | H | methoxy | OH |
| ethyl | sulfonate | H | methoxy | OH |
| ethyl | sulfonamide | H | methoxy | OH |
| ethyl | phosphonate | H | methoxy | OH |
| n-propyl | OH | H | methoxy | OH |
| n-propyl | Br | H | methoxy | OH |
| n-propyl | Cl | H | methoxy | OH |
| n-propyl | F | H | methoxy | OH |
| n-propyl | I | H | methoxy | OH |
| n-propyl | acetyloxy | H | methoxy | OH |
| n-propyl | PH₃P | H | methoxy | OH |
| n-propyl | choline | H | methoxy | OH |
| n-propyl | N,N,N-methyldiethyl-2- | H | methoxy | OH |
| | (11-oxidaneyl)ethan-1- | | | |
| | aminium | | | |
| n-propyl | tosyl | H | methoxy | OH |
| n-propyl | brosyl | H | methoxy | OH |
| n-propyl | mesyl | H | methoxy | OH |
| n-propyl | sulfonate | H | methoxy | OH |
| n-propyl | sulfonamide | H | methoxy | OH |
| n-propyl | phosphonate | H | methoxy | OH |
| choline | OH | H | methoxy | OH |
| choline | Br | H | methoxy | OH |
| choline | Cl | H | methoxy | OH |
| choline | F | H | methoxy | OH |
| choline | I | H | methoxy | OH |
| choline | acetyloxy | H | methoxy | OH |
| choline | PH₃P | H | methoxy | OH |
| choline | choline | H | methoxy | OH |
| choline | N,N,N-methyldiethyl-2- | H | methoxy | OH |
| | (11-oxidaneyl)ethan-1- | | | |
| | aminium | | | |
| choline | tosyl | H | methoxy | OH |
| choline | brosyl | H | methoxy | OH |
| choline | mesyl | H | methoxy | OH |
| choline | sulfonate | H | methoxy | OH |
| choline | sulfonamide | H | methoxy | OH |
| choline | phosphonate | H | methoxy | OH |
| H | OH | H | methoxy | OH |
| H | Br | H | methoxy | OH |
| H | Cl | H | methoxy | OH |
| H | F | H | methoxy | OH |
| H | I | H | methoxy | OH |
| H | acetyloxy | H | methoxy | OH |
| H | PH₃P | H | methoxy | OH |
| H | choline | H | methoxy | OH |
| H | N,N,N-methyldiethyl-2- | H | methoxy | OH |
| | (11-oxidaneyl)ethan-1- | | | |
| | aminium | | | |
| H | tosyl | H | methoxy | OH |
| H | brosyl | H | methoxy | OH |
| H | mesyl | H | methoxy | OH |
| H | sulfonate | H | methoxy | OH |
| H | sulfonamide | H | methoxy | OH |
| H | phosphonate | H | methoxy | OH |

TABLE 35
Pericosine Analogues - Structural Formulas V, VI, and VII

| X | Y | W | V | U |
|---|---|---|---|---|
| methyl | OH | H | ethoxy | OH |
| methyl | Br | H | ethoxy | OH |
| methyl | Cl | H | ethoxy | OH |
| methyl | F | H | ethoxy | OH |
| methyl | acetyloxy | H | ethoxy | OH |
| methyl | PH₃P | H | ethoxy | OH |
| methyl | N,N,N-methyldiethyl-2- | H | ethoxy | OH |
| | (11-oxidaneyl)ethan-1- | | | |
| | aminium | | | |
| methyl | I | H | ethoxy | OH |
| methyl | tosyl | H | ethoxy | OH |
| methyl | brosyl | H | ethoxy | OH |
| methyl | mesyl | H | ethoxy | OH |
| methyl | sulfonate | H | ethoxy | OH |
| methyl | sulfonamide | H | ethoxy | OH |
| methyl | choline | H | ethoxy | OH |
| methyl | phosphonate | H | ethoxy | OH |
| ethyl | OH | H | ethoxy | OH |
| ethyl | Br | H | ethoxy | OH |
| ethyl | Cl | H | ethoxy | OH |
| ethyl | F | H | ethoxy | OH |
| ethyl | I | H | ethoxy | OH |
| ethyl | acetyloxy | H | ethoxy | OH |
| ethyl | PH₃P | H | ethoxy | OH |
| ethyl | choline | H | ethoxy | OH |
| ethyl | N,N,N-methyldiethyl-2- | H | ethoxy | OH |
| | (11-oxidaneyl)ethan-1- | | | |
| | aminium | | | |
| ethyl | tosyl | H | ethoxy | OH |
| ethyl | brosyl | H | ethoxy | OH |
| ethyl | mesyl | H | ethoxy | OH |
| ethyl | sulfonate | H | ethoxy | OH |
| ethyl | sulfonamide | H | ethoxy | OH |
| ethyl | phosphonate | H | ethoxy | OH |
| n-propyl | OH | H | ethoxy | OH |
| n-propyl | Br | H | ethoxy | OH |
| n-propyl | Cl | H | ethoxy | OH |
| n-propyl | F | H | ethoxy | OH |
| n-propyl | I | H | ethoxy | OH |
| n-propyl | acetyloxy | H | ethoxy | OH |
| n-propyl | PH₃P | H | ethoxy | OH |
| n-propyl | choline | H | ethoxy | OH |
| n-propyl | N,N,N-methyldiethyl-2- | H | ethoxy | OH |
| | (11-oxidaneyl)ethan-1- | | | |
| | aminium | | | |
| n-propyl | tosyl | H | ethoxy | OH |
| n-propyl | brosyl | H | ethoxy | OH |
| n-propyl | mesyl | H | ethoxy | OH |
| n-propyl | sulfonate | H | ethoxy | OH |
| n-propyl | sulfonamide | H | ethoxy | OH |
| n-propyl | phosphonate | H | ethoxy | OH |
| choline | OH | H | ethoxy | OH |
| choline | Br | H | ethoxy | OH |
| choline | Cl | H | ethoxy | OH |
| choline | F | H | ethoxy | OH |
| choline | I | H | ethoxy | OH |
| choline | acetyloxy | H | ethoxy | OH |
| choline | PH₃P | H | ethoxy | OH |
| choline | choline | H | ethoxy | OH |
| choline | N,N,N-methyldiethyl-2- | H | ethoxy | OH |
| | (11-oxidaneyl)ethan-1- | | | |
| | aminium | | | |
| choline | tosyl | H | ethoxy | OH |
| choline | brosyl | H | ethoxy | OH |
| choline | mesyl | H | ethoxy | OH |
| choline | sulfonate | H | ethoxy | OH |
| choline | sulfonamide | H | ethoxy | OH |
| choline | phosphonate | H | ethoxy | OH |
| H | OH | H | ethoxy | OH |
| H | Br | H | ethoxy | OH |
| H | Cl | H | ethoxy | OH |
| H | F | H | ethoxy | OH |
| H | I | H | ethoxy | OH |
| H | acetyloxy | H | ethoxy | OH |
| H | PH₃P | H | ethoxy | OH |
| H | choline | H | ethoxy | OH |
| H | N,N,N-methyldiethyl-2- | H | ethoxy | OH |
| | (11-oxidaneyl)ethan-1- | | | |
| | aminium | | | |
| H | tosyl | H | ethoxy | OH |
| H | brosyl | H | ethoxy | OH |
| H | mesyl | H | ethoxy | OH |
| H | sulfonate | H | ethoxy | OH |
| H | sulfonamide | H | ethoxy | OH |
| H | phosphonate | H | ethoxy | OH |

TABLE 36
Pericosine Analogues - Structural Formulas V, VI, and VII

| X | Y | W | V | U |
|---|---|---|---|---|
| methyl | OH | H | methyl | OH |
| methyl | Br | H | methyl | OH |
| methyl | Cl | H | methyl | OH |
| methyl | F | H | methyl | OH |
| methyl | acetyloxy | H | methyl | OH |
| methyl | PH₃P | H | methyl | OH |
| methyl | N,N,N-methyldiethyl-2- | H | methyl | OH |
| | (11-oxidaneyl)ethan-1- | | | |
| | aminium | | | |
| methyl | I | H | methyl | OH |
| methyl | tosyl | H | methyl | OH |
| methyl | brosyl | H | methyl | OH |
| methyl | mesyl | H | methyl | OH |
| methyl | sulfonate | H | methyl | OH |
| methyl | sulfonamide | H | methyl | OH |
| methyl | choline | H | methyl | OH |
| methyl | phosphonate | H | methyl | OH |
| ethyl | OH | H | methyl | OH |
| ethyl | Br | H | methyl | OH |
| ethyl | Cl | H | methyl | OH |
| ethyl | F | H | methyl | OH |
| ethyl | I | H | methyl | OH |
| ethyl | acetyloxy | H | methyl | OH |
| ethyl | PH₃P | H | methyl | OH |
| ethyl | choline | H | methyl | OH |
| ethyl | N,N,N-methyldiethyl-2- | H | methyl | OH |
| | (11-oxidaneyl)ethan-1- | | | |
| | aminium | | | |
| ethyl | tosyl | H | methyl | OH |
| ethyl | brosyl | H | methyl | OH |
| ethyl | mesyl | H | methyl | OH |
| ethyl | sulfonate | H | methyl | OH |
| ethyl | sulfonamide | H | methyl | OH |
| ethyl | phosphonate | H | methyl | OH |
| n-propyl | OH | H | methyl | OH |
| n-propyl | Br | H | methyl | OH |
| n-propyl | Cl | H | methyl | OH |
| n-propyl | F | H | methyl | OH |
| n-propyl | I | H | methyl | OH |
| n-propyl | acetyloxy | H | methyl | OH |
| n-propyl | PH₃P | H | methyl | OH |
| n-propyl | choline | H | methyl | OH |
| n-propyl | N,N,N-methyldiethyl-2- | H | methyl | OH |
| | (11-oxidaneyl)ethan-1- | | | |
| | aminium | | | |
| n-propyl | tosyl | H | methyl | OH |
| n-propyl | brosyl | H | methyl | OH |
| n-propyl | mesyl | H | methyl | OH |
| n-propyl | sulfonate | H | methyl | OH |
| n-propyl | sulfonamide | H | methyl | OH |
| n-propyl | phosphonate | H | methyl | OH |
| choline | OH | H | methyl | OH |
| choline | Br | H | methyl | OH |
| choline | Cl | H | methyl | OH |
| choline | F | H | methyl | OH |
| choline | I | H | methyl | OH |
| choline | acetyloxy | H | methyl | OH |
| choline | PH₃P | H | methyl | OH |
| choline | choline | H | methyl | OH |
| choline | N,N,N-methyldiethyl-2- | H | methyl | OH |
| | (11-oxidaneyl)ethan-1- | | | |
| | aminium | | | |
| choline | tosyl | H | methyl | OH |
| choline | brosyl | H | methyl | OH |
| choline | mesyl | H | methyl | OH |
| choline | sulfonate | H | methyl | OH |
| choline | sulfonamide | H | methyl | OH |
| choline | phosphonate | H | methyl | OH |
| H | OH | H | methyl | OH |
| H | Br | H | methyl | OH |
| H | Cl | H | methyl | OH |
| H | F | H | methyl | OH |
| H | I | H | methyl | OH |
| H | acetyloxy | H | methyl | OH |
| H | PH₃P | H | methyl | OH |
| H | choline | H | methyl | OH |
| H | N,N,N-methyldiethyl-2- | H | methyl | OH |
| | (11-oxidaneyl)ethan-1- | | | |
| | aminium | | | |
| H | tosyl | H | methyl | OH |
| H | brosyl | H | methyl | OH |
| H | mesyl | H | methyl | OH |
| H | sulfonate | H | methyl | OH |
| H | sulfonamide | H | methyl | OH |
| H | phosphonate | H | methyl | OH |

TABLE 37
Pericosine Analogues - Structural Formulas V, VI, and VII

| X | Y | W | V | U |
|---|---|---|---|---|
| methyl | OH | H | ethyl | OH |
| methyl | Br | H | ethyl | OH |
| methyl | Cl | H | ethyl | OH |
| methyl | F | H | ethyl | OH |
| methyl | acetyloxy | H | ethyl | OH |
| methyl | PH₃P | H | ethyl | OH |
| methyl | N,N,N-methyldiethyl-2- | H | ethyl | OH |
| | (11-oxidaneyl)ethan-1- | | | |
| | aminium | | | |
| methyl | I | H | ethyl | OH |
| methyl | tosyl | H | ethyl | OH |
| methyl | brosyl | H | ethyl | OH |
| methyl | mesyl | H | ethyl | OH |
| methyl | sulfonate | H | ethyl | OH |
| methyl | sulfonamide | H | ethyl | OH |
| methyl | choline | H | ethyl | OH |
| methyl | phosphonate | H | ethyl | OH |
| ethyl | OH | H | ethyl | OH |
| ethyl | Br | H | ethyl | OH |
| ethyl | Cl | H | ethyl | OH |
| ethyl | F | H | ethyl | OH |
| ethyl | I | H | ethyl | OH |
| ethyl | acetyloxy | H | ethyl | OH |
| ethyl | PH₃P | H | ethyl | OH |
| ethyl | choline | H | ethyl | OH |
| ethyl | N,N,N-methyldiethyl-2- | H | ethyl | OH |
| | (11-oxidaneyl)ethan-1- | | | |
| | aminium | | | |
| ethyl | tosyl | H | ethyl | OH |
| ethyl | brosyl | H | ethyl | OH |
| ethyl | mesyl | H | ethyl | OH |
| ethyl | sulfonate | H | ethyl | OH |
| ethyl | sulfonamide | H | ethyl | OH |
| ethyl | phosphonate | H | ethyl | OH |
| n-propyl | OH | H | ethyl | OH |
| n-propyl | Br | H | ethyl | OH |
| n-propyl | Cl | H | ethyl | OH |
| n-propyl | F | H | ethyl | OH |
| n-propyl | I | H | ethyl | OH |
| n-propyl | acetyloxy | H | ethyl | OH |
| n-propyl | PH₃P | H | ethyl | OH |
| n-propyl | choline | H | ethyl | OH |
| n-propyl | N,N,N-methyldiethyl-2- | H | ethyl | OH |
| | (11-oxidaneyl)ethan-1- | | | |
| | aminium | | | |
| n-propyl | tosyl | H | ethyl | OH |
| n-propyl | brosyl | H | ethyl | OH |
| n-propyl | mesyl | H | ethyl | OH |
| n-propyl | sulfonate | H | ethyl | OH |
| n-propyl | sulfonamide | H | ethyl | OH |
| n-propyl | phosphonate | H | ethyl | OH |
| choline | OH | H | ethyl | OH |
| choline | Br | H | ethyl | OH |
| choline | Cl | H | ethyl | OH |
| choline | F | H | ethyl | OH |
| choline | I | H | ethyl | OH |
| choline | acetyloxy | H | ethyl | OH |
| choline | PH₃P | H | ethyl | OH |
| choline | choline | H | ethyl | OH |
| choline | N,N,N-methyldiethyl-2- | H | ethyl | OH |
| | (11-oxidaneyl)ethan-1- | | | |
| | aminium | | | |
| choline | tosyl | H | ethyl | OH |
| choline | brosyl | H | ethyl | OH |
| choline | mesyl | H | ethyl | OH |
| choline | sulfonate | H | ethyl | OH |
| choline | sulfonamide | H | ethyl | OH |
| choline | phosphonate | H | ethyl | OH |
| H | OH | H | ethyl | OH |
| H | Br | H | ethyl | OH |
| H | Cl | H | ethyl | OH |
| H | F | H | ethyl | OH |
| H | I | H | ethyl | OH |
| H | acetyloxy | H | ethyl | OH |
| H | PH₃P | H | ethyl | OH |
| H | choline | H | ethyl | OH |
| H | N,N,N-methyldiethyl-2- | H | ethyl | OH |
| | (11-oxidaneyl)ethan-1- | | | |
| | aminium | | | |
| H | tosyl | H | ethyl | OH |
| H | brosyl | H | ethyl | OH |
| H | mesyl | H | ethyl | OH |
| H | sulfonate | H | ethyl | OH |
| H | sulfonamide | H | ethyl | OH |
| H | phosphonate | H | ethyl | OH |

TABLE 38
Pericosine Analogues - Structural Formulas V, VI, and VII

| X | Y | W | V | U |
|---|---|---|---|---|
| methyl | OH | H | OH | methoxy |
| methyl | Br | H | OH | methoxy |
| methyl | Cl | H | OH | methoxy |
| methyl | F | H | OH | methoxy |
| methyl | acetyloxy | H | OH | methoxy |
| methyl | PH₃P | H | OH | methoxy |
| methyl | N,N,N-methyldiethyl-2- | H | OH | methoxy |
| | (11-oxidaneyl)ethan-1- | | | |
| | aminium | | | |
| methyl | I | H | OH | methoxy |
| methyl | tosyl | H | OH | methoxy |
| methyl | brosyl | H | OH | methoxy |
| methyl | mesyl | H | OH | methoxy |
| methyl | sulfonate | H | OH | methoxy |
| methyl | sulfonamide | H | OH | methoxy |
| methyl | choline | H | OH | methoxy |
| methyl | phosphonate | H | OH | methoxy |
| ethyl | OH | H | OH | methoxy |
| ethyl | Br | H | OH | methoxy |
| ethyl | Cl | H | OH | methoxy |
| ethyl | F | H | OH | methoxy |
| ethyl | I | H | OH | methoxy |
| ethyl | acetyloxy | H | OH | methoxy |
| ethyl | PH₃P | H | OH | methoxy |
| ethyl | choline | H | OH | methoxy |
| ethyl | N,N,N-methyldiethyl-2- | H | OH | methoxy |
| | (11-oxidaneyl)ethan-1- | | | |
| | aminium | | | |
| ethyl | tosyl | H | OH | methoxy |
| ethyl | brosyl | H | OH | methoxy |
| ethyl | mesyl | H | OH | methoxy |
| ethyl | sulfonate | H | OH | methoxy |
| ethyl | sulfonamide | H | OH | methoxy |
| ethyl | phosphonate | H | OH | methoxy |
| n-propyl | OH | H | OH | methoxy |
| n-propyl | Br | H | OH | methoxy |
| n-propyl | Cl | H | OH | methoxy |
| n-propyl | F | H | OH | methoxy |
| n-propyl | I | H | OH | methoxy |
| n-propyl | acetyloxy | H | OH | methoxy |
| n-propyl | PH₃P | H | OH | methoxy |
| n-propyl | choline | H | OH | methoxy |
| n-propyl | N,N,N-methyldiethyl-2- | H | OH | methoxy |
| | (11-oxidaneyl)ethan-1- | | | |
| | aminium | | | |
| n-propyl | tosyl | H | OH | methoxy |
| n-propyl | brosyl | H | OH | methoxy |
| n-propyl | mesyl | H | OH | methoxy |
| n-propyl | sulfonate | H | OH | methoxy |
| n-propyl | sulfonamide | H | OH | methoxy |
| n-propyl | phosphonate | H | OH | methoxy |
| choline | OH | H | OH | methoxy |
| choline | Br | H | OH | methoxy |
| choline | Cl | H | OH | methoxy |
| choline | F | H | OH | methoxy |
| choline | I | H | OH | methoxy |
| choline | acetyloxy | H | OH | methoxy |
| choline | PH₃P | H | OH | methoxy |
| choline | choline | H | OH | methoxy |
| choline | N,N,N-methyldiethyl-2- | H | OH | methoxy |
| | (11-oxidaneyl)ethan-1- | | | |
| | aminium | | | |
| choline | tosyl | H | OH | methoxy |
| choline | brosyl | H | OH | methoxy |
| choline | mesyl | H | OH | methoxy |
| choline | sulfonate | H | OH | methoxy |
| choline | sulfonamide | H | OH | methoxy |
| choline | phosphonate | H | OH | methoxy |
| H | OH | H | OH | methoxy |
| H | Br | H | OH | methoxy |
| H | Cl | H | OH | methoxy |
| H | F | H | OH | methoxy |
| H | I | H | OH | methoxy |
| H | acetyloxy | H | OH | methoxy |
| H | PH₃P | H | OH | methoxy |
| H | choline | H | OH | methoxy |
| H | N,N,N-methyldiethyl-2- | H | OH | methoxy |
| | (11-oxidaneyl)ethan-1- | | | |
| | aminium | | | |
| H | tosyl | H | OH | methoxy |
| H | brosyl | H | OH | methoxy |
| H | mesyl | H | OH | methoxy |
| H | sulfonate | H | OH | methoxy |
| H | sulfonamide | H | OH | methoxy |
| H | phosphonate | H | OH | methoxy |

TABLE 39
Pericosine Analogues - Structural Formulas V, VI, and VII

| X | Y | W | V | U |
|---|---|---|---|---|
| methyl | OH | H | OH | ethoxy |
| methyl | Br | H | OH | ethoxy |
| methyl | Cl | H | OH | ethoxy |
| methyl | F | H | OH | ethoxy |
| methyl | acetyloxy | H | OH | ethoxy |
| methyl | PH₃P | H | OH | ethoxy |
| methyl | N,N,N-methyldiethyl-2- | H | OH | ethoxy |
| | (11-oxidaneyl)ethan-1- | | | |
| | aminium | | | |
| methyl | I | H | OH | ethoxy |
| methyl | tosyl | H | OH | ethoxy |
| methyl | brosyl | H | OH | ethoxy |
| methyl | mesyl | H | OH | ethoxy |
| methyl | sulfonate | H | OH | ethoxy |
| methyl | sulfonamide | H | OH | ethoxy |
| methyl | choline | H | OH | ethoxy |
| methyl | phosphonate | H | OH | ethoxy |
| ethyl | OH | H | OH | ethoxy |
| ethyl | Br | H | OH | ethoxy |
| ethyl | Cl | H | OH | ethoxy |
| ethyl | F | H | OH | ethoxy |
| ethyl | I | H | OH | ethoxy |
| ethyl | acetyloxy | H | OH | ethoxy |
| ethyl | PH₃P | H | OH | ethoxy |
| ethyl | choline | H | OH | ethoxy |
| ethyl | N,N,N-methyldiethyl-2- | H | OH | ethoxy |
| | (11-oxidaneyl)ethan-1- | | | |
| | aminium | | | |
| ethyl | tosyl | H | OH | ethoxy |
| ethyl | brosyl | H | OH | ethoxy |
| ethyl | mesyl | H | OH | ethoxy |
| ethyl | sulfonate | H | OH | ethoxy |
| ethyl | sulfonamide | H | OH | ethoxy |
| ethyl | phosphonate | H | OH | ethoxy |
| n-propyl | OH | H | OH | ethoxy |
| n-propyl | Br | H | OH | ethoxy |
| n-propyl | Cl | H | OH | ethoxy |
| n-propyl | F | H | OH | ethoxy |
| n-propyl | I | H | OH | ethoxy |
| n-propyl | acetyloxy | H | OH | ethoxy |
| n-propyl | PH₃P | H | OH | ethoxy |
| n-propyl | choline | H | OH | ethoxy |
| n-propyl | N,N,N-methyldiethyl-2- | H | OH | ethoxy |
| | (11-oxidaneyl)ethan-1- | | | |
| | aminium | | | |
| n-propyl | tosyl | H | OH | ethoxy |
| n-propyl | brosyl | H | OH | ethoxy |
| n-propyl | mesyl | H | OH | ethoxy |
| n-propyl | sulfonate | H | OH | ethoxy |
| n-propyl | sulfonamide | H | OH | ethoxy |
| n-propyl | phosphonate | H | OH | ethoxy |
| choline | OH | H | OH | ethoxy |
| choline | Br | H | OH | ethoxy |
| choline | Cl | H | OH | ethoxy |
| choline | F | H | OH | ethoxy |
| choline | I | H | OH | ethoxy |
| choline | acetyloxy | H | OH | ethoxy |
| choline | PH₃P | H | OH | ethoxy |
| choline | choline | H | OH | ethoxy |
| choline | N,N,N-methyldiethyl-2- | H | OH | ethoxy |
| | (11-oxidaneyl)ethan-1- | | | |
| | aminium | | | |
| choline | tosyl | H | OH | ethoxy |
| choline | brosyl | H | OH | ethoxy |
| choline | mesyl | H | OH | ethoxy |
| choline | sulfonate | H | OH | ethoxy |
| choline | sulfonamide | H | OH | ethoxy |
| choline | phosphonate | H | OH | ethoxy |
| H | OH | H | OH | ethoxy |
| H | Br | H | OH | ethoxy |
| H | Cl | H | OH | ethoxy |
| H | F | H | OH | ethoxy |
| H | I | H | OH | ethoxy |
| H | acetyloxy | H | OH | ethoxy |
| H | PH₃P | H | OH | ethoxy |
| H | choline | H | OH | ethoxy |
| H | N,N,N-methyldiethyl-2- | H | OH | ethoxy |
| | (11-oxidaneyl)ethan-1- | | | |
| | aminium | | | |
| H | tosyl | H | OH | ethoxy |
| H | brosyl | H | OH | ethoxy |
| H | mesyl | H | OH | ethoxy |
| H | sulfonate | H | OH | ethoxy |
| H | sulfonamide | H | OH | ethoxy |
| H | phosphonate | H | OH | ethoxy |

TABLE 40
Pericosine Analogues - Structural Formulas V, VI, and VII

| X | Y | W | V | U |
|---|---|---|---|---|
| methyl | OH | H | OH | methyl |
| methyl | Br | H | OH | methyl |
| methyl | Cl | H | OH | methyl |
| methyl | F | H | OH | methyl |
| methyl | acetyloxy | H | OH | methyl |
| methyl | PH₃P | H | OH | methyl |
| methyl | N,N,N-methyldiethyl-2- | H | OH | methyl |
| | (11-oxidaneyl)ethan-1- | | | |
| | aminium | | | |
| methyl | I | H | OH | methyl |
| methyl | tosyl | H | OH | methyl |
| methyl | brosyl | H | OH | methyl |
| methyl | mesyl | H | OH | methyl |
| methyl | sulfonate | H | OH | methyl |
| methyl | sulfonamide | H | OH | methyl |
| methyl | choline | H | OH | methyl |
| methyl | phosphonate | H | OH | methyl |
| ethyl | OH | H | OH | methyl |
| ethyl | Br | H | OH | methyl |
| ethyl | Cl | H | OH | methyl |
| ethyl | F | H | OH | methyl |
| ethyl | I | H | OH | methyl |
| ethyl | acetyloxy | H | OH | methyl |
| ethyl | PH₃P | H | OH | methyl |
| ethyl | choline | H | OH | methyl |
| ethyl | N,N,N-methyldiethyl-2- | H | OH | methyl |
| | (11-oxidaneyl)ethan-1- | | | |
| | aminium | | | |
| ethyl | tosyl | H | OH | methyl |
| ethyl | brosyl | H | OH | methyl |
| ethyl | mesyl | H | OH | methyl |
| ethyl | sulfonate | H | OH | methyl |
| ethyl | sulfonamide | H | OH | methyl |
| ethyl | phosphonate | H | OH | methyl |
| n-propyl | OH | H | OH | methyl |
| n-propyl | Br | H | OH | methyl |
| n-propyl | Cl | H | OH | methyl |
| n-propyl | F | H | OH | methyl |
| n-propyl | I | H | OH | methyl |
| n-propyl | acetyloxy | H | OH | methyl |
| n-propyl | PH₃P | H | OH | methyl |
| n-propyl | choline | H | OH | methyl |
| n-propyl | N,N,N-methyldiethyl-2- | H | OH | methyl |
| | (11-oxidaneyl)ethan-1- | | | |
| | aminium | | | |
| n-propyl | tosyl | H | OH | methyl |
| n-propyl | brosyl | H | OH | methyl |
| n-propyl | mesyl | H | OH | methyl |
| n-propyl | sulfonate | H | OH | methyl |
| n-propyl | sulfonamide | H | OH | methyl |
| n-propyl | phosphonate | H | OH | methyl |
| choline | OH | H | OH | methyl |
| choline | Br | H | OH | methyl |
| choline | Cl | H | OH | methyl |
| choline | F | H | OH | methyl |
| choline | I | H | OH | methyl |
| choline | acetyloxy | H | OH | methyl |
| choline | PH₃P | H | OH | methyl |
| choline | choline | H | OH | methyl |
| choline | N,N,N-methyldiethyl-2- | H | OH | methyl |
| | (11-oxidaneyl)ethan-1- | | | |
| | aminium | | | |
| choline | tosyl | H | OH | methyl |
| choline | brosyl | H | OH | methyl |
| choline | mesyl | H | OH | methyl |
| choline | sulfonate | H | OH | methyl |
| choline | sulfonamide | H | OH | methyl |
| choline | phosphonate | H | OH | methyl |
| H | OH | H | OH | methyl |
| H | Br | H | OH | methyl |
| H | Cl | H | OH | methyl |
| H | F | H | OH | methyl |
| H | I | H | OH | methyl |
| H | acetyloxy | H | OH | methyl |
| H | PH₃P | H | OH | methyl |
| H | choline | H | OH | methyl |
| H | N,N,N-methyldiethyl-2- | H | OH | methyl |
| | (11-oxidaneyl)ethan-1- | | | |
| | aminium | | | |
| H | tosyl | H | OH | methyl |
| H | brosyl | H | OH | methyl |
| H | mesyl | H | OH | methyl |
| H | sulfonate | H | OH | methyl |
| H | sulfonamide | H | OH | methyl |
| H | phosphonate | H | OH | methyl |

TABLE 41
Pericosine Analogues - Structural Formulas V, VI, and VII

| X | Y | W | V | U |
|---|---|---|---|---|
| methyl | OH | H | OH | ethyl |
| methyl | Br | H | OH | ethyl |
| methyl | Cl | H | OH | ethyl |
| methyl | F | H | OH | ethyl |
| methyl | acetyloxy | H | OH | ethyl |
| methyl | PH₃P | H | OH | ethyl |
| methyl | N,N,N-methyldiethyl-2- | H | OH | ethyl |
| | (11-oxidaneyl)ethan-1- | | | |
| | aminium | | | |
| methyl | I | H | OH | ethyl |
| methyl | tosyl | H | OH | ethyl |
| methyl | brosyl | H | OH | ethyl |
| methyl | mesyl | H | OH | ethyl |
| methyl | sulfonate | H | OH | ethyl |
| methyl | sulfonamide | H | OH | ethyl |
| methyl | choline | H | OH | ethyl |
| methyl | phosphonate | H | OH | ethyl |
| ethyl | OH | H | OH | ethyl |
| ethyl | Br | H | OH | ethyl |
| ethyl | Cl | H | OH | ethyl |
| ethyl | F | H | OH | ethyl |
| ethyl | I | H | OH | ethyl |
| ethyl | acetyloxy | H | OH | ethyl |
| ethyl | PH₃P | H | OH | ethyl |
| ethyl | choline | H | OH | ethyl |
| ethyl | N,N,N-methyldiethyl-2- | H | OH | ethyl |
| | (11-oxidaneyl)ethan-1- | | | |
| | aminium | | | |
| ethyl | tosyl | H | OH | ethyl |
| ethyl | brosyl | H | OH | ethyl |
| ethyl | mesyl | H | OH | ethyl |
| ethyl | sulfonate | H | OH | ethyl |
| ethyl | sulfonamide | H | OH | ethyl |
| ethyl | phosphonate | H | OH | ethyl |
| n-propyl | OH | H | OH | ethyl |
| n-propyl | Br | H | OH | ethyl |
| n-propyl | Cl | H | OH | ethyl |
| n-propyl | F | H | OH | ethyl |
| n-propyl | I | H | OH | ethyl |
| n-propyl | acetyloxy | H | OH | ethyl |
| n-propyl | PH₃P | H | OH | ethyl |
| n-propyl | choline | H | OH | ethyl |
| n-propyl | N,N,N-methyldiethyl-2- | H | OH | ethyl |
| | (11-oxidaneyl)ethan-1- | | | |
| | aminium | | | |
| n-propyl | tosyl | H | OH | ethyl |
| n-propyl | brosyl | H | OH | ethyl |
| n-propyl | mesyl | H | OH | ethyl |
| n-propyl | sulfonate | H | OH | ethyl |
| n-propyl | sulfonamide | H | OH | ethyl |
| n-propyl | phosphonate | H | OH | ethyl |
| choline | OH | H | OH | ethyl |
| choline | Br | H | OH | ethyl |
| choline | Cl | H | OH | ethyl |
| choline | F | H | OH | ethyl |
| choline | I | H | OH | ethyl |
| choline | acetyloxy | H | OH | ethyl |
| choline | PH₃P | H | OH | ethyl |
| choline | choline | H | OH | ethyl |
| choline | N,N,N-methyldiethyl-2- | H | OH | ethyl |
| | (11-oxidaneyl)ethan-1- | | | |
| | aminium | | | |
| choline | tosyl | H | OH | ethyl |
| choline | brosyl | H | OH | ethyl |
| choline | mesyl | H | OH | ethyl |
| choline | sulfonate | H | OH | ethyl |
| choline | sulfonamide | H | OH | ethyl |
| choline | phosphonate | H | OH | ethyl |
| H | OH | H | OH | ethyl |
| H | Br | H | OH | ethyl |
| H | Cl | H | OH | ethyl |
| H | F | H | OH | ethyl |
| H | I | H | OH | ethyl |
| H | acetyloxy | H | OH | ethyl |
| H | PH₃P | H | OH | ethyl |
| H | choline | H | OH | ethyl |
| H | N,N,N-methyldiethyl-2- | H | OH | ethyl |
| | (11-oxidaneyl)ethan-1- | | | |
| | aminium | | | |
| H | tosyl | H | OH | ethyl |
| H | brosyl | H | OH | ethyl |
| H | mesyl | H | OH | ethyl |
| H | sulfonate | H | OH | ethyl |
| H | sulfonamide | H | OH | ethyl |
| H | phosphonate | H | OH | ethyl |

TABLE 42
Pericosine Analogues - Structural Formulas V, VI, and VII

| X | Y | W | V | U |
|---|---|---|---|---|
| methyl | OH | methoxy | OH | methyl |
| methyl | Br | methoxy | OH | methyl |
| methyl | Cl | methoxy | OH | methyl |
| methyl | F | methoxy | OH | methyl |
| methyl | acetyloxy | methoxy | OH | methyl |
| methyl | PH₃P | methoxy | OH | methyl |
| methyl | N,N,N-methyldiethyl-2- | methoxy | OH | methyl |
| | (11-oxidaneyl)ethan-1- | | | |
| | aminium | | | |
| methyl | I | methoxy | OH | methyl |
| methyl | tosyl | methoxy | OH | methyl |
| methyl | brosyl | methoxy | OH | methyl |
| methyl | mesyl | methoxy | OH | methyl |
| methyl | sulfonate | methoxy | OH | methyl |
| methyl | sulfonamide | methoxy | OH | methyl |
| methyl | choline | methoxy | OH | methyl |
| methyl | phosphonate | methoxy | OH | methyl |
| ethyl | OH | methoxy | OH | methyl |
| ethyl | Br | methoxy | OH | methyl |
| ethyl | Cl | methoxy | OH | methyl |
| ethyl | F | methoxy | OH | methyl |
| ethyl | I | methoxy | OH | methyl |
| ethyl | acetyloxy | methoxy | OH | methyl |
| ethyl | PH₃P | methoxy | OH | methyl |
| ethyl | choline | methoxy | OH | methyl |
| ethyl | N,N,N-methyldiethyl-2- | methoxy | OH | methyl |
| | (11-oxidaneyl)ethan-1- | | | |
| | aminium | | | |
| ethyl | tosyl | methoxy | OH | methyl |
| ethyl | brosyl | methoxy | OH | methyl |
| ethyl | mesyl | methoxy | OH | methyl |
| ethyl | sulfonate | methoxy | OH | methyl |
| ethyl | sulfonamide | methoxy | OH | methyl |
| ethyl | phosphonate | methoxy | OH | methyl |
| n-propyl | OH | methoxy | OH | methyl |
| n-propyl | Br | methoxy | OH | methyl |
| n-propyl | Cl | methoxy | OH | methyl |
| n-propyl | F | methoxy | OH | methyl |
| n-propyl | I | methoxy | OH | methyl |
| n-propyl | acetyloxy | methoxy | OH | methyl |
| n-propyl | PH₃P | methoxy | OH | methyl |
| n-propyl | choline | methoxy | OH | methyl |
| n-propyl | N,N,N-methyldiethyl-2- | methoxy | OH | methyl |
| | (11-oxidaneyl)ethan-1- | | | |
| | aminium | | | |
| n-propyl | tosyl | methoxy | OH | methyl |
| n-propyl | brosyl | methoxy | OH | methyl |
| n-propyl | mesyl | methoxy | OH | methyl |
| n-propyl | sulfonate | methoxy | OH | methyl |
| n-propyl | sulfonamide | methoxy | OH | methyl |
| n-propyl | phosphonate | methoxy | OH | methyl |
| choline | OH | methoxy | OH | methyl |
| choline | Br | methoxy | OH | methyl |
| choline | Cl | methoxy | OH | methyl |
| choline | F | methoxy | OH | methyl |
| choline | I | methoxy | OH | methyl |
| choline | acetyloxy | methoxy | OH | methyl |
| choline | PH₃P | methoxy | OH | methyl |
| choline | choline | methoxy | OH | methyl |
| choline | N,N,N-methyldiethyl-2- | methoxy | OH | methyl |
| | (11-oxidaneyl)ethan-1- | | | |
| | aminium | | | |
| choline | tosyl | methoxy | OH | methyl |
| choline | brosyl | methoxy | OH | methyl |
| choline | mesyl | methoxy | OH | methyl |
| choline | sulfonate | methoxy | OH | methyl |
| choline | sulfonamide | methoxy | OH | methyl |
| choline | phosphonate | methoxy | OH | methyl |
| H | OH | methoxy | OH | methyl |
| H | Br | methoxy | OH | methyl |
| H | Cl | methoxy | OH | methyl |
| H | F | methoxy | OH | methyl |
| H | I | methoxy | OH | methyl |
| H | acetyloxy | methoxy | OH | methyl |
| H | PH₃P | methoxy | OH | methyl |
| H | choline | methoxy | OH | methyl |
| H | N,N,N-methyldiethyl-2- | methoxy | OH | methyl |
| | (11-oxidaneyl)ethan-1- | | | |
| | aminium | | | |
| H | tosyl | methoxy | OH | methyl |
| H | brosyl | methoxy | OH | methyl |
| H | mesyl | methoxy | OH | methyl |
| H | sulfonate | methoxy | OH | methyl |
| H | sulfonamide | methoxy | OH | methyl |
| H | phosphonate | methoxy | OH | methyl |

TABLE 43
Pericosine Analogues - Structural Formulas V, VI, and VII

| X | Y | W | V | U |
|---|---|---|---|---|
| methyl | OH | ethoxy | OH | ethyl |
| methyl | Br | ethoxy | OH | ethyl |
| methyl | Cl | ethoxy | OH | ethyl |
| methyl | F | ethoxy | OH | ethyl |
| methyl | acetyloxy | ethoxy | OH | ethyl |
| methyl | PH₃P | ethoxy | OH | ethyl |
| methyl | N,N,N-methyldiethyl-2- | ethoxy | OH | ethyl |
| | (11-oxidaneyl)ethan-1- | | | |
| | aminium | | | |
| methyl | I | ethoxy | OH | ethyl |
| methyl | tosyl | ethoxy | OH | ethyl |
| methyl | brosyl | ethoxy | OH | ethyl |
| methyl | mesyl | ethoxy | OH | ethyl |
| methyl | sulfonate | ethoxy | OH | ethyl |
| methyl | sulfonamide | ethoxy | OH | ethyl |
| methyl | choline | ethoxy | OH | ethyl |
| methyl | phosphonate | ethoxy | OH | ethyl |
| ethyl | OH | ethoxy | OH | ethyl |
| ethyl | Br | ethoxy | OH | ethyl |
| ethyl | Cl | ethoxy | OH | ethyl |
| ethyl | F | ethoxy | OH | ethyl |
| ethyl | I | ethoxy | OH | ethyl |
| ethyl | acetyloxy | ethoxy | OH | ethyl |
| ethyl | PH₃P | ethoxy | OH | ethyl |
| ethyl | choline | ethoxy | OH | ethyl |
| ethyl | N,N,N-methyldiethyl-2- | ethoxy | OH | ethyl |
| | (11-oxidaneyl)ethan-1- | | | |
| | aminium | | | |
| ethyl | tosyl | ethoxy | OH | ethyl |
| ethyl | brosyl | ethoxy | OH | ethyl |
| ethyl | mesyl | ethoxy | OH | ethyl |
| ethyl | sulfonate | ethoxy | OH | ethyl |
| ethyl | sulfonamide | ethoxy | OH | ethyl |
| ethyl | phosphonate | ethoxy | OH | ethyl |
| n-propyl | OH | ethoxy | OH | ethyl |
| n-propyl | Br | ethoxy | OH | ethyl |
| n-propyl | Cl | ethoxy | OH | ethyl |
| n-propyl | F | ethoxy | OH | ethyl |
| n-propyl | I | ethoxy | OH | ethyl |
| n-propyl | acetyloxy | ethoxy | OH | ethyl |
| n-propyl | PH₃P | ethoxy | OH | ethyl |
| n-propyl | choline | ethoxy | OH | ethyl |
| n-propyl | N,N,N-methyldiethyl-2- | ethoxy | OH | ethyl |
| | (11-oxidaneyl)ethan-1- | | | |
| | aminium | | | |
| n-propyl | tosyl | ethoxy | OH | ethyl |
| n-propyl | brosyl | ethoxy | OH | ethyl |
| n-propyl | mesyl | ethoxy | OH | ethyl |
| n-propyl | sulfonate | ethoxy | OH | ethyl |
| n-propyl | sulfonamide | ethoxy | OH | ethyl |
| n-propyl | phosphonate | ethoxy | OH | ethyl |
| choline | OH | ethoxy | OH | ethyl |
| choline | Br | ethoxy | OH | ethyl |
| choline | Cl | ethoxy | OH | ethyl |
| choline | F | ethoxy | OH | ethyl |
| choline | I | ethoxy | OH | ethyl |
| choline | acetyloxy | ethoxy | OH | ethyl |
| choline | PH₃P | ethoxy | OH | ethyl |
| choline | choline | ethoxy | OH | ethyl |
| choline | N,N,N-methyldiethyl-2- | ethoxy | OH | ethyl |
| | (11-oxidaneyl)ethan-1- | | | |
| | aminium | | | |
| choline | tosyl | ethoxy | OH | ethyl |
| choline | brosyl | ethoxy | OH | ethyl |
| choline | mesyl | ethoxy | OH | ethyl |
| choline | sulfonate | ethoxy | OH | ethyl |
| choline | sulfonamide | ethoxy | OH | ethyl |
| choline | phosphonate | ethoxy | OH | ethyl |
| H | OH | ethoxy | OH | ethyl |
| H | Br | ethoxy | OH | ethyl |
| H | Cl | ethoxy | OH | ethyl |
| H | F | ethoxy | OH | ethyl |
| H | I | ethoxy | OH | ethyl |
| H | acetyloxy | ethoxy | OH | ethyl |
| H | PH₃P | ethoxy | OH | ethyl |
| H | choline | ethoxy | OH | ethyl |
| H | N,N,N-methyldiethyl-2- | ethoxy | OH | ethyl |
| | (11-oxidaneyl)ethan-1- | | | |
| | aminium | | | |
| H | tosyl | ethoxy | OH | ethyl |
| H | brosyl | ethoxy | OH | ethyl |
| H | mesyl | ethoxy | OH | ethyl |
| H | sulfonate | ethoxy | OH | ethyl |
| H | sulfonamide | ethoxy | OH | ethyl |
| H | phosphonate | ethoxy | OH | ethyl |

TABLE 44
Pericosine Analogues - Structural Formulas V, VI, and VII

| X | Y | W | V | U |
|---|---|---|---|---|
| methyl | OH | methyl | OH | methoxy |
| methyl | Br | methyl | OH | methoxy |
| methyl | Cl | methyl | OH | methoxy |
| methyl | F | methyl | OH | methoxy |
| methyl | acetyloxy | methyl | OH | methoxy |
| methyl | PH₃P | methyl | OH | methoxy |
| methyl | N,N,N-methyldiethyl-2- | methyl | OH | methoxy |
| | (11-oxidaneyl)ethan-1- | | | |
| | aminium | | | |
| methyl | I | methyl | OH | methoxy |
| methyl | tosyl | methyl | OH | methoxy |
| methyl | brosyl | methyl | OH | methoxy |
| methyl | mesyl | methyl | OH | methoxy |
| methyl | sulfonate | methyl | OH | methoxy |
| methyl | sulfonamide | methyl | OH | methoxy |
| methyl | choline | methyl | OH | methoxy |
| methyl | phosphonate | methyl | OH | methoxy |
| ethyl | OH | methyl | OH | methoxy |
| ethyl | Br | methyl | OH | methoxy |
| ethyl | Cl | methyl | OH | methoxy |
| ethyl | F | methyl | OH | methoxy |
| ethyl | I | methyl | OH | methoxy |
| ethyl | acetyloxy | methyl | OH | methoxy |
| ethyl | PH₃P | methyl | OH | methoxy |
| ethyl | choline | methyl | OH | methoxy |
| ethyl | N,N,N-methyldiethyl-2- | methyl | OH | methoxy |
| | (11-oxidaneyl)ethan-1- | | | |
| | aminium | | | |
| ethyl | tosyl | methyl | OH | methoxy |
| ethyl | brosyl | methyl | OH | methoxy |
| ethyl | mesyl | methyl | OH | methoxy |
| ethyl | sulfonate | methyl | OH | methoxy |
| ethyl | sulfonamide | methyl | OH | methoxy |
| ethyl | phosphonate | methyl | OH | methoxy |
| n-propyl | OH | methyl | OH | methoxy |
| n-propyl | Br | methyl | OH | methoxy |
| n-propyl | Cl | methyl | OH | methoxy |
| n-propyl | F | methyl | OH | methoxy |
| n-propyl | I | methyl | OH | methoxy |
| n-propyl | acetyloxy | methyl | OH | methoxy |
| n-propyl | PH₃P | methyl | OH | methoxy |
| n-propyl | choline | methyl | OH | methoxy |
| n-propyl | N,N,N-methyldiethyl-2- | methyl | OH | methoxy |
| | (11-oxidaneyl)ethan-1- | | | |
| | aminium | | | |
| n-propyl | tosyl | methyl | OH | methoxy |
| n-propyl | brosyl | methyl | OH | methoxy |
| n-propyl | mesyl | methyl | OH | methoxy |
| n-propyl | sulfonate | methyl | OH | methoxy |
| n-propyl | sulfonamide | methyl | OH | methoxy |
| n-propyl | phosphonate | methyl | OH | methoxy |
| choline | OH | methyl | OH | methoxy |
| choline | Br | methyl | OH | methoxy |
| choline | Cl | methyl | OH | methoxy |
| choline | F | methyl | OH | methoxy |
| choline | I | methyl | OH | methoxy |
| choline | acetyloxy | methyl | OH | methoxy |
| choline | PH₃P | methyl | OH | methoxy |
| choline | choline | methyl | OH | methoxy |
| choline | N,N,N-methyldiethyl-2- | methyl | OH | methoxy |
| | (11-oxidaneyl)ethan-1- | | | |
| | aminium | | | |
| choline | tosyl | methyl | OH | methoxy |
| choline | brosyl | methyl | OH | methoxy |
| choline | mesyl | methyl | OH | methoxy |
| choline | sulfonate | methyl | OH | methoxy |
| choline | sulfonamide | methyl | OH | methoxy |
| choline | phosphonate | methyl | OH | methoxy |
| H | OH | methyl | OH | methoxy |
| H | Br | methyl | OH | methoxy |
| H | Cl | methyl | OH | methoxy |
| H | F | methyl | OH | methoxy |
| H | I | methyl | OH | methoxy |
| H | acetyloxy | methyl | OH | methoxy |
| H | PH₃P | methyl | OH | methoxy |
| H | choline | methyl | OH | methoxy |
| H | N,N,N-methyldiethyl-2- | methyl | OH | methoxy |
| | (11-oxidaneyl)ethan-1- | | | |
| | aminium | | | |
| H | tosyl | methyl | OH | methoxy |
| H | brosyl | methyl | OH | methoxy |
| H | mesyl | methyl | OH | methoxy |
| H | sulfonate | methyl | OH | methoxy |
| H | sulfonamide | methyl | OH | methoxy |
| H | phosphonate | methyl | OH | methoxy |

TABLE 45
Pericosine Analogues - Structural Formulas V, VI, and VII

| X | Y | W | V | U |
|---|---|---|---|---|
| methyl | OH | ethyl | OH | ethoxy |
| methyl | Br | ethyl | OH | ethoxy |
| methyl | Cl | ethyl | OH | ethoxy |
| methyl | F | ethyl | OH | ethoxy |
| methyl | acetyloxy | ethyl | OH | ethoxy |
| methyl | PH₃P | ethyl | OH | ethoxy |
| methyl | N,N,N-methyldiethyl-2- | ethyl | OH | ethoxy |
| | (11-oxidaneyl)ethan-1- | | | |
| | aminium | | | |
| methyl | I | ethyl | OH | ethoxy |
| methyl | tosyl | ethyl | OH | ethoxy |
| methyl | brosyl | ethyl | OH | ethoxy |
| methyl | mesyl | ethyl | OH | ethoxy |
| methyl | sulfonate | ethyl | OH | ethoxy |
| methyl | sulfonamide | ethyl | OH | ethoxy |
| methyl | choline | ethyl | OH | ethoxy |
| methyl | phosphonate | ethyl | OH | ethoxy |
| ethyl | OH | ethyl | OH | ethoxy |
| ethyl | Br | ethyl | OH | ethoxy |
| ethyl | Cl | ethyl | OH | ethoxy |
| ethyl | F | ethyl | OH | ethoxy |
| ethyl | I | ethyl | OH | ethoxy |
| ethyl | acetyloxy | ethyl | OH | ethoxy |
| ethyl | PH₃P | ethyl | OH | ethoxy |
| ethyl | choline | ethyl | OH | ethoxy |
| ethyl | N,N,N-methyldiethyl-2- | ethyl | OH | ethoxy |
| | (11-oxidaneyl)ethan-1- | | | |
| | aminium | | | |
| ethyl | tosyl | ethyl | OH | ethoxy |
| ethyl | brosyl | ethyl | OH | ethoxy |
| ethyl | mesyl | ethyl | OH | ethoxy |
| ethyl | sulfonate | ethyl | OH | ethoxy |
| ethyl | sulfonamide | ethyl | OH | ethoxy |
| ethyl | phosphonate | ethyl | OH | ethoxy |
| n-propyl | OH | ethyl | OH | ethoxy |
| n-propyl | Br | ethyl | OH | ethoxy |
| n-propyl | Cl | ethyl | OH | ethoxy |
| n-propyl | F | ethyl | OH | ethoxy |
| n-propyl | I | ethyl | OH | ethoxy |
| n-propyl | acetyloxy | ethyl | OH | ethoxy |
| n-propyl | PH₃P | ethyl | OH | ethoxy |
| n-propyl | choline | ethyl | OH | ethoxy |
| n-propyl | N,N,N-methyldiethyl-2- | ethyl | OH | ethoxy |
| | (11-oxidaneyl)ethan-1- | | | |
| | aminium | | | |
| n-propyl | tosyl | ethyl | OH | ethoxy |
| n-propyl | brosyl | ethyl | OH | ethoxy |
| n-propyl | mesyl | ethyl | OH | ethoxy |
| n-propyl | sulfonate | ethyl | OH | ethoxy |
| n-propyl | sulfonamide | ethyl | OH | ethoxy |
| n-propyl | phosphonate | ethyl | OH | ethoxy |
| choline | OH | ethyl | OH | ethoxy |
| choline | Br | ethyl | OH | ethoxy |
| choline | Cl | ethyl | OH | ethoxy |
| choline | F | ethyl | OH | ethoxy |
| choline | I | ethyl | OH | ethoxy |
| choline | acetyloxy | ethyl | OH | ethoxy |
| choline | PH₃P | ethyl | OH | ethoxy |
| choline | choline | ethyl | OH | ethoxy |
| choline | N,N,N-methyldiethyl-2- | ethyl | OH | ethoxy |
| | (11-oxidaneyl)ethan-1- | | | |
| | aminium | | | |
| choline | tosyl | ethyl | OH | ethoxy |
| choline | brosyl | ethyl | OH | ethoxy |
| choline | mesyl | ethyl | OH | ethoxy |
| choline | sulfonate | ethyl | OH | ethoxy |
| choline | sulfonamide | ethyl | OH | ethoxy |
| choline | phosphonate | ethyl | OH | ethoxy |
| H | OH | ethyl | OH | ethoxy |
| H | Br | ethyl | OH | ethoxy |
| H | Cl | ethyl | OH | ethoxy |
| H | F | ethyl | OH | ethoxy |
| H | I | ethyl | OH | ethoxy |
| H | acetyloxy | ethyl | OH | ethoxy |
| H | PH₃P | ethyl | OH | ethoxy |
| H | choline | ethyl | OH | ethoxy |
| H | N,N,N-methyldiethyl-2- | ethyl | OH | ethoxy |
| | (11-oxidaneyl)ethan-1- | | | |
| | aminium | | | |
| H | tosyl | ethyl | OH | ethoxy |
| H | brosyl | ethyl | OH | ethoxy |
| H | mesyl | ethyl | OH | ethoxy |
| H | sulfonate | ethyl | OH | ethoxy |
| H | sulfonamide | ethyl | OH | ethoxy |
| H | phosphonate | ethyl | OH | ethoxy |

TABLE 46
Pericosine Analogues - Structural Formulas V, VI, and VII

| X | Y | W | V | U |
|---|---|---|---|---|
| methyl | OH | methoxy | OH | ethyl |
| methyl | Br | methoxy | OH | ethyl |
| methyl | Cl | methoxy | OH | ethyl |
| methyl | F | methoxy | OH | ethyl |
| methyl | acetyloxy | methoxy | OH | ethyl |
| methyl | PH₃P | methoxy | OH | ethyl |
| methyl | N,N,N-methyldiethyl-2- | methoxy | OH | ethyl |
| | (11-oxidaneyl)ethan-1- | | | |
| | aminium | | | |
| methyl | I | methoxy | OH | ethyl |
| methyl | tosyl | methoxy | OH | ethyl |
| methyl | brosyl | methoxy | OH | ethyl |
| methyl | mesyl | methoxy | OH | ethyl |
| methyl | sulfonate | methoxy | OH | ethyl |
| methyl | sulfonamide | methoxy | OH | ethyl |
| methyl | choline | methoxy | OH | ethyl |
| methyl | phosphonate | methoxy | OH | ethyl |
| ethyl | OH | methoxy | OH | ethyl |
| ethyl | Br | methoxy | OH | ethyl |
| ethyl | Cl | methoxy | OH | ethyl |
| ethyl | F | methoxy | OH | ethyl |
| ethyl | I | methoxy | OH | ethyl |
| ethyl | acetyloxy | methoxy | OH | ethyl |
| ethyl | PH₃P | methoxy | OH | ethyl |
| ethyl | choline | methoxy | OH | ethyl |
| ethyl | N,N,N-methyldiethyl-2- | methoxy | OH | ethyl |
| | (11-oxidaneyl)ethan-1- | | | |
| | aminium | | | |
| ethyl | tosyl | methoxy | OH | ethyl |
| ethyl | brosyl | methoxy | OH | ethyl |
| ethyl | mesyl | methoxy | OH | ethyl |
| ethyl | sulfonate | methoxy | OH | ethyl |
| ethyl | sulfonamide | methoxy | OH | ethyl |
| ethyl | phosphonate | methoxy | OH | ethyl |
| n-propyl | OH | methoxy | OH | ethyl |
| n-propyl | Br | methoxy | OH | ethyl |
| n-propyl | Cl | methoxy | OH | ethyl |
| n-propyl | F | methoxy | OH | ethyl |
| n-propyl | I | methoxy | OH | ethyl |
| n-propyl | acetyloxy | methoxy | OH | ethyl |
| n-propyl | PH₃P | methoxy | OH | ethyl |
| n-propyl | choline | methoxy | OH | ethyl |
| n-propyl | N,N,N-methyldiethyl-2- | methoxy | OH | ethyl |
| | (11-oxidaneyl)ethan-1- | | | |
| | aminium | | | |
| n-propyl | tosyl | methoxy | OH | ethyl |
| n-propyl | brosyl | methoxy | OH | ethyl |
| n-propyl | mesyl | methoxy | OH | ethyl |
| n-propyl | sulfonate | methoxy | OH | ethyl |
| n-propyl | sulfonamide | methoxy | OH | ethyl |
| n-propyl | phosphonate | methoxy | OH | ethyl |
| choline | OH | methoxy | OH | ethyl |
| choline | Br | methoxy | OH | ethyl |
| choline | Cl | methoxy | OH | ethyl |
| choline | F | methoxy | OH | ethyl |
| choline | I | methoxy | OH | ethyl |
| choline | acetyloxy | methoxy | OH | ethyl |
| choline | PH₃P | methoxy | OH | ethyl |
| choline | choline | methoxy | OH | ethyl |
| choline | N,N,N-methyldiethyl-2- | methoxy | OH | ethyl |
| | (11-oxidaneyl)ethan-1- | | | |
| | aminium | | | |
| choline | tosyl | methoxy | OH | ethyl |
| choline | brosyl | methoxy | OH | ethyl |
| choline | mesyl | methoxy | OH | ethyl |
| choline | sulfonate | methoxy | OH | ethyl |
| choline | sulfonamide | methoxy | OH | ethyl |
| choline | phosphonate | methoxy | OH | ethyl |
| H | OH | methoxy | OH | ethyl |
| H | Br | methoxy | OH | ethyl |
| H | Cl | methoxy | OH | ethyl |
| H | F | methoxy | OH | ethyl |
| H | I | methoxy | OH | ethyl |
| H | acetyloxy | methoxy | OH | ethyl |
| H | PH₃P | methoxy | OH | ethyl |
| H | choline | methoxy | OH | ethyl |
| H | N,N,N-methyldiethyl-2- | methoxy | OH | ethyl |
| | (11-oxidaneyl)ethan-1- | | | |
| | aminium | | | |
| H | tosyl | methoxy | OH | ethyl |
| H | brosyl | methoxy | OH | ethyl |
| H | mesyl | methoxy | OH | ethyl |
| H | sulfonate | methoxy | OH | ethyl |
| H | sulfonamide | methoxy | OH | ethyl |
| H | phosphonate | methoxy | OH | ethyl |

TABLE 47
Pericosine Analogues - Structural Formulas V, VI, and VII

| X | Y | W | V | U |
|---|---|---|---|---|
| methyl | OH | ethoxy | OH | methyl |
| methyl | Br | ethoxy | OH | methyl |
| methyl | Cl | ethoxy | OH | methyl |
| methyl | F | ethoxy | OH | methyl |
| methyl | acetyloxy | ethoxy | OH | methyl |
| methyl | PH₃P | ethoxy | OH | methyl |
| methyl | N,N,N-methyldiethyl-2- | ethoxy | OH | methyl |
| | (11-oxidaneyl)ethan-1- | | | |
| | aminium | | | |
| methyl | I | ethoxy | OH | methyl |
| methyl | tosyl | ethoxy | OH | methyl |
| methyl | brosyl | ethoxy | OH | methyl |
| methyl | mesyl | ethoxy | OH | methyl |
| methyl | sulfonate | ethoxy | OH | methyl |
| methyl | sulfonamide | ethoxy | OH | methyl |
| methyl | choline | ethoxy | OH | methyl |
| methyl | phosphonate | ethoxy | OH | methyl |
| ethyl | OH | ethoxy | OH | methyl |
| ethyl | Br | ethoxy | OH | methyl |
| ethyl | Cl | ethoxy | OH | methyl |
| ethyl | F | ethoxy | OH | methyl |
| ethyl | I | ethoxy | OH | methyl |
| ethyl | acetyloxy | ethoxy | OH | methyl |
| ethyl | PH₃P | ethoxy | OH | methyl |
| ethyl | choline | ethoxy | OH | methyl |
| ethyl | N,N,N-methyldiethyl-2- | ethoxy | OH | methyl |
| | (11-oxidaneyl)ethan-1- | | | |
| | aminium | | | |
| ethyl | tosyl | ethoxy | OH | methyl |
| ethyl | brosyl | ethoxy | OH | methyl |
| ethyl | mesyl | ethoxy | OH | methyl |
| ethyl | sulfonate | ethoxy | OH | methyl |
| ethyl | sulfonamide | ethoxy | OH | methyl |
| ethyl | phosphonate | ethoxy | OH | methyl |
| n-propyl | OH | ethoxy | OH | methyl |
| n-propyl | Br | ethoxy | OH | methyl |
| n-propyl | Cl | ethoxy | OH | methyl |
| n-propyl | F | ethoxy | OH | methyl |
| n-propyl | I | ethoxy | OH | methyl |
| n-propyl | acetyloxy | ethoxy | OH | methyl |
| n-propyl | PH₃P | ethoxy | OH | methyl |
| n-propyl | choline | ethoxy | OH | methyl |
| n-propyl | N,N,N-methyldiethyl-2- | ethoxy | OH | methyl |
| | (11-oxidaneyl)ethan-1- | | | |
| | aminium | | | |
| n-propyl | tosyl | ethoxy | OH | methyl |
| n-propyl | brosyl | ethoxy | OH | methyl |
| n-propyl | mesyl | ethoxy | OH | methyl |
| n-propyl | sulfonate | ethoxy | OH | methyl |
| n-propyl | sulfonamide | ethoxy | OH | methyl |
| n-propyl | phosphonate | ethoxy | OH | methyl |
| choline | OH | ethoxy | OH | methyl |
| choline | Br | ethoxy | OH | methyl |
| choline | Cl | ethoxy | OH | methyl |
| choline | F | ethoxy | OH | methyl |
| choline | I | ethoxy | OH | methyl |
| choline | acetyloxy | ethoxy | OH | methyl |
| choline | PH₃P | ethoxy | OH | methyl |
| choline | choline | ethoxy | OH | methyl |
| choline | N,N,N-methyldiethyl-2- | ethoxy | OH | methyl |
| | (11-oxidaneyl)ethan-1- | | | |
| | aminium | | | |
| choline | tosyl | ethoxy | OH | methyl |
| choline | brosyl | ethoxy | OH | methyl |
| choline | mesyl | ethoxy | OH | methyl |
| choline | sulfonate | ethoxy | OH | methyl |
| choline | sulfonamide | ethoxy | OH | methyl |
| choline | phosphonate | ethoxy | OH | methyl |
| H | OH | ethoxy | OH | methyl |
| H | Br | ethoxy | OH | methyl |
| H | Cl | ethoxy | OH | methyl |
| H | F | ethoxy | OH | methyl |
| H | I | ethoxy | OH | methyl |
| H | acetyloxy | ethoxy | OH | methyl |
| H | PH₃P | ethoxy | OH | methyl |
| H | choline | ethoxy | OH | methyl |
| H | N,N,N-methyldiethyl-2- | ethoxy | OH | methyl |
| | (11-oxidaneyl)ethan-1- | | | |
| | aminium | | | |
| H | tosyl | ethoxy | OH | methyl |
| H | brosyl | ethoxy | OH | methyl |
| H | mesyl | ethoxy | OH | methyl |
| H | sulfonate | ethoxy | OH | methyl |
| H | sulfonamide | ethoxy | OH | methyl |
| H | phosphonate | ethoxy | OH | methyl |

TABLE 48
Pericosine Analogues - Structural Formulas V, VI, and VII

| X | Y | W | V | U |
|---|---|---|---|---|
| methyl | OH | methyl | OH | ethoxy |
| methyl | Br | methyl | OH | ethoxy |
| methyl | Cl | methyl | OH | ethoxy |
| methyl | F | methyl | OH | ethoxy |
| methyl | acetyloxy | methyl | OH | ethoxy |
| methyl | PH₃P | methyl | OH | ethoxy |
| methyl | N,N,N-methyldiethyl-2- | methyl | OH | ethoxy |
| | (11-oxidaneyl)ethan-1- | | | |
| | aminium | | | |
| methyl | I | methyl | OH | ethoxy |
| methyl | tosyl | methyl | OH | ethoxy |
| methyl | brosyl | methyl | OH | ethoxy |
| methyl | mesyl | methyl | OH | ethoxy |
| methyl | sulfonate | methyl | OH | ethoxy |
| methyl | sulfonamide | methyl | OH | ethoxy |
| methyl | choline | methyl | OH | ethoxy |
| methyl | phosphonate | methyl | OH | ethoxy |
| ethyl | OH | methyl | OH | ethoxy |
| ethyl | Br | methyl | OH | ethoxy |
| ethyl | Cl | methyl | OH | ethoxy |
| ethyl | F | methyl | OH | ethoxy |
| ethyl | I | methyl | OH | ethoxy |
| ethyl | acetyloxy | methyl | OH | ethoxy |
| ethyl | PH₃P | methyl | OH | ethoxy |
| ethyl | choline | methyl | OH | ethoxy |
| ethyl | N,N,N-methyldiethyl-2- | methyl | OH | ethoxy |
| | (11-oxidaneyl)ethan-1- | | | |
| | aminium | | | |
| ethyl | tosyl | methyl | OH | ethoxy |
| ethyl | brosyl | methyl | OH | ethoxy |
| ethyl | mesyl | methyl | OH | ethoxy |
| ethyl | sulfonate | methyl | OH | ethoxy |
| ethyl | sulfonamide | methyl | OH | ethoxy |
| ethyl | phosphonate | methyl | OH | ethoxy |
| n-propyl | OH | methyl | OH | ethoxy |
| n-propyl | Br | methyl | OH | ethoxy |
| n-propyl | Cl | methyl | OH | ethoxy |
| n-propyl | F | methyl | OH | ethoxy |
| n-propyl | I | methyl | OH | ethoxy |
| n-propyl | acetyloxy | methyl | OH | ethoxy |
| n-propyl | PH₃P | methyl | OH | ethoxy |
| n-propyl | choline | methyl | OH | ethoxy |
| n-propyl | N,N,N-methyldiethyl-2- | methyl | OH | ethoxy |
| | (11-oxidaneyl)ethan-1- | | | |
| | aminium | | | |
| n-propyl | tosyl | methyl | OH | ethoxy |
| n-propyl | brosyl | methyl | OH | ethoxy |
| n-propyl | mesyl | methyl | OH | ethoxy |
| n-propyl | sulfonate | methyl | OH | ethoxy |
| n-propyl | sulfonamide | methyl | OH | ethoxy |
| n-propyl | phosphonate | methyl | OH | ethoxy |
| choline | OH | methyl | OH | ethoxy |
| choline | Br | methyl | OH | ethoxy |
| choline | Cl | methyl | OH | ethoxy |
| choline | F | methyl | OH | ethoxy |
| choline | I | methyl | OH | ethoxy |
| choline | acetyloxy | methyl | OH | ethoxy |
| choline | PH₃P | methyl | OH | ethoxy |
| choline | choline | methyl | OH | ethoxy |
| choline | N,N,N-methyldiethyl-2- | methyl | OH | ethoxy |
| | (11-oxidaneyl)ethan-1- | | | |
| | aminium | | | |
| choline | tosyl | methyl | OH | ethoxy |
| choline | brosyl | methyl | OH | ethoxy |
| choline | mesyl | methyl | OH | ethoxy |
| choline | sulfonate | methyl | OH | ethoxy |
| choline | sulfonamide | methyl | OH | ethoxy |
| choline | phosphonate | methyl | OH | ethoxy |
| H | OH | methyl | OH | ethoxy |
| H | Br | methyl | OH | ethoxy |
| H | Cl | methyl | OH | ethoxy |
| H | F | methyl | OH | ethoxy |
| H | I | methyl | OH | ethoxy |
| H | acetyloxy | methyl | OH | ethoxy |
| H | PH₃P | methyl | OH | ethoxy |
| H | choline | methyl | OH | ethoxy |
| H | N,N,N-methyldiethyl-2- | methyl | OH | ethoxy |
| | (11-oxidaneyl)ethan-1- | | | |
| | aminium | | | |
| H | tosyl | methyl | OH | ethoxy |
| H | brosyl | methyl | OH | ethoxy |
| H | mesyl | methyl | OH | ethoxy |
| H | sulfonate | methyl | OH | ethoxy |
| H | sulfonamide | methyl | OH | ethoxy |
| H | phosphonate | methyl | OH | ethoxy |

TABLE 49
Pericosine Analogues - Structural Formulas V, VI, and VII

| X | Y | W | V | U |
|---|---|---|---|---|
| methyl | OH | ethyl | OH | methoxy |
| methyl | Br | ethyl | OH | methoxy |
| methyl | Cl | ethyl | OH | methoxy |
| methyl | F | ethyl | OH | methoxy |
| methyl | acetyloxy | ethyl | OH | methoxy |
| methyl | PH₃P | ethyl | OH | methoxy |
| methyl | N,N,N-methyldiethyl-2- | ethyl | OH | methoxy |
| | (11-oxidaneyl)ethan-1- | | | |
| | aminium | | | |
| methyl | I | ethyl | OH | methoxy |
| methyl | tosyl | ethyl | OH | methoxy |
| methyl | brosyl | ethyl | OH | methoxy |
| methyl | mesyl | ethyl | OH | methoxy |
| methyl | sulfonate | ethyl | OH | methoxy |
| methyl | sulfonamide | ethyl | OH | methoxy |
| methyl | choline | ethyl | OH | methoxy |
| methyl | phosphonate | ethyl | OH | methoxy |
| ethyl | OH | ethyl | OH | methoxy |
| ethyl | Br | ethyl | OH | methoxy |
| ethyl | Cl | ethyl | OH | methoxy |
| ethyl | F | ethyl | OH | methoxy |
| ethyl | I | ethyl | OH | methoxy |
| ethyl | acetyloxy | ethyl | OH | methoxy |
| ethyl | PH₃P | ethyl | OH | methoxy |
| ethyl | choline | ethyl | OH | methoxy |
| ethyl | N,N,N-methyldiethyl-2- | ethyl | OH | methoxy |
| | (11-oxidaneyl)ethan-1- | | | |
| | aminium | | | |
| ethyl | tosyl | ethyl | OH | methoxy |
| ethyl | brosyl | ethyl | OH | methoxy |
| ethyl | mesyl | ethyl | OH | methoxy |
| ethyl | sulfonate | ethyl | OH | methoxy |
| ethyl | sulfonamide | ethyl | OH | methoxy |
| ethyl | phosphonate | ethyl | OH | methoxy |
| n-propyl | OH | ethyl | OH | methoxy |
| n-propyl | Br | ethyl | OH | methoxy |
| n-propyl | Cl | ethyl | OH | methoxy |
| n-propyl | F | ethyl | OH | methoxy |
| n-propyl | I | ethyl | OH | methoxy |
| n-propyl | acetyloxy | ethyl | OH | methoxy |
| n-propyl | PH₃P | ethyl | OH | methoxy |
| n-propyl | choline | ethyl | OH | methoxy |
| n-propyl | N,N,N-methyldiethyl-2- | ethyl | OH | methoxy |
| | (11-oxidaneyl)ethan-1- | | | |
| | aminium | | | |
| n-propyl | tosyl | ethyl | OH | methoxy |
| n-propyl | brosyl | ethyl | OH | methoxy |
| n-propyl | mesyl | ethyl | OH | methoxy |
| n-propyl | sulfonate | ethyl | OH | methoxy |
| n-propyl | sulfonamide | ethyl | OH | methoxy |
| n-propyl | phosphonate | ethyl | OH | methoxy |
| choline | OH | ethyl | OH | methoxy |
| choline | Br | ethyl | OH | methoxy |
| choline | Cl | ethyl | OH | methoxy |
| choline | F | ethyl | OH | methoxy |
| choline | I | ethyl | OH | methoxy |
| choline | acetyloxy | ethyl | OH | methoxy |
| choline | PH₃P | ethyl | OH | methoxy |
| choline | choline | ethyl | OH | methoxy |
| choline | N,N,N-methyldiethyl-2- | ethyl | OH | methoxy |
| | (11-oxidaneyl)ethan-1- | | | |
| | aminium | | | |
| choline | tosyl | ethyl | OH | methoxy |
| choline | brosyl | ethyl | OH | methoxy |
| choline | mesyl | ethyl | OH | methoxy |
| choline | sulfonate | ethyl | OH | methoxy |
| choline | sulfonamide | ethyl | OH | methoxy |
| choline | phosphonate | ethyl | OH | methoxy |
| H | OH | ethyl | OH | methoxy |
| H | Br | ethyl | OH | methoxy |
| H | Cl | ethyl | OH | methoxy |
| H | F | ethyl | OH | methoxy |
| H | I | ethyl | OH | methoxy |
| H | acetyloxy | ethyl | OH | methoxy |
| H | PH₃P | ethyl | OH | methoxy |
| H | choline | ethyl | OH | methoxy |
| H | N,N,N-methyldiethyl-2- | ethyl | OH | methoxy |
| | (11-oxidaneyl)ethan-1- | | | |
| | aminium | | | |
| H | tosyl | ethyl | OH | methoxy |
| H | brosyl | ethyl | OH | methoxy |
| H | mesyl | ethyl | OH | methoxy |
| H | sulfonate | ethyl | OH | methoxy |
| H | sulfonamide | ethyl | OH | methoxy |
| H | phosphonate | ethyl | OH | methoxy |

In at least certain embodiments, the present disclosure is directed to the compositions, kits, devices, and methods described in of the following non-limiting clauses.

Clause 1. A composition comprising at least one shikimate analogue, and one or more secondary compounds, the at least one shikimate analogue comprising Structural Formula I or Structural Formula II:

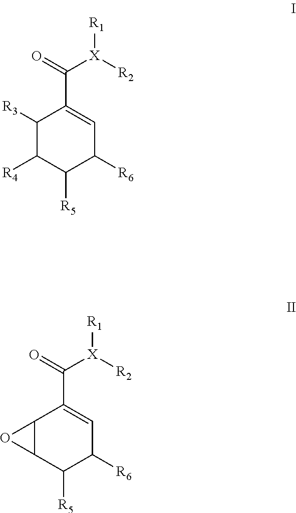

wherein,

X is O, N, S, or is absent;

R₁ is selected from the group consisting of H, (C1-C8)alkyl, (C1-C8)alkenyl, (C1-C8)alkynyl, cyano, halo, nitro, thio, substituted phenyl, phenylmethyl, and substituted naphthalenyl;

R₂ is selected from the group consisting of H, (C1-C8)alkyl, (C1-C8)alkenyl, (C1-C8)alkynyl, cyano, halo, nitro, thio, substituted phenyl, phenylmethyl, and substituted naphthalenyl, or is absent;

R₃ is selected from the group consisting of fluoro, chloro, bromo, iodo, hydroxyl, substituted phosphate, —O-tosyl, —O-mesyl, (C1-C8)alkoxy, (C2-C8)acyloxy, substituted phenoxy, substituted naphthalenyloxy, substituted naphthalenylmethoxy, (C1-C12)primary amino, (C1-C12)secondary amino, (C1-C12)tertiary amino, and (C1-C12)cyclic amino, (C1-C8)ammonio, (C1-C8)carboxamino, (C1-C8)imino, azido, (C1-C8)azo, cyanato, isocyanato, nitrooxy, cyano, isocyano, nitrosooxy, nitro, nitroso, (C1-C8)substituted carbamoyl, hydroxyamino, morpholino, anilino, indol, pyrrol, imidazole, benzimidazol, pyrazol, guanidino, piperazino, polyamino, and N-methylated polyamino;

R₄ is selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl) ethenyl]oxy, and (1-carboxyethenyl)oxy;

R₅ selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl) ethenyl]oxy, and (1-carboxyethenyl)oxy; and

R₆ is selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl) ethenyl]oxy, and (1-carboxyethenyl)oxy.

Clause 2. The composition of clause 1, wherein at least one of the one or more secondary compounds is not water.

Clause 3. The composition of either of clauses 1 or 2, wherein the at least one shikimate analogue has Structural Formula I, wherein, X═O, R₁ is (C1-C8)alkyl, R₂ is absent, R₃ is selected from the group consisting of fluoro, chloro, bromo, iodo, and (C1-C8)alkoxy, R₄═OH, R₅═OH, and R₆═OH.

Clause 4. The composition of any one of clauses 1-3, wherein the at least one shikimate analogue has Structural Formula I, wherein X═O, R₁═CH₃, R₂ is absent, R₃═Cl, R₄═OH, R₅═OH, and R₆═OH (Pericosine A).

Clause 5. The composition of any one of clauses 1-3, wherein the at least one shikimate analogue has Structural Formula I, wherein X═O, R₁═CH₃, R₂ is absent, R₃═OCH₃, R₄═OH, R₅═OH, and R₆═OH (Pericosine C).

Clause 6. The composition of any one of clauses 1-3, wherein the at least one shikimate analogue is selected from the group consisting of Pericosine A, Pericosine B, Pericosine C, and Pericosine D.

Clause 7. The composition of any one of clauses 1-6, wherein at least one of the one or more secondary compounds is not methanol, ethanol, or a propanol.

Clause 8. The composition of any one of clauses 1-7, wherein the one or more secondary compounds is selected from the group consisting of organic bases, inorganic bases, and organic/inorganic bases.

Clause 9. The composition of any one of clauses 1-8, wherein the one or more secondary compounds is an amine or a salt thereof.

Clause 10. The composition of clause 9, wherein the amine is an alkyl amine selected from the group consisting of methyl amine, ethyl amine, dimethyl amine, diethyl amine, trimethyl amine, triethyl amine, diethanolamine, triethanolamine, and trimethylammonia.

Clause 11. The composition of clause 9, wherein the amine is an acyclic or cyclic polyamine selected from the group consisting of spermine, spermidine, tris(2-aminoethyl)amine, cyclen, cyclam, 1,4,7-triazacyclononane, 1,1,1-tris(aminomethyl)ethane, ethylenediamine, 1,4-diazabicyclo[2.2.2]octane (DABCO), diethylenetriamine, triethylenetetramine, 1,3-diaminopropane, putrescine, cadaverine, sym-norspermidine, sym-homospermidine, norspermine, thermospermine, carboxyspermidine, norcarboxyspermidine, caldopentamine, caldohexamine, ethylenediamine, 1,2-diaminopropane, 1,3-diaminopropane, N-methylethylenediamine, 1,4-diaminobutane, 3-(methylamino)propylamine, N,N′-dimethylethylenediamine, N-methyl-1,3-diaminopropane, 1-dimethylamino-2-propylamine, 3-(dimethylamino)-1-propylamine, N,N,N′,N′-tetramethyldiaminomethane, N,N,N′-trimethylethylenediamine, N-isopropylethylenediamine, N-propylethylenediamine, 2-(aminomethyl)-2-methyl-1,3-propanediamine, 1,2-diamino-5-bromo-3-chlorobenzene, 3,5-dichloro-1,2-diaminobenzene, 4-bromo-1,2-diaminobenzene, 4,5-dichloro-o-phenylenediamine, 4-chloro-1,3-diaminobenzene, 2-nitro-1,4-phenylenediamine, 3-nitro-1,2-phenylenediamine, 4-nitro-o-phenylenediamine, m-phenylenediamine, o-phenylenediamine, p-phenylenediamine, trans-4-cyclohexene-1,2-diamine, cis-4-cyclohexene-1,2-diamine, hexamethylenetetramine, 4-aminobenzylamine, N,N′-bis(2-aminoethyl)-1,3-propanediamine, methyl 3,4-diaminobenzoate, 1,2-diamino-3,5-dimethylbenzene, 4,5-dimethyl-1,2-phenylenediamine, 4-(2-aminoethyl)aniline, aniline, m-xylylenediamine, N-phenylethylenediamine, o-xylylenediamine, p-xylylenediamine, 1,8-diaminooctane, N,N-dimethyldipropylenetriamine, 1,2-bis(3-aminopropylamino)ethane, N-tosylethylenediamine, 2,2,4(2,4,4)-trimethyl-1,6-hexanediamine, 1,4-diaminonaphthalene, 1,5-diaminonaphthalene, 1,8-diaminonaphthalene, 4-tert-butyl-2,6-diaminoanisole, 2,2′-oxydianiline, 4,4′-oxydianiline, 3,3′-diaminobenzidine, 3,4′-diaminodiphenylmethane, 4,4′-diaminodiphenylmethane, 4,4′-ethylenedianiline, 2,4,6-triethyl-1,3,5-benzenetrimethanamine, and 1,8-anthracenedimethanamine.

Clause 12. The composition of clause 9, wherein the amine is selected from the group consisting monoamines, polyamines, or polymeric amines coated on or crosslinked with a polymer particle.

Clause 13. The composition of clause 9, wherein the amine is selected from the group consisting monoamines, polyamines, or polymeric amines coated on or crosslinked with a polymeric film.

Clause 14. The composition of clause 9, wherein the amine is selected from the group consisting monoamines, polyamines, or polymeric amines functionalized on or infused into a hydrogel.

Clause 15. The composition of clause 9, wherein the amine is selected from the group consisting cyclic polyamine homopolymers and heteropolymers comprising at least one of polyvinylamine, polyethyleneimine, polyvicinalamine, polyamidoamine, polyallyamines, and polyetheramines.

Clause 16. The composition of any one of clauses 1-8, wherein the one or more secondary compounds is a pyridine or pyridinium selected from the group consisting of pyridine, alkylated pyridines, 2,6-lutidine, 2,4-lutidine, pyridazine, pyrimidine, pyrazine, 4-bromopyridine, 2,2′-bipyridine, 2,6-di-tert-butylpyridine, pyridine-2,6-dicarboxylic acid, pyridinium cations, and salts of the above.

Clause 17. The composition of any one of clauses 1-8, wherein the one or more secondary compounds is an imidazole or alkylated imidazole.

Clause 18. The composition of any one of clauses 1-8, wherein the one or more secondary compounds is a pyrrole or alkylated pyrrole.

Clause 19. The composition of any one of clauses 1-8, wherein the one or more secondary compounds is selected from the group consisting of sodium metasilicate, chlorhexidine, borates, zinc pyrithione, trimethyl barium hydroxide, 9-Azajulolidine, sodium iodide, potassium iodide, ammonia; ammonium hydroxide; tetramethylammonium hydroxide; tetrabutylammonium hydroxide; tetrabutylammonium hydroxide; sodium hydroxide; soda lime; potassium hydroxide; magnesium hydroxide; magnesium carbonate; calcium carbonate; aluminum hydroxide; sodium bicarbonate, sodium carbonate, potassium carbonate, potassium bicarbonate, or other carbonates or bicarbonates, choline; histamine lysine; tris; and linear or branched dendrimers; calcium hydroxide, Lugol's Iodine, iodine tincture, povidone-iodine, benzalkonium chloride, cetrimonium bromide, Brilliant Green, triarylmethane dyes, Malachite green, octenidine dihydrochloride, phenoxyethanol, USP Tincture of Iodine, USP Strong Iodine Tincture, 1,3-dibromo-5,5-dimethylhydantoin (DBDMH), morpholine, and 3-(N-morpholino)propane sulfonic acid (MOPS).

Clause 20. The composition of any one of clauses 1-7, wherein the one or more secondary compounds is selected from the group consisting of alcohols, alditols, glycols, polyols, saccharides, and polysaccharides.

Clause 21. The composition of clause 20, wherein the alcohols, alditols, glycols, polyols, saccharides, and polysaccharides are selected from the group consisting of cyclitol, acarviocin, aminocyclitol, bornesitol, ciceritol, conduritol, decahydroxycyclopentane, 5-deoxyinositol, dodecahydroxycyclohexane, ononitol, pinitol, pinpollitol, quebrachitol, theogallin, 3,4,5-tri-O-galloylquinic acid, inositol, inositol pentakisphosphate, cis-inositol, D-chiro-inositol, L-chiro-inositol, epi-inositol, neo-inositol, muco-inositol, neo-inositol, scyllo-inositol, sorbitol, threitol, arabitol, galactitol, iditol, volemitol, sorbitol, fucitol, xylitol, lactitol, erythritol, lactitol, maltitol, phytic acid, quinic acid, propylene glycol, 1,2-propanediol, ethylene glycol, low molecular weight polyethylene glycols (e.g., C2-C10), vegetable glycerine, dipropylene glycol, erythulose, glycerol, panthenol, arabinose, bis-HPPP, cellobiose, mannitol, mannose, glucose, allose, altrose, gulose, idose, lactose, maltose, dextrose, galactose, talose, psicose, fructose, sorbose, tagatose, β-d-ribopyranose, a-d-ribopyranose, β-d-ribofuranose, a-d-ribofuranose, sucrose, xylose, trehalose, cytosine glycol, cyclohexane-1,2-diol, aminomethanol, ethyleneglycol, 1,3-propanediol, 1,4-butanediol, 2,2-dimethyl-1-butanol, ethanol, propanol, butanol, pentanol, hexanol, ethynol, acetylenediol, fenticlor, fucitol, gluconic acid, glucic acid, 2-heptanol, 3-heptanol, 2-hexanol, 3-hexanol, ribitol, ethylhexylglycerin, octoxyglycerin, glucuronic acid, glyceraldehyde, glyceric acid, glycerol 3-phosphate, glycerol monostearate, 2-octanediol, pinacol, racemic acid, tartaric acid, uronic acid, xylosan, 1,2-butanediol, 1,3-butanediol, 1,4-butanediol, 2,3-butanediol, 2-methyl-2,4-pentanediol, neopentyl glycol, maltodextrins, raffinose, stachyose, fructo-oligosaccharides, amylose, amylopectin, starch, glycogen, cellulose, hemicellulose, methyl cellulose, methyl ethyl cellulose, pectins, hydrocolloids, sucralose, isomalt, isomaltooligosaccharide, isomaltulose, maltodextrin, and polydextrose.

Clause 22. The composition of any one of clauses 1-21, further comprising a carrier or vehicle selected from the group consisting of sticks, soaps, bars of soap, balms, creams, pastes, gums, lotions, gels, foams, ointments, emulsions, suspensions, aqueous solutions, eye drops, aerosols, sprays, inhalants, body washes, face washes, rinses, oral tinctures, gel beads, pellets, cat litter, pet wipes, sheets, sachets, towelettes, breath strips, paper towels, napkins, sponges, sanitary napkins, tampons, baby diapers, adult diapers, undershirts and other items of clothing.

Clause 23. The composition of any one of clauses 1-22, further comprising at least one of a fragrance, a preservative, and a surfactant.

Clause 24. The composition of clause 23, wherein the fragrance is selected from the group consisting of eucalyptol, limonene, and isopentyl acetate, the preservative is selected from the group consisting of camphor, methylisothiazolinone, 2-phenoxyethanol, diazolidinyl urea, poluquatenium-2, and quaternium-15, and the surfactant is selected from the group consisting of sodium palmitate, sodium stearate, palmitic acid, and stearic acid.

Clause 25. The composition of any one of clauses 1-24, comprising about 0.01 to about 1000 milligrams of said at least one shikimate analogue per ml of said one or more secondary compound.

Clause 26. The composition of any one of clauses 1-25, comprising about 1 wt % to about 90 wt % of said at least one shikimate analogue and about 10 wt % to about 99 wt % of said one or more secondary compounds.

Clause 27. A kit, comprising:

a first container containing at least one shikimate analogue comprising Structural Formula I or Structural Formula II:

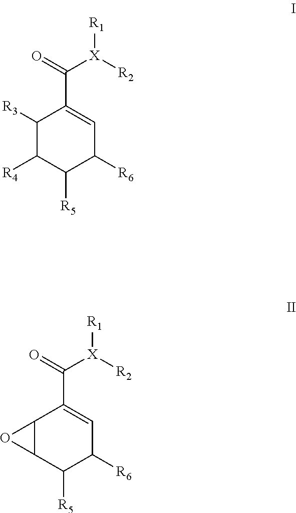

wherein,

X is O, N, S, or is absent;

R₁ is selected from the group consisting of H, (C1-C8)alkyl, (C1-C8)alkenyl, (C1-C8)alkynyl, cyano, halo, nitro, thio, substituted phenyl, phenylmethyl, substituted naphthalenyl;

R₂ is selected from the group consisting of H, (C1-C8)alkyl, (C1-C8)alkenyl, (C1-C8)alkynyl, cyano, halo, nitro, thio, substituted phenyl, phenylmethyl, substituted naphthalenyl, or is absent;

R₃ is selected from the group consisting of fluoro, chloro, bromo, iodo, hydroxyl, substituted phosphate, —O-tosyl, —O-mesyl, (C1-C8)alkoxy, (C2-C8)acyloxy, substituted phenoxy, substituted naphthalenyloxy, substituted naphthalenylmethoxy, (C1-C12)primary amino, (C1-C12) secondary amino, (C1-C12)tertiary amino, and (C1-C12)cyclic amino, (C1-C8)ammonio, (C1-C8)carboxamino, (C1-C8)imino, azido, (C1-C8)azo, cyanato, isocyanato, nitrooxy, cyano, isocyano, nitrosooxy, nitro, nitroso, (C1-C8)substituted carbamoyl, hydroxyamino, morpholino, anilino, indol, pyrrol, imidazole, benzimidazol, pyrazol, guanidino, piperazino, polyamino, and N-methylated polyamino;

R₄ is selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl) ethenyl]oxy, (1-carboxyethenyl)oxy;

R₅ selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl) ethenyl]oxy, (1-carboxyethenyl)oxy; and

R₆ is selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl) ethenyl]oxy, (1-carboxyethenyl)oxy; and at least one second container containing one or more secondary compounds, wherein the at least one shikimate analogue in the first container and the at least one or more secondary compound in the second container can be combined to form a mixture, and optionally wherein the kit is a point-of-use kit, wherein the first container with the at least one shikimate analogue and the at least one second container with the at least one secondary compound are configured so the at least one shikimate analogue and the at least one secondary compound can be combined at a point-of-use to form a mixture for application to a treatment surface.

Clause 28. The kit of clause 27, wherein first container contains a plurality of shikimate analogues comprising Structural Formula I and/or Structural Formula II.

Clause 29. The kit of clause 27 or 28, comprising the shikimate analogue of any one of clauses 3-7.

Clause 30. The kit of any one of clauses 27-29, wherein at least one of the one or more secondary compounds is not water.

Clause 31. The kit of any one of clauses 27-30, wherein at least one of the one or more secondary compounds is not methanol, ethanol, or a propanol.

Clause 32. The kit of any one of clauses 27-31, comprising the one or more secondary compounds of any one of clauses 8-21.

Clause 33. The kit of any one of clauses 27-32, comprising the carrier or vehicle of clause 22.

Clause 34. The kit of any one of clauses 27-33, comprising at least one of a fragrance, a preservative, and a surfactant.

Clause 35. The kit of clause 34, wherein the fragrance is selected from the group consisting of eucalyptol, limonene, and isopentyl acetate, the preservative is selected from the group consisting of camphor, methylisothiazolinone, 2-phenoxyethanol, diazolidinyl urea, poluquatenium-2, and quaternium-15, and the surfactant is selected from the group consisting of sodium palmitate, sodium stearate, palmitic acid, and stearic acid.

Clause 36. The kit of any one of clauses 27-35, wherein the mixture comprises about 0.01 to about 1000 milligrams of said at least one shikimate analogue per ml of said one or more secondary compound.

Clause 37. The kit of any one of clauses 27-36, wherein the mixture comprises about 1 wt % to about 90 wt % of said at least one shikimate analogue and about 10 wt % to about 99 wt % of the one or more secondary compounds.

Clause 38. The kit of any one of clauses 27-37, comprising a set of instructions for using the kit to treat an irritant or malodorous compound.

Clause 39. A method of treating an irritant or malodorous compound, comprising applying to the irritant or malodorous compound at least one shikimate analogue and one or more secondary compounds, the at least one shikimate analogue comprising Structural Formula I or Structural Formula II:

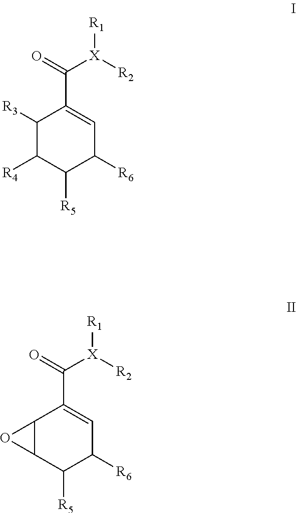

wherein,

X is O, N, S, or is absent;

R₁ is selected from the group consisting of H, (C1-C8)alkyl, (C1-C8)alkenyl, (C1-C8)alkynyl, cyano, halo, nitro, thio, substituted phenyl, phenylmethyl, and substituted naphthalenyl;

R₂ is selected from the group consisting of H, (C1-C8)alkyl, (C1-C8)alkenyl, (C1-C8)alkynyl, cyano, halo, nitro, thio, substituted phenyl, phenylmethyl, and substituted naphthalenyl, or is absent;

R₃ is selected from the group consisting of fluoro, chloro, bromo, iodo, hydroxyl, substituted phosphate, —O-tosyl, —O-mesyl, (C1-C8)alkoxy, (C2-C8)acyloxy, substituted phenoxy, substituted naphthalenyloxy, substituted naphthalenylmethoxy, (C1-C12)primary amino, (C1-C12)secondary amino, (C1-C12)tertiary amino, and (C1-C12)cyclic amino, (C1-C8)ammonio, (C1-C8)carboxamino, (C1-C8)imino, azido, (C1-C8)azo, cyanato, isocyanato, nitrooxy, cyano, isocyano, nitrosooxy, nitro, nitroso, (C1-C8)substituted carbamoyl, hydroxyamino, morpholino, anilino, indol, pyrrol, imidazole, benzimidazol, pyrazol, guanidino, piperazino, polyamino, and N-methylated polyamino;

R₄ is selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl) ethenyl]oxy, and (1-carboxyethenyl)oxy;

R₅ selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl) ethenyl]oxy, and (1-carboxyethenyl)oxy; and

R₆ is selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl) ethenyl]oxy, and (1-carboxyethenyl)oxy.

Clause 40. The method of clause 39, further comprising treating the irritant or malodorous compound by applying the at least one shikimate analogue and one or more secondary compounds to a surface or an atmosphere comprising the irritant or malodorous compound.

Clause 41. The method of clause 40, wherein the at least one shikimate analogue and the one or more secondary compounds are applied to the surface or atmosphere as a mixture.

Clause 42. The method of any one of clauses 40-41, wherein the at least one shikimate analogue and the one or more secondary compounds are maintained in separate compartments until formed into the mixture at a point of application to the surface or atmosphere.

Clause 43. The method of any one of clauses 40-42, wherein the surface is an animate surface selected from the group consisting of an epithelial surface of an animal, and a surface of a plant.

Clause 44. The method of clause 43, wherein the epithelial surface is selected from the group consisting of epithelia, skin, hair, fur, nails, internal or external body surfaces, surfaces of body cavities, and surfaces of hollow organs that are connected to underlying tissues in a human or animal body and constitute the corpus or partial corpus of said human or animal.

Clause 45. The method of either clause 43 or 44, wherein the epithelial surface has contact dermatitis.

Clause 46. The method of clause 45, wherein the contact dermatitis is induced by exposure to a urushiol.

Clause 47. The method of clause 45, wherein the contact dermatitis is induced by exposure to stinging nettle.

Clause 48. The method of any one of clauses 40-42, wherein the surface is an inanimate surface of an object selected from synthetic materials, structures, articles of manufacture, textiles, fabrics, couches, chairs, beds, floors, rugs, seats, solid materials, porous materials, soils, minerals, stones, rocks, concrete, walls, wood object, resins, metals, polymers, plastics, and composite materials.

Clause 49. The method of any one of clauses 40-42, wherein the atmosphere is selected from the group consisting of air, gaseous components of a room, building, vehicle, outdoor environment, sealed container, box, bag, or other open or sealed or semipermeable package, container, or vessel.

Clause 50. The method of any one of clauses 40-42, wherein the atmosphere comprises gaseous compounds at or near their natural levels or at levels modified by the actions of humans, animals, plants, or microorganisms.

Clause 51. The method of any one of clauses 39-50, wherein the irritant or malodorous compound that is treated is selected from the group consisting of (a) organosulfur and thiol-containing compounds, (b) organic, carboxylic acids and carboxylic-acid-containing compounds; (c) amines, amine-containing compounds, and nitrogen-containing compounds; and (d) inorganic sulfur-containing and nitrogen-containing compounds.

Clause 52. The method of clause 51, wherein the organosulfur or thiol-containing compound is selected from the group consisting of methanethiol, ethanethiol, propane-1-thiol, propane-2-thiol, thioglycolic acid, 2-naphthalenethiol, 3-methylbutane-1-thiol, 2-methylbutane-1-thiol, but-3-ene-1-thiol, (E)-but-2-ene-1-thiol, (z)-but-2-ene-1-thiol, butanethiol, benzenethiol, phenylmethanethiol, quinoline-2-thiol, quinoline-3-thiol, quinoline-4-thiol, quinolin-2-ylmethanethiol, quinolin-3-ylmethanethiol, quinolin-4-ylmethanethiol, furfuryl mercaptan, 3-methylbut-2-ene-1-thiol, sulfinic acids, sulfenic acids, sulfonic acids, sulfonate esters, sulfoxides, sulfones, selenols, diselenides, disulfides, hydrogen sulfide, organic sulfides, dimethyl trisulfide, and combinations thereof, and precursors thereof.

Clause 53. The method of clause 51, wherein the organic, carboxylic acid or carboxylic-acid-containing compound is selected from the group consisting of geosmin, propionic acid, butyric acid, valeric acid, and caproic acid, and combinations thereof.

Clause 53. The method of clause 51, wherein the amine, amine-containing compound, and nitrogen-containing compounds is selected from the group consisting of dimethyl amine, diethyl amine, dipropyl amine, ethanolamine, putrescine, cadaverine, skatole, urea, Ripple's oil, Neatsfoot oil, and combinations thereof.

Clause 54. The method of clause 51, wherein the inorganic sulfur-containing or nitrogen-containing compound is selected from the group consisting of hydrogen sulfide, ammonium sulfide, and ammonia and combinations thereof.

Clause 55. The method of any one of clauses 39-54, comprising the shikimate analogue of any one of clauses 3-7.

Clause 56. The method of any one of clauses 39-55, wherein at least one of the one or more secondary compounds is not water.

Clause 57. The method of any one of clauses 39-56, wherein at least one of the one or more secondary compounds is not methanol, ethanol, or a propanol.

Clause 58. The method of any one of clauses 39-57, comprising the one or more secondary compounds of any one of clauses 8-21.

Clause 59. The method of any one of clauses 41-58, wherein the mixture comprises the carrier or vehicle of clause 22.

Clause 60. The method of any one of clauses 41-59, wherein the mixture comprises at least one of a fragrance, a preservative, and a surfactant.

Clause 61. The method of clause 60, wherein the fragrance is selected from the group consisting of eucalyptol, limonene, and isopentyl acetate, the preservative is selected from the group consisting of camphor, methylisothiazolinone, 2-phenoxyethanol, diazolidinyl urea, poluquatenium-2, and quaternium-15, and the surfactant is selected from the group consisting of sodium palmitate, sodium stearate, palmitic acid, and stearic acid.

Clause 62. The method of any one of clauses 41-61, wherein the mixture comprises about 0.01 to about 1000 milligrams of said at least one shikimate analogue per ml of said one or more secondary compound.

Clause 63. The method of any one of clauses 41-62, wherein the mixture comprises about 1 wt % to about 90 wt % of said at least one shikimate analogue and about 10 wt % to about 99 wt % of the one or more secondary compounds.

While the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the examples described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the present disclosure. Changes may be made in the formulation of the various compositions described herein, the methods described herein or in the steps or the sequence of steps of the methods described herein without departing from the spirit and scope of the present disclosure. Further, while various embodiments of the present disclosure have been described in claims herein below, it is not intended that the present disclosure be limited to these particular claims.

What is claimed is:

1. A method of deodorizing a malodorus compound on a treatment surface, the method comprising the steps of:
providing a point-of-use kit comprising a container containing at least one shikimate analogue comprising Structural Formula I or Structural Formula II: 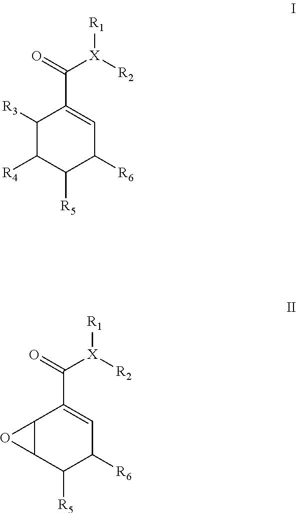
wherein,
X is O, N, S, or is absent;
R₁ is selected from the group consisting of H, (C1-C8)alkyl, (C1-C8)alkenyl, (C1-C8)alkynyl, cyano, halo, nitro, thio, substituted phenyl, phenylmethyl, substituted naphthalenyl;
R₂ is selected from the group consisting of H, (C1-C8)alkyl, (C1-C8)alkenyl, (C1-C8)alkynyl, cyano, halo, nitro, thio, substituted phenyl, phenylmethyl, substituted naphthalenyl, or is absent;
R₃ is selected from the group consisting of fluoro, chloro, bromo, iodo, hydroxyl, substituted phosphate, —O-tosyl, —O-mesyl, (C1-C8)alkoxy, (C2-C8)acyloxy, substituted phenoxy, substituted naphthalenyloxy, substituted naphthalenylmethoxy, (C1-C12)primary amino, (C1-C12) secondary amino, (C1-C12)tertiary amino, and (C1-C12)cyclic amino, (C1-C8)ammonio, (C1-C8)carboxamino, (C1-C8)imino, azido, (C1-C8)azo, cyanato, isocyanato, nitrooxy, cyano, isocyano, nitrosooxy, nitro, nitroso, (C1-C8)substituted carbamoyl, hydroxyamino, morpholino, anilino, indol, pyrrol, imidazole, benzimidazol, pyrazol, guanidino, piperazino, polyamino, and N-methylated polyamino;
R₄ is selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl)ethenyl]oxy, (1-carboxyethenyl)oxy;
R₅ selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl)ethenyl]oxy, (1-carboxyethenyl)oxy; and
R₆ is selected from the group consisting of H, (C1-C8)alkyl, (C1-C8)alkenyl, (C1-C8)alkynyl, cyano, halo, nitro, thio, substituted phenyl, hydroxyl, (C1-C8)alkoxy, (C2-C8)acyloxy, (C1-C8)carboxamino, substituted phenoxy, phenylmethoxy, [1-(methoxycarbonyl)ethenyl]oxy, (1-carboxyethenyl)oxy; and
at least one or more secondary compounds;
combining the at least one shikimate analogue and the at least one secondary compound at a point-of-use to form a treatment mixture; and
applying the treatment mixture to the treatment surface thereby deodorizing the malodorus compound on the treatment surface.

2. The method of claim 1, wherein the point-of-use kit further comprises an applicator for applying the treatment mixture to the treatment surface.

3. The method of claim 2, wherein the applicator comprises a spraying device.

4. The method of claim 1, wherein the at least one shikimate analogue has Structural Formula I, wherein X═O, R₁ is (C1-C8)alkyl, R₂ is absent, R₃ is selected from the group consisting of fluoro, chloro, bromo, iodo, and (C1-C8)alkoxy, R₄═OH, R₅═OH, and R₆═OH.

5. The method of claim 1, wherein the at least one shikimate analogue has Structural Formula I, is selected from the group consisting of Pericosine A, wherein X═O, R₁═CH₃, R₂ is absent, R₃═Cl, R₄═OH, R₅═OH, and R₆═OH; Pericosine B; Pericosine C, wherein X═O, R₁═CH₃, R₂ is absent, R₃═OCH₃, R₄═OH, R₅═OH, and R₆═OH; and Pericosine D.

6. The method of claim 1, wherein at least one of the one or more secondary compounds is not water, methanol, ethanol, or a propanol.

7. The method of claim 1, wherein the one or more secondary compounds is selected from the group consisting of organic bases, inorganic bases, and organic/inorganic bases.

8. The method of claim 1, wherein the one or more secondary compounds is an amine or a salt thereof.

9. The method of claim 8, wherein the amine is an alkyl amine.

10. The method of claim 9 wherein the alkyl amine is selected from the group consisting of methyl amine, ethyl amine, dimethyl amine, diethyl amine, trimethyl amine, triethyl amine, diethanolamine, triethanolamine, and trimethylammonia.

11. The method of claim 8, wherein the amine is an acyclic or cyclic polyamine.

12. The method of claim 11, wherein the acyclic or cyclic polyamine is selected from the group consisting of spermine, spermidine, tris(2-aminoethyl)amine, cyclen, cyclam, 1,4,7-triazacyclononane, 1,1,1-tris(aminomethyl)ethane, ethylenedi amine, 1,4-diazabicyclo[2.2.2]octane (DABCO), diethylenetriamine, triethylenetetramine, 1,3-diaminopropane, putrescine, cadaverine, sym-norspermidine, sym-homospermidine, norspermine, thermospermine, carboxyspermidine, norcarboxyspermidine, caldopentamine, caldohexamine, ethylenediamine, 1,2-diaminopropane, 1,3-diaminopropane, N-methylethylenediamine, 1,4-diaminobutane, 3-(methylamino)propylamine, N,N′-dimethylethylenediamine, N-methyl-1,3-diaminopropane, 1-dimethylamino-2-propylamine, 3-(dimethylamino)-1-propylamine, N,N,N′,N′-tetramethyldiaminomethane, N,N,N′-trimethylethylenediamine, N-isopropylethylenediamine, propylethylenediamine, 2-(aminotriethyl)-2-methyl-1,3-propanediamine, 1,2-diamino-5-bromo-3-chlorobenzene, 3,5-dichloro-1,2-diaminobenzene, 4-bromo-1,2-diaminobenzene, 4,5-dichloro-o-phenylenediamine, 4-chloro-1,3-diaminobenzene, 2-nitro-1,4-phenylenediamine, 3-nitro-1,2-phenylenediamine, 4-nitro-o-phenylenediamine, m-phenylenediamine, o-phenylenediamine, p-phenylenediamine, trans-4-cyclohexene-1,2-diamine, cis-4-cyclohexene-1,2-diamine, hexamethylenetetramine, 4-aminobenzylamine, N,N′-bis(2-aminoethyl)-1,3-propanediamine, methyl 3,4-diaminobenzoate, 1,2-diamino-3,5-dimethylbenzene, 4,5-dimethyl-1,2-phenylenediamine, 4-(2-aminoethyl)aniline, aniline, m-xylylenediamine, N-phenylethylenediamine, o-xylylenediamine, p-xylylenediamine, 1,8-diaminooctane, N,N-dimethyldipropylenetriamine, 1,2-bis(3-aminopropylamino)ethane, N-tosylethylenediamine, 2,2,4(2,4,4)-trimethyl-1,6-hexanediamine, 1,4-diaminonaphthalene, 1,5-aminonaphthalene, 1,8-diaminonaphthalene, 4-tert-butyl-2,6-diaminoanisole, 2,2′-oxydianiline, 4,4′-oxydianiline, 3,3′-diaminobenzidine, 3,4′-diaminodiphenylmethane, 4,4′-diaminodiphenylmethane, 4,4′-ethylenedianiline, 2,4,6-triethyl-1,3,5-benzenetrimethanamine, and 1,8-anthracenedimethanamine.

13. The method of claim 8, wherein the amine is selected from the group consisting monoamines, polyamines, oar polymeric mines coated on or crosslinked with a polymer particle and/or a polymeric film.

14. The method of claim 8, wherein the amine is selected from the group consisting monoamines, polyamines, or polymeric amines functionalized on or infused into a hydrogel.

15. The method of claim 8, wherein the amine is selected from the group consisting cyclic polyamine homopolymers and heteropolymers comprising at least one of polyvinylamine, polyethyleneimine, polyvicinalamine, polyamidoamine, polyallyamines, and polyetheramines.

16. The method of claim 1, wherein the one or more secondary compounds is a pyridine or pyridinium selected from the group consisting of pyridine, alkylated pyridines, 2,6-lutidine, 2,4-lutidine, pyridazine, pyrimidine, pyrazine, 4-bromopyridine, 2,2′-bipyridine, 2,6-di-tert-butylpyridine, pyridine-2,6-dicarboxylic acid, pyridinium cations, and salts of the above.

17. The method of claim 1, wherein the one or more secondary compounds is an imidazole, an alkylated imidazole, a pyrrole or an alkylated pyrrole.

18. The method of claim 1, wherein the one or more secondary compounds is selected from the group consisting of sodium metasilicate, chlorhexidine, borates, zinc pyrithione, trimethyl barium hydroxide, 9-Azajulolidine, sodium iodide, potassium iodide, ammonia; ammonium hydroxide; tetramethylammonium hydroxide; tetrabutylammonium hydroxide; tetrabutylammonium hydroxide; sodium hydroxide; soda lime: potassium hydroxide; magnesium hydroxide; magnesium carbonate; calcium carbonate; aluminum hydroxide; sodium bicarbonate, sodium carbonate, potassium carbonate, potassium bicarbonate, or other carbonates or bicarbonates, choline; histamine; lysine; tris; and linear or branched dendiimers; calcium hydroxide, Lugol's Iodine, iodine tincture, povidone-iodine, benzalkonium chloride, cetrimonium bromide, Brilliant Green, triarylmethane dyes, Malachite green, octenidine dihydrochloride, phenoxyethanol, USP Tincture of Iodine, USP Strong Iodine Tincture, 1,3-dibromo-5,5-dimethylhydantoin (DBDMH), morpholine, and 3-(N-morpholino)propane sulfonic acid (MOPS).

19. The method of claim 1, wherein the one or more secondary compounds is selected from the group consisting of alcohols, alditols, glycols, polyols, saccharides, and polysaccharides.

20. The method of claim 19, wherein the alcohols, alditols, glycols, polyols, saccharides, and polysaccharides are selected from the group consisting of cyclitol, acarviocin, aminocyclitol, bornesitol, ciceritol, conduritol, decahydroxycyclopentane, 5-deoxyinositol, dodecahydroxycyclohexane, ononitol, pinitol, pinpollitol, quebrachitol, theogallin, 3,4,5-tri-O-galloylquinic acid, inositol, inositol pentakisphosphate, cis-inositol, D-chiro-inositol, L-chiro-inositol, epi-inositol, neo-inositol, muco-inositol, neo-inositol, scyllo-inositol, sorbitol, threitol, arabitol, galactitol, iditol, volemitol, sorbitol, fucitol, xylitol, lactitol, erythritol, lactitol, maltitol, phytic acid, quinic acid, propylene glycol, 1,2-propanediol, ethylene glycol, low molecular weight polyethylene glycols (e.g., C2-C10), vegetable glycerine, dipropylene glycol, erythulose, glycerol, panthenol, arabinose, bis-HPPP, cellobiose, mannitol, mannose, glucose, allose, altrose, gulose, idose, lactose, maltose, dextrose, galactose, talose, psicose, fructose, sorbose, tagatose, β-d-ribopyranose, α-d-ribopyranose, β-d-ribofuranose, α-d-ribofuranose, sucrose, xylose, trehalose, cytosine glycol, cyclohexane-1,2-diol, aminomethanol, ethyleneglycol, 1,3-propanediol, 1,4-butanediol, 2,2-dimethyl-1-butanol, ethanol, propanol, butanol, pentanol, hexanol, ethynol, acetylenediol, fenticlor, fucitol, gluconic acid, glucic acid, 2-heptanol, 3-heptanol, 2-hexanol, 3-hexanol, ribitol, ethylhexylglycerin, octoxyglycerin, glucuronic acid, glyceraldehyde, glyceric acid, glycerol 3-phosphate, glycerol monostearate, 2-octanediol, pinacol, racemic acid, tartaric acid, uronic acid, xylosan, 1,2-butanediol, 1,3-butanediol, 1,4-butanediol, 2,3-butanediol, 2-methyl-2,4-pentanediol, neopentyl glycol, maltodextrins, raffinose, stachyose, fructo-oligosaccharides, amylose, amylopectin, starch, glycogen, cellulose, hemicellulose, methyl cellulose, methyl ethyl cellulose, pectins, hydrocolloids, sucralose, isomalt, isomaltooligosaccharide, isomaltulose, maltodextrin, and polydextrose.

21. The method of claim 1, wherein the point-of-use kit comprises a carrier or vehicle selected from the group consisting of sticks, soaps, bars of soap, balms, creams, pastes, gums, lotions, gels, foams, ointments, emulsions, suspensions, aqueous solutions, eye drops, aerosols, sprays, inhalants, body washes, face washes, rinses, and oral tinctures.

22. The method of claim 1, wherein the at least one shikimate analogue and the one or more secondary compounds are maintained in separate compartments until formed into the mixture for application to the treatment surface at the point of use.

23. The method of claim 1, wherein the treatment surface is an animate surface selected from the group consisting of an epithelial surface of an animal, and a surface of a plant.

24. The method of claim 23, wherein the epithelial surface is selected from the group consisting of epithelia, skin, hair, fur, nails, internal or external body surfaces, surfaces of body cavities, and surfaces of hollow organs that are connected to underlying tissues in a human or animal body and constitute the corpus or partial corpus of said human or animal.

25. The method of claim 1, wherein the treatment surface is an inanimate surface of an object selected from synthetic materials, structures, articles of manufacture, textiles, fabrics, couches, chairs, beds, floors, rugs, seats, solid materials, porous materials, soils, minerals, stones, rocks, concrete, walls, wood object, resins, metals, polymers, plastics, and composite materials.

26. The method of claim 1, wherein malodorous compound is selected from the group consisting of (a) organosulfur and thiol-containing compounds, (b) organic, carboxylic acids and carboxylic-acid-containing compounds; (c) amines, amine-containing compounds, and nitrogen-containing compounds; and (d) inorganic sulfur-containing and nitrogen-containing compounds.
